(19) United States
(12) Patent Application Publication
(10) Pub. No.: US 2024/0344130 A1
(43) Pub. Date: Oct. 17, 2024

(54) ISOTHERMAL AMPLIFICATION-BASED DIAGNOSIS AND TREATMENT OF ACUTE INFECTION

(71) Applicant: Inflammatix, Inc., Sunnyvale, CA (US)

(72) Inventors: Melissa Remmel, Sunnyvale, CA (US); Yuan Yuan, Sunnyvale, CA (US); Sabrina Coyle, Sunnyvale, CA (US); Dave Rawling, Sunnyvale, CA (US); Timothy Sweeney, Truckee, CA (US)

(73) Assignee: Inflammatix, Inc., Sunnyvale, CA (US)

(21) Appl. No.: 18/293,648

(22) PCT Filed: Jul. 29, 2022
(86) PCT No.: PCT/US22/38834

§ 371 Date: Jan. 30, 2024

Related U.S. Application Data
(60) Provisional application No. 63/229,032, filed on Aug. 3, 2021.

(51) Int. Cl. C12Q 1/6883; C12Q 1/6851

(52) U.S. Cl. CPC C12Q 1/6883; C12Q 1/6851; C12Q 2600/158

(57) ABSTRACT

This invention provides primers and primer combinations that function efficiently in LAMP-based and/or other amplification systems and are useful for the diagnosis and subsequent treatment of acute infectious disease.

### CROSS-REFERENCES TO RELATED APPLICATIONS

This application claims priority to U.S. Provisional Application No. 63/229,032, filed 3 Aug. 2021, the disclosure of which is hereby incorporated by reference in its entirety for all purposes.

### BACKGROUND

Approximately 30% of antibiotic prescriptions are administered to treat infections for which they are either not needed or not indicated. Misuse and over-prescription of antibiotics contributes to the emergence of antimicrobial resistant pathogens, and can result in patients experiencing myriad side effects for no clinical benefit. Rapid diagnostics that can aid in decision making by classifying causal pathogens are needed to mitigate over-prescription of antimicrobials.

To address this need, previous work has used a multicohort analysis to identify and validate a “bacterial/viral metascore,” for discriminating between bacterial and viral etiologies. The metascore relies on determining the relative abundances of a set of seven host response biomarkers (human immune mRNAs quantitated from whole blood) for discriminating between bacterial and viral etiologies. These informative biomarkers comprise a set of four mRNAs: four “bacterial” genes-CTSB, GPAA1, HK3, and TNIP1—for which transcription is primarily up-regulated when experiencing a bacterial infection (CTSB, GPAA1, HK3, and TNIP1), and a set of three “viral” genes-IFI27, JUP, and LAX1—which are up-regulated in response to viral infection (IFI27, JUP, and LAX1). To calculate the bacterial/viral metascore, the normalized abundance of mRNA transcripts corresponding to each informative gene must be measured in a human whole blood sample. The diagnostic metascore can then be calculated by determining the difference in geometric means of normalized mRNA abundance measurements between bacterial and viral gene sets relative to reference or “housekeeping” genes.

Numerous technologies exist for gene expression profiling, including qRT-PCR, fluorescence barcode-based digital counting (NanoString), microarrays, RNA-Seq, and others. However, the majority of antibiotic prescriptions occur in outpatient settings, and the average duration of an outpatient consultation is less than 30 minutes; therefore, a successful point of care diagnostic should provide a result within this time frame to integrate seamlessly with physicians' workflows and should be kept as inexpensive as possible. Unfortunately, most of the aforementioned technologies suffer from long turnaround times and high costs associated with either reagents, instrumentation, or both. Even qRT-PCR, which is a relatively rapid technology and can quantitatively measure small sets of biomarkers in parallel typically has turnaround times of >45 minutes, and is therefore still not suitable for integration at the point of care.

Accordingly, there is a need for rapid, simple, and inexpensive methods for molecular diagnostics that profile the host gene response, while maintaining high levels of specificity and sensitivity. Given the importance of extremely rapid diagnoses for patients in order to enable decisions regarding an appropriate course of action, early, accurate and rapid diagnosis is critical to guide the choice of antimicrobial treatment, improve patient outcome, and ensure antimicrobial stewardship. The present invention addresses these and other needs.

### BRIEF SUMMARY

The present disclosure is based upon the surprising discovery that certain combinations of primers can be effectively used in quantitative isothermal amplification assays, e.g., in reverse-transcription loop-mediated isothermal amplification (RT-LAMP), for the rapid, accurate, and efficient amplification of polynucleotides such as reverse-transcribed mRNA from biomarkers in a clinical setting.

In one aspect, the present disclosure provides a method of treating an acute illness in a subject, comprising the steps of: a. selecting a patient presenting clinical symptoms of an acute illness and having a biomarker gene score exceeding a threshold value indicating the presence of a bacterial or a viral infection in the patient, wherein the biomarker gene score is based on measured expression levels in blood from the patient of at least two biomarker genes selected from the group consisting of IFI27, JUP, LAX1, CTSB, GPAA1, HK3, and TNIP1; (i) wherein the expression levels of the two or more biomarker genes are quantitatively determined by amplification and detection of subsequences of mRNAs encoding the two or more biomarker genes, (ii) wherein the amplification of the subsequence is performed by Reverse-Transcription Loop-Mediated Amplification (RT-LAMP) using a biomarker RT-LAMP primer combination comprising a plurality of biomarker core (FIP, BIP, F3, and B3) primers; (iii) wherein the plurality of biomarker core primers is selected from the group consisting of: For IFI27: tgctcccagtgactgcagagtaattgccaatgggggtgga (IFI27_64_FIP), tgcgaggttctactagctccctttctcccctggcatggtt (IFI27_65_BIP), agcagccaagatgatgtcc (IFI27_64_F3), and gatagttggctcctcgctg (IFI27_65_B3); For JUP: accccaagttcctggccatc (PD JUPv9 F3), tcccaccagcctccacaatg (PD JUPv9 B3), gatctgcacgagggccttgcagctcctggcctac (PD JUPv9 FIP), atgcgtaactacagttatgaaaagctgcgcttattgctgggacacacggatag PD JUPv9_BIP; For LAX1: gaaataaagaccagatcaccaacatctt (PD LAX1v9 F3), gaggaggctctcagtactgaaaat (PD LAX1V9 B3), gcatgacggtaactcggagcgttgcggttttctgcatc (PD LAX1V9 FIP), and tgactttgccacaaaccagacactcatgtctccccaggtctt (PD LAX1V9_BIP); For CTSB: cggccatgatgtccttctcgcaacaggacaagcactacgga (CTSB_27_FIP), tctgtgagcctggctacag (CTSB_27_F3), acaaaaacggccccgtggagacgtgttggtacactcctga (CTSB_715_BIP), and catggccacccatcatctc (CTSB_715_B3); For GPAA1: gtggaggagcagtttgcg (GPAA1_23 F3), ttggtgcccgacaccata (GPAA1_23 B3), gttcaagccaggccactggccttttgcccgggacttcg (GPAA1_23 FIP), gatgcggtcagtagggctggacgctcgtgggtctcatct (GPAA1_23_BIP); For HK3: acctgaggagagtgactagcttct (PD HK3v4 F3), gcctgctccatggaacccaaga (PD HK3v4 B3), tcagagcaactcagggtttcttccccactgtggaagctcatggac (PD HK3v4 FIP), and tcagagctggtgcaggagtgcgctggcttggatctgctgtagc (PD HK3v4_BIP); and For TNIP1: ggatcagctgagcccact (PD TNIP1V21-1 F3), cagcaactcattctgcgtga (PD TNIP1V21-1 B3), gtgcttcctccagggccttgacccgacagcgtgagtac (PD TNIP1V21-1 FIP), and ccaaaccccgccatcatctccccagctcctgtttccttagg (PD TNIP1V21-1_BIP); including variants of the sets wherein one or more of the biomarker core primers contains 1, 2, or 3 nucleotide substitutions relative to any one of the above sequences; and b. treating the selected patient with an antimicrobial agent in an amount sufficient to reduce the clinical symptoms of the acute illness.

In some embodiments, the biomarker RT-LAMP primer combination further comprises a pair of biomarker loop (LF and LB) primers. In some embodiments, the pair of biomarker loop primers is selected from the group consisting of: For IFI27: tgggtctgccattgcgg (IFI27_64_LF-4), ccctcgccctgcagagaaga (IFI27_65_LB-1); For JUP: gatcagcttgctctcctggtt (PD JUPv9 FL), accaccagtcgtgtgctcaag (PD JUPv9 BL); For LAX1: gtcgcttcttccgtttattccaat (PD LAX19 FL), agccaaaaatatttatgacatcttgcct (PD LAX1V9 BL); For CTSB: atgttaaggatgtcgcagaggt (CTSB_LF_27-1), ggagctttctctgtgtattcgg CTSB_LB_715-1; For GPAA1: ccccgacttcttgcggt (GPAA1_23-1 FL), gcagagtttctcccggaaac (GPAA1_23-1 BL); For HK3: ccgcaaccctgaagaccca (PD HK3v4 FL), gcagttcaaggtgacaagggcac (PD HK3v4 BL); and For TNIP1: ccgctggatctccttttcctg (PD TNIP1v21-1 FL), caacagcatttgggagcccag (PD TNIP1v21-1 BL), including variants of the pairs wherein one or more of the biomarker loop primers contains 1, 2, or 3 nucleotide substitutions relative to one or more of the above sequences.

In some embodiments, the determination of the biomarker gene score is based on relative expression levels of the at least two biomarkers in the biological sample as compared to expression levels of one or more reference genes. In some embodiments, the one or more reference genes comprise KPNA6, RREB1, or YWHAB. In some embodiments, the expression levels of the one or more reference genes are determined by amplification and detection of one or more subsequences of one or more reference gene mRNAs encoding the one or more reference genes, wherein the amplification of the one or more subsequences of the one or more reference gene mRNAs is performed by RT-LAMP using a reference gene RT-LAMP primer combination comprising a plurality of reference gene core (FIP, BIP, F3, B3) primers; and wherein the plurality of reference gene core primers is selected from the group consisting of: For KPNA6: ccacttgttgagcagtcccaagga (PD KPNA6v6 B3), agtgacgatgttacccacggctctattggtagagctgctgatgcacaa (PD KPNA6v6 FIP), tcttaactgttcagccctaccttgagtccagcaagcttccttccggat (PD KPNA6v6_BIP), For RREB1: gccattttgattccttttccggaacaagt (PD RREB1v7 F3), gccaggttcagccccccaata (PD RREB1v7 B3), acacagtcggagcaacggccctcctcggtctctccctgaagc (PD RREB1V7 FIP), gttccaggagtggtggctctgagactgttttctttgtgttatcaagctgcccand (PD RREB1v7_BIP), and For YWHAB: tgcatgatcagagtgctgtctttataaaacggcatttgatgaagc (PE YWHABv145 FIP), ctgaaaaggcctgtagcc (PE YWHABv145 F3), ctgtggacatcggaaaaccagtcacaaagcacgagaaaca (PE YWHABV145_BIP), cagagtgacactgaacaga (PE YWHABv145 B3), including variants of the pluralities wherein one or more of the reference gene core primers contains 1, 2, or 3 nucleotide substitutions relative to one or more of the above sequences.

In some embodiments, the reference gene RT-LAMP primer combination further comprises a pair of reference gene loop (LF and LB) primers. In some embodiments, the pair of reference gene loop primers is selected from the group consisting of: For KPNA6: atttgagccctgttgccagcagta (PD KPNA6v6 FL), cagggcaggagaagccactttgta (PD KPNA6v6 BL), For RREB1: cggagtagaaaatgagtctgtgttgacctctt (PD RREB1V7 FL), ctccctggcatgatgcgttgg (PD RREB1V7 BL), and For YWHAB: tcagcgtatccaattcagcaat (PE YWHABv145-1 FL), gagacgaaggagacgctggg (PE YWHABv145-1 BL), including variants of the pairs wherein one or more of the reference gene loop primers contains 1, 2, or 3 nucleotide substitutions relative to one or more of the above sequences. In some embodiments, the antimicrobial agent is an antiviral agent. In some such embodiments, the measured expression level of IFI27, JUP, and/or LAX is elevated in the biological sample relative to an expression level representative of an individual without a viral infection. In some embodiments, the antimicrobial agent is an antibacterial agent. In some such embodiments, the measured expression level of CTSB, GPAA1, HK3, and/or TNIP1 is elevated in the biological sample relative to an expression level representative of an individual without a bacterial infection. In some embodiments, the biological sample is a blood sample.

In another aspect, the present disclosure provides a genetic amplification system for diagnosing an acute infection, comprising a multiplicity of reaction vessels and a blood sample from a patient presenting clinical symptoms of an acute infection, wherein the system is configured to measure the expression levels of at least two biomarker genes by Reverse-Transcription Loop-Mediated Amplification (RT-LAMP) and detection of subsequences of mRNAs encoding the biomarker genes, wherein a score generated from the measured expression levels is indicative of a likelihood of the presence of a bacterial or a viral infection in the patient, wherein the biomarker genes are selected from the group consisting of IFI27, JUP, LAX1, CTSB, GPAA1, HK3, and TNIP1, wherein the reaction vessels comprise biomarker RT-LAMP primer combinations for amplification of the biomarker genes, and wherein the biomarker RT-LAMP primer combination used to amplify the biomarker genes comprises a plurality of biomarker core primers selected from the group consisting of: For IFI27: tgctcccagtgactgcagagtaattgccaatgggggtgga (IFI27_64_FIP), gcgaggttctactagctccctttctcccctggcatggtt (IFI27_65_BIP), agcagccaagatgatgtcc (IFI27_64_F3), and gatagttggctcctcgctg (IFI27_65_B3); For JUP: accccaagttcctggccatc (PD JUPv9 F3), tcccaccagcctccacaatg (PD JUPv9 B3), gatctgcacgagggccttgcagctcctggcctac (PD JUPv9 FIP), atgcgtaactacagttatgaaaagctgcgcttattgctgggacacacggatag PD JUPv9_BIP; For LAX1: gaaataaagaccagatcaccaacatctt (PD LAX1V9 F3), gaggaggctctcagtactgaaaat (PD LAX1V9 B3), gcatgacggtaactcggagcgttgcggttttctgcatc (PD LAX1V9 FIP), and tgactttgccacaaaccagacactcatgtctccccaggtctt (PD LAX1V9_BIP); For CTSB: cggccatgatgtccttctcgcaacaggacaagcactacgga (CTSB_27_FIP), tctgtgagcctggctacag (CTSB_27_F3), acaaaaacggccccgtggagacgtgttggtacactcctga (CTSB_715_BIP), and catggccacccatcatctc (CTSB_715_B3); For GPAA1: gtggaggagcagtttgcg (GPAA1_23 F3), ttggtgcccgacaccata (GPAA1_23 B3), gttcaagccaggccactggccttttgccegggacttcg (GPAA1_23 FIP), gatgcggtcagtagggctggacgctcgtgggtctcatct (GPAA1_23_BIP); For HK3: acctgaggagagtgactagcttct (PD HK3v4 F3), gcctgctccatggaacccaaga (PD HK3V4 B3), tcagagcaactcagggtttcttccccactgtggaagctcatggac (PD HK3v4 FIP), and tcagagctggtgcaggagtgcgctggcttggatctgctgtagc (PD HK3v4_BIP); and For TNIP1: ggatcagctgagcccact (PD TNIP1V21-1 F3), cagcaactcattctgcgtga (PD TNIP1V21-1 B3), gtgcttcctccagggccttgacccgacagcgtgagtac (PD TNIP1V21-1 FIP), and ccaaaccccgccatcatctccccagctcctgtttccttagg (PD TNIP1v21-1_BIP); including variants of the pluralities wherein one or more of the biomarker core primers within the combination contains 1, 2, or 3 nucleotide substitutions relative to any one of the above sequences. In some embodiments, the biomarker RT-LAMP primer combination further comprises a pair of biomarker loop primers selected from the group consisting of: For IFI27: tgggtctgccattgcgg (IFI27_64_LF-4), ccctcgccctgcagagaaga (IFI27_65_LB-1); For JUP: gatcagcttgctctcctggtt (PD JUPv9 FL), accaccagtcgtgtgctcaag (PD JUPv9 BL); For LAX1: gtcgcttcttccgtttattccaat (PD LAX1V9 FL), agccaaaaatatttatgacatcttgcct (PD LAX1V9 BL); For CTSB: atgttaaggatgtcgcagaggt (CTSB_LF_27-1), ggagctttctctgtgtattcgg CTSB_LB_715-1; For GPAA1: ccccgacttcttgcggt (GPAA1_23-1 FL), gcagagtttctcccggaaac (GPAA1_23-1 BL); For HK3: ccgcaaccctgaagaccca (PD HK3v4 FL), gcagttcaaggtgacaagggcac (PD HK3v4 BL); and For TNIP1: ccgctggatctccttttcctg (PD TNIP1v21-1 FL), caacagcatttgggagcccag (PD TNIP1V21-1 BL), including variants of the pairs wherein one or more of the biomarker loop primers contains 1, 2, or 3 nucleotide substitutions relative to one or more of the above sequences.

In some embodiments, the reaction vessels further comprise a reference gene RT-LAMP primer combination for amplification of one or more reference genes, and the reference gene RT-LAMP primer combination comprises a plurality of reference gene core primers selected from the group consisting of: For KPNA6: ccacttgttgagcagtcccaagga (PD KPNA6v6 B3), agtgacgatgttacccacggctctattggtagagctgctgatgcacaa (PD KPNA6v6 FIP), tcttaactgttcagccctaccttgagtccagcaagcttccttccggat (PD KPNA6v6_BIP), For RREB1: gccattttgattccttttccggaacaagt (PD RREB1V7 F3), gccaggttcagccccccaata (PD RREB1v7 B3), acacagtcggagcaacggccctcctcggtctctccctgaagc (PD RREB1V7 FIP), gttccaggagtggtggctctgagactgttttctttgtgttatcaagctgcccand (PD RREB1V7_BIP), and For YWHAB: tgcatgatcagagtgctgtctttataaaacggcatttgatgaagc (PE YWHABv145 FIP), ctgaaaaggcctgtagcc (PE YWHABv145 F3), ctgtggacatcggaaaaccagtcacaaagcacgagaaaca (PE YWHABv145_BIP), cagagtgacactgaacaga (PE YWHABv145 B3), including variants of the pluralities wherein one or more of the reference gene core primers contains 1, 2, or 3 nucleotide substitutions relative to one or more of the above sequences. In some embodiments, the reference gene RT-LAMP primer combination further comprises a pair of reference gene loop primers selected from the group consisting of: For KPNA6:

atttgagccctgttgccagcagta (PD KPNA6v6 FL), cagggcaggagaagccactttgta (PD KPNA6v6 BL), For RREB1: cggagtagaaaatgagtctgtgttgacctctt (PD RREB1V7 FL), ctccctggcatgatgcgttgg (PD RREB1v7 BL), and For YWHAB: tcagcgtatccaattcagcaat (PE YWHABv145-1 FL), gagacgaaggagacgctggg (PE YWHABv145-1 BL), including variants of the pairs wherein one or more of the reference gene loop primers contains 1, 2, or 3 nucleotide substitutions relative to one or more of the above sequences. In some embodiments, two or more of the biomarker and/or reference genes are amplified in the same reaction vessel.

In another aspect, the present disclosure provides a method of diagnosing a bacterial or viral infection in a patient with symptoms of an acute infection, comprising: a. selecting a blood sample from a patient presenting clinical symptoms of an acute infection, and quantitatively determining a diagnostic score indicative of a bacterial or viral infection based on measured levels in the patient sample of at least two biomarker genes selected from the group consisting of IFI27, JUP, LAX1, CTSB, GPAA1, HK3, and TNIP1; (i) where the levels of the biomarker genes are measured by the amplification and detection of subsequences of mRNAs encoding the biomarker genes and wherein the diagnostic score exceeds a threshold indicative of a bacterial or a viral infection, wherein the threshold value is generated by a quantitative comparison of biomarker gene expression level scores of at least 100 patients known to have a diagnosis of a bacterial or a viral infection, and 100 healthy controls; (ii) wherein the amplification is performed by Reverse-Transcription Loop-Mediated Amplification (RT-LAMP) using a biomarker RT-LAMP primer combination comprising a plurality of biomarker core (FIP, BIP, F3, and B3) primers, and (iii) wherein the plurality of biomarker core primers is selected from the group consisting of: For IFI27: tgctcccagtgactgcagagtaattgccaatgggggtgga (IFI27_64_FIP), tgcgaggttctactagctccctttctcccctggcatggtt (IFI27_65_BIP), agcagccaagatgatgtcc (IFI27_64_F3), and gatagttggctcctcgctg (IFI27_65_B3); For JUP: accccaagttcctggccatc (PD JUPv9 F3), tcccaccagcctccacaatg (PD JUPv9 B3), gatctgcacgagggccttgcagctcctggcctac (PD JUPv9 FIP), atgcgtaactacagttatgaaaagctgcgcttattgctgggacacacggatag PD JUPv9_BIP; For LAX1: gaaataaagaccagatcaccaacatctt (PD LAX1V9 F3), gaggaggctctcagtactgaaaat (PD LAX1V9 B3), gcatgacggtaactcggagcgttgcggttttctgcatc (PD LAX1V9 FIP), and tgactttgccacaaaccagacactcatgtctccccaggtctt (PD LAX1V9_BIP); For CTSB: cggccatgatgtccttctcgcaacaggacaagcactacgga (CTSB_27_FIP), tctgtgagcctggctacag (CTSB_27_F3), acaaaaacggccccgtggagacgtgttggtacactcctga (CTSB_715_BIP), and catggccacccatcatctc (CTSB_715_B3); For GPAA1: gtggaggagcagtttgcg (GPAA1_23 F3), ttggtgcccgacaccata (GPAA1_23 B3), gttcaagccaggccactggccttttgcccgggacttcg (GPAA1_23 FIP), gatgcggtcagtagggctggacgctcgtgggtctcatct (GPAA1_23_BIP); For HK3: acctgaggagagtgactagcttct (PD HK3v4 F3), gcctgctccatggaacccaaga (PD HK3v4 B3), tcagagcaactcagggtttcttccccactgtggaagctcatggac (PD HK3V4 FIP), and tcagagctggtgcaggagtgcgctggcttggatctgctgtagc (PD HK3v4_BIP); and For TNIP1: ggatcagctgagcccact (PD TNIP1V21-1 F3), cagcaactcattctgcgtga (PD TNIP1V21-1 B3), gtgcttcctccagggccttgacccgacagcgtgagtac (PD TNIP1V21-1 FIP), and ccaaaccccgccatcatctccccagctcctgtttccttagg (PD TNIP1V21-1_BIP); including variants of the pluralities wherein one or more of the biomarker core primers contains 1, 2, or 3 nucleotide substitutions relative to any one of the above sequences.

In some embodiments, the biomarker RT-LAMP primer combination further comprises a pair of biomarker loop primers. In some embodiments, the pair of biomarker loop primers is selected from the group consisting of: For IFI27: tgggtctgccattgcgg (IFI27_64_LF-4), ccctcgccctgcagagaaga (IFI27_65_LB-1); For JUP: gatcagcttgctctcctggtt (PD JUPv9 FL), accaccagtcgtgtgctcaag (PD JUPv9 BL); For LAX1: gtcgcttcttccgtttattccaat (PD LAX1v9 FL), agccaaaaatatttatgacatcttgcct (PD LAX1V9 BL); For CTSB: atgttaaggatgtcgcagaggt (CTSB_LF_27-1), ggagctttctctgtgtattcgg CTSB_LB_715-1; For GPAA1: ccccgacttcttgcggt (GPAA1_23-1 FL), gcagagtttctcccggaaac (GPAA1_23-1 BL); For HK3: ccgcaaccctgaagaccca (PD HK3v4 FL), gcagttcaaggtgacaagggcac (PD HK3V4 BL); and For TNIP1: ccgctggatctccttttcctg (PD TNIP1v21-1 FL), caacagcatttgggagcccag (PD TNIP1V21-1 BL), including variants of the pairs wherein one or more of the biomarker loop primers contains 1, 2, or 3 nucleotide substitutions relative to one or more of the above sequences.

In some embodiments, the determination of the biomarker gene score is based on relative expression levels of the at least two biomarkers in the biological sample as compared to expression levels of one or more reference genes. In some embodiments, the one or more reference genes comprise KPNA6, RREB1, or YWHAB. In some embodiments, the expression levels of the one or more reference genes are determined by amplification and detection of one or more subsequences of one or more mRNAs encoding the one or more reference genes, wherein the amplification of the one or more subsequences of one or more mRNAs is performed by RT-LAMP using a reference gene RT-LAMP primer combination comprising a plurality of reference gene core (FIP, BIP, F3, B3) primers; and wherein the plurality of reference gene core is selected from the group consisting of: For KPNA6: ccacttgttgagcagtcccaagga (PD KPNA6v6 B3), agtgacgatgttacccacggctctattggtagagctgctgatgcacaa (PD KPNA6v6 FIP), tcttaactgttcagccctaccttgagtccagcaagcttccttccggat (PD KPNA6v6_BIP), For RREB1: gccattttgattccttttccggaacaagt (PD RREB1v7 F3), gccaggttcagccccccaata (PD RREB1V7 B3), acacagtcggagcaacggccctccteggtctctccctgaagc (PD RREB1V7 FIP), gttccaggagtggtggctctgagactgttttctttgtgttatcaagctgcccand (PD RREB1V7_BIP), and For YWHAB: tgcatgatcagagtgctgtctttataaaacggcatttgatgaagc (PE YWHABV145 FIP), ctgaaaaggcctgtagcc (PE YWHABv145 F3), ctgtggacatcggaaaaccagtcacaaagcacgagaaaca (PE YWHABv145_BIP), cagagtgacactgaacaga (PE YWHABv145 B3), including variants of the pluralities wherein one or more of the reference gene core primers contains 1, 2, or 3 nucleotide substitutions relative to one or more of the above sequences. In some embodiments, the reference gene RT-LAMP primer combination further comprises a pair of reference gene loop primers. In some embodiments, the pair of reference gene loop primers is selected from the group consisting of: For KPNA6: atttgagccctgttgccagcagta (PD KPNA6v6 FL), cagggcaggagaagccactttgta (PD KPNA6v6 BL), For RREB1:

cggagtagaaaatgagtctgtgttgacctctt (PD RREB1V7 FL), ctccctggcatgatgcgttgg (PD RREB1V7 BL), and For YWHAB: tcagcgtatccaattcagcaat (PE YWHABV145-1 FL), gagacgaaggagacgctggg (PE YWHABv145-1 BL) including variants of the pairs wherein one or more of the reference loop primers contains 1, 2, or 3 nucleotide substitutions relative to one or more of the above sequences.

In another aspect, the present disclosure provides a method of treating an acute illness in a subject, comprising the steps of: a. selecting a patient presenting clinical symptoms of an acute illness and having a biomarker gene score exceeding a threshold value indicating the presence of a bacterial or a viral infection in the patient and/or the severity of an infection in the patient, wherein the biomarker gene score is based on measured expression levels in blood from the patient of at least two biomarker genes selected from the group consisting of ARG1, BATF, C3AR1, C9orf95/NMRK1, CD163, CEACAM1, CTSB, CTSL1, DEFA4, FURIN, GADD45A, GNA15, HK3, HLA-DMB, IFI27, ISG15, JUP, KCNJ2, KIAA1370, KPNA6, LY86, OASL, OLFM4, PDE4B, PER1, PSMB9, RAPGEF1, RREB1, S100A12, TGFBI, YWHAB, and ZDHHC19; (i) wherein the expression levels of the two or more biomarker genes are quantitatively determined by amplification and detection of subsequences of mRNAs encoding the two or more biomarker genes, (ii) wherein amplification of the subsequences is performed by Reverse-Transcriptase Loop-Mediated Amplification (RT-LAMP) using a biomarker RT-LAMP primer combination comprising a plurality of biomarker core (FIP, BIP, F3, and B3) primers and a pair of biomarker loop (LF and LB) primers; (iii) wherein the biomarker RT-LAMP primer combination is a set of RT-LAMP primers listed in Table 10, including variants of the sets wherein one or more of the biomarker core or loop primers within the set contains 1, 2, or 3 nucleotide substitutions relative to any one of the sequences included in Table 10; and b. treating the selected patient with an antimicrobial agent in an amount sufficient to reduce the clinical symptoms of the acute illness.

In another aspect, the present disclosure provides a genetic amplification system for diagnosing an acute infection, comprising a multiplicity of reaction vessels and a blood sample from a patient presenting clinical symptoms of an acute infection, wherein the system is configured to measure the expression levels of at least two biomarker genes by Reverse-Transcription Loop-Mediated Amplification (RT-LAMP) and detection of subsequences of mRNAs encoding the biomarker genes, wherein a score generated from the measured expression levels is indicative of a likelihood of the presence of a bacterial or a viral infection in the patient, wherein the biomarker genes are selected from the group consisting of ARG1, BATF, C3AR1, C9orf95/NMRK1, CD163, CEACAM1, CTSB, CTSL1, DEFA4, FURIN, GADD45A, GNA15, HK3, HLA-DMB, IFI27, ISG15, JUP, KCNJ2, KIAA1370, KPNA6, LY86, OASL, OLFM4, PDE4B, PER1, PSMB9, RAPGEF1, RREB1, S100A12, TGFBI, YWHAB, and ZDHHC19, wherein the reaction vessels comprise biomarker RT-LAMP primer combinations for amplification of the biomarker genes, and (iii) wherein the biomarker RT-LAMP primer combination is a set of RT-LAMP primers listed in Table 10, including variants of the sets wherein one or more of the biomarker core or loop primers within the set contains 1, 2, or 3 nucleotide substitutions relative to any one of the sequences included in Table 10.

Such primers and primer combinations allow the rapid performance of amplification assays, with reactions and returned results regarding specific biomarkers (e.g., loci of interest described) provided within 20 minutes, within 15 minutes, within 10 minutes, within 9 minutes, within 8 minutes, within 7 minutes, within 6 minutes, within 5 minutes, or within another suitable duration of time. Such results can be provided using the materials described, with no observable loss in accuracy or precision (e.g., with respect to abilities to discriminate between expression profiles associated with bacterial and viral etiologies, with respect to providing point-of-care (POC) diagnostic results).

Assays performed according to the invention(s) described further exhibited no or negligible amplification from a nominal amount of gDNA (10 ng) within the assay duration, and exhibited no or negligible amplification in non-templated reactions within 15 minutes.

Assays performed according to the invention(s) described are also high performance in dynamic range, and exhibit a dynamic range of at least 2-fold, 3-fold, 4-fold, 5-fold, or another suitable dynamic range in discerning changes in target abundance, for each biomarker involved, and with suitable effective resolution (e.g., resolution greater than 1-fold, resolution greater than 2-fold, resolution greater than 3-fold, etc.).

Assays performed according to the invention(s) described can be performed in a highly multiplexed or otherwise parallel manner, with the ability to detect at least 5 targets, 10 targets, 15 targets, 20 targets, 30 targets, 40 targets, 50 targets, 100 targets, or another suitable number of targets (e.g., loci of interest) for characterizations.

### INCORPORATION BY REFERENCE

All publications, patents, and patent applications mentioned in this specification are herein incorporated by reference in their entireties for all purposes and to the same extent as if each individual publication, patent, or patent application was specifically and individually indicated to be incorporated by reference. Furthermore, where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

### BRIEF DESCRIPTION OF THE DRAWINGS

FIGS. 1A-1E: Proof of concept RT-LAMP assay design. (FIG. 1A) Schematic diagram describing key steps in the loop-mediated isothermal amplification mechanism. Template and primer sequences are color-coded to represent complementarity; red, F1/B1; blue F2/B2, green, F3 B3; purple, LF/LB. Target sites for exon: exon junctions within the amplicon sequence are denoted with translucent gray bars labeled “ex: ex.” (FIGS. 1B, 1C) Amplification plots showing the change in fluorescent signal (ARFU) as a function of time for reactions containing core primer sets (F2/B2/F3/B3) targeted to (FIG. 1B) CTSB and (FIG. 1C) IFI27 mRNA tested using 10 ng of cDNA (red) or gDNA (blue) as a template. (FIGS. 1D, 1E) Amplification plots for full primer sets (core primers plus LF/LB) targeting (FIG. 1D) CTSB and (FIG. 1E) IFI27 tested using 10 ng of total RNA as a template.

FIGS. 2A-2B: Using the nCounter Sprint to define acceptance criteria. (FIG. 2A) Raw measured abundance in “counts” of informative biomarkers for the Fever diagnostic test evaluated in total RNA extracted from stabilized blood comprising a cohort of 57 clinical samples plotted against an arbitrary numbers designating unique samples within the cohort. (FIG. 2B). Diagrammatic representation of a theoretical assay standard curve to describe resolution as defined in this report. Mean Tt values are shown by red circles, with light red ovals representing the 95% confidence intervals (95% CI) of the measurements. The diagram shows three measurements made at input levels a, b, and c spaced at equal input intervals. Whereas the 95% CI at inputs b and c overlap (upper dashed arrow), those for a and c do not (lower dashed arrow). Therefore, the theoretical assay could not resolve the fold-change from inputs b to c (b/c), but could resolve the difference between a and c (a/c). Hence, the resolution of the assay, r, is greater than b/c (lower resolution), but less than a/c (higher resolution) as denoted by the inset relationship.

FIGS. 3A-3M: Assay screening and analytical performance testing. (FIGS. 3A-3J, Left Panels) Summary time to result plots for full primer sets selected as best performers. Results shown here represent time to positive for optimized RT-LAMP reactions performed in triplicate across 1 to 3 experiments to test different commercial genomic DNA stocks. Either 10 ng of total RNA (red) or 10 ng gDNA (blue) was used as template, or the reaction contained no template (green). Bars indicate mean across all replicates and experiments; error bars represent standard deviation. The biomarker targeted by each assay is denoted above the plot. (FIGS. 3A-3J, Right Panels) Plots of mean Tt vs Log 10 of input template copy number across three technical replicates, with error bars showing standard deviation. Templates comprised in vitro transcribed RNA specific for the assay denoted above the plot. Black lines describe models generated for each relationship using a robust linear regression that identifies outliers and reduces their assigned weights during fitting. (FIG. 3K) Plot of y-intercept values determined for each biomarker in linear fits. (FIG. 3L) Plot of slope values determined for each biomarker in linear fits. (FIG. 3M) Plot of the mean of standard deviations observed across three replicate measurements for all assays at each template input level vs Log 10 input level. In vitro transcribed RNA specific for each assay was used as template. Black line describes a model generated for the relationship using a non-linear regression fit to a standard exponential decay function.

FIGS. 4A-4H: Accuracy of individual qRT-LAMP assays in clinical samples. (FIGS. 4A-4G, Left Panels) Correlation plots showing the mean of abundance measurements across three replicates made by qRT-LAMP normalized to the geometric mean of housekeeping gene measurements vs normalized abundance measurements made by nCounter SPRINT Profiler. The Pearson correlation coefficient determined for each relationship is inset. Solid black lines describe models generated for each relationship using a simple linear regression, with dashed black lines representing the 95% confidence intervals of each model. (FIGS. 4A-4G, Right Panels) Residual plots for each correlation plot showing the distance of each normalized Tt measurement from the value predicted by the linear model vs the predicted value. (FIG. 4H) Plot showing the fraction of raw Tt measurements made for each assay where the measured Tt is greater than the mean Tt determined at the estimated limit of quantitation (red) or less than the Tt determined at LoQ (blue).

FIGS. 5A-5B: Diagnostic performance of qRT-LAMP assays in clinical samples. (FIG. 5A) Plot of diagnostic scores calculated based on qRT-LAMP measurements made in clinical samples from patients suffering bacterial (B) and viral (V) infections per physicians' adjudication (see Supplementary Information). Boxes enclose the 25th to 75th percentiles and whiskers show the maximum and minimum measured values. (FIG. 5B) Correlation plot showing diagnostic scores calculated using qRT-LAMP measurements vs scores calculated using the nCounter SPRINT. The Pearson correlation coefficient determined for the relationship is inset. The solid black line describes a model generated to describe the relationship using a simple linear regression, with dashed black lines representing the 95% confidence interval of the model.

FIGS. 6A-6T: Optimization of the qRT-LAMP formulation. (FIGS. 6A-6H) Summary results showing threshold times (Tt, min) for CTSB (orange) and IFI27 (purple) assays performed using the indicated polymerase enzyme with accompanying assay buffer or recommended formulation tested using 10 ng cDNA, 10 ng gDNA or in non-templated reactions. (FIGS. 61-6Q) Summary results showing threshold times (Tt, min) for CTSB (orange) and IFI27 (purple) assays performed using the indicated reverse transcriptase enzyme paired with GspSSD2.0 polymerase and reaction buffer tested using 10 ng total RNA, 10 ng gDNA or in non-templated reactions. (FIG. 6R) Plot of Tt vs PH of the Tris buffer component for of the assay master mix for IFI27 assays performed using 10 ng total RNA as template. (FIG. 6S) Plot of Tt vs the concentration of ammonium sulfate (AmSO4) measured in reactions containing varying concentrations of potassium chloride (KCl). (FIG. 6T) Plot of Tt vs the total units of GspSSD2.0 present per reaction measured in reactions containing varying total units of WarmStart RTx reverse transcriptase.

FIGS. 7A-7B: Measurement of background RNA contamination in four commercial genomic DNA samples shown as total concentration (ng/μL) (FIG. 7A) and RNA as a percent of total dsDNA (FIG. 7B).

FIGS. 8A-8G: Amplification plots from full primer sets of qRT-LAMP reactions for informative biomarkers tested using optimized reaction chemistry. Each plot shows amplification from n=3 replicates with 10 ng total RNA (red) or 200 ng genomic DNA (blue) used as template, or in non-templated reactions (green).

FIG. 9. Decision tree for evaluating candidate RT-LAMP primers.

FIG. 10 illustrates a measurement system 1000 according to an embodiment of the present disclosure.

FIG. 11 shows a block diagram of an example computer system usable with systems and methods according to embodiments of the present disclosure.

### DETAILED DESCRIPTION

### A. Introduction

The present disclosure provides for primers and primer combinations that are useful for the diagnosis and subsequent treatment of acute infections. The described primers and primer combinations are derived from subsequences of mRNA encoding biomarker genes of patient origin. The primers and primer combinations are selected for their ability to efficiently, accurately, and rapidly amplify target sequences using Loop-Mediated Amplification (LAMP), e.g., Reverse-Transcription Loop-Mediated Amplification (RT-LAMP) including quantitative Reverse-Transcription LAMP (qRT-LAMP). Methods of using the primers and primer combinations are also provided, e.g. for selecting and treating patients presenting symptoms of an acute infection, as are genetic amplification systems that can be used for diagnosing and treating patients.

Variations of primers and primer combinations described can also be used for rapid, efficient, and accurate amplification of targets using other amplification processes (e.g., associated with polymerase chain reaction (PCR), such as digital PCR, quantitative PCR, emulsion PCR, etc.).

Approaches to diagnosing different forms of acute infection, i.e., of bacterial or viral origin, of differing severity, of differing likelihoods to lead to sepsis, can rely on methods of detecting mRNA levels of specific biomarker genes to evaluate host response. Such approaches can provide rapid and accurate indications of the etiology of the infection, outperforming other techniques such as the direct detection of pathogens.

The present disclosure is based on the surprising discovery that certain RT-LAMP primers and RT-LAMP primer combinations (e.g., forward and backward inner primers (FIP, BIP), forward and backward outer primers (F3, B3), forward and backward loop primers (LF, LB) are particularly effective at amplifying subsequences of reverse transcribed mRNA from biomarkers in biological samples from patients.

Such primers and primer combinations allow the rapid performance of amplification assays, with reactions and returned results provided within 20 minutes, within 15 minutes, within 10 minutes, within 9 minutes, within 8 minutes, within 7 minutes, or within another suitable duration of time.

Such primers and primer combinations also allow the specific (e.g., no significant non-specific amplification such as gDNA or NTC amplification, and no significant off-target amplification) performance of amplification assays.

Such primers and primer combinations also allow the efficient (e.g., no significant primer: primer interactions) performance of RT-LAMP amplification assays, e.g., in a clinical setting and/or in a research setting. The measured biomarker expression levels can then be compared to the levels of baseline housekeeping genes, e.g., by measuring the expression levels of the housekeeping genes in the biological sample and used to form biomarker scores that permit a determination of, e.g., whether an acute illness is due to an infection, whether an infection is bacterial or viral, the severity of an infection, the likelihood of the infection leading to sepsis, etc. allowing appropriate treatment regimens to be instituted rapidly.

### B. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

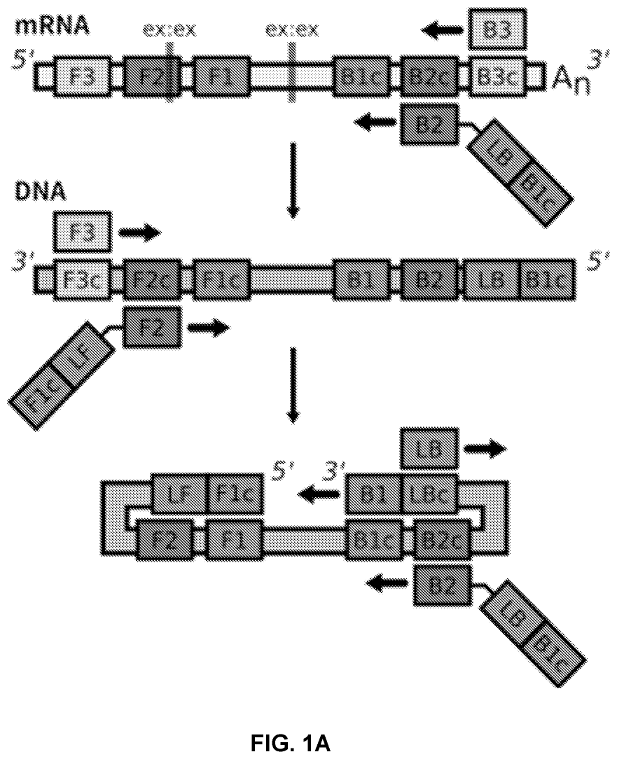

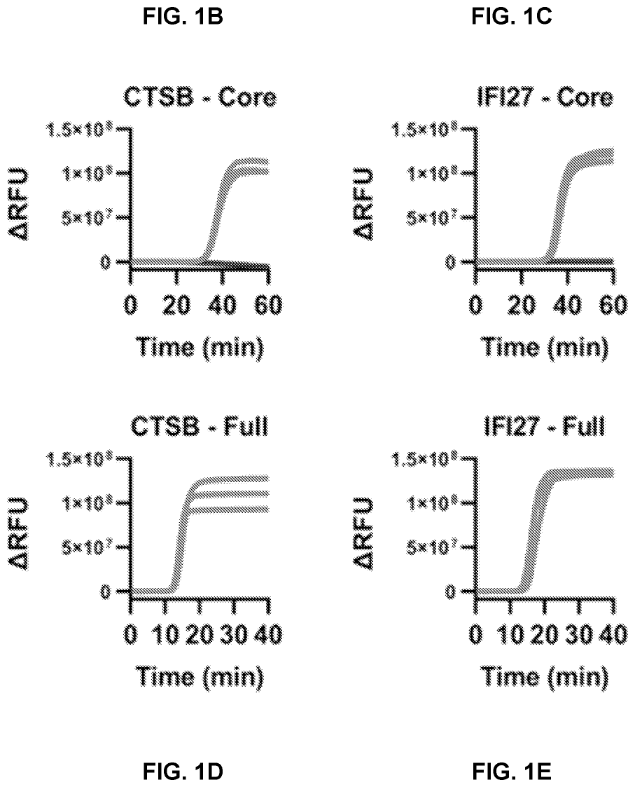

As used herein, “LAMP primers” or LAMP primer “combinations” or “sets” refers to polynucleotides that can be used together in Loop-mediated (isothermal) amplification (LAMP) assays, and particularly Reverse-Transcription Loop-Mediated Amplification (RT-LAMP) to amplify and quantify subsequences of host biomarkers, e.g., quantify biomarker mRNA levels in biological samples. In particular, the term refers to the sets of “core primers” and “loop primers” that are used to perform RT-LAMP. The “core primers” include forward and backward inner and outer primers, i.e., FIP, BIP, F3, and B3 primers (see, e.g., FIGS. 1A-1E). The “loop primers” include forward and backward primers, e.g., as shown as LB and FB in FIG. 1A. Suitable sets of LAMP (e.g., RT-LAMP) primers for use in the present methods include, but are not limited to, the primer sets shown in, e.g., Tables 3, 7, 9, and 10. The polynucleotides can have the exact sequences as shown in Tables 3, 7, 9, and 10, but they can also include variants and derivatives of the sequences shown in Tables 3, 7, 9, and 10, including substitutions, deletions, and insertions, e.g., sequences with 95%, 96%, 97%, 98%, 99%, or more sequence identity, or with 1, 2, 3 or more nucleotide substitutions, with any of the sequences shown in Tables 3, 7, 9, and 10, and in any combination within a given set of primers (i.e., the set of core and loop primers for amplifying a given biomarker), e.g., one, two, or all of the sequences within a given set can have the sequences shown in Tables 3, 7, 9, and 10, or can be a variant showing 95%, 96%, 97%, 98%, 99%, or more sequence identity (e.g., with 1, 2, 3 or more nucleotide substitutions) with any of the sequences of Tables 3, 7, 9, and 10. Sequences that are complementary to the sequences shown in Tables 3, 7, 9, and 10, or to derivatives or variants thereof, can be used as well. One of skill in the art can readily assess the suitability of any variant or derivative for use in the present methods. In particular, as long as the sequence can be used to efficiently and quantitatively amplify mRNA corresponding to a subsequence of one of the herein-described biomarker genes in an RT-LAMP assay as described herein, it can be used.

An “antimicrobial” refers to any compound or therapy that can be used to treat microbial infections, including “antibiotic” or “antibacterial” agents to treat bacterial infections, and “antiviral” agents to treat viral infections. For example, the present methods and compositions can be used to determine the presence of an infection in patients, and, further, to diagnose a viral or bacterial infection. Once such a diagnosis has been made, and in view of other clinical data, an antimicrobial agent, e.g., antibiotic or antiviral agent, can be administered to treat the bacterial or viral infection.

As used herein, the term “likelihood” is used as a measure of whether subjects with a particular biomarker score actually have a condition (or not) based on a given mathematical model. An increased likelihood for example can be relative or absolute and can be expressed qualitatively or quantitatively. For instance, an increased risk can be expressed as simply determining the subject's biomarker score and placing the test subject in an “increased risk” category, based upon previous population studies. Alternatively, a numerical expression of the test subject's increased risk can be determined based upon a biomarker score analysis.

As used herein, the term “probability” refers strictly to the probability of class membership for a sample as determined by a given mathematical model and is construed to be equivalent to likelihood in this context.

As used herein, the term “likelihood ratio” is the probability that a given test result would be observed in a subject with a condition of interest divided by the probability that that same result would be observed in a patient without the condition of interest. See below for more details.

The term “nucleic acid” or “polynucleotide” refers to primers, probes, oligonucleotides, template RNA or cDNA, genomic DNA, amplified subsequences of biomarker genes, or any polynucleotide composed of deoxyribonucleic acids (DNA), ribonucleic acids (RNA), or any other type of polynucleotide which is an N-glycoside of a purine or pyrimidine base, or modified purine or pyrimidine bases in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). “Nucleic acid”, “DNA” “polynucleotides, and similar terms also include nucleic acid analogs. The polynucleotides are not necessarily physically derived from any existing or natural sequence, but can be generated in any manner, including chemical synthesis, DNA replication, reverse transcription or a combination thereof.

“Primer” as used herein refers to an oligonucleotide, whether occurring naturally or produced synthetically, that is capable of acting as a point of initiation of synthesis when placed under conditions in which synthesis of a primer extension product which is complementary to a nucleic acid strand is induced i.e., in the presence of nucleotides and an agent for polymerization such as DNA polymerase and at a suitable temperature and buffer. Such conditions include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.), and at a suitable temperature. The primer is preferably single-stranded for maximum efficiency in amplification such as isothermal amplification, e.g., the real-time quantitative RT-LAMP of the invention. The primers herein are selected to be substantially complementary to the different strands of each specific sequence to be amplified, and a given set of primers, e.g., comprising the core and loop primers for a given biomarker, will act together to amplify a subsequence of the corresponding biomarker gene.

The term “gene” refers to the segment of DNA involved in producing a polypeptide chain. It can include regions preceding and following the coding region (leader and trailer) as well as intervening sequences (introns) between individual coding segments (exons).

As used herein, a “biomarker gene” or “biomarker” refers to a gene whose expression is correlated with the presence of absence of an infection, e.g., viral or bacterial infection, or of one or more symptoms of an infection, or with an infection with a particular degree of severity, or with a likelihood of an individual with an infection developing sepsis, etc. It will be appreciated that the biomarker gene expression need not be correlated with any of these features in all patients; rather, a correlation will exist at the population level, such that the level of expression, as measured, e.g., as a Ct or a delta Ct vis-a-vis the expression level of a housekeeping gene, is sufficiently correlated within the overall population of individuals with an infection (or other trait), that it can be combined with the expression levels of other biomarker genes and used to calculate a biomarker gene score. Preferred biomarker genes for the purposes of the present invention include IFI27, JUP, LAX1, CTSB, GPAA1, HK3, and TNIP1, as well as the biomarkers shown in Tables 10 and 11.

A “biomarker gene score” or “biomarker score” or “diagnostic score” refers to the value that is calculated from the measured expression levels of a plurality of biomarker genes, e.g., 2, 3, 4, 5, 6, 7, 8, 9 10 or more individual biomarker genes. The biomarker score can be calculated from, e.g., the Ct values or delta Ct values of the individual biomarker genes, for example by taking the geometric mean of the delta Ct values for all of the included biomarker genes, but it can be calculated in a number of other ways known to those of skill in the art. The “biomarker gene score” can be used to determine the likelihood, e.g., the likelihood ratio, of a given patient having a viral infection, a bacterial infection, being free of infection, having an infection with low, intermediate, or high severity, etc. by virtue of the score surpassing or not a given threshold value for the value in question, as described in more detail elsewhere herein.

“Conservatively modified variants” refers to nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants can have an increased stability, assembly, or activity.

As used in herein, the terms “identical” or percent “identity,” in the context of describing two or more polynucleotide sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. The identity can exists over a region that is at least about 10, 15, 20, 25, 30, 35, 40, 45, 50, or more nucleotides in length. In some embodiments, percent identity is determined over the full-length of the nucleic acid sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window”, as used herein, includes reference to a segment of any one of a number of contiguous positions, e.g. a segment of 10, 11, 12, 13, 14, 15, 16, 17, 18, 19, 20, 21, 22, 23, 24, 25, 30, 35, 40, 45, or 50 nucleotides, in which a sequence can be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215:403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. Tis referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### 1. Subjects

The present methods can be performed with any patient who presents one or more clinical features of an acute illness, or where there is any suspicion for any reason of the presence of or potential for an acute illness by a medical professional. Such symptoms include, inter alia: fever, chills, sweating, coughing, abdominal pain, malaise, sore throat, shortness of breath, nasal congestion, muscle aches, stiff neck, burning or pain with urination, redness, soreness, or swelling, diarrhea, vomiting, pain, tachycardia, tachypnea, abnormal white blood cell count, and others.

To select a patient for the present methods, when the patient shows one or more, preferably two or more, symptoms of an acute infection, an assessment is made as to whether the patient has a biomarker score that exceeds a threshold indicating their infection status (i.e., presence or absence of infection, viral or bacterial infection, severity of infection, etc.).

In particular embodiments, the subject is present in a medical context, e.g., emergency care context (emergency room, urgent care facility), hospital, or any other clinical setting where diagnosis may take place. A clinical setting does not necessarily indicate that the patient is physically present in a hospital or clinical facility, however. For example, the patient may be at home but has provided a respiratory sample using an at-home testing kit, or at a local or drive-up testing facility. The results of the methods described herein can allow a determination of the optimal next step or plan of action for the subject's care. In some embodiments, a determination that the subject has a bacterial or viral infection can indicate specific treatment such as antibiotic or anti-viral medications, additional testing to identify the specific bacteria or virus causing the infection, and/or admittance to an ICU or other clinical facility, and/or administration of any of the treatments or procedures described herein. In some cases, a negative result for a bacterial or viral infection may indicate that the subject can be discharged from the hospital or emergency room, e.g., to return home for monitoring or to go to another, non-emergency ward.

In some embodiments, the subject is asymptomatic at the time of testing but is known to be at risk of or is suspected of having a viral infection, e.g., following close contact with an individual known to be infected. In such cases, the present methods can also be used to detect a viral infection in the subject, even though the subject is potentially presymptomatic. A negative result for a viral infection in such subjects may indicate that no infection has taken place, e.g. during the close contact, and that that the subject is therefore free of infection. A positive result would indicate a need for quarantine and/or follow-up testing.

### 2. Sample Preparation

To assess the biomarker status of the patient, a biological sample is obtained from the patient, e.g. a blood sample is taken by a phlebotomist, in a way that allows the RNA to be collected and preserved. For example, in a preferred embodiment, a blood sample is collected directly into a tube prefilled with a solution that can immediately stabilize RNA from blood cells within the sample. One suitable tube is the PAXgene Blood RNA Tube (QIAGEN, BD cat. No. 762165), although any tube capable of preserving RNA can be used, a number of which are known to those of skill in the art. Using the PAXgene Blood RNA Tube, RNA can be preserved, e.g., for three days at room temperature, for five days at 4° C., and for up to eight years when frozen. In addition to blood, e.g., whole blood, peripheral blood, or serum, other biological samples that can be used for the purposes of the invention, including, inter alia, plasma, saliva, urine, sweat, nasal swab, rectal swab, ascitic fluid, peritoneal fluid, synovial fluid, amniotic fluid, cerebrospinal fluid, and tissue biopsy. Typically, the biological sample comprises whole blood, or blood cells such as mature, immature or developing leukocytes, including lymphocytes, polymorphonuclear leukocytes, neutrophils, monocytes, reticulocytes, basophils, coelomocytes, hemocytes, eosinophils, megakaryocytes, macrophages, dendritic cells natural killer cells, or fraction of such cells (e.g., a nucleic acid or protein fraction).

Once blood has been collected and preserved, in some embodiments RNA can be extracted to allow the preservation of the RNA for subsequent reverse transcription and LAMP amplification so as to determine the relative expression levels of the biomarker genes described herein and of any control genes to be used, e.g., housekeeping genes used for the calculation of the delta Ct values for the biomarkers and subsequent determination of the biomarker score. In other particular embodiments, the RNA is not extracted, and the expression levels of the biomarkers and/or reference genes are determined directly through cell lysis and subsequent reverse transcrioption and amplification of mRNA.

Suitable housekeeping genes are well known in the art and may include, e.g., 18S (18S rRNA, e.g., HGNC (Human Genome Nomenclature Committee) nos. 44278-44281, 37657), ACTB (Actin beta, e.g., HGNC no. 132)), KPNA6 (Karyopherin subunit alpha 6, e.g., HGNC no. 6399), or RREB1 (ras-responsive element binding protein 1, e.g., HGNC no. 10449), YWHAB, Chromosome 1 open reading frame 43 (Ciorf43), Charged multivesicular body protein 2A (CHMP2A), ER membrane protein complex subunit 7 (EMC7), Glucose-6-phosphate isomerase (GPI), Proteasome subunit, beta type, 2 (PSMB2), Proteasome subunit, beta type, 4 (PSMB4), Member RAS oncogene family (RAB7A), Receptor accessory protein 5 (REEP5), small nuclear ribonucleoprotein D3 (SNRPD3), Valosin containing protein (VCP) and vacuolar protein sorting 29 homolog (VPS29). at In some embodiments, any housekeeping gene provided www/tau/ac/il˜elieis/HKG/may be used.

### 3. Amplification and Detection of Biomarker Expression

The levels of transcripts of the biomarker genes, or their levels relative to one another, and/or their levels relative to a reference gene such as a housekeeping gene, are determined from the amount of mRNA, or polynucleotides derived therefrom, present in a biological sample. In particular embodiments, the mRNA is reverse transcribed to cDNA and amplified in a quantitative real-time RT-LAMP assay in order to determine the expression level of the biomarkers in question.

The primers of the disclosure can be obtained in any of a number of ways that are well known to those of skill in the art. For example, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

The amplification reactions as described herein are performed with particular primer combinations that enable efficient, rapid, and accurate amplification of subsequences of the biomarkers. For example, in some embodiments, the primer combinations allow quantitative amplification in less than 15 minutes. In some embodiments, the primer combinations allow amplification of the target biomarker without showing significant amplification of genomic DNA (gDNA). In some embodiments, the primer combinations allow amplification of the target biomarker without showing significant amplification in the absence of a template (NTC, or no template control). In some embodiments, the primer combinations allow amplification of the target biomarker without showing significant non-specific amplification, e.g., off-target amplification. In some embodiments, the primer combinations do not show significant primer: primer interactions. It will be appreciated that the vast majority of potential primers for amplifying, in RT-LAMP assays, subsequences within the biomarkers disclosed herein, i.e., IFI27, JUP, LAX1, CTSB, GPAA1, HK3, and TNIP1, or any of the biomarkers shown in Tables 3, 7, 9, and 10, do not permit efficient amplification in RT-LAMP assays, and that the specific primer sets disclosed herein have been specifically identified based on this ability. The primers disclosed herein for use in the present methods have been validated to give no or only insignificant background on NTC (no template control), to give no, or only insignificant levels of, background on gDNA, to give no, or only insignificant levels of, off-target amplification, to show no, or only insignificant levels of, primer: primer interactions, and to allow rapid amplification of the cDNA target.

In some embodiments, the primers for use in the present methods are as shown in Table 3, Table 7, Table 9, or Table 10. In particular embodiments, the primers for use in the present methods are as shown in Table 3 or Table 10.

It will be appreciated, however, that derivatives and variants of any of these sequences can also be used, including sequences with 95%, 96%, 97%, 98%, 99%, or higher sequence identity to any of the sequences shown in Table 3, Table 7, Table 9, or Table 10, including substitutions, e.g., conservative substitutions, deletions, and insertions, and including natural or modified nucleotides, as well as sequences that are complementary to any of the sequences shown in Table 3, Table 7, Table 9, or Table 10, and substitutions, deletions, insertions, and other derivatives and variants of sequences complementary to any of the sequences shown in Table 3, Table 7, Table 9, or Table 10.

The biomarkers used in the present methods correspond to genes whose expression levels correlate with, e.g., the presence or absence of an infection in patients showing symptoms of an acute infection, and, among those with an infection, with a viral or bacterial origin of infection. The biomarkers can also correlate with different features of an infection, e.g., the severity of the infection, the likelihood of the infection leading to sepsis, etc. It will be appreciated that the expression level of the individual biomarkers can be elevated or depressed in individuals with an infection relative to in healthy individuals; what is important is that the expression level of the biomarker is positively or inversely correlated with the presence or absence of an infection in the overall population of individuals with the infection, or with a viral or bacterial cause of infection, or with a similar degree of severity of infection, etc., and that the expression levels as measured using the herein described methods, and as expressed as, e.g., a Ct value or a Delta Ct value, can be combined with the levels of other biomarker genes to generate a biomarker score that can be used for the diagnostic or therapeutic purposes described herein.

The levels of at least two of the biomarker genes as assessed using the herein-described primer combinations are then combined to generate a biomarker score that will be used to assess the infection status of the patient, e.g., whether the acute illness symptoms are due to an infection and, if so, whether the infection is of viral or bacterial origin, and thus to guide treatment decisions for the patient. At least 2 of the biomarkers disclosed herein will be used to generate the biomarker score, but in numerous embodiments more than 2 will be used, e.g., 3, 4, 5, 6, 7, 8, 9, 10, 11, 12, 13, 14, 15, 16, 17, 18, 19, 20, or more of the biomarkers. It will be understood that any combination of 2, 3, 4, 5, 6, 7, 8, 9, 10, 11, 12, 13, 14, 15, 16, 17, 18, 19, 20 or more of the herein-described biomarkers can be used, and that the measured levels of any 2 or more of them can be combined with the measured expression levels of other biomarkers. For example, the measured levels of 2 of the biomarkers disclosed herein can be combined with the measured levels of 2, 3, 4, 5, 6, 7, 8, 9, 10, 11, 12, 13, 14, 15, 16, 17, 18, 19, 20 or more other biomarkers (i.e. biomarkers not disclosed herein) to generate a biomarker score. In particular embodiments, the measured biomarkers include CTSB (Cathepsin B, see, e.g., NCBI Gene ID 1508), GPAA1 (glycosylphosphatidylinositol anchor attachment 1, see, e.g. NCBI Gene ID 8733), HK3 (Hexokinase 3, see, e.g., NCBI Gene ID 3101), TNIP1 (TNFAIP3 interacting protein 1, see, e.g., NCBI Gene ID 10318), IFI27 (Interferon alpha inducible protein, see, e.g., NCBI Gene ID 3429), JUP (junction plakoglobin, see, e.g., NCBI 3728), and/or LAX1 (lymphocyte transmembrane adaptor 1, see, e.g, NCBI Gene ID 54900).

In the methods described herein, biomarker gene expression is determined using isothermal amplification. Isothermal amplification is a process in which a target nucleic acid is amplified using a constant, single, amplification temperature (e.g., from about 30° C. to about 95° C.). Unlike standard PCR, an isothermal amplification reaction does not include multiple cycles of denaturation, hybridization, and extension, of an annealed oligonucleotide to form a population of amplified target nucleic molecules (i.e., amplicons). There are various types of isothermal application known in the art, including but not limited to, loop-mediated isothermal amplification (LAMP), nucleic acid sequence based amplification NASBA, recombinase polymerase amplification (RPA), rolling circle amplification (RCA), nicking enzyme amplification reaction (NEAR), and helicase dependent amplification (HDA).

In particular embodiments, the isothermal amplification is real-time quantitative isothermal amplification, in which a target nucleic acid is amplified at a constant temperature and the target nucleic acid rate of amplification is monitored by fluorescence, turbidity, or similar measures (e.g., NEAR or LAMP). In some cases, RNA (e.g., mRNA) is isolated from a biological sample and is used as a template to synthesize cDNA by reverse-transcription. cDNA molecules are amplified under isothermal amplification conditions such that the production of amplified target nucleic acid can be detected and quantitated.

In particular embodiments, the isothermal amplification is Loop-Mediated Isothermal Amplification (LAMP), and particularly Reverse-Transcription Loop-Mediated Isothermal Amplification (RT-LAMP). LAMP offers selectivity and employs a polymerase and a set of specially designed primers that recognize distinct sequences in the target nucleic acid (see, e.g., Nixon et al., (2014) Bimolecular Detection and Quantitation, 2:4-10; Schuler et al., (2016) Anal Methods., 8:2750-2755; and Schoepp et al., (2017) Sci. Transl. Med., 9: eaal3693). Unlike PCR, the target nucleic acid is amplified at a constant temperature (e.g., 60-65° C.) using multiple inner and outer primers and a polymerase having strand displacement activity. In some instances, an inner primer pair containing a nucleic acid sequence complementary to a portion of the sense and antisense strands of the target nucleic acid initiate LAMP. Following strand displacement synthesis by the inner primers, strand displacement synthesis primed by an outer primer pair can cause release of a single-stranded amplicon. The single-stranded amplicon may serve as a template for further synthesis primed by a second inner and second outer primer that hybridize to the other end of the target nucleic acid and produce a stem-loop nucleic acid structure. In subsequent LAMP cycling, one inner primer hybridizes to the loop on the product and initiates displacement and target nucleic acid synthesis, yielding the original stem-loop product and a new stem-loop product with a stem twice as long. Additionally, the 3′ terminus of an amplicon loop structure serves as initiation site for self-templating strand synthesis, yielding a hairpin-like amplicon that forms an additional loop structure to prime subsequent rounds of self-templated amplification. The amplification continues with accumulation of many copies of the target nucleic acid. The final products of the LAMP process are stem-loop nucleic acids with concatenated repeats of the target nucleic acid in cauliflower-like structures with multiple loops formed by annealing between alternately inverted repeats of a target nucleic acid sequence in the same strand.

In some embodiments, the isothermal amplification assay comprises a digital reverse-transcription loop-mediated isothermal amplification (dRT-LAMP) reaction for quantifying the target nucleic acid (see, e.g., Khorosheva et al., (2016) Nucleic Acid Research, 44:2 e10). Typically, LAMP assays produce a detectable signal (e.g., fluorescence) during the amplification reaction. In some embodiments, fluorescence can be detected and quantified. Any suitable method for detecting and quantifying florescence can be used. In some instances, a device such as Applied Biosystem's QuantStudio can be used to detect and quantify fluorescence from the isothermal amplification assay.

In some embodiments, quantitative real-time isothermal amplification of a target nucleic acid in a test sample is determined by detecting of one or more different (distinct) fluorescent labels attached to nucleotides or nucleotide analogs incorporated during isothermal amplification of the target nucleic acid (e.g., 5-FAM (522 nm), ROX (608 nm), FITC (518 nm) and Nile Red (628 nm). In another embodiment, quantitative real-time isothermal amplification of a target nucleic acid in a test sample can be determined by detection of a single fluorophore species (e.g., ROX (608 nm)) attached to nucleotides or nucleotide analogs incorporated during isothermal amplification of the target nucleic acid. In some embodiments, each fluorophore species used emits a fluorescent signal that is distinct from any other fluorophore species, such that each fluorophore can be readily detected among other fluorophore species present in the assay.

In some embodiments, methods of detecting amplification of a target nucleic acid in a test sample by quantitative real-time isothermal amplification can include using intercalating fluorescent dyes, such as SYTO dyes (SYTO 9 or SYTO 82). In some embodiments, methods of detecting amplification of a target nucleic acid in a test sample by quantitative real-time isothermal amplification can include using unlabeled primers to isothermally amplify the target nucleic acid in the test sample, and a labeled probe (e.g., having a fluorophore) to detect isothermal amplification of the target nucleic acid in the test sample. In some embodiments, unlabeled primers are used to isothermally amplify a target nucleic acid present in the test sample, and a probe is used having a 5-FAM dye label on the 5′ end and a minor groove binder (MGB) and non-fluorescent quencher on the 3′ end to detect isothermal amplification of the target nucleic acid (e.g., TaqMan Gene Expression Assays from ThermoFisher Scientific).

In some embodiments, detecting amplification of the target nucleic acid in the test sample is performed using a one-step, or two-step, quantitative real-time isothermal amplification assay. In a one-step quantitative real-time isothermal amplification assay, reverse transcription is combined with quantitative isothermal amplification to form a single quantitative real-time isothermal amplification assay. A one-step assay reduces the number of hands-on manipulations as well as the total time to process a test sample. A two-step assay comprises a first-step, where reverse transcription is performed, followed by a second-step, where quantitative isothermal amplification is performed. It is within the scope of the skilled artisan to determine whether a one-step or two-step assay should be performed.

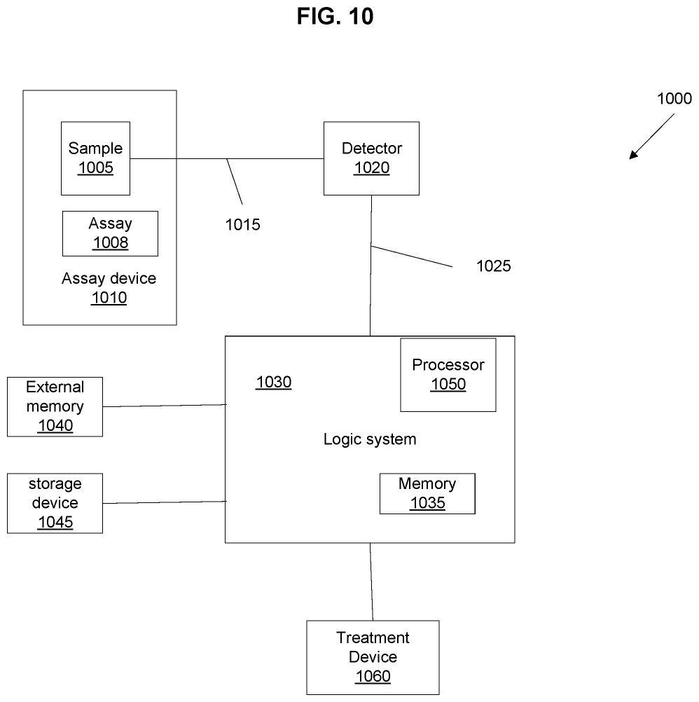

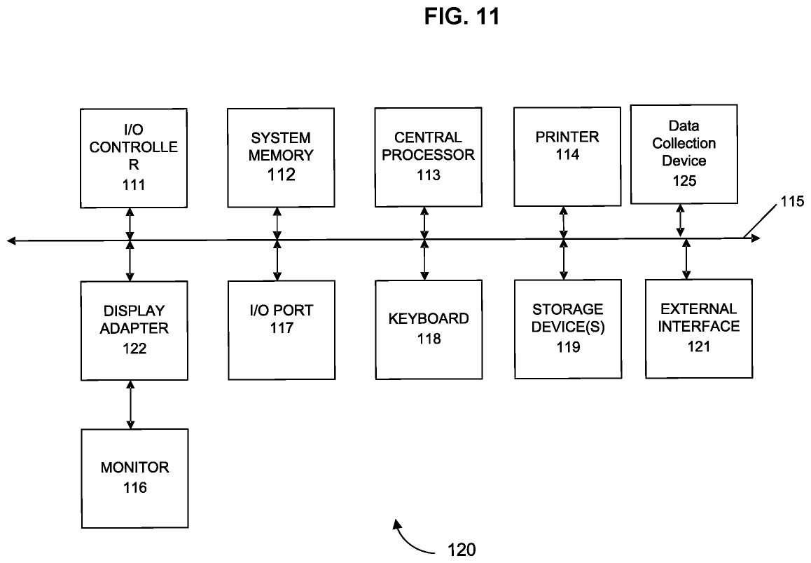

In some embodiments, the amplification and/or detection is carried out in whole or in part using an integrated measurement system, as illustrated in FIG. 10, which may also comprise a computer system as described elsewhere herein (see, e.g., FIG. 11).

In some embodiments, viral or biomarker scores are calculated based on the Tt (time to threshold) values for each of the tested biomarkers. This may be accomplished by, e.g., establishing standard curves for the isothermal or other amplification of the target nucleic acid (e.g., biomarker) and the reference nucleic acid (e.g., housekeeping gene). The standard curves can be obtained by performing real-time isothermal amplification assays using quantitated calibrator samples with multiple known input concentrations. Appropriate methods are provided in, e.g., PCT Publication No. WO 2020/061217, the entire disclosure of which is herein incorporated by reference.

For example, in some embodiments, to generate a standard curve, quantitated calibrator samples are obtained by performing serial dilutions of a quantitated material. For example, a template is serially diluted in a buffer at 10-fold concentration intervals yielding templates covering a range of concentrations. The precise concentration of each calibrator sample can be determined using methods known in the art.

To obtain a standard curve, a real-time amplification assay is performed for each aliquot with a known quantity of a respective calibrator sample with a respective concentration of the target nucleic acid. In a real-time amplification assay for each respective calibrator sample, the intensity of the fluorescence emitted by intercalating fluorescent dyes (e.g., dsDNA dyes) or fluorescent labels for the target nucleic acid is measured as a function of time. For example, a plot can be generated of fluorescence intensity as a function of time in a real-time quantitative amplification assay. A dashed line can be used to represent a pre-determined threshold intensity, and the elapsed time from the moment when the amplification is started is the time-to-threshold Tt. A respective time-to-threshold value can be determined from each respective fluorescence curve as a function of time. Thus, time-to-threshold values Ttₙ, Ttₙ₊₁, Ttₙ₊₂, etc., are obtained for the different calibrator samples.

For exponential amplifications, the time-to-threshold is linearly proportional to the logarithm (e.g., logarithm to base 10) of the starting copy number (also referred to as template abundance). A scatter plot of data points can be generated from the fluorescence curves. Each data point represents a data pair [Log₁₀ (CopyNumber), Tt] (note that CopyNumber refers to starting number of copies of a nucleic acid in an amplification assay). In some embodiments, the data points fall approximately on a straight line. A linear regression is then performed on the data points in the plot to obtain the straight line that best fits the data points with the least amount of total deviations. The result of the linear regression is a straight line represented by the following equation,

$$Tt = m \times {Log}_{10} ( CopyNumber ) + b , \qquad ( 1 )$$

where m is the slope of the line, and b is y-intercept. The slope m represents the efficiency of the isothermal amplification of the target nucleic acid; b represents a time-to-threshold as template copy number approaches zero. The straight line represented by Equation (1) is referred to as the standard curve.

In some embodiments, replicates (e.g., triplicates) of isothermal amplification assays may be run for each sample in order to gain a higher level of confidence in the data. Replicate time-to-threshold values can be averaged, and standard deviations can be calculated.

Once the standard curve is established for a given isothermal amplification assay, the standard curve can be used to convert a time-to-threshold value to a starting copy number for future runs of the amplification assay of unknown starting numbers of copies of the target nucleic acid, using the following equation,

$$CopyNumber = {10}^{\frac{Tt - b}{m}} . \qquad ( 2 )$$

Normally, the data points for low copy numbers or very high copy numbers may fall off of the straight line. The range of copy numbers within which the data points can be represented by the straight line is referred to as the dynamic range of the standard curve. The linear relationship between the time-to-threshold and the logarithmic of copy number represented by the standard curve would be valid only within the dynamic range.

If the amplification efficiencies for a target nucleic acid and a reference nucleic acid are different for a given isothermal amplification assay, it may be necessary to obtain separate standard curves for the target nucleic acid and the reference nucleic acid. Thus, two sets of real-time isothermal amplification assays may be performed, one set for establishing the standard curve for the target nucleic acid, the other set for establishing the standard curve for the reference nucleic acid. In cases where multiple target nucleic acids are considered (e.g., for a panel of 5, 6, 7, 8, 9, 10 or more biomarkers), a standard curve for each target nucleic acid may be obtained.

In some embodiments, the standard curves are generated prior to obtaining a test sample. That is, the standard curves are not generated on-board with the quantitative isothermal amplification of the test sample. Such standard curves may be referred to as off-board standard curves. Off-board standard curves may be used for estimating relative abundance values. For example, for a test sample of unknown input concentration of a target nucleic acid, a first real-time amplification assay is performed for a first aliquot of the test sample to obtain a first time-to-threshold value with respect to the target nucleic acid. A second real-time isothermal amplification assay is then performed for a second aliquot of the test sample to obtain a second time-to-threshold value with respect to a reference nucleic acid. The first aliquot and the second aliquot contain substantially the same amount of the test sample. The first time-to-threshold value may then be converted into starting number of copies of the target nucleic acid using the standard curve of the target nucleic acid. Similarly, the second time-to-threshold value may be converted into starting number of copies of the reference nucleic acid using the standard curve of the reference nucleic. The starting number of copies of the target nucleic acid is then normalized against that of the reference nucleic acid to obtain a relative abundance value.

In cases where the amplification efficiencies for a target nucleic acid and a reference nucleic acid have approximately the same value that is known, relative abundance may be obtained directly from time-to-threshold values without using standard curves.

### 4. Calculating Biomarker Scores

To determine the likelihood of a bacterial or viral infection (or degree of severity, etc.), a model (e.g., a model with the hyperparameter configuration providing a maximum AUC) is applied to the biomarker expression data from the subject to determine a score, e.g., a “diagnostic score” or “biomarker score”, that is indicative of the probability of an infection. This score can be used, e.g., to classify the subject into any of a number of bins, e.g., 2 bins corresponding to the probable presence or absence of an infection, or 3 bins with a “low”, “intermediate” or “indeterminate”, and “high” likelihood of an infection. In a particular embodiment, the model uses logistic regression and the selected biomarker genes, e.g., IFI27, JUP, LAX1, CTSB, GPAA1, HK3, and TNIP1, to calculate the score. The probability of an infection as determined using the model is then used to determine the optimal treatment of the subject, as described in more detail elsewhere herein.

The biomarker genes selected for use and measured as described herein will be combined to generate a biomarker score. A score would be calculated by either taking the sum, product, or quotient of the gene levels, taken in terms of their absolute levels or their relative levels as compared to control genes, e.g., housekeeping genes, or by inputting them into a linear or nonlinear algorithm that incorporates at least the measured gene levels, e.g., the measured levels of 2, 3, 4, 5, 6, 7, 8, 9, 10 or more biomarker genes, into an interpretable score.

It will be appreciated that it is not necessary that all of the biomarkers will be elevated or depressed relative to control levels in a given patient to give rise to a determination of infection, or of an infection of bacterial or viral origin; for example, for a given biomarker level there can be some overlap between individuals falling into different infection categories. However, collectively the combined levels for all of the biomarker genes included in the assay will give rise to a biomarker score that, if it surpasses a threshold, e.g., a threshold derived from at least 50, 100, 150, 200, 250, 300, 350, 400, 500 or more patients with, e.g., an infection of bacterial or viral origin, and/or of 50, 100, 150, 200, 250, 300, 350, 400, 500 or more control individuals without an infection, that allows a determination concerning the infection status of the patient, or of a likelihood or probability concerning the infection status of the patient. For example, for a diagnosis of a bacterial infection, the threshold could be such that at across a population of at least 100 healthy controls and 100 patients with a bacterial infection, at least 90% of the patients with a bacterial infection are above the threshold. In certain embodiments, the biomarker score is calculated, based on the measured levels of the biomarkers in patients with bacterial or viral infections, and/or non-infectious (e.g., individuals showing symptoms of acute illness but with no infection) and in healthy controls, such that a score for a patient that surpasses the threshold indicates that the patient has a likelihood ratio of 1.5, 2, 2.5, 3, 3.5, 4, or more for the presence of a bacterial or viral infection, or for an absence of both a bacterial and viral infection, compared to a reference population, or that there is a likelihood or probability of at least 70%, 80%, 85%, 90%, 95%, 96%, 97%, 98%, 99%, or higher of the patient having, e.g., an infection, or having specifically a bacterial or viral infection. It will be appreciated that in any given assay there can be more than one threshold, e.g., a threshold in one direction that indicates a bacterial infection, and a threshold in the other direction that indicates a viral infection.

In semi-quantitative methods, a threshold or cut-off value is suitably determined, and is optionally a predetermined value. In particular embodiments, the threshold value is predetermined in the sense that it is fixed, for example, based on previous experience with the assay and/or a population of affected and/or unaffected subjects, e.g., with a population of 50, 100, 150, 200, 250, 300, 350, 400, 450, 500, or more affected and/or unaffected subjects. Alternatively, the predetermined value can also indicate that the method of arriving at the threshold is predetermined or fixed even if the particular value varies among assays or can even be determined for every assay run.

For the statistical analyses described herein, e.g., for the selection of biomarkers to be included in the calculation of a score or in the calculation of a probability or likelihood of a particular infection status in a patient, as well as for diagnostic or therapeutic assessments made in view of a given biomarker score, other relevant information will also be considered, such as clinical data regarding one or more conditions suffered by each individual. This can include demographic information such as age, race, and sex; information regarding a presence, absence, degree, stage, severity or progression of a condition, phenotypic information, such as details of phenotypic traits, genetic or genetically regulated information, amino acid or nucleotide related genomics information, results of other tests including imaging, biochemical and hematological assays, other physiological scores such as a SOFA (Sequential Organ Failure Assessment) score, or the like.

In some embodiments, likelihood is assessed by comparing the level or abundance of individual biomarkers to one or more preselected or threshold levels, or of an overall biomarker score to one or more such levels. Threshold values can be selected that provide an acceptable ability to predict diagnosis, prognostic risk, treatment success, etc. In illustrative examples, receiver operating characteristic (ROC) curves are calculated by plotting the value of a variable versus its relative frequency in two populations in which a first population has a first condition or risk and a second population has a second condition or risk (called arbitrarily, for example, “healthy condition” and “infection,” “healthy condition” and “bacterial infection,” “healthy condition” and “viral infection,” “low severity infection,” and “high severity infection,” etc.).

For any particular biomarker, a distribution of biomarker levels for subjects with and without a disease will likely overlap, and some overlap will be present for biomarker scores as well. Under such conditions, a test does not absolutely distinguish a first condition and a second condition with 100% accuracy, and the area of overlap indicates where the test cannot distinguish the first condition and the second condition. A threshold value is selected, above which (or below which, depending on how a biomarker or biomarker score changes with a specified condition or prognosis) the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143:29-36 (1982).

Alternatively, or in addition, threshold values can be established by obtaining an earlier biomarker expression level, or a biomarker score, from the same patient, to which later results can be compared. In these embodiments, the individual in effect acts as their own “control group.” In biomarker gene levels or biomarker scores that increase with condition severity or prognostic risk, an increase over time in the same patient can indicate a worsening of the condition or a failure of a treatment regimen, while a decrease over time can indicate remission of the condition or success of a treatment regimen.

In some embodiments, a positive likelihood ratio, negative likelihood ratio, odds ratio, and/or AUC or receiver operating characteristic (ROC) values are used as a measure of a method's ability to predict risk or to diagnose a disease or condition. As used herein, the term “likelihood ratio” is the probability that a given test result would be observed in a subject with a condition of interest divided by the probability that that same result would be observed in a patient without the condition of interest. Thus, a positive likelihood ratio is the probability of a positive result observed in subjects with the specified condition divided by the probability of a positive results in subjects without the specified condition. A negative likelihood ratio is the probability of a negative result in subjects without the specified condition divided by the probability of a negative result in subjects with specified condition. The term “odds ratio,” as used herein, refers to the ratio of the odds of an event occurring in one group (e.g., a healthy condition group) to the odds of it occurring in another group (e.g., an infection negative group, or a group with bacterial infections or viral infection), or to a data-based estimate of that ratio. The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., a healthy condition biomarker gene level or score and a score for viral or bacterial infection). ROC curves are useful for plotting the performance of a particular feature (e.g., any of the biomarker expression levels or biomarker scores described herein and/or any item of additional biomedical information) in distinguishing or discriminating between two populations (e.g., cases having a condition and controls without the condition). Typically, the feature data across the entire population (e.g., the cases and controls) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls.

Although this refers to scenarios in which a feature is elevated in cases compared to controls, it also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to produce a single value, and this single value can be plotted in a ROC curve. Additionally, any combination of multiple features, in which the combination derives a single output value, can be plotted in a ROC curve. These combinations of features can comprise a test. The ROC curve is the plot of the sensitivity of a test against the specificity of the test, where sensitivity is traditionally presented on the vertical axis and specificity is traditionally presented on the horizontal axis. Thus, “AUC ROC values” are equal to the probability that a classifier will rank a randomly chosen positive instance higher than a randomly chosen negative one.

As described above, the abundance values for the individual biomarker genes in cells of the biological sample can be combined using a mathematical formula or a machine learning or other algorithm to produce a single diagnostic score, such as the viral score that can indicate the presence or absence (or probability) of an infection in a subject. In these embodiments, the produced score carries more predictive power than any individual gene level alone (e.g., has a greater area under the receiver-operating-characteristic curve for discrimination of infection or non-infection).

In some embodiments, types of algorithms for integrating multiple biomarkers into a single diagnostic score may include, but not limited to, a difference of geometric means, a difference of arithmetic means, a difference of sums, a simple sum, and the like. In some embodiments, a diagnostic score may be estimated based on the relative abundance values of multiple biomarkers using machine-learning models, such as a regression model, a tree-based machine-learning model, a support vector machine (SVM) model, an artificial neural network (ANN) model, or the like.

Biomarker data may also be analyzed by a variety of methods to determine the statistical significance of differences in observed levels of biomarkers between test and reference expression profiles in order to evaluate the infection status or probability of an infection in a subject. In certain embodiments, patient data is analyzed by one or more methods including, but not limited to, multivariate linear discriminant analysis (LDA), receiver operating characteristic (ROC) analysis, principal component analysis (PCA), ensemble data mining methods, significance analysis of microarrays (SAM), cell specific significance analysis of microarrays (csSAM), spanning-tree progression analysis of density-normalized events (SPADE), and multi-dimensional protein identification technology (MUDPIT) analysis. (See, e.g., Hilbe (2009) Logistic Regression Models, Chapman & Hall/CRC Press; Mclachlan (2004) Discriminant Analysis and Statistical Pattern Recognition. Wiley Interscience; Zweig et al. (1993) Clin. Chem. 39:561-577; Pepe (2003) The statistical evaluation of medical tests for classification and prediction, New York, N.Y.: Oxford; Sing et al. (2005) Bioinformatics 21:3940-3941; Tusher et al. (2001) Proc. Natl. Acad. Sci. U.S.A. 98:5116-5121; Oza (2006) Ensemble data mining, NASA Ames Research Center, Moffett Field, Calif., USA; English et al. (2009) J. Biomed. Inform. 42 (2): 287-295; Zhang (2007) Bioinformatics 8:230; Shen-Orr et al. (2010) Journal of Immunology 184:144-130; Qiu et al. (2011) Nat. Biotechnol. 29 (10): 886-891; Ru et al. (2006) J. Chromatogr. A. 1111 (2): 166-174, Jolliffe Principal Component Analysis (Springer Series in Statistics, 2.sup.nd edition, Springer, N Y, 2002), Koren et al. (2004) IEEE Trans Vis Comput Graph 10:459-470; herein incorporated by reference in their entireties.)

In some embodiments, at least two (e.g., 2, 3, 4, 5, 6, 7, 8, 9, 10, or more) biomarker genes are selected to discriminate between subjects with a first condition and subjects with a second condition with at least about 70%, 75%, 80%, 85%, 90%, 95% accuracy or having a C-statistic of at least about 0.70, 0.75, 0.80, 0.85, 0.90, 0.95.

In the case of a positive likelihood ratio, a value of 1 indicates that a positive result is equally likely among subjects in both the “condition” and “control” groups; a value greater than 1 indicates that a positive result is more likely in the condition group; and a value less than 1 indicates that a positive result is more likely in the control group. In this context, “condition” is meant to refer to a group having one characteristic (e.g., the presence of a healthy condition, bacterial infection, viral infection) and “control” group lacking the same characteristic. In the case of a negative likelihood ratio, a value of 1 indicates that a negative result is equally likely among subjects in both the “condition” and “control” groups; a value greater than 1 indicates that a negative result is more likely in the “condition” group; and a value less than 1 indicates that a negative result is more likely in the “control” group. In the case of an odds ratio, a value of 1 indicates that a positive result is equally likely among subjects in both the condition” and “control” groups; a value greater than 1 indicates that a positive result is more likely in the “condition” group; and a value less than 1 indicates that a positive result is more likely in the “control” group. In the case of an AUC ROC value, this is computed by numerical integration of the ROC curve. The range of this value can be 0.5 to 1.0. A value of 0.5 indicates that a classifier (e.g., a biomarker level or score) cannot discriminate between cases and controls, while 1.0 indicates perfect diagnostic accuracy. In certain embodiments, biomarker gene levels and/or biomarker scores are selected to exhibit a positive or negative likelihood ratio of at least about 1.5 or more or about 0.67 or less, at least about 2 or more or about 0.5 or less, at least about 5 or more or about 0.2 or less, at least about 10 or more or about 0.1 or less, or at least about 20 or more or about 0.05 or less.

In certain embodiments, the biomarker gene levels and/or biomarker scores are selected to exhibit an odds ratio of at least about 2 or more or about 0.5 or less, at least about 3 or more or about 0.33 or less, at least about 4 or more or about 0.25 or less, at least about 5 or more or about 0.2 or less, or at least about 10 or more or about 0.1 or less. In certain embodiments, biomarker gene levels and/or biomarker scores are selected to exhibit an AUC ROC value of greater than 0.5, preferably at least 0.6, more preferably 0.7, still more preferably at least 0.8, even more preferably at least 0.9, and most preferably at least 0.95.

In some cases, multiple thresholds can be determined in so-called “tertile,” “quartile,” or “quintile” analyses. In these methods, the “diseased” and “control groups” (or “high risk” and “low risk”) groups are considered together as a single population, and are divided into 3, 4, or 5 (or more) “bins” having equal numbers of individuals. The boundary between two of these “bins” can be considered “thresholds.” A risk (of a particular diagnosis or prognosis for example) can be assigned based on which “bin” a test subject falls into.

The phrases “assessing the likelihood” and “determining the likelihood,” as used herein, refer to methods by which the skilled artisan can predict the presence or absence of a condition (e.g., a condition selected from healthy condition, infection, viral infection, bacterial infection) in a patient. The skilled artisan will understand that this phrase includes within its scope an increased probability that a condition is present or absent in a patient; that is, that a condition is more likely to be present or absent in a subject. For example, the probability that an individual identified as having a specified condition actually has the condition can be expressed as a “positive predictive value” or “PPV.” Positive predictive value can be calculated as the number of true positives divided by the sum of the true positives and false positives. PPV is determined by the characteristics of the predictive methods of the present invention as well as the prevalence of the condition in the population analyzed. The statistical algorithms can be selected such that the positive predictive value in a population having a condition prevalence is in the range of 70% to 99% and can be, for example, at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that an individual identified as not having a specified condition actually does not have that condition can be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of the disease in the population analyzed. The statistical methods and models can be selected such that the negative predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a subject is determined to have a significant likelihood of having or not having a specified condition. By “significant likelihood” is meant that the subject has a reasonable probability (0.6, 0.7, 0.8, 0.9 or more) of having, or not having, a specified condition.

In some embodiments, data sets corresponding to the biomarker gene expression levels and biomarker scores of the invention are used to create a diagnostic or predictive rule or model based on the application of a statistical and machine learning algorithm, which produces the diagnostic score. Such an algorithm uses relationships between a biomarker profile and a condition selected from healthy condition, infection, viral infection, bacterial infection, etc. observed in control subjects or typically cohorts of control subjects (sometimes referred to as training data), which provides combined control or reference biomarker profiles for comparison with the biomarker profiles of a subject. The data are used to infer relationships that are then used to predict the status of a subject, including the presence or absence of one of the conditions referred to above.

The term “correlating” generally refers to determining a relationship between one type of data with another or with a state. In various embodiments, correlating a given biomarker level or score with the presence or absence of a condition (e.g., a condition selected from a healthy condition, infection, viral infection, bacterial infection, etc.) comprises determining the presence, absence or amount of at least one biomarker in a subject that suffers from that condition; or in persons known to be free of that condition. In specific embodiments, a set of biomarker levels, absences or presences is correlated to a global probability or a particular outcome, using receiver operating characteristic (ROC) curves.

In typical embodiments of the present methods, the scores are calculated based on the Ct (Cycle Threshold) values for each of the tested biomarkers. Ct values and their calculation are well known in the art, and they can be calculated, e.g., by the software of the real-time PCR thermal cycler. Typically, in addition to the Ct values for the selected biomarkers, Ct values are also generated for one or more housekeeping (HK) gene, i.e. a uniformly expressed gene that shows low variance under all conditions. The HK gene is used to normalize the RNA input in each PCR reaction. A Ct value is also generated for the HK gene or genes, and for each tested biomarker a normalized value, referred to as Delta Ct (corresponding to CtBiomarker-CtHK), is calculated. In preferred embodiments, the Delta Ct values for the different biomarker genes are used to calculate the biomarker score, e.g., using a custom validated algorithm. For example, the biomarker score can be generated using the geometric mean of the Delta Ct values for the different biomarkers.

### 5. Treatment

In view of a given biomarker score in a patient, e.g., when a biomarker score is calculated that suggests a relative likelihood of a particular infection status (such as healthy condition, infection, viral infection, bacterial infection, etc.), methods are also provided for the management of the condition, for the prevention of further progression of the condition, or for the assessment of the efficacy of therapies in subjects for the condition. The management of an infection can include, e.g., the use of therapeutic compounds such as, antimicrobial agents, antibiotics, antiviral compounds, steroids, immune-modulating small molecules or proteins, or others. In addition, palliative therapies as described for example in Cohen and Glauser (1991, Lancet 338:736-739) aimed at restoring and protecting organ function can be used such as intravenous fluids and oxygen and tight glycemic control.

Typically, the therapeutic agents will be administered in pharmaceutical (or veterinary) compositions together with a pharmaceutically acceptable carrier and in an effective amount to achieve their intended purpose. The dose of active compounds administered to a subject should be sufficient to achieve a beneficial response in the subject over time such as a reduction in, or relief from, the symptoms of the acute infection. The quantity of the pharmaceutically active compounds(s) to be administered can depend on the subject to be treated inclusive of the age, sex, weight and general health condition thereof. In this regard, precise amounts of the active compound(s) for administration will depend on the judgment of the practitioner. In determining the effective amount of the active compound(s) to be administered in the treatment or prevention of a viral or bacterial infection, the medical practitioner or veterinarian can evaluate severity of any symptom associated with the presence of an infection, including, e.g., inflammation, blood pressure anomaly, tachycardia, tachypnea fever, chills, vomiting, diarrhea, skin rash, headaches, confusion, muscle aches, seizures. In any event, those of skill in the art can readily determine suitable dosages of the therapeutic agents and suitable treatment regimens without undue experimentation.

The therapeutic agents can be administered in concert with adjunctive (palliative) therapies to increase oxygen supply to major organs, increase blood flow to major organs and/or to reduce the inflammatory response. Illustrative examples of such adjunctive therapies include non-steroidal anti-inflammatory drugs (NSAIDs), intravenous saline and oxygen.

Thus, the present invention contemplates the use of the methods and compositions described above and elsewhere herein in methods for treating, preventing or inhibiting the development of a viral or bacterial infection in a subject. These methods generally comprise (1) correlating a reference biomarker score with the presence or absence of a condition selected from a healthy condition, infection positive, viral infection, bacterial infection, etc., wherein the reference biomarker score evaluates at least two (e.g., 2, 3, 4, 5, 6, etc.) of the herein-described biomarker genes; (2) calculating a biomarker score of a sample from a patient; (3) determining a likelihood of the subject having or not having the condition based on the sample biomarker score and the reference biomarker score, and administering to the subject, on the basis that the subject has an increased likelihood of having an infection, e.g. bacterial infection or viral infection, an effective amount of an agent that treats or ameliorates the symptoms or reverses or inhibits the development of the bacterial or viral infection.

The present invention can be practiced in the field of predictive medicine for the purposes of diagnosis or monitoring the presence or development of a condition selected from an infection, viral infection, and bacterial infection in a subject, and/or monitoring response to therapy efficacy.

As used herein, the term “treatment regimen” refers to prophylactic and/or therapeutic (i.e., after onset of a specified condition) treatments, unless the context specifically indicates otherwise. The term “treatment regimen” encompasses natural substances and pharmaceutical agents (i.e., “drugs”) as well as any other treatment regimen including but not limited to dietary treatments, physical therapy or exercise regimens, surgical interventions, and combinations thereof. In preferred embodiments, the treatment regimens of the invention will include the administration of antibacterial or antiviral compounds for the treatment of bacterial or viral infections, respectively.

The invention can also be practiced to evaluate whether a subject is responding (i.e., a positive response) or not responding (i.e., a negative response) to a treatment regimen. This aspect of the invention provides methods of correlating a biomarker score with a positive and/or negative response to a treatment regimen. These methods generally comprise: (a) calculating a biomarker score from a subject with a viral or bacterial infection following commencement of the treatment regimen, wherein the biomarker score is based on the expression levels of at least two (e.g., 2, 3, 4, 5, 6, etc.) of the herein-disclosed biomarker genes; and (b) correlating the biomarker score from the subject with a positive and/or negative response to the treatment regimen.

In some embodiments, the methods further comprise determining a first biomarker score from the patient prior to commencing the treatment regimen (i.e., a baseline profile), wherein the first biomarker score evaluates at least two (e.g., 2, 3, 4, 5, 6, etc.) of the herein-described biomarkers; and comparing the first sample biomarker score with a second sample biomarker score from the subject after commencement of the treatment regimen, wherein the second sample biomarker score evaluates for an individual biomarker in the first sample biomarker score a corresponding biomarker.

This aspect of the invention can be practiced to identify responders or non-responders relatively early in the treatment process, i.e., before clinical manifestations of efficacy. In this way, the treatment regimen can optionally be discontinued, a different treatment protocol can be implemented, and/or supplemental therapy can be administered. Thus, in some embodiments, a sample biomarker score is obtained within about 30 minutes, 1 hour, 2 hours, 4 hours, 6 hours, 12 hours, 1 day, 2 days, 3 days, 4 days, 5 days, 1 week, 2 weeks, 3 weeks, 4 weeks, 6 weeks, 8 weeks, 10 weeks, 12 weeks, 4 months, six months or longer of commencing therapy.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### 6. Kits and Systems

### a) Kits

In one aspect, kits are provided for the detection of a respiratory viral infection in a subject, wherein the kits can be used to detect the biomarkers described herein. For example, the kits can be used to detect any one or more of the biomarkers described herein, which are differentially expressed in samples from subjects with viral or bacterial infections and from subjects without an infection. The kit may include one or more agents for the detection of biomarkers, a container for holding a biological sample isolated from a human subject suspected of having an infection; and instructions for reacting agents with the biological sample or a portion of the biological sample to detect the presence or amount of at least one biomarker in the biological sample. The agents may be packaged in separate containers. The kit may further comprise one or more control reference samples and reagents for performing isothermal amplification, e.g., qRT-LAMP, e.g., reference samples from subjects with or without an infection. The kit may also comprise one or more devices or implements for carrying out any of the herein devices, e.g., 96-well plates, microfluidic cartridges, single-well multiplex assays, etc.

In certain embodiments, the kit comprises agents for measuring the levels of at least five or six biomarkers of interest. For example, the kit may include agents, e.g., primers, for detecting biomarkers of a panel comprising a CTSB polynucleotide, a GPAA1 polynucleotide, an HK3 polynucleotide, a TNIP1 polynucleotide, an IFI27 polynucleotide, a JUP polynucleotide, and a LAX1 polynucleotide, or for detecting any one or more biomarkers listed in Table 3, Table 7, Table 9, or Table 10.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing a viral infection.

### C. Measurement Systems for Detecting and Recording Biomarker Expression

In one aspect, a measurement system is provided. Such systems allow, e.g., the detection of biomarker gene expression in a sample and the recording of the data resulting from the detection. The stored data can then be analyzed as described elsewhere herein to determine the virus infection status of a subject. Such systems can comprise assay systems (e.g., comprising an assay device and detector), which can transmit data to a logic system (such as a computer or other system or device for capturing, transforming, analyzing, or otherwise processing data from the detector). The logic system can have any one or more of multiple functions, including controlling elements of the overall system such as the assay system, sending data or other information to a storage device or external memory, and/or issuing commands to a treatment device.

An exemplary measurement system is shown in FIG. 10. The system as shown includes a sample 1005, an assay device 1010, where an assay 1008 can be performed on sample 1005. For example, sample 1005 can be contacted with reagents of assay 508 to provide a signal of a physical characteristic 1015. An example of an assay device can be a flow cell that includes probes and/or primers of an assay or a tube through which a droplet moves (with the droplet including the assay). Physical characteristic 1015 (e.g., a fluorescence intensity, a voltage, or a current), from the sample is detected by detector 1020. Detector 1020 can take a measurement at intervals (e.g., periodic intervals) to obtain data points that make up a data signal. In one embodiment, an analog-to-digital converter converts an analog signal from the detector into digital form at a plurality of times. Assay device 1010 and detector 1020 can form an assay system, e.g., an amplification and detection system that measures biomarker gene expression according to embodiments described herein. A data signal 1025 is sent from detector 1020 to logic system 1030. As an example, data signal 1025 can be used to determine expression levels for selected biomarkers. Data signal 1025 can include various measurements made at a same time, e.g., different colors of fluorescent dyes or different electrical signals for different molecules of sample 1005, and thus data signal 1025 can correspond to multiple signals. Data signal 1025, either directly or after online processing by Processor 1050, may be stored in a local memory 1035, an external memory 1040, or a storage device 1045. System 1000 may also include a treatment device 1060, which can provide a treatment to the subject. Treatment device 1060 can determine a treatment and/or be used to perform a treatment. Examples of such treatment can include surgery, radiation therapy, chemotherapy, immunotherapy, targeted therapy, hormone therapy, and stem cell transplant. Logic system 1030 may be connected to treatment device 1060, e.g., to provide results of a method described herein. The treatment device may receive inputs from other devices, such as an imaging device and user inputs (e.g., to control the treatment, such as controls over a robotic system).

### D. Computer Systems and Diagnostic Systems

Certain aspects of the herein-described methods may be totally or partially performed with a computer system including one or more processors, which can be configured to perform the steps. Thus, embodiments are directed to computer systems configured to perform the steps of methods described herein, potentially with different components performing a respective step or a respective group of steps. The computer systems of the present disclosure can be part of a measuring system as described above, or can be independent of any measuring systems. In some embodiments, the present disclosure provides a computer system that calculates a viral score based on inputted biomarker expression (and optionally other) data, and determines the viral infection status of a subject.

An exemplary computer system is shown in FIG. 11. Any of the computer systems may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. The subsystems shown in FIG. 11 are interconnected via a system bus 65. Additional subsystems such as a printer 114, keyboard 118, storage device(s) 119, monitor 116 (e.g., a display screen, such as an LED), which is coupled to display adapter 72, and others are shown. Peripherals and input/output (I/O) devices, which couple to I/O controller 61, can be connected to the computer system by any number of means known in the art such as input/output (I/O) port 117 (e.g., USB, FireWire®). For example, I/O port 117 or external interface 121 (e.g. Ethernet, Wi-Fi, etc.) can be used to connect computer system 120 to a wide area network such as the Internet, a mouse input device, or a scanner. The interconnection via system bus 115 allows the central processor 113 to communicate with each subsystem and to control the execution of a plurality of instructions from system memory 112 or the storage device(s) 119 (e.g., a fixed disk, such as a hard drive, or optical disk), as well as the exchange of information between subsystems. The system memory 112 and/or the storage device(s) 119 may embody a computer readable medium. Another subsystem is a data collection device 115, such as a camera, microphone, accelerometer, and the like. Any of the data mentioned herein can be output from one component to another component and can be output to the user. A computer system can include a plurality of the same components or subsystems, e.g., connected together by external interface 121, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

In one aspect, the present disclosure provides a computer implemented method for determining the presence or absence of an infection in a patient. The computer performs steps comprising, e.g.: receiving inputted patient data comprising values for the levels of one or more biomarkers in a biological sample from the patient; analyzing the levels of one or more biomarkers and optionally comparing them to respective reference values, e.g., to a housekeeping reference gene for normalization; calculating a biomarker score for the patient based on the levels of the biomarkers and comparing the score to one or more threshold values to assign the patient to an infection status category; and displaying information regarding the infection status or probability of an infection in the patient. In certain embodiments, the inputted patient data comprises values for the levels of a plurality of biomarkers in a biological sample from the patient. In one embodiment, the inputted patient data comprises values for the levels of CTSB, GPAA1, HK3, TNIP1, IFI27, JUP, and/or LAX1 polynucleotides. Such computer-implemented methods can return results characterizing aspects of the plurality of biomarkers described for determining presence or absence of an infection, with extremely high performance (e.g., accuracy greater than 80%, duration of time to results less than 10 minutes, etc.).

In a further aspect, a diagnostic system is included for performing the computer implemented method, as described. A diagnostic system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining the infection status (i.e., infected or uninfected) of the subject. For example, the storage component includes instructions for calculating the biomarker score for the subject based on biomarker expression levels, as described herein. In addition, the storage component may further comprise instructions for performing multivariate linear discriminant analysis (LDA), receiver operating characteristic (ROC) analysis, principal component analysis (PCA), ensemble data mining methods, cell specific significance analysis of microarrays (csSAM), or multi-dimensional protein identification technology (MUDPIT) analysis. The computer processor is coupled to the storage component and configured to execute the instructions stored in the storage component in order to receive patient data and analyze patient data according to one or more algorithms. The display component displays information regarding the diagnosis of the patient. The storage component may be of any type capable of storing information accessible by the processor, such as a hard-drive, memory card, ROM, RAM, DVD, CD-ROM, USB Flash drive, write-capable, and read-only memories.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data. In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel. In one aspect, computer is a server communicating with one or more client computers. Each client computer may be configured similarly to the server, with a processor, storage component and instructions. Although the client computers and may comprise a full-sized personal computer, many aspects of the system and method are particularly advantageous when used in connection with mobile devices capable of wirelessly exchanging data with a server over a network such as the Internet.

### E. Examples

### Example 1. Diagnostic Host Gene Expression Analysis by Quantitative Reverse Transcription

### 1. Abstract

Early and accurate diagnosis of acute infections can help minimize the over-prescription of antibiotics and improve patient outcomes. In previous work, we have described a method for discriminating between bacterial and viral etiologies in acute infection based on changes in host gene expression. Unfortunately, established technologies used for gene expression profiling are typically expensive and slow, confounding integration with physicians' workflows. Here we report the development of an ultra-rapid test system for host gene expression profiling based on quantitative reverse transcription followed by loop-mediated isothermal amplification. We designed and developed ten mRNA-specific qRT-LAMP assays targeting 7 informative biomarkers for diagnosis of infectious etiology and 3 housekeeping reference genes. We optimized assay formulations to achieve a turnaround time of about 12 minutes without sacrificing specificity or precision. We verified the accuracy of the test system by performing gene expression profiling on a cohort of 57 clinical samples and comparing qRT-LAMP results to profiles obtained using an orthogonal reference technology, the NanoString nCounter SPRINT. Finally, we discuss considerations for the development of other qRT-LAMP gene expression profiling assays.

### 2. Introduction

Here we report the design and optimization of a qRT-LAMP assay system capable of executing rapid and accurate gene expression profiling primed for adaptation to low complexity instrumentation. We determined to leverage LAMP (e.g., RT-LAMP) as a quantitative technology for relative gene expression analysis owing to the improved turnaround time and lower instrument complexity required relative to qRT-PCR-based technologies. We reasoned that a rapid, simple quantitative test system would enable host gene expression profiling near the point of care, making it possible to integrate such a diagnostic seamlessly with physicians' workflows. We then demonstrate the performance of this assay system in discriminating between bacterial and viral etiologies using a cohort of prospectively collected human whole blood samples by comparing LAMP assay performance to established gold standard methods.

To overcome the turnaround time limitation of common gene expression profiling technologies and to minimize the complexity of instrumentation required to run the test, we developed a quantitative assay system based on reverse transcription followed by loop-mediated isothermal amplification (qRT-LAMP) technology. LAMP assays typically achieve target amplification in 10 to 30 minutes, depending on the reaction formulation and template input concentration. As with PCR, numerous approaches for optimizing LAMP assay chemistry, include optimization of ionic strength and character of the reaction, use of alternative enzymes and the incorporation of various additives in the reaction buffer to minimize template secondary structure and stabilize the active enzymes. Leveraging these strategies, the invention(s) described are further optimizedin the context of target sample type and generation of an assay system capable of meeting the stringent performance criteria imposed by the need to integrate with physicians' workflows near the point of care.

### 3. Materials and Methods

### a) Automated Total RNA Extraction

Automated total RNA extraction uses a modified version of the RNeasy Micro total RNA extraction kit executed on the QIAcube instrument (both Qiagen). Human whole blood was collected in PAXgene blood RNA tubes (PreAnalytiX), then frozen and stored at −80° C. For processing, samples are thawed in a biosafety cabinet until reaching room temperature. A 1 mL aliquot of each sample to be tested is transferred to a 2 mL processing tube. A 1 mL aliquot of 1×PBS, pH 7.5 is added to the blood sample, and mixed by inversion. The sample is centrifuged at 3500×g for 10 minutes to pellet precipitated RNA. Supernatant is discarded and the pellet is resuspended in 2 mL of nuclease-free water. The sample is centrifuged at 3500×g for 10 minutes, and the supernatant is discarded. The sample is resuspended in 350 μL of Buffer RLT Plus, included with the RNeasy Micro kit. The sample is then loaded onto the QIAcube and a version of the RNeasy Micro extraction protocol modified to include a DNA removal step via centrifugation through a gDNA Eliminator spin column (Qiagen) is performed to purify the RNA. The RNA is eluted in 14 μL of nuclease-free water.

### b) Fluorescence-Based RNA Quantitation

RNA quantitation is performed using the Quant-iT RNA Assay Kit and Qubit 4 Fluorimeter (both ThermoFisher). Quantitation is executed per the manufacturer's protocol.

c) Gene Expression Analysis by NanoString nCounter SPRINT Profiler

For gene expression analysis, a 150 ng sample of total RNA isolated from human whole blood using the automated total RNA extraction protocol described above is combined with a capture and reporter probe cocktail that is designed and supplied by NanoString. This cocktail contains probe pairs specific for all bacterial/viral target biomarkers—CTSB, GPAA1, HK3, TNIP1, IFI27, JUP, LAX1-in addition to multiple reference genes, including KPNA6, RREB1 and YWHAB. Each probe comprises a 50 base pair (bp) sequence complimentary to the target mRNA biomarker (probe sequences are available upon request). These probes are hybridized to target biomarkers by incubation at 65° C. for 16 hours in a proprietary hybridization buffer also supplied by NanoString. After hybridization is complete, samples are incubated at 4° C. for no longer than 6 hours.

Post hybridization, samples are further diluted with the addition of nuclease-free water per the manufacturer's instructions. Samples are then loaded into a NanoString SPRINT cartridge and placed in the nCounter SPRINT Profiler for analysis. Results are exported by the instrument as RCC files, which are analyzed using the nSolver 4.0 software provided by NanoString. The abundance of each target transcript is reported as “counts.” Each count represents a single instance of the instrument identifying a molecular barcode corresponding to a given target biomarker.

### d) LAMP Primers

LAMP amplification proceeds in two phases as diagrammed in FIG. 1A (27, 28, 36). In the first phase, a primary transcript is produced by amplification from one of the two “inner” primers, FIP/BIP, bound at F2/B2 sequences within the target. This is followed by sequential displacement of the product transcript by amplification from F3/B3 primers. The primary transcript serves as a template for a second round of amplification by the complimentary inner primer, BIP/FIP. The resulting transcript adopts a dumbbell secondary structure mediated by F1: Fic and B1: Bic sequence interactions. In the second, exponential phase of LAMP, the dumbbell template enables auto-amplification at the 3′ end of the transcript, amplification by primers binding at F2/B2 sequences, and through rate enhancing primers (LF/LB) that recognize sequences within the loop region of the secondary transcript. RT-LAMP requires an initial reverse transcription (RT) event to generate a primary DNA transcript from an RNA template.

In the system we report here, BIP primers serve as the site for RT initiation, as FIP primers are designed to correspond to sense strand sequences for each mRNA target. Because amplification from F2/B2 sites initiates productive transcript formation and must proceed through sequences falling between these sites, we reasoned that we could impart specificity for mRNA over genomic DNA in LAMP assays by designing primers such that exon: exon junctions fall either within the F2/B2 sequences of FIP/BIP primers, or between the F1 and B1 sequences if intervening introns are sufficiently large to prohibit read-through amplification. To generate proof of concept assays for CTSB and IFI27, target mRNA isoforms were selected based on measured tissue-specific abundance as reported by the Genotype-Tissue Expression project (GTEx). The highest abundance isoform in whole blood was selected as the target sequence for assay development. Regions of up to 500 bp that contain exon: exon junctions flanking introns of at least 1000 bp (where possible) were selected from within the target isoform sequences as inputs for assay design. These sequences were uploaded to Primer Explorer V5.0 (https://primerexplorer.jp/e/) and primer outputs were manually screened to identify solutions with exon: exon junction(s) falling in the desired region of primer or amplicon sequences.

### e) Commercial RT-LAMP System for Proof-of-Concept Assay Development

Assay development using the WarmStart LAMP kit from New England Biolabs is carried out in a 25 μL assay volumes in 96-well PCR plates, with 12.5 μL of 2× master mix and 0.5 μL of the fluorescent dye provided with the kit for amplicon detection. Assay primers are added such that FIP and BIP primers are at a final concentration of 1.6 UM, F3 and B3 primers are at a final concentration of 200 nM, and loop primers are at a final concentration of 400 nM. A 2 μL sample aliquot is added for each reaction, and nuclease-free water is added to bring the final reaction volume to 25 μL.

Real-time amplification and fluorescent monitoring are carried out on QuantStudio5/6 Real-time PCR instruments (ThermoFisher). Assays are brought to 65° C. and the temperature is maintained throughout the duration of the assay (20-60 minutes depending on the application). Fluorescent readings are performed every 20 seconds; each 20 second increment is considered a “cycle,” although no temperature cycling takes place in the reaction. The time required to reach a predetermined fluorescent threshold is reported in terms of these cycle times, with each 20 second cycle considered as 1 “Threshold time” (Tt).”

f) Optimized qRT-LAMP

qRT-LAMP assays using an optimized formulation are carried out in 20 μL reaction volumes in standard 96-well PCR plates. The reaction mixture contains 5× assay buffer {250 mM Tris, 200 mM KCL, 100 mM (NH4) 2SO4, 0.5% Triton X-100, pH 8.3}, 8 mM MgSO4, 1 M Betaine, 1.4 mM dNTP mix, 4 μM SYTO9 dye (ThermoFisher), 8 U GspSSD2.0 polymerase (Optigene), and 2 U of WarmStart RTx reverse transcriptase (NEB). Assay primers are added such that FIP and BIP primers are at a final concentration of 1.6 UM, F3 and B3 primers are at a final concentration of 200 nM, and loop primers are at a final concentration of 400 nM. A 2 μL sample aliquot is added for each reaction, and nuclease-free water is added to bring the final reaction volume to 20 μL.

Real-time amplification and fluorescent monitoring are carried out on QuantStudio5/6 Real-time PCR instruments (ThermoFisher). Assays are brought to 65° C. and the temperature is maintained throughout the duration of the assay (20-60 minutes depending on the application). Fluorescent readings are performed every 20 seconds; each 20 second increment is considered a “cycle,” although no temperature cycling takes place in the reaction. The time required to reach a predetermined fluorescent threshold is reported in terms of these cycle times, with each 20 second cycle considered 1 “Tt.”

### g) In Vitro RNA Transcription (IVT)

IVT reactions are performed using the HiScribe T7 High Yield RNA Synthesis kit (NEB) per the manufacturer's protocol. Reactions are templated with 50 ng of synthetic, double-stranded DNA (dsDNA) obtained commercially from http://idtdna.com. Templates contain a Ty promoter sequence at the 5′ terminus of the sense strand, followed by 0.5-1.5 kB of sequence to be transcribed, and are provided blunt-ended. Reactions are allowed to proceed at 37° C. between 2-16 hours (overnight) in a forced air shaker/incubator. After transcription, RNA transcripts are purified from residual IVT material using the RNA Clean and Concentrator-5 kit (Zymo Research) per the manufacturer's protocol. RNA transcripts are eluted into 50 μL of nuclease-free water. Transcripts are quantitated using both the Qubit 4 Fluorimeter and UV/Vis spectroscopy.

h) SPRI-Based RNA Extraction

Rapid, centrifugation-free extraction of total RNA from a human whole blood sample stabilized in PAXgene Blood RNA tubes is carried out using the Agencourt RNAdvance Blood Kit (Beckman Coulter), with the protocol modified to exclude DNA removal steps. A 1.5 mL aliquot of stabilized blood sample is transferred to a 5 mL tube. 50 U of Qiagen Protease is added to the sample, followed by 1.2 mL of Agencourt Lysis reagent. Reagents are mixed by inversion, then incubated at 55° C. for 2 minutes. The sample is removed from heat, then 1875 μL of Bind 1 (SPRI beads)/Isopropanol solution {75 μL of Agencourt Bind 1 reagent, 1800 μL of 100% Isopropanol} is added. Reagents are mixed with the sample by pipetting thoroughly, then incubated for 1 minute at room temperature. A magnet is then applied to collect the SPRI beads, after which the supernatant is removed and discarded. The SPRI beads are resuspended in 800 μL of Agencourt Wash reagent and mixed by pipetting. A magnetic is applied to collect the SPRI beads and the supernatant is removed. This procedure is repeated for an additional 2 rounds of washing using 70% ethanol in place of the Agencourt Wash reagent. After washing is complete, bound nucleic acid is eluted by resuspending the SPRI beads in nuclease-free water. A magnet is applied to collect the beads, and the supernatant containing purified total RNA is removed and retained.

i) Prospective Collection of Clinical Infected Whole Blood Samples

Infected whole blood samples were prospectively collected as part of several clinical studies spanning multiple institutions. All samples were collected in PAXgene Blood RNA tubes per the manufacturer's protocol, then frozen, stored and shipped at −80° C.

### j) Acquisition of Control Healthy Whole Blood Samples

Healthy control sample sourcing Blood RNA tubes were prospectively collected from healthy controls (HC) through a commercial vendor (BioIVT) under IRB approval (Western IRB #2016165) using informed consent. Donors were verbally screened to have no inflammation, infection, illness symptoms, (including no fever or antibiotics within 3 days of sampling) or to be immunocompromised. All samples were tested and negative for HIV, West Nile, Hepatitis B, and Hepatitis C by molecular or antibody-based testing. The age (median and interquartile range (IR) was 36 (29-45.25) and was 70.8% male.

### k) Normalized Gene Expression Analysis and Fever Scorebacterial/Viral Metascore Calculation

A detailed description of how the diagnostic biomarkers were identified and clinical evidence for the utility of the diagnostic score have previously reported by Sweeney et al. (13). The simple Fever scorebacterial/viral metascore can be calculated by determining the geometric mean of abundance measurements for markers that are up-regulated as a result of bacterial infection (CTSB, GPAA1, HK3 and TNIP1) and subtracting the geometric mean of abundance measurements for markers that are up-regulated as a result of viral infection (IFI27, JUP, LAX1). In order to compare these scores across samples, abundance measurements made for these informative markers are input-normalized using abundance measurements made in parallel for transcripts of a housekeeping gene or genes-mRNA transcripts for which the abundance does not change as a function of infection. When multiple housekeeping genes are used for normalization, abundance measurements of informative biomarkers are normalized to the geometric mean of abundance measurements for all housekeeping genes as in Eq. 1:

$$Input Normalized Abundance = {A}_{i} - geomean ( {HK}_{i} , … , {HK}_{n} ) \qquad ( 1 )$$

where geomean (X) is the geometric mean of all arguments X, Ax represents the abundance measurement for the biomarker X, and HKx represents the abundance measurement of housekeeping gene X. Fever scorebacterial/viral metascores are determined by calculating the geometric mean of input normalized abundance measurements across all biomarkers up-regulated in bacterial infection and the geometric mean across all biomarkes up-regulated in viral infection, and then calculating the difference between these values (13), as in Eq. 2:

$$bacterial / viral metascore = \{ geomean [ {B}_{i} - geomean ( {HK}_{i} , … , {HK}_{n} ) ] , … , [ {B}_{n} - geomean ( {HK}_{i} , … , {HK}_{n} ) ] \} - \{ geomean [ {V}_{i} - geomean ( {HK}_{i} , … , {HK}_{n} ) ] , … , [ {V}_{n} - geomean ( {HK}_{i} , … , {HK}_{n} ) ] \} \qquad ( 2 )$$

Where geomean (X) is the geometric mean of all arguments X, Bx represents the abundance measurement of biomarker X which is upregulated upon bacterial infection, Vx represents the abundance measurement of biomarker X which is upregulated upon viral infection, and HKx represents abundance measurement of housekeeping gene X.

Both Tt values determined by qRT-LAMP and transcript “counts” determined by NanoString nCounter SPRINT serve as inputs for Fever Scorebacterial/viral metascore determination. However, because Tt measurements are made on an exponential scale relative to template input, whereas counts are made on a linear scale relative to template input, Tt measurements are best compared directly to the logarithm of measured counts, and Fever Scorebacterial/viral metascores determined using the same. All fever scorebacterial/viral metascores and measurement comparisons performed herein are based on the logarithm of nCounter SPRINT measurements.

Further, because it is impossible to determine a geometric mean of sets containing negative values, if the value determined for the geometric mean of housekeeper abundance is larger than the value determined for a given informative biomarker, the normalized geometric mean for that biomarker will be negative, and the Fever Scorebacterial/viral metascore cannot be calculated. To circumvent this complication, an arbitrary value of 10 is added to all normalized abundance measurements, which ensures all factors remain positive throughout the analysis.

### 4. Results

### a) Proof of Concept RT-LAMP Assay Development

We determined to leverage LAMP as a quantitative technology for relative gene expression analysis owing to the improved turnaround time and lower instrument complexity required relative to qRT-PCR-based technologies. We reasoned that a rapid, simple quantitative test system would enable host gene expression profiling near the point of care, making it possible to integrate such a diagnostic seamlessly with physicians' workflows.

In order to maximize the utility of host gene expression analyses for practical diagnostic applications, the ideal qRT-LAMP assay system will be designed to minimize turnaround time and maximize cost-effectiveness. We identified two means of optimizing standard LAMP technology toward these metrics: (i) to develop assays that are mRNA-specific (selective against genomic DNA amplification), obviating the need for DNA removal in upstream sample processing, thereby saving associated time and costs, and (ii) to specifically optimize the assay formulation to minimize the time to result without sacrificing technical precision. Two proof of concept targets from the diagnostic biomarker panel developed by Sweeney et al. (13), CTSB and IFI27, were selected as test cases for a first round of assay development.

LAMP amplification proceeds in two phases as diagrammed in FIG. 1A (27, 28, 36). In the first phase, a primary transcript is produced by amplification from one of the two “inner” primers, FIP/BIP, bound at F2/B2 sequences within the target. This is followed by sequential displacement of the product transcript by amplification from F3/B3 primers. The primary transcript serves as a template for a second round of amplification by the complimentary inner primer, BIP/FIP. The resulting transcript adopts a dumbbell secondary structure mediated by F1: Fic and B1: Bic sequence interactions. In the second, exponential phase of LAMP, the dumbbell template enables auto-amplification at the 3′ end of the transcript, amplification by primers binding at F2/B2 sequences, and through rate enhancing primers (LF/LB) that recognize sequences within the loop region of the secondary transcript. RT-LAMP requires an initial reverse transcription (RT) event to generate a primary DNA transcript from an RNA template.

In the system we report here, BIP primers serve as the site for RT initiation, as FIP primers are designed to correspond to sense strand sequences for each mRNA target. Because amplification from F2/B2 sites initiates productive transcript formation and must proceed through sequences falling between these sites, we reasoned that we could impart specificity for mRNA over genomic DNA in LAMP assays by designing primers such that exon: exon junctions fall either within the F2/B2 sequences of FIP/BIP primers, or between the F1 and B1 sequences if intervening introns are sufficiently large to prohibit read-through amplification. To generate proof of concept assays for CTSB and IFI27, target mRNA isoforms were selected based on measured tissue-specific abundance as reported by the Genotype-Tissue Expression project (GTEx). The highest abundance isoform in whole blood was selected as the target sequence for assay development. Regions of up to 500 bp that contain contain exon: exon junctions flanking introns of at least 1000 bp (where possible) were selected from within the target isoform sequences as inputs for assay design. These sequences were uploaded to the Primer Explorer V5.0 website (primerexplorer.jp/e/) and primer outputs were manually screened to identify solutions with exon: exon junction(s) falling in the desired region of primer or amplicon sequences.

Assay solutions comprising the four core LAMP primers—FIP, BIP, F3, and B3—were identified for CTSB and IFI27 mRNAs and screened for specificity by performing amplification reactions using the WarmStart LAMP kit (NEB) and commercially available human cDNA (Biosettia) and human genomic DNA (Genscript) as templates. A primer solution that selectively amplified cDNA was identified for each biomarker (FIGS. 1B, 1C). Primer Explorer software was then used to identify rate enhancing primer solutions for each core primer set, which were similarly screened and down-selected (all primer sequences are found in Table 3). The best complete primer sets-comprising core, LF and LB primers-were then tested for the ability to amplify mRNA transcripts from total RNA extracted from pooled human leukocytes (Takara), with both CTSB and IFI27 primer sets displaying successful amplification (FIGS. 1D, 1E). Having successfully developed proof of concept assays showing selective mRNA amplification, these assays were carried forward for use in qRT-LAMP formulation optimization.

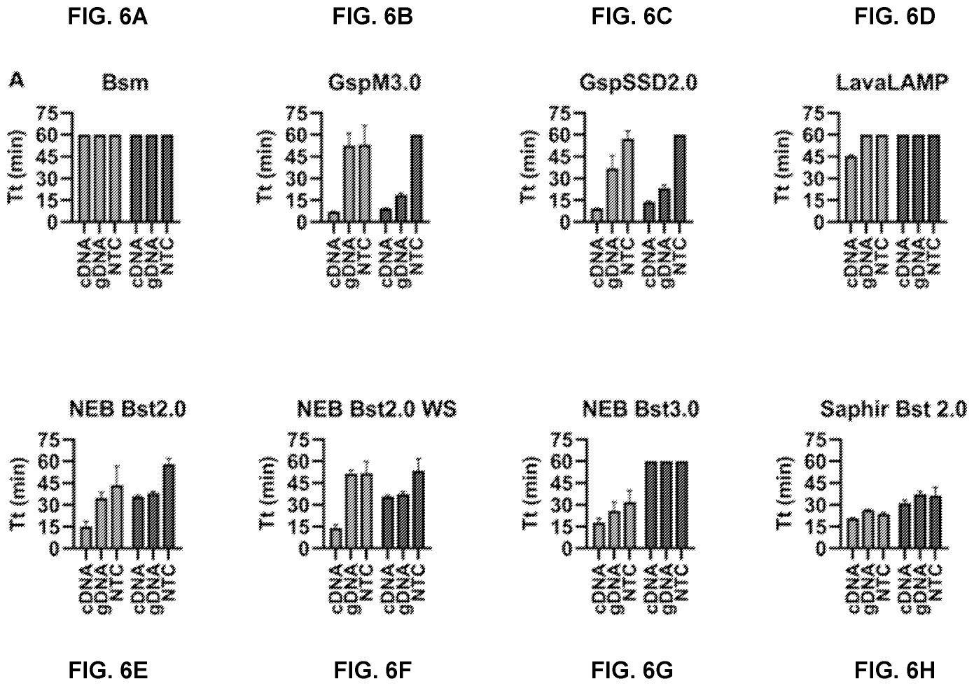

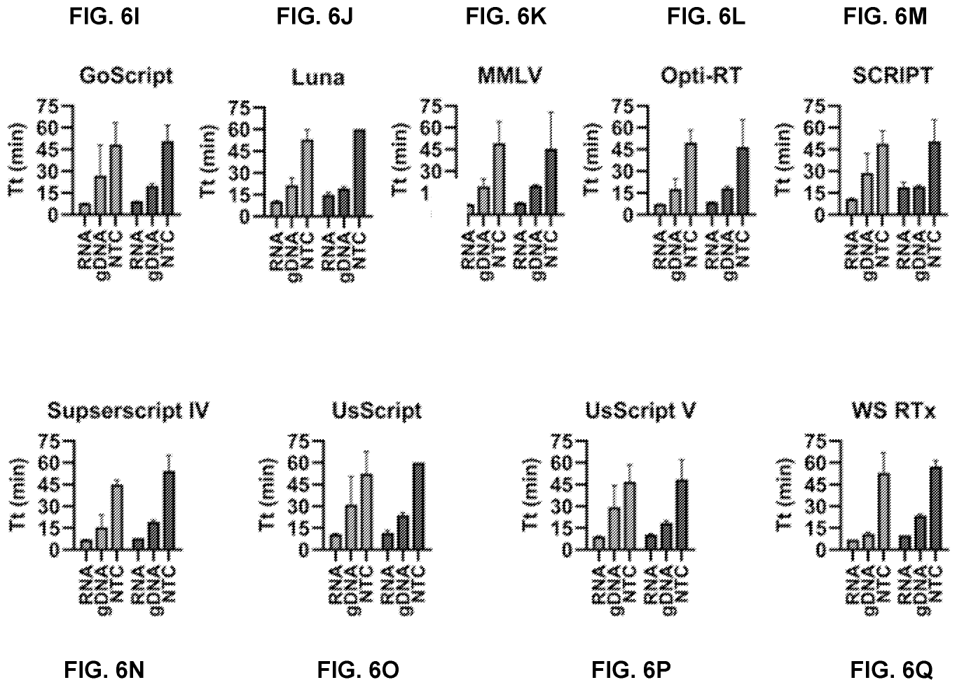

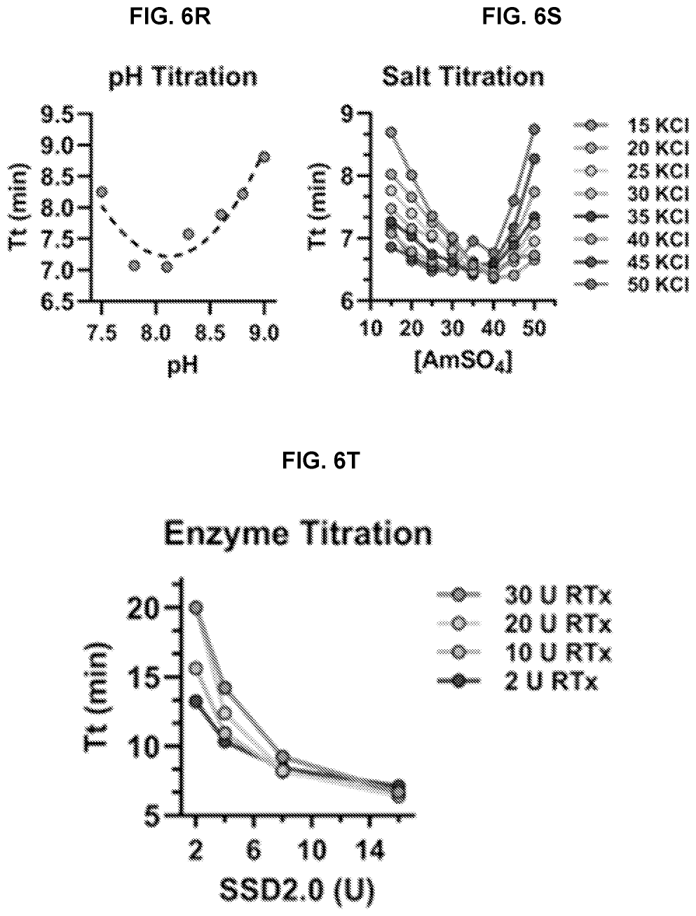

To optimize this formulation, a series of polymerase and reverse transcriptase enzymes were screened for best performance in terms of time to result, precision, and specificity (FIGS. 6A-6Q, Table 4). Based on this data, we selected the GspSSD2.0 polymerase (Optigene) and the WarmStart RTx reverse transcriptase (New England Biolabs) as a best performing enzyme pair. Using these enzymes, we further optimized the LAMP reaction chemistry formulation to improve performance (FIGS. 6R-6T, Table 4). In sum, we were able to decrease turnaround times from approximately 13 and 15 minutes for CTSB and IFI27 respectively, to about 7 to 10 minutes with no observed loss in precision.

b) Defining Performance Requirements for qRT-LAMP Assays

Prior to initiating screening and development of LAMP assays for the complete panel of host gene expression biomarkers, we defined a set of acceptance criteria to maximize the likelihood that these assays would provide sufficient accuracy and precision to discriminate between expression profiles associated with bacterial and viral etiologies.

Based on a target turnaround time of 30 minutes to enable point of care diagnosis with these assays, and to allow sufficient time for RNA extraction ahead of gene expression profiling, we allotted 15 minutes for the completion of isothermal amplification. Therefore, assays were required to achieve a time to threshold of <15 minutes as input approaches 0 template copies. We also aimed to eliminate the need for DNA removal as part of sample preparation, making it necessary for assays to be specific for mRNA. Ideally, assays should exhibit no amplification from a nominal amount of gDNA (10 ng) within the 15-minute assay duration, however assays that exhibit at least a 1000-fold preference for mRNA over gDNA were also be deemed acceptable, as the abundance of target biomarkers is not anticipated to be <1 transcript per cell (implying an expected ratio of mRNA to gDNA of ≥1:2). Assays should also exhibit no amplification in non-templated reactions within 15 minutes.

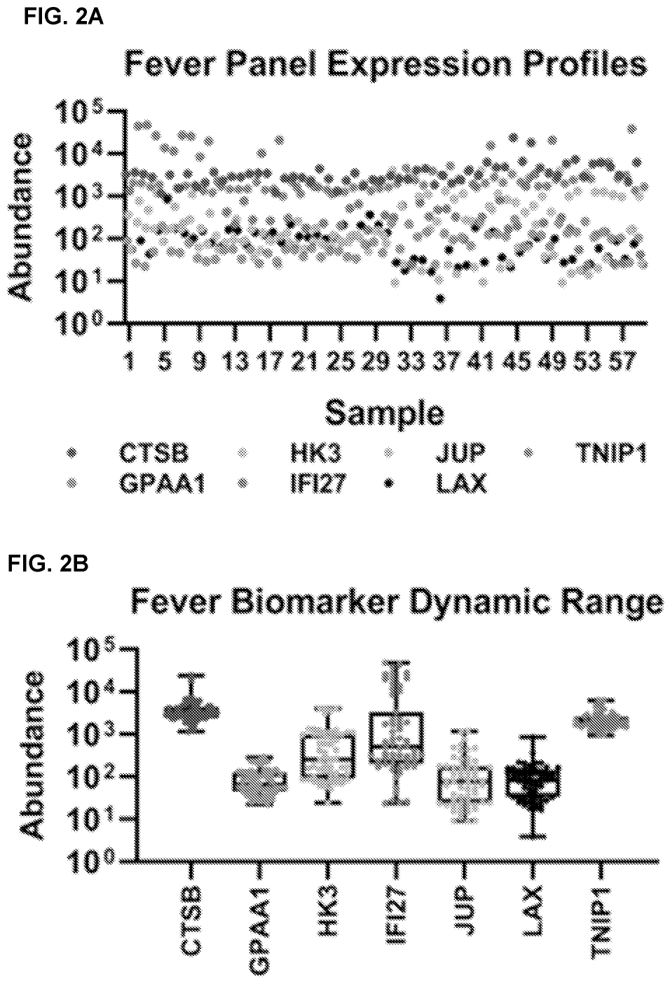

Assays must provide sufficient quantitative precision to discriminate between target abundance levels associated with each etiology. To estimate a precision threshold, we selected a cohort of 57 stabilized blood specimens for which gene expression data had already been collected through automated total RNA extraction followed by gene expression profiling by NanoString nCounter SPRINT, our gold standard (FIG. 2A). Samples were chosen to ensure effective representation of both infection classes—bacterial and viral infections—as well as healthy controls, and to capture transcript abundance levels near biological extrema. We defined the working dynamic range of abundance observed for each biomarker across all samples as the fold change between the 90th and 10th percentiles across the cohort (FIG. 2B). TNIP1 exhibited the smallest dynamic range at 2.90-fold; we therefore determined that assays must be able to reliably discern changes in mRNA abundance with at least 2.90-fold resolution.

We define the effective resolution (hereafter simply resolution) as the minimum fold-difference in template input levels for which the 95% confidence intervals of Tt measurements do not overlap. Using the standard definition for determining confidence intervals, assay resolution can be calculated as:

$$Resolution = {e}^{[ 2 * Z * ( σ / ] n ) ]} < 2.9 - fold$$

Where r≡amplification rate; the fold-change in amplicon generated per unit time, Z=1.96 for a 95% confidence interval, σ≡the standard deviation of the measurement, and n≡the number of replicate measurements performed. Assay variance (standard deviation anticipated for any measurement) is inferred by performing repeated measurements under conditions mimicking those expected for clinical samples. To determine r, it is necessary to determine the change in Tt per change in template input, which is accomplished by generating standard curves for each assay using serial dilutions of control material. The final acceptance criteria therefore depends on both the amplification rate and variability of each assay; all acceptance criteria can be found in Table 1.
c) Development and Characterization of qRT-LAMP Diagnostic Assays

Our optimized RT-LAMP formulation was used in the second round of design and screening to develop assays for the remaining five informative biomarkers-GPAA1, HK3, JUP, LAX1, and TNIP1—and a set of three housekeeping targets—KPNA6, RREB1 and YWHAB, and to search for alternative primer solutions for CTSB and IFI27 with increased mRNA specificity. Target mRNA isoforms were identified based on measured abundance in human whole blood as reported by GTEx, and sequence regions were down-selected such that exon: exon junctions could be incorporated into the amplicon as previously described.

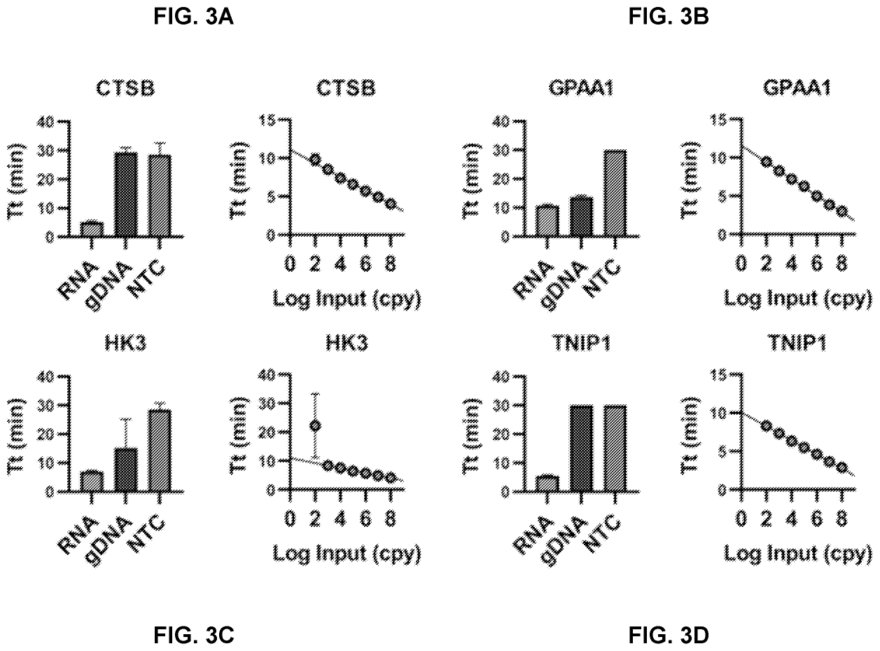

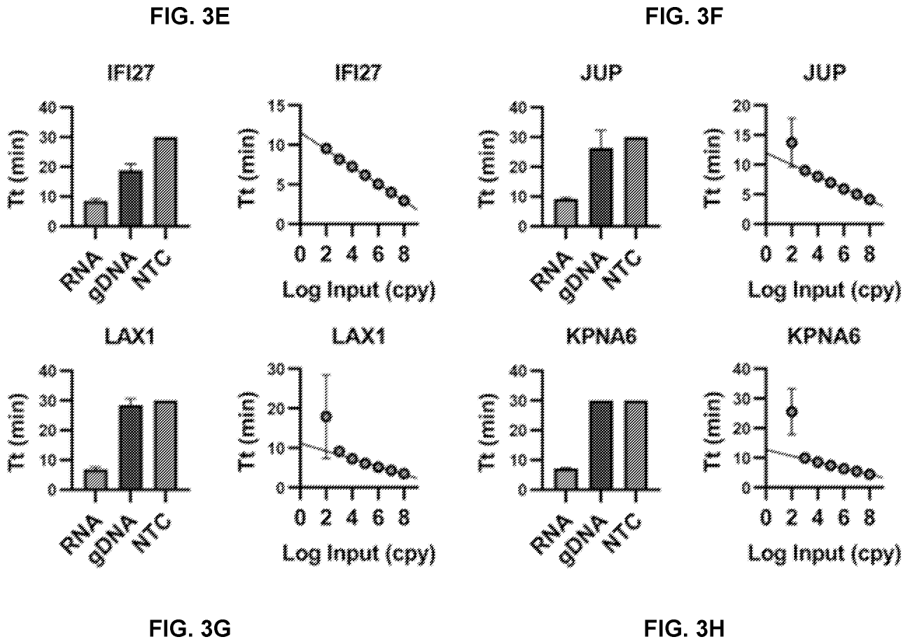

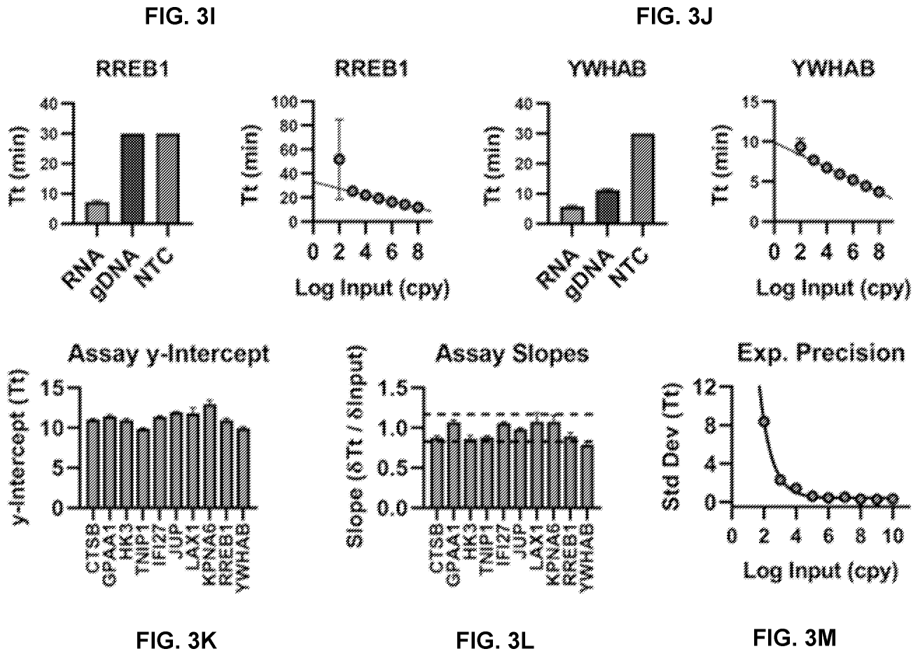

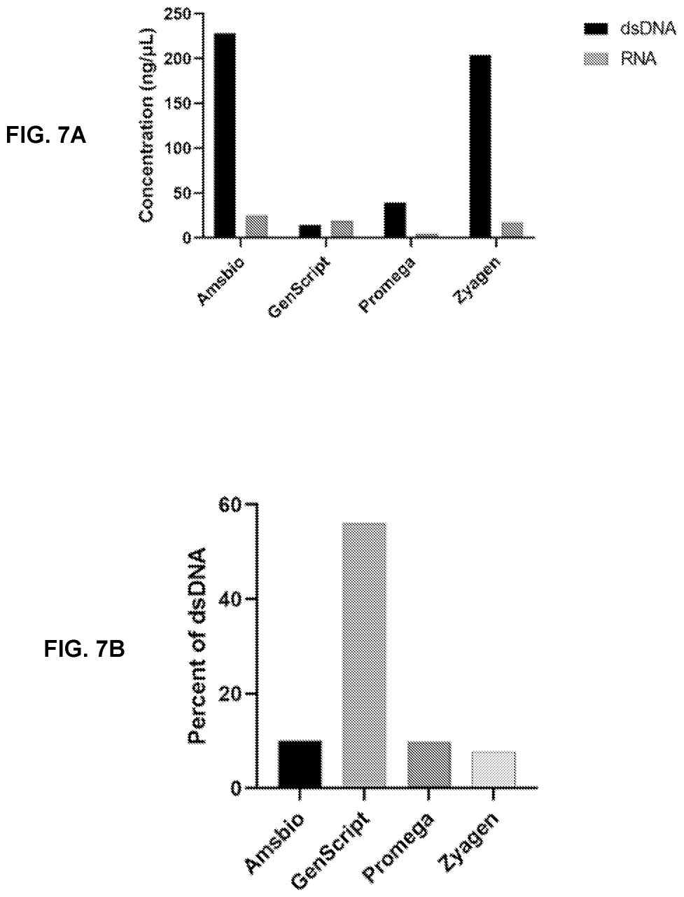

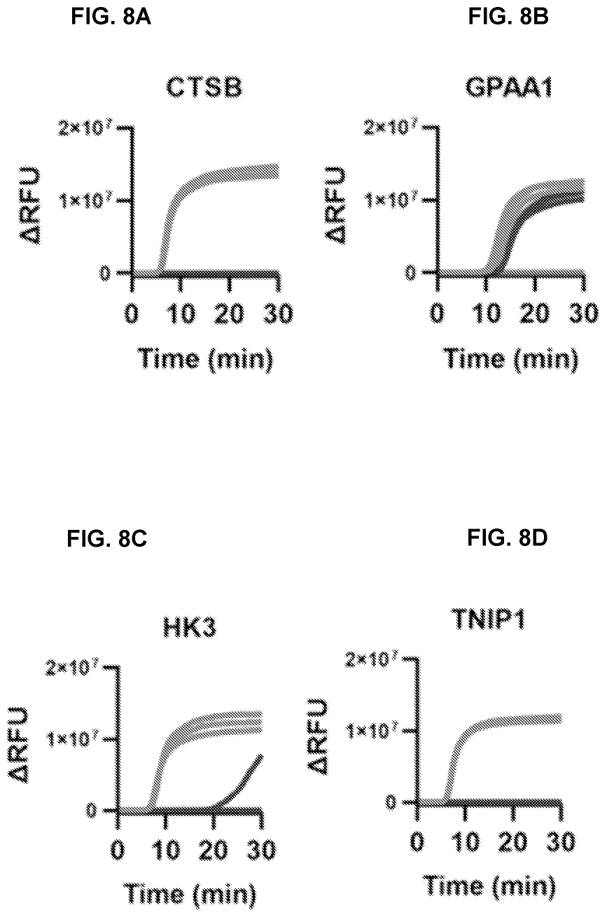

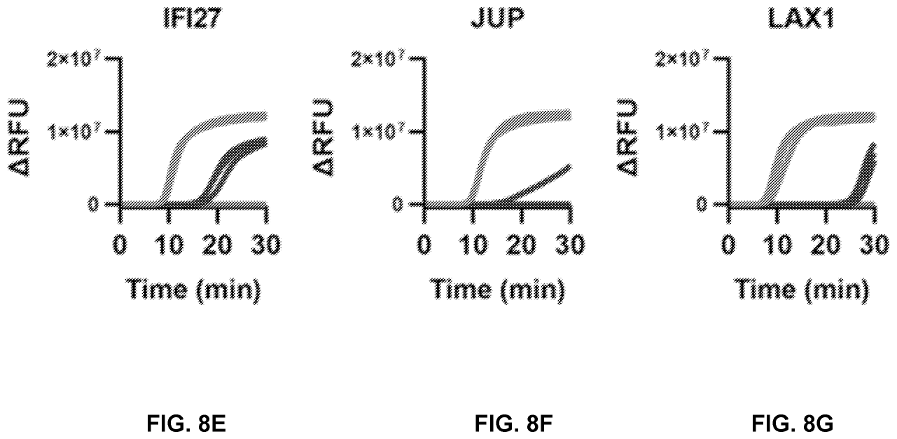

Complete primer sets-including core and rate enhancing primers-were screened against cDNA or gDNA templates, or in NTC's until at least one specific solution was identified (FIGS. 3A-3J, left panels). Intriguingly, we observed considerable variability in genomic DNA amplification times, and found that these differences were related to the vendor and lot of material used. We assayed commercial gDNA stocks for RNA contamination and found significant background RNA present in materials acquired from multiple vendors, even in material that had purportedly been subjected to enzymatic RNA degradation during preparation (FIG. 7). In an attempt to correct for sample-specific background amplification in genomic DNA samples caused by RNA contamination, we used the mean time to result across up to 3 different gDNA sample measurements acquired using material from different vendors. For most assays, the inconsistency of gDNA amplification across samples despite an identical total gDNA load indicated that the assays were likely not amplifying genomic material.

Even after correcting for residual RNA contamination, we were not able to identify a solution for GPAA1 with the desired specificity for mRNA, as introns within the genomic sequence are short and readily permit “read through” amplification of genomic sequences. Further, FIP/BIP sequences could not be successfully positioned over exon: exon junctions due to thermodynamic constraints of primer design. We therefore selected and proceeded with the best solution that was identified, although it did not meet our acceptance criteria for specificity.

For best assays emerging from screening, standard curves were generated using in vitro transcribed (IVT) RNA transcripts specific to each biomarker as the titrated template. Transcripts were evaluated from 1×10⁸ to 1×10² copies per reaction (FIGS. 3A-3J, right panels). A linear fit was modeled for each assay between the highest input copy number and the next highest input level, then extending through each input level thereafter down to 10². Each assay demonstrated a log-linear relationship between input copy number and Tt over the majority of input levels tested. We define the limit of quantitation (LoQ) as the input level at which the Re for the fit falls below 0.95. By this method, a LoQ of 10³ copies per well was determined for most assays, although CTSB, GPAA1 and IFI27 demonstrated LoQ's of 10² copies per well or fewer. Because we cannot measure the absolute abundance of individual transcripts within a total RNA sample, the Tt value determined at the LoQ (Tt_{LoQ}) serves as a benchmark for whether the amount of template in a reaction falls within the quantitative dynamic range for the assay; e.g. measured Tt<Tt_{LoQ}. Importantly, the linear models for all assays predict Tt≤12 min as input approaches 0 copies (represented by the y-intercept), indicating that any quantitative measurement will be complete within 12 minutes, easily meeting our target turnaround time (FIG. 3K). The slopes of linear fits (representing the change in Tt per unit time) were also determined for each assay (FIG. 3L) and used to calculate amplification rates (fold-change in amplicon per unit time) to enable calculation of assay resolutions using Eq. 3 (Table 2).

Finally, we evaluated the precision of assays for informative biomarkers by performing repeated measurements across three independent experiments. We and others have observed that assay variance is related to the input copy number (37, 38), with variance increasing dramatically near the LoQ. Therefore, assay precision was assessed across three independent trials for 10 template input concentrations ranging from 1010 to 101 copies per reaction (Table 5). Below the LoQ, measurements become highly variable, and frequently exhibit no amplification at all. This complicates determination of variance because as a higher proportion of measurements yield no amplification, all imputed Tt's approach 90, yielding lower and lower variability. Therefore, to assess variance as a function of RNA input, measurements where no amplification was observed or where amplification was observed in only a single replicate were excluded. All assays exhibited similar levels of precision within the quantitative dynamic range, with mean standard deviations across quantitative measurements ranging from 0.16 Tt to 0.58 Tt. The mean standard deviation across all assays is plotted as a function of template input in FIG. 3M.

Using these data in conjunction with the previously determined amplification rates, we calculated theoretical assay resolutions within the quantitative dynamic range using Eq. 3, and determined that all assays were theoretically capable of resolving abundance differences within the biologically relevant dynamic range for each biomarker (Table 2). We next turned to evaluating empirical assay performance in banked clinical samples.

d) qRT-LAMP Host Gene Expression Analysis in Stabilized Whole Blood

The performance of qRT-LAMP assays was assessed using the cohort of 57 stabilized blood specimens for which data had already been collected using gold standard methods (FIG. 3A). To enable gene expression profiling on a time scale that would be relevant for point of care diagnostics, a rapid total RNA extraction procedure was employed for use with qRT-LAMP, instead of the more costly, time intensive procedure used as our gold standard. The rapid protocol comprises a version of the Agencourt RNAdvance kit (Beckman-Coulter) in which the DNase treatment step has been removed (Materials and Methods), enabling completion of the protocol in approximately 15 minutes. Total RNA was extracted from aliquots of the banked clinical samples and split evenly across all assay wells, with resulting inputs ranging from about 8 ng to 38 ng of total RNA per reaction.

qRT-LAMP measurements were collected in triplicate for each biomarker for each sample, and the mean and standard deviation was determined across replicates. Mean abundance measurements for informative biomarkers and housekeeping genes were then used to calculate input normalized measurements for the informative biomarkers (see Materials and Methods).

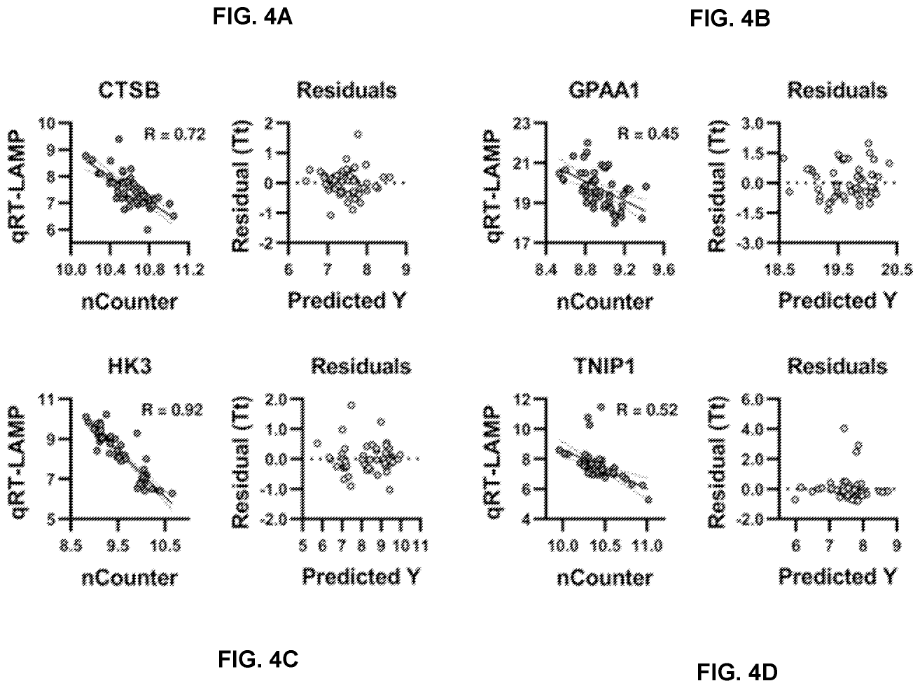

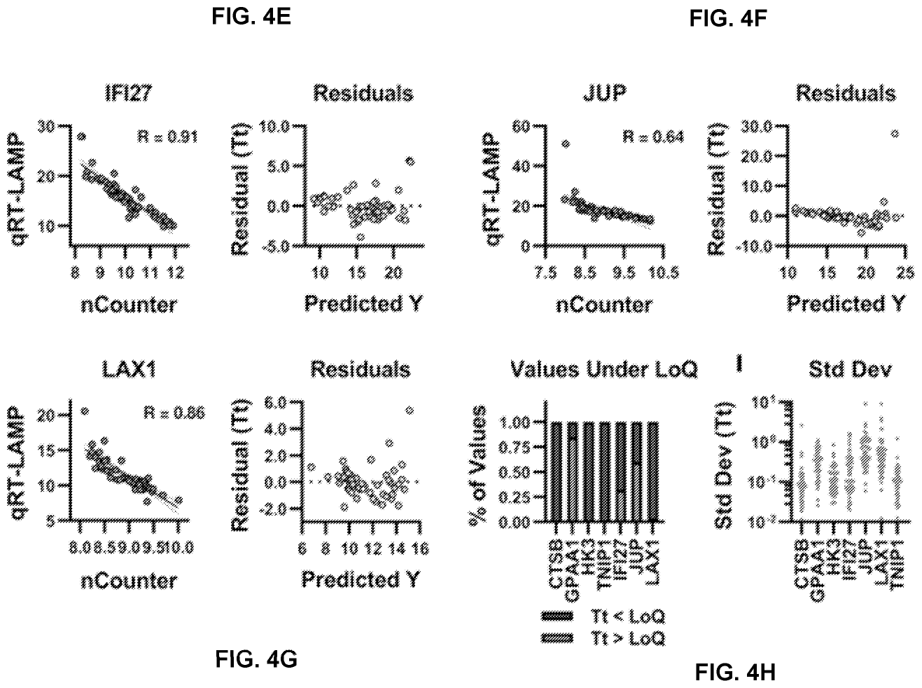

To assess the accuracy of qRT-LAMP relative to our gold standard, we determined the Pearson correlation coefficient at the level of individual assays between normalized abundance measurements made using each approach (FIGS. 4A-4G, left panels). While several assays were well correlated with the reference, coefficients for TNIP1 and GPAA1 were low at 0.45 and 0.52 respectively, and coefficients for JUP and CTSB were intermediate, at 0.62 and 0.71 respectively.

We therefore proceeded to evaluate potential root causes for variable correlations observed at the assay level with the goal of developing generalizable strategies to improve qRT-LAMP performance in gene expression profiling. As a first step, we considered the effect of outliers on overall performance. To define outliers, we determined linear fits describing the relationship between normalized qRT-LAMP and nCounter measurements for each assay, then calculated residuals associated with each measurement (FIGS. 4A-4G, right panels). The mean (μres) and standard deviation (σres) of residual distance was then determined for each assay, and outliers were defined as measurements with residuals >μres+/−2σres. When outliers were removed from consideration for TNIP1 and JUP, correlations of both markers improved to 0.85, indicating excellent concordance with the gold standard. Global performance of the remaining biomarkers is not significantly affected by outliers; most assays only show an incremental (0.01-0.03) improvement in correlation coefficient when outliers are not considered. We therefore considered other explanations for the poor (0.45) and intermediate (0.72) correlations observed for GPAA1 and CTSB respectively.

Imprecision in qRT-LAMP measurements increases near the limit of quantitation, and it is possible that total insufficient RNA inputs resulted in a significant proportion of measurements falling outside the quantitative dynamic range for a given assay. Indeed we found this to be the case for GPAA1, for which 85% of measurements exhibited Tt>TtLoQ (FIG. 4H). However, we expect measurements made near or below LoQ to exhibit significant variability across replicates (Table 5), but we observed a standard deviation of <1 Tt for 98% of GPAA1 measurements, with a median and range of standard deviations comparable to those observed for IFI27, JUP and LAX1, all of which demonstrated superior accuracy. We therefore do not favor technical imprecision as a principal failure mode for GPAA1. Based on the high frequency of measured Tt's falling outside the LoQ, it is possible that there are so few copies of GPAA1 template present in the range of total RNA inputs tested that sampling error plays a role in discordance with the gold standard, but this explanation is not entirely satisfying given the observed precision. Nevertheless, it is probable that maintaining total RNA input >8 ng would favor a further increase in precision and minimize potential sampling error and associated outlier measurements.

The range of abundance levels of CTSB and GPAA1 templates is compressed relative to most other markers in this cohort. The majority of samples fall within a 7-fold window of abundance of GPAA1, and a 3-fold window for CTSB, which is nearing the resolution limit of the assay (FIG. 2B). However, the majority of samples for TNIP1 also fall within about a 3-fold window, and we have seen that this assay is highly accurate for most samples. Further, while GPAA1 may be subject to sampling error, this is unlikely to be the case for CTSB, which exhibited no measurements with Tt<TtLoQ. We therefore note that, while the concordance of a given assay is likely to decrease as the range of values measured approaches the resolution of the assay, there is no reason to believe that 3- or 7-fold discrimination is beyond the capability of qRT-LAMP technology.

A key difference between these three assays may be the susceptibility to interference by genomic DNA. We retrospectively evaluated the gDNA content of a subset of the 57 samples tested, and found that as much as 14-fold more gDNA than total RNA was recovered by mass (Table 6). As noted above, we were unable to identify a primer solution for GPAA1 that was fully selective against 10 ng of genomic DNA, and other assays may be subject to interference from gDNA present at sufficiently high concentrations. We tested this hypothesis by evaluating amplification by each assay using 200 ng of genomic DNA, and found that GPAA1 demonstrated robust amplification, CTSB amplified the material in 1 of 3 replicates, and TNIP1 showed no amplification whatsoever. While not conclusive, these results support the hypothesis that assays do exhibit differential susceptibility for gDNA interference, which would likely contribute to discordance relative to the gold standard.

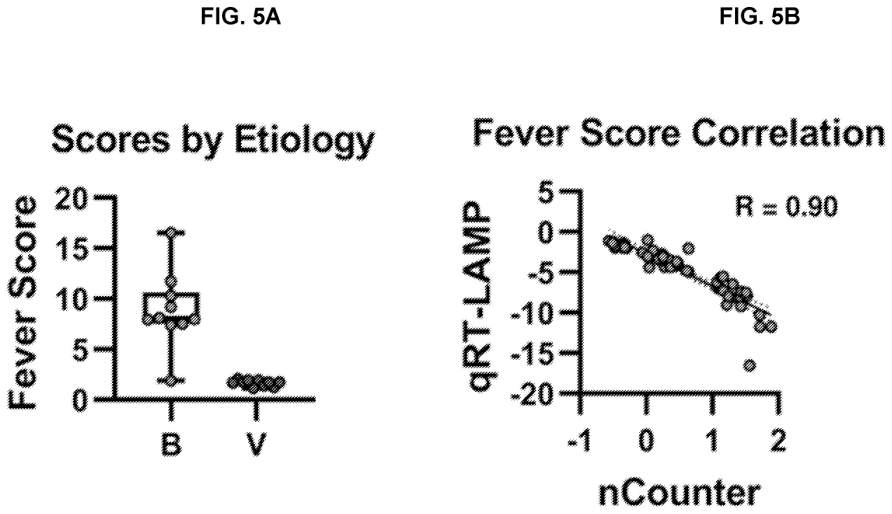

Despite the challenges observed at the level of individual assays, we calculated Fever Scorebacterial/viral metascores for each sample using the normalized abundance measurements made by each technology (see Materials and Methods). We found that Fever Scorebacterial/viral metascores determined using qRT-LAMP measurements cluster well and exhibit minimal overlap between the two infection classes (FIG. 5A), indicating that the assays are sufficiently accurate to discriminate between etiologies in the cohort tested. Further, we observed excellent correlation between Fever Scorebacterial/viral metascores generated by qRT-LAMP and nCounter, with a Pearson coefficient of 0.90 (FIG. 5B), indicating excellent diagnostic agreement between the two technologies. This demonstrates the sufficiency of assay performance in terms of diagnostic accuracy, and also shows the robustness of the diagnostic algorithm, as several qRT-LAMP assays do not demonstrate a degree of accuracy that we would deem exceptional when considered independently.

We therefore conclude that qRT-LAMP assays can provide sufficiently accurate gene expression profiling data to enable discrimination between bacterial and viral etiologies using an established set of biomarkers and classification algorithm. We posit that experimental and/or sampling error were likely the source of a very few outlier measurements that artificially deflated the apparent accuracy of JUP and TNIP1 assays, although verification of this hypothesis will need to be achieved through further rounds of testing. The range of total RNA inputs tested here may not provide sufficient material to ensure all measurements are performed within the quantitative range of the assays, but evidence for this is equivocal as observed precision is consistent with measurements made within the LoQ. Resolution limits may be challenged in attempting to discriminate between the many abundance levels that fall within a smaller window of the global dynamic range for CTSB and GPAA1. Finally, although most assays are highly selective against genomic DNA amplification, the presence of gDNA still has an impact on assay performance, especially at very high concentrations.

### 5. Discussion

In this work, we developed a rapid workflow for host gene expression analysis leveraging loop-mediated isothermal amplification technology with assay designs and formulation specifically tailored to the purpose. We showed that by selectively designing LAMP assays to incorporate exon: exon junctions within F2/B2 primer sequences or within the target amplicon, it is possible to achieve specificity for RNA over genomic DNA. By screening and selecting best performing enzymes and then performing a modest optimization of buffer components, we were able to achieve a theoretical maximum turnaround time of 12 minutes for all assays, while maintaining sufficient precision to achieve theoretical resolutions of 1.2-to 2.2-fold differences in target abundance. Finally, we demonstrated that most of these assays demonstrated good accuracy relative to an amplification-independent gold standard, and that the diagnostic scores developed by these assays were in excellent agreement with the reference technology.

For a diagnostic assay informing on infectious etiology to be useful in outpatient settings, it must be complete within 30 minutes. Further, it is beneficial that the assay technology be chosen to minimize cost and complexity not only of the assay itself, but of the equipment that will be required to execute the test. Selecting an isothermal amplification technology means that any instrument designed to execute the assay will not require costly apparatus to enable thermal cycling. And since we have demonstrated that the assays are accurate even when performed in parallel reactions, there is no need for multi-wavelength detection or expensive fluorescent probes. Further, by achieving a <12-minute time to result with the qRT-LAMP assays, we allow up to 18 minutes for upstream sample preparation while still meeting the 30 minute overall turnaround time. To our surprise, we were unable to identify a study reporting single-reaction RT-LAMP assays that had been developed with inherent specificity for host mRNA to the exclusion of genomic DNA. We were gratified to find that LAMP assays can be designed with a strong preference for mRNA using the same rationale as in qPCR. Leveraging this finding, we reduce the sample preparation burden by removing the time-consuming process of DNA degradation, and indeed we demonstrate that this process is not necessary to achieve good accuracy for diagnostic outcomes relative to our reference standard.

Within this study, we have evaluated biomarkers with dynamic expression levels spanning from 3-fold to several orders of magnitude. We determined that our assays were theoretically capable of resolving template abundance levels falling near the observed extrema. Because assay resolution is a function of both precision and reaction rate, we hypothesize that it is possible to exchange assay speed for higher resolution by lowering LAMP primer concentrations to reduce reaction efficiency.

We report here that increased polymerase and reverse transcriptase input can result in Tt's 30-45 seconds earlier than those reported for the clinical samples. We have nevertheless shown a high correlation-0.90-between diagnostic scores generated by qRT-LAMP and an established gold standard, which demonstrates the considerable potential for ultra-rapid, point of care applications leveraging this technology.

TABLE 1
List of acceptable performance criteria for down-selection of RT-LAMP assays during
screening and development. The key assay attributes to be evaluated are listed
under Performance Characteristics, with the accompanying Metrics to be measured
and and Acceptable Range of outputs for an assay. A short description of the rationale
for each Metric and Acceptable Range is provided in the last column.

| Performance | | | |
|---|---|---|---|
| Characteristic | Metric | Acceptable Range | Rationale |
| Speed | Tt at fixed RNA input | Tt < 15 min @ 10 ng RNA | Ensure assays can achieve |
| | | input | amplification within time |
| | | | constraints imposed by the |
| | | | proposed application |
| Specificity | Tt at fixed gDNA input | Tt > 15 min @ 10 ng genomic | Ensure an approximate |
| | | DNA input, or Tt delayed by 3 | 1,000-fold selectivity |
| | | min for gDNA relative to 10 ng | against gDNA carryover |
| | | total RNA | |
| Specificity | Tt at no template input | Tt > 15 min @ 0 ng nucleic | Ensure minimal primer |
| | | acid input | dimer or other non- |
| | | | templated amplification |
| Resolution | Smallest fold-difference | r < dynamic range of | Ensure assays can |
| | in RNA inputs for | abundance for target | discriminate between |
| | which 95% CI's of | biomarkers | transcript abundance |
| | measurements | | levels falling within a |
| | do not overlap | | biologically relevant |
| | | | dynamic range |

TABLE 2
List of parameters for calculation and calculated value of assay
resolution for all informative biomarkers comprising the Fever diagnostic
panel. The Biomarker is listed along with the accompanying Slope
determined by linear fit to a standard curve generated using a serial
dilution of input control material and the Mean Standard Deviation
observed across all measurements performed at input levels falling
within the quantitative dynamic range for the assay. The final column
lists the theoretical Resolution of the assay calculated using the
values in the second and third columns.

| | Slope | Mean Standard | Resolution |
|---|---|---|---|
| Assay | (δTt/δInput) | Deviation (Tt) | (Fold) |
| CTSB | 0.887 | 0.125 | 1.5 |
| GPAA1 | 1.087 | 0.168 | 1.6 |
| HK3 | 0.874 | 0.128 | 1.5 |
| TNIP1 | 0.916 | 0.240 | 2.0 |
| IFI27 | 1.082 | 0.245 | 2.1 |
| JUP | 0.992 | 0.145 | 1.5 |
| LAX1 | 0.976 | 0.068 | 1.2 |

TABLE 3
List of best oligonucleotide primers for qRT-LAMP detection of informative biomarkers and
housekeeping genes.

| Marker | Primer ID | Primer Sequence |
|---|---|---|
| CTSB | CTSB_27_FIP | cggccatgatgtccttctcgcaacaggacaagcactacgga |
| CTSB | CTSB_27_F3 | tctgtgagcctggctacag |
| CTSB | CTSB_715_BIP | acaaaaacggccccgtggagacgtgttggtacactcctga |
| CTSB | CTSB_715_B3 | catggccacccatcatctc |
| CTSB | CTSB_LF_27-1 | atgttaaggatgtcgcagaggt |
| CTSB | CTSB_LB_715-1 | ggagctttctctgtgtattcgg |
| GPAA1 | GPAA1_23-1 FL | ccccgacttcttgcggt |
| GPAA1 | GPAA1_23-1 BL | gcagagtttctcccggaaac |
| GPAA1 | GPAA1_23 F3 | gtggaggagcagtttgcg |
| GPAA1 | GPAA1_23 B3 | ttggtgcccgacaccata |
| GPAA1 | GPAA1_23 FIP | gttcaagccaggccactggccttttgcccgggacttcg |
| GPAA1 | GPAA1_23 BIP | gatgcggtcagtagggctggacgctcgtgggtctcatct |
| HK3 | PD HK3v4 F3 | acctgaggagagtgactagcttct |
| HK3 | PD HK3v4 FL | ccgcaaccctgaagaccca |
| HK3 | PD HK3v4 BL | gcagttcaaggtgacaagggcac |
| HK3 | PD HK3v4 B3 | gcctgctccatggaacccaaga |
| HK3 | PD HK3v4 FIP | tcagagcaactcagggtttcttccccactgtggaagctcatggac |
| HK3 | PD HK3v4 BIP | tcagagctggtgcaggagtgcgctggcttggatctgctgtagc |
| IFI27 | IFI27_64_FIP | tgctcccagtgactgcagagtaattgccaatgggggtgga |
| IFI27 | IFI27_65_BIP | tgcgaggttctactagctccctttctcccctggcatggtt |
| IFI27 | IFI27_64_F3 | agcagccaagatgatgtcc |
| IFI27 | IFI27_65_B3 | gatagttggctcctcgctg |
| IFI27 | IFI27_64_LF-4 | tgggtctgccattgcgg |
| IFI27 | IFI27_65_LB-1 | ccctcgccctgcagagaaga |
| JUP | PD JUPv9 F3 | accccaagttcctggccatc |
| JUP | PD JUPv9 B3 | tcccaccagcctccacaatg |
| JUP | PD JUPv9 FL | gatcagcttgctctcctggtt |
| JUP | PD JUPv9 BL | accaccagtcgtgtgctcaag |
| JUP | PD JUPv9 FIP | gatctgcacgagggccttgcagctcctggcctac |
| JUP | PD JUPv9 BIP | atgcgtaactacagttatgaaaagctgcgcttattgctgggacacacggatag |
| KPNA6 | PD KPNA6v6 FL | atttgagccctgttgccagcagta |
| KPNA6 | PD KPNA6v6 BL | cagggcaggagaagccactttgta |
| KPNA6 | PD KPNA6v6 B3 | ccacttgttgagcagtcccaagga |
| KPNA6 | PD KPNA6v6 FIP | agtgacgatgttacccacggctctattggtagagctgctgatgcacaa |
| KPNA6 | PD KPNA6v6 BIP | tcttaactgttcagccctaccttgagtccagcaagcttccttccggat |
| LAX1 | PD LAX1v9 F3 | gaaataaagaccagatcaccaacatctt |
| LAX1 | PD LAX1v9 B3 | gaggaggctctcagtactgaaaat |
| LAX1 | PD LAX1v9 FL | gtcgcttcttccgtttattccaat |
| LAX1 | PD LAX1v9 BL | agccaaaaatatttatgacatcttgcct |
| LAX1 | PD LAX1v9 FIP | gcatgacggtaactcggagcgttgcggttttctgcatc |
| LAX1 | PD LAX1v9 BIP | tgactttgccacaaaccagacactcatgtctccccaggtctt |
| RREB1 | PD RREB1v7 F3 | gccattttgattccttttccggaacaagt |
| RREB1 | PD RREB1v7 FL | cggagtagaaaatgagtctgtgttgacctctt |
| RREB1 | PD RREB1v7 BL | ctccctggcatgatgcgttgg |
| RREB1 | PD RREB1v7 B3 | gccaggttcagccccccaata |
| RREB1 | PD RREB1v7 FIP | acacagtcggagcaacggccctcctcggtctctccctgaagc |
| RREB1 | PD RREB1v7 BIP | gttccaggagtggtggctctgagactgttttctttgtgttatcaagctgccc |
| TNIP1 | PD TNIP1v21-1 F3 | ggatcagctgagcccact |
| TNIP1 | PD TNIP1v21-1 FL | ccgctggatctccttttcctg |
| TNIP1 | PD TNIP1v21-1 BL | caacagcatttgggagcccag |
| TNIP1 | PD TNIP1v21-1 B3 | cagcaactcattctgcgtga |
| TNIP1 | PD TNIP1v21-1 FIP | gtgcttcctccagggccttgacccgacagcgtgagtac |
| TNIP1 | PD TNIP1v21-1 BIP | ccaaaccccgccatcatctccccagctcctgtttccttagg |
| YWHAB | PE YWHABv145 | tgcatgatcagagtgctgtctttataaaacggcatttgatgaagc |
| | FIP | |
| YWHAB | PE YWHABv145 F3 | ctgaaaaggcctgtagcc |
| YWHAB | PE YWHABv145 | ctgtggacatcggaaaaccagtcacaaagcacgagaaaca |
| | BIP | |
| YWHAB | PE YWHABv145 B3 | cagagtgacactgaacaga |
| YWHAB | PE YWHABv145-1 | tcagcgtatccaattcagcaat |
| | FL | |
| YWHAB | PE YWHABv145-1 | gagacgaaggagacgctggg |
| | BL | |

TABLE 4
Calculated mean and standard deviation of Tt values measured
in n = 4 replicates of RT-LAMP reactions using proof of
concept CTSB and IFI27 assays along with various polymerase
and reverse transcriptase enzymes against positive and negative
control templates as listed. Reactions were carried out in
20 μL volumes with 2 μL template input at concentrations
such that cDNA, total RNA, and genomic DNA were present at
10 ng per reaction, 8 mM Magnesium Acetate, 1.4 mM dNTP's,
4 μM SYTO9 nucleic acid stain. For polymerase screens, master
mixes were formulated using buffers and additives provided
by the enzyme manufacturer at concentrations recommended in
the accompanying instructions for use. Reverse transcriptase
enzymes were screened in the context of the GspSSD2.0 master
mix formulation recommended by Optigene Ltd.

| | | CTSB | | IFI27 | |
|---|---|---|---|---|---|
| Template | Enzyme | Mean | Std Dev | Mean | Std Dev |

Polymerase Screen

| | | | | | |
|---|---|---|---|---|---|
| cDNA | GspM3.0 | 21.6 | 0.2 | 27.8 | 0.8 |
| | GspSSD2.0 | 28.0 | 0.2 | 41.2 | 1.7 |
| | Sahpir | 62.0 | 0.7 | 93.4 | 5.3 |
| | Bsm | 180.0 | 0.0 | 180.0 | 0.0 |
| | NEB Bst2.0 WS | 45.0 | 9.7 | 107.2 | 2.6 |
| | NEB Bst2.0 | 41.7 | 6.1 | 107.2 | 3.0 |
| | NEB Bst3.0 | 53.0 | 7.7 | 180.0 | 0.0 |
| | LavaLAMP | 136.2 | 2.0 | 180.0 | 0.0 |
| gDNA | GspM3.0 | 158.2 | 21.3 | 55.8 | 3.2 |
| | GspSSD2.0 | 110.7 | 23.0 | 69.7 | 5.6 |
| | Sahpir | 79.3 | 1.1 | 112.4 | 5.3 |
| | Bsm | 180.0 | 0.0 | 180.0 | 0.0 |
| | NEB Bst2.0 WS | 104.1 | 10.2 | 114.0 | 3.4 |
| | NEB Bst2.0 | 154.7 | 5.7 | 112.4 | 4.0 |
| | NEB Bst3.0 | 77.6 | 15.8 | 180.0 | 0.0 |
| | LavaLAMP | 180.0 | 0.0 | 180.0 | 0.0 |
| NTC | GspM3.0 | 159.6 | 34.1 | 180.0 | 0.0 |
| | GspSSD2.0 | 171.9 | 14.0 | 180.0 | 0.0 |
| | Sahpir | 71.1 | 2.7 | 109.0 | 14.5 |
| | Bsm | 180.0 | 0.0 | 180.0 | 0.0 |
| | NEB Bst2.0 WS | 131.0 | 33.3 | 174.2 | 10.0 |
| | NEB Bst2.0 | 155.5 | 21.0 | 160.4 | 21.1 |
| | NEB Bst3.0 | 95.8 | 20.8 | 180.0 | 0.0 |
| | LavaLAMP | 180.0 | 0.0 | 180.0 | 0.0 |

Reverse Transcriptase Screen

| | | | | | |
|---|---|---|---|---|---|
| RNA | Opti-RT | 22.1 | 0.1 | 26.4 | 0.1 |
| | RTx | 20.9 | 0.1 | 30.1 | 0.1 |
| | Script | 32.5 | 1.4 | 57.0 | 8.5 |
| | Luna | 30.5 | 2.0 | 44.7 | 4.3 |
| | UsScriptV | 27.2 | 0.9 | 31.2 | 1.9 |
| | SSIV | 21.1 | 0.2 | 24.2 | 0.1 |
| | GoScript | 23.7 | 0.6 | 27.7 | 0.1 |
| | MMLV | 21.7 | 0.1 | 25.3 | 0.1 |
| | UsScript | 32.2 | 1.0 | 34.8 | 4.5 |
| gDNA | Opti-RT | 53.4 | 17.9 | 55.3 | 2.6 |
| | RTx | 32.7 | 2.9 | 70.3 | 2.8 |
| | Script | 86.6 | 35.1 | 58.4 | 2.0 |
| | Luna | 64.7 | 13.0 | 57.3 | 3.5 |
| | UsScriptV | 88.3 | 38.7 | 55.7 | 4.0 |
| | SSIV | 46.6 | 22.3 | 57.8 | 2.7 |
| | GoScript | 80.7 | 54.5 | 59.5 | 3.8 |
| | MMLV | 58.3 | 14.0 | 59.8 | 1.7 |
| | UsScript | 93.5 | 50.6 | 71.4 | 4.5 |
| NTC | Opti-RT | 149.5 | 21.6 | 140.3 | 48.3 |
| | RTx | 159.2 | 36.0 | 172.4 | 10.4 |
| | Script | 146.9 | 22.8 | 152.1 | 38.1 |
| | Luna | 159.0 | 17.2 | 180.0 | 0.0 |
| | UsScriptV | 141.1 | 30.0 | 145.6 | 35.1 |
| | SSIV | 135.2 | 7.2 | 162.8 | 28.3 |
| | GoScript | 145.2 | 38.9 | 152.5 | 27.9 |
| | MMLV | 148.1 | 38.4 | 136.6 | 65.3 |
| | UsScript | 157.5 | 39.1 | 180.0 | 0.0 |

TABLE 5
Calculated mean and standard deviation of Tt values measured in n = 3 replicates of RT-LAMP
reactions using assays for all informative biomarkers and the optimized reaction chemistry. Serial
dilutions of IVT RNA transcripts were used as template and evaluated at the input concentrations
as listed at the top of each column. Where ≥2 measurements showed no amplification, standard
deviations were not calculated (gray fill). The mean across standard deviation measurements falling
within the quantitative dynamic range for the assay (as defined in the body of the manuscript)
was calculated for each biomarker. This value was used to calculate theoretical assay resolution.

| | Tt | RNA Template Copies per Reaction | | | | | | | | | | Mean SD |
|---|---|---|---|---|---|---|---|---|---|---|---|---|
| Assay | Metric | 10¹⁰ | 10⁹ | 10⁸ | 10⁷ | 10⁶ | 10⁵ | 10⁴ | 10³ | 10² | 10¹ | Within LoQ |
| CTSB | Mean | 2.17 | 2.92 | 3.73 | 4.56 | 5.33 | 6.11 | 7.05 | 8.33 | 30.00 | 29.68 | 0.125 |
| | StdDev | 0.15 | 0.15 | 0.05 | 0.04 | 0.10 | 0.17 | 0.09 | 0.24 | | | |
| GPAA1 | Mean | 2.87 | 3.84 | 4.86 | 6.41 | 7.21 | 8.40 | 10.00 | 12.69 | 14.29 | 23.76 | 0.168 |
| | StdDev | 0.17 | 0.15 | 0.19 | 0.20 | 0.03 | 0.18 | 0.25 | 2.05 | 1.29 | | |
| HK3 | Mean | 2.71 | 3.54 | 4.37 | 5.53 | 6.35 | 7.02 | 8.08 | 11.37 | 18.16 | 30.00 | 0.128 |
| | StdDev | 0.09 | 0.06 | 0.08 | 0.20 | 0.26 | 0.12 | 0.09 | 1.08 | 8.57 | | |
| TNIP1 | Mean | 3.05 | 3.99 | 4.98 | 6.02 | 7.25 | 8.01 | 9.61 | 12.15 | 24.82 | 30.00 | 0.240 |
| | StdDev | 0.21 | 0.18 | 0.15 | 0.28 | 0.32 | 0.29 | 0.25 | 1.03 | | | |
| IFI27 | Mean | 3.84 | 4.93 | 5.96 | 7.52 | 8.10 | 9.22 | 11.26 | 14.07 | 24.94 | 30.00 | 0.245 |
| | StdDev | 0.10 | 0.10 | 0.12 | 0.53 | 0.18 | 0.12 | 0.54 | 0.26 | 6.60 | | |
| JUP | Mean | 2.96 | 3.73 | 4.47 | 5.34 | 6.10 | 6.97 | 8.58 | 9.19 | 17.96 | 30.00 | 0.145 |
| | StdDev | 0.01 | 0.03 | 0.05 | 0.00 | 0.09 | 0.04 | 0.41 | 0.53 | 8.85 | | |
| LAX1 | Mean | 2.02 | 2.60 | 3.25 | 3.91 | 4.51 | 5.16 | 5.85 | 14.55 | 15.36 | 29.60 | 0.068 |
| | StdDev | 0.05 | 0.04 | 0.05 | 0.07 | 0.08 | 0.12 | 0.06 | 10.93 | 10.37 | | |

TABLE 6
Genomic DNA carryover after sample preparation assessed using
the Qubit 4 Fluorimeter (ThermoFisher) and the high sensitivity
DNA assay kit. DNA content was assessed in a subset of clinical
samples for which sufficient remnant was available.

| | | RNA | gDNA | |
|---|---|---|---|---|
| Sample | | Concentration | Concentration | Ratio |
| Number | Sample ID | (ng/μl) | (ng/μl) | DNA/RNA |
| 1 | BRH1562566 | 21 | 98 | 4.7 |
| 2 | BRH1562567 | 15.7 | 87.2 | 5.6 |
| 3 | BRH1562568 | 22.9 | 92.4 | 4.0 |
| 4 | BRH1562569 | 19.6 | 161 | 8.2 |
| 5 | BRH1562571 | 16.4 | 92 | 5.6 |
| 6 | BRH1562572 | 16.9 | 98.4 | 5.8 |
| 7 | BRH1562574 | 16.7 | 89.6 | 5.4 |
| 8 | BRH1562575 | 30.5 | 55.6 | 1.8 |
| 9 | BRH1562577 | 15.4 | 91.4 | 5.9 |
| 10 | BRH1562579 | 20.4 | 64.4 | 3.2 |
| 11 | BRH1562583 | 18.7 | 79.2 | 4.2 |
| 12 | BRH1562584 | 14.9 | 89.2 | 6.0 |
| 13 | BRH1562585 | 16.3 | 78.4 | 4.8 |
| 14 | BRH1562586 | 18.8 | 79.8 | 4.2 |
| 15 | BRH1562587 | 12.8 | 94.8 | 7.4 |
| 16 | BRH1562588 | 16.9 | 120 | 7.1 |
| 17 | BRH1562589 | 15.7 | 118 | 7.5 |
| 18 | BRH1562591 | 21.3 | 230 | 10.8 |
| 19 | BRH1562592 | 17.3 | 32.2 | 1.9 |
| 20 | BRH1562594 | 14.4 | 92.4 | 6.4 |
| 27 | 130 | 16.6 | 102 | 6.1 |
| 31 | 3007031 | 17.6 | 262 | 14.9 |
| 38 | 95 | 15.5 | 246 | 15.9 |
| 42 | 115 | 46.7 | 41.6 | 0.9 |
| 54 | 44 | 39.3 | 84 | 2.1 |

### Example 2. Identification of Primers Suitable for LAMP Amplification of Biomarkers

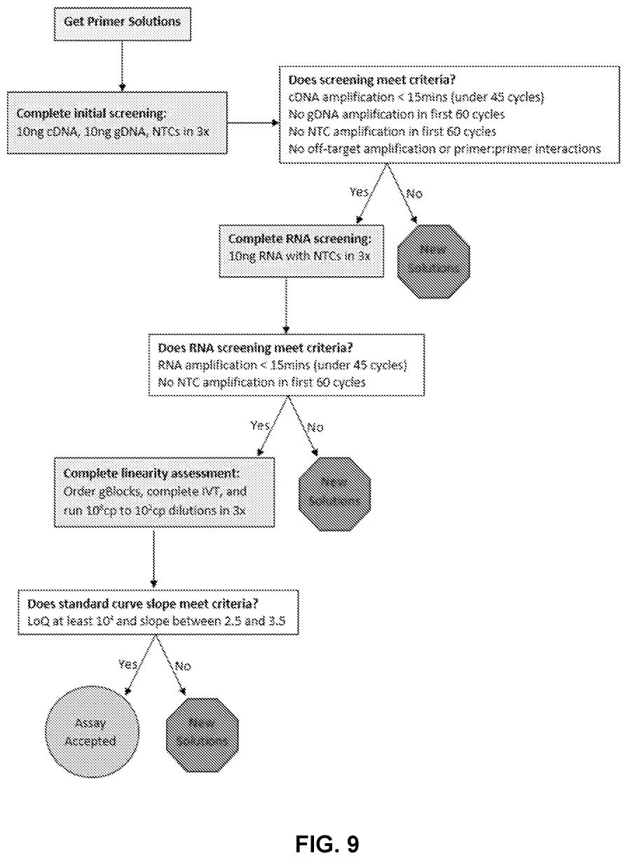

Candidate primers were assessed using a set of screening criteria to identify primers with suitable properties regarding, e.g., speed of amplification, specificity of amplification, relative absence of primer: primer interactions, and linearity of amplification. See, e.g., FIG. 9.

Biomarkers assessed included ARG1, BATF, C3AR1, C9orf95/NMRK1, CD163, CEACAM1, CTSB, CTSL1, DEFA4, FURIN, GADD45A, GNA15, HK3, HLA-DMB, IFI27, ISG15, JUP, KCNJ2, KIAA1370, KPNA6, LY86, OASL, OLFM4, PDE4B, PER1, PSMB9, RAPGEF1, RREB1, S100A12, TGFBI, YWHAB, and ZDHHC19. For each target, sets of candidate LAMP primers were designed, including FIP (Forward Inner Primer), BIP (Backward Inner Primer), F3 (or Forward Outer Primer), B3 (or Backward Outer Primer), and the loop primers LF and LB. All of the candidate primers that were tested using the herein-described methods are presented in Table 7.

TABLE 7
Complete list of candidate LAMP primers assessed in Example 1.

| Primer | | | |
|---|---|---|---|
| ID | Sequence | Primer ID | Sequence |
| ARG1- | actgagggttgactgactgg | PD HK3v4 | gcagttcaaggtgacaagggcac |
| F3v1 | | BL | |
| ARG1- | cacatcacactcttgttctttaagtttctca | PD HK3v4 | gcctgctccatggaacccaaga |
| B3v1 | | B3 | |
| ARG1- | ctccaataatccctatggttctggacttttttagagc | PD HK3v4 | tcagagcaactcagggtttcttccccactgtgg |
| FIPv1 | tcaagtgcagcaaagag | FIP | aagctcatggac |
| ARG1- | ctttctcaaagggacagccacgtttttagcagaccag | PD HK3v4 | tcagagctggtgcaggagtgcgctggcttggat |
| BIPv1 | cctttctcaatact | BIP | ctgctgtagc |
| ARG1- | gcgctcatgctctgacactt | PD HK3v5 | acctgaggagagtgactagcttct |
| LFv1 | | F3 | |
| ARG1- | aggggtggaagaaggcccta | PD HK3v5 | ccgcaaccctgaagaccca |
| LBv1 | | FL | |
| ARG1- | actgagggttgactgactgg | PD HK3v5 | gcagttcaaggtgacaagggcac |
| F3v1 | | BL | |
| ARG1- | cacatcacactcttgttctttaagtttctca | PD HK3v5 | gcctgctccatggaacccaaga |
| B3v1 | | B3 | |
| ARG1- | ctccaataatccctatggttctggactagagctcaag | PD HK3v5 | gagcaactcagggtttcttccccccactgtgga |
| FIP2 | tgcagcaaagag | FIP | agctcatggac |
| ARG1- | ctttctcaaagggacagccacgagcagaccagccttt | PD HK3v5 | tcagagctggtgcaggagtgcgctggcttggat |
| BIPv2 | ctcaatact | BIP | ctgctgtagc |
| ARG1- | gcgctcatgctctgacactt | PD HK3v6 | cagttcaaggtgacaagggcac |
| LFv1 | | F3 | |
| ARG1- | aggggtggaagaaggcccta | PD HK3v6 | cttctgggctccaccctatgc |
| LBv1 | | B3 | |
| C3AR1- | ggaccagacaggactcgtg | PD HK3v6 | cccacgtatgtaggcagcatcc |
| F3v1 | | FL | |
| C3AR1- | cgctgcatcttcaggcca | PD HK3v6 | gggcctcactgcgtgtt |
| B3v1 | | BL | |
| C3AR1- | tgagagtaggtcagttgaattggtcttttttccaggt | PD HK3v6 | tcagtgccatgtggggtggacagccagcctctt |
| FIPv1 | gctgaagccttc | FIP | gggttccat |
| C3AR1- | atggaatgagcccccagtaattcttttttgcctggca | PD HK3v6 | gagacttcgtggtgctggagcccctcaatgcca |
| BIPv1 | atcccagta | BIP | gttagagtcacc |
| C3AR1- | gaaagacgccattgctaaacttca | PD JUPv6 | ccgctgtcgtgcgtaccatg |
| LFv1 | | F3 | |
| C3AR1- | ccatggtcattctcagccttact | PD JUPv6 | gccacccgacttgaagatggc |
| LBv1 | | FL | |
| C3AR1- | ggaccagacaggactcgtg | PD JUPv6 | tcaccacgctgcacaacctg |
| F3v1 | | BL | |
| C3AR1- | cgctgcatcttcaggcca | PD JUPv6 | gggcaccatcttttgcagcc |
| B3v1 | | B3 | |
| C3AR1- | tgagagtaggtcagttgaattggtctccaggtgctga | PD JUPv6 | gagctgagcatgcggaccagaccagcatcctg |
| FIPv2 | agccttc | FIP | cacaacctct |
| C3AR1- | atggaatgagcccccagtaattctgcctggcaatccc | PD JUPv6 | ccctgtggagtcggtcctgttcttggcgccctc |
| BIPv2 | agta | BIP | ctggtaca |
| C3AR1- | gaaagacgccattgctaaacttca | PD JUPv7 | gccattgtgcatctcatcaactaccag |
| LFv1 | | F3 | |
| C3AR1- | ccatggtcattctcagccttact | PD JUPv7 | gttgtgcaggatgctggt |
| LBv1 | | B3 | |
| CTSL1- | cgacctccgcaaccttgag | PD JUPv7 | gggtcctcgtcgttgagcagt |
| F3v1 | | FL | |
| CTSL1- | cttggtccactgtgcctctaaac | PD JUPv7 | ccgctgtcgtgcgtaccatg |
| B3v1 | | BL | |
| CTSL1- | aaacctactcgaccgcgtcctttttctacgaccgcag | PD JUPv7 | caatcatggccgccttggtcactgcccgagctc |
| FIPv1 | caggaa | FIP | accaa |
| CTSL1- | taaaacatgaatcctacactcatccttttttttgtga | PD JUPv7 | tgaaccagctgtcgaagaaggagggtgtccag |
| BIPv1 | tcaaatgttagagtagctgagg | BIP | gtcgctggtattctg |
| CTSL1- | ctcttccagtccctgtccgg | PD JUPv8 | gaccacgccgagctcagttc |
| LFv1 | | F3 | |
| CTSL1- | gccttttgcctgggaattg | PD JUPv8 | ggtggttttcttgagcgtgtactgg |
| LBv1 | | B3 | |
| CTSL1- | cgacctccgcaaccttgag | PD JUPv8 | gttcatcacctccatcgtggctact |
| F3v1 | | FL | |
| CTSL1- | cttggtccactgtgcctctaaac | PD JUPv8 | tacgactcgggtatccactcggg |
| B3v1 | | BL | |
| CTSL1- | aaacctactcgaccgcgtccctacgaccgcagcagg | PD JUPv8 | caccttgataggctgctccatcagcggtcaggc |
| FIPv2 | aa | FIP | cccatact |
| CTSL1- | taaaacatgaatcctacactcatcctttgtgatcaaa | PD JUPv8 | actgagtggcagcagacatacaccctcctccat |
| BIPv2 | tgttagagtagctgagg | BIP | gatgcccttg |
| CTSL1- | ctcttccagtccctgtccgg | PD CTSBv5 | agatgattggcaggtggatctaggat |
| LFv1 | | F3 | |
| CTSL1- | gccttttgcctgggaattg | PD CTSBv5 | cacccaggaaggtaccacat |
| LBv1 | | B3 | |
| FURIN- | tgaggccctggttgctat | PD CTSBv5 | ctccgggcattggccaaca |
| F3v1 | | FL | |
| FURIN- | tcactcctcgatgccagaa | PD CTSBv5 | caggccgggcacaacttcta |
| B3v1 | | BL | |
| FURIN- | ttggtgaagaccttctggcgcaacaggaaccttggtc | PD CTSBv5 | agctcatccgacaggggatccggcttccaaca |
| FIPv1 | c | FIP | tgtggca |
| FURIN- | caacagtgtggcacggaaggtaatagtccccgaaga | PD CTSBv5 | actatgtcaacaaacggaataccacgtgcctct |
| BIPv1 | tctgg | BIP | tcaagtagctcatgtccacgtt |
| FURIN- | cctgagcatcagctgctag | PD PER1v4 | atgacagcacttcgagagctcaagctt |
| LFv1 | | F3 | |
| FURIN- | gcatgggttcctcaacctg | PD PER1v4 | gctgcctgctccgaaatgtagacgat |
| LBv1 | | FL | |
| FURIN- | aagcatgggttcctcaacc | PD PER1v4 | gctccactgctggtagtattcctggtt |
| F3v2 | | BL | |
| FURIN- | ctgagtgacaccagacaggta | PD PER1v4 | ggagcacatcacgtctgagtacacactt |
| B3v2 | | B3 | |
| FURIN- | ctgtacttgaggctccctctgccagatcttcggggac | PD PER1v4 | atggagcaaggctcgccctccgcactggcctgt |
| FIPv2 | tatt | FIP | gtcaagc |
| FURIN- | ggaacagcaggtggcaaagctgaggaaacttgggg | PD PER1v4 | atgtccacctataccctggaggagctcagccac |
| BIPv2 | tc | BIP | tgagaaggtatcctggttct |
| FURIN- | ctcctcgatgccagaagtg | PD | gtgcagacaccttcagggatacaacc |
| LFv2 | | ZDHHC19- | |
| | | 1v6 F3 | |
| FURIN- | ctaaacgggacgtgtaccag | PD | ccgtgtggaactcctggagagct |
| LBv2 | | ZDHHC19- | |
| | | 1v6 FL | |
| FURIN- | attaccacttctggcatcgag | PD | gggtcccagtggtgcacaaattgtt |
| F3v3 | | ZDHHC19- | |
| | | 1v6 BL | |
| FURIN- | gtccagaatggagaccacaat | PD | agccccaacctctgggt |
| B3v3 | | ZDHHC19- | |
| | | 1v6 B3 | |
| FURIN- | ctggtacacgtcccgtttagagagggagcctcaagta | PD | atgtccagtcaggccccaccagggctgtgcca |
| FIPv3 | cag | ZDHHC19- | gcaactggtattt |
| | | 1v6 FIP | |
| FURIN- | gaccccaagtttcctcagccgccttcacattcaggtc | PD | atgccgaatctgcaccctccaatggggtccctt |
| BIPv3 | c | ZDHHC19- | ccctgctttgtagg |
| | | 1v6 BIP | |
| FURIN- | tttgccacctgctgttcc | PD | acaccttcagggatacaaccccttcga |
| LFv3 | | ZDHHC19- | |
| | | 1v7 F3 | |
| FURIN- | ctgtctggtgtcactcagc | PD | gcccgtgtggaactcct |
| LBv3 | | ZDHHC19- | |
| | | 1v7 FL | |
| FURIN- | tgaggccctggttgctat | PD | tgggtcccagtggtgcacaaattgtt |
| F3v1 | | ZDHHC19- | |
| | | 1v7 BL | |
| FURIN- | tcactcctcgatgccagaa | PD | agccccaacctctgggt |
| B3v1 | | ZDHHC19- | |
| | | 1v7 B3 | |
| FURIN- | ttggtgaagaccttctggctttttgcaacaggaacct | PD | atgtccagtcaggccccaccaccagggctgtg |
| FIPv1 + | tggtcc | ZDHHC191- | ccagcaactggtat |
| t | | v7 FIP | |
| FURIN- | caacagtgtggcacggaatttttggtaatagtccccg | PD | atgccgaatctgcaccctccaatgtgggtccct |
| BIPv1 + | aagatctgg | ZDHHC191- | tccctgctttgtagg |
| t | | v7 BIP | |
| FURIN- | cctgagcatcagctgctag | PD C3AR1v6 | gcccttctcgctggctcactt |
| LFv1 | | F3 | |
| FURIN- | gcatgggttcctcaacctg | PD C3AR1v6 | ggtacacgaacacaggaatgcacatca |
| LBv1 | | FL | |
| FURIN- | aagcatgggttcctcaacc | PD C3AR1v6 | gggatgagcttgcataggaacctgc |
| F3v2 | | BL | |
| FURIN- | ctgagtgacaccagacaggta | PD C3AR1v6 | cgctgtcttgtggtattcaagccaatct |
| B3v2 | | B3 | |
| FURIN- | ctgtacttgaggctccctcttttttgccagatcttcg | PD C3AR1v6 | tggcaaacatgttgaggacaatgatctctccag |
| FIPv2 + | gggactatt | FIP | ggacagtggcccta |
| t | | | |
| FURIN- | ggaacagcaggtggcaaatttttgctgaggaaacttg | PD C3AR1v6 | ttcctgcttactgccattagcctggacatccct |
| BIPv2 + | gggtc | BIP | acattgcgatgattctgacacc |
| t | | | |
| FURIN- | ctcctcgatgccagaagtg | PD C3AR1v7 | gcaggttcctatgcaagctcatcc |
| LFv2 | | F3 | |
| FURIN- | ctaaacgggacgtgtaccag | PD C3AR1v7 | tgagctggagagaccaaatttgtagcca |
| LBv2 | | FL | |
| FURIN- | attaccacttctggcatcgag | PD C3AR1v7 | tccaggctaatggcagtaagcaggaa |
| F3v3 | | BL | |
| FURIN- | gtccagaatggagaccacaat | PD C3AR1v7 | tggtggcttttgtgatgtgcattcct |
| B3v3 | | B3 | |
| FURIN- | ctggtacacgtcccgtttagtttttagagggagcctc | PD C3AR1v7 | agattggcttgaataccacaagacagcgccat |
| FIPv3 + | aagtacag | FIP | cattgtcctcaacatgtttgccagt |
| t | | | |
| FURIN- | gaccccaagtttcctcagctttttcgccttcacattc | PD C3AR1v7 | gtgtcagaatcatcgcaatgtagggatggctgt |
| BIPv3 + | aggtcc | BIP | agtgaagatttcccggtacacgaaca |
| t | | | |
| FURIN- | tttgccacctgctgttcc | PD | ggcaaaacaacactggctaagaatttgcag |
| LFv3 | | NMRK1v3 F3 | |
| FURIN- | ctgtctggtgtcactcagc | PD | gcttctattccatatagtgtcaaggggcttat |
| LBv3 | | NMRK1v3 FL | |
| FURIN- | agagggagcctcaagtacag | PD | ctatctcagactctggcttgaagaaatcatcc |
| F3v4 | | NMRK1v3 BL | |
| FURIN- | caggatcataattgcctgccaa | PD | aagcgcaagacactctgtggtatcaac |
| B3v4 | | NMRK1v3 B3 | |
| FURIN- | gctgagtgacaccagacagggcaaagcgacggact | PD | gcttcaagcacategtactgcaaaaatccacac |
| FIPv4 | aa | NMRK1v3 | ctcccaaattgcagtgtcatatctca |
| | | FIP | |
| FURIN- | ggacctgaatgtgaaggcgtggttcttctcgatgcca | PD | tgtcagccatttcctgctggatgggggaatttc |
| BIPv4 | tc | NMRK1v3 | ctcagcactttcctggtc |
| | | BIP | |
| FURIN- | gctgaggaaacttggggtc | PD GNA15v4 | gtccatgcgggccatgatcga |
| LFv4 | | F3 | |
| FURIN- | cggcattgtggtctccattc | PD GNA15v4 | ggacgtagccctcctcggtgat |
| LBv4 | | FL | |
| FURIN- | ctaaacgggacgtgtaccag | PD GNA15v4 | cttgctctcgggcctgctgaat |
| F3v5 | | BL | |
| FURIN- | cattcatctgtgtgtaccgagg | PD GNA15v4 | ggcatccgggcctgctatga |
| B3v5 | | B3 | |
| FURIN- | gaatggagaccacaatgccgcacagaccccaagttt | PD GNA15v4 | ggctcatgaccaggctagcgtgccatggagcg |
| FIPv5 | cctc | FIP | gctgcagattcc |
| FURIN- | gatggcatcgagaagaaccactggtcattgacatca | PD GNA15v4 | gtgaccacgtttgagaagcgctacgctgaatc |
| BIPv5 | aaactgg | BIP | gagcaggtggaattcccg |
| FURIN- | ctgagtgacaccagacaggta | PD GNA15v5 | ggcatccgggcctgctatga |
| LFv5 | | F3 | |
| FURIN- | acttggcaggcaattatgatcc | PD GNA15v5 | ccatttcttacgctctgacttctggcc |
| LBv5 | | FL | |
| FURIN- | gaccccaagtttcctcagc | PD GNA15v5 | ggacaggtagtacacggctgaatcgag |
| F3v6 | | BL | |
| FURIN- | cattcatctgtgtgtaccgagg | PD GNA15v5 | tgcccaccactggcatcaac |
| B3v6 | | B3 | |
| FURIN- | atcgtccagaatggagaccactgtctggtgtcactca | PD GNA15v5 | tcctcggtgatgcgctccagcgtcggcgggaat |
| FIPv6 | gc | FIP | tccacct |
| FURIN- | gcatcgagaagaaccacccctggtcattgacatcaa | PD GNA15v5 | cacagctcaggacgtgctccgatccgcaggtt |
| BIPv6 | aactgg | BIP | ggttttctgcac |
| FURIN- | cgccttcacattcaggtcc | PD BATFv3 | ctgagtgtgagagcccggaagattt |
| LFv6 | | F3 | |
| FURIN- | acttggcaggcaattatgatcc | PD BATFv3 | tgttcagcaccgacgtgaagtactt |
| LBv6 | | FL | |
| FURIN- | actcagcgggacctgaat | PD BATFv3 | catcagatgagtcctgtttgccagg |
| F3v7 | | BL | |
| FURIN- | cgttgtaggccacaccta | PD BATFv3 | gcacctggagagcgaagacct |
| B3v7 | | B3 | |
| FURIN- | atcataattgcctgccaagtccacggcattgtggtct | PD BATFv3 | tacgatttttctccctcctctgaactcttcagc |
| FIPv7 | ccat | FIP | agtgactccagcttcagc |
| FURIN- | ccagttttgatgtcaatgaccagccgtgcctgttgtc | PD BATFv3 | gaagagccgacagaggcagtgcttgatctcct |
| BIPv7 | attc | BIP | tgcgtagagcc |
| FURIN- | cttctcgatgccatcgtc | PD BATFv5 | cttccagccctgagcttcc |
| LFv7 | | F3 | |
| FURIN- | agcctcggtacacacagat | PD BATFv5 | tgccatgggacttgagcatct |
| LBv7 | | FL | |
| CTSL1- | cgacctccgcaaccttgag | PD BATFv5 | tccgggtctccaggtggaca |
| F3v1 | | BL | |
| CTSL1- | cttggtccactgtgcctctaaac | PD BATFv5 | cagtgccgcagcgtttcgag |
| B3v1 | | B3 | |
| CTSL1- | aaacctactcgaccgcgtcctttttctacgaccgcag | PD BATFv5 | ttcactccttgtccaggcctctgtgggagggg |
| FIPv1 | caggaa | FIP | acacagactgt |
| CTSL1- | taaaacatgaatcctacactcatccttttttttgtga | PD BATFv5 | gaactgtcacgactggaagggcgtgggcctgc |
| BIPv1 | tcaaatgttagagtagctgagg | BIP | aacccacagtg |
| CTSL1- | ctcttccagtccctgtccgg | PD IFI27v7 | gccaggattgctacagttgtga |
| LFv1 | | F3 | |
| CTSL1- | gccttttgcctgggaattg | PD IFI27v7 | gctagtagaacctcgcaatgaca |
| LBv1 | | B3 | |
| GADD45 | cctgtgagtgagtgcagaaa | PD IFI27v7 | cggacatcatcttggctgc |
| A-F3v1 | | FL | |
| GADD45 | accccgacagtgatcgtg | PD IFI27v7 | tctccggattgaccaagttcat |
| A-B3v1 | | BL | |
| GADD45 | ttcggtcttctgctctccatttttgcgacctgcagtt | PD IFI27v7 | agagtagccacaaggctgcagtgccatgggct |
| A-FIPv1 | tgcaata | FIP | tcact |
| GADD45 | ggatggataaggtgggggatttttgctgactcagggc | PD IFI27v7 | agtcactgggagcaactggcgcaatggcagac |
| A-BIPv1 | tttgc | BIP | ccaat |
| GADD45 | gccgagaattcctccaaagt | PD IFI27v8 | ggaatcgcctcgtcctccatag |
| A-LFv1 | | F3 | |
| GADD45 | ccctggaggaagtgctca | PD IFI27v8 | ctcctcgctgggtcaggat |
| A-LBv1 | | B3 | |
| GADD45 | cctgtgagtgagtgcagaaa | PD IFI27v8 | gtcaatccggagagtccagttg |
| A-F3v1 | | FL | |
| GADD45 | accccgacagtgatcgtg | PD IFI27v8 | cctcgccctgcagagaa |
| A-B3v1 | | BL | |
| GADD45 | ttcggtcttctgctctccagcgacctgcagtttgcaa | PD IFI27v8 | caatggagcccaggatgaacttgggcagcctt |
| A-FIPv2 | ta | FIP | gtggctac |
| GADD45 | ggatggataaggtgggggagctgactcagggctttg | PD IFI27v8 | attgcgaggttctactagctccctcttctcccc |
| A-BIPv2 | c | BIP | tggcatggttc |
| GADD45 | gccgagaattcctccaaagt | PD JUPv9 | accccaagttcctggccatc |
| A-LFv1 | | F3 | |
| GADD45 | ccctggaggaagtgctca | PD JUPv9 | tcccaccagcctccacaatg |
| A-LBv1 | | B3 | |
| GADD45 | cacttcaccctgatccagg | PD JUPv9 | gatcagcttgctctcctggtt |
| A-F3v3 | | FL | |
| GADD45 | tgatccatgtagcgactttcc | PD JUPv9 | accaccagtcgtgtgctcaag |
| A-B3v3 | | BL | |
| GADD45 | tctccaagagcaggagctcgttttgctgcgagaacga | PD JUPv9 | gatctgcacgagggccttgcagctcctggccta |
| A-FIPv3 | | FIP | c |
| GADD45 | gctggtgacgaatccacatcggcaaaaacaaataag | PD JUPv9 | atgcgtaactacagttatgaaaagctgcgctta |
| A-BIPv3 | ttgacttaag | BIP | ttgctgggacacacggatag |
| GADD45 | gacgcgcaggatgttgat | PD JUPv10 | cgacgggctgcaaaagat |
| A-LFv3 | | F3 | |
| GADD45 | catctcaatggaaggatcctgc | PD JUPv10 | ctgggacacacggatagca |
| A-LBv3 | | B3 | |
| HLA- | tgaactcccggcatctttac | PD JUPv10 | gtggtgatggccaggaactt |
| DMB- | | FL | |
| F3v1 | | | |
| HLA- | cgccaagctattcagcac | PD JUPv10 | gttatgaaaagctgctctggacc |
| DMB- | | BL | |
| B3v1 | | | |
| HLA- | aatcctttggagtcccagctttttatcacattcctgc | PD JUPv10 | atgatcagcttgctctcctgggtgcccctgctc |
| DMB- | cgctg | FIP | aacaaga |
| FIPv1 | | | |
| HLA- | tactgcatctccttcaacaaggtttttaattcgcaag | PD JUPv10 | tcgtgcagatcatgcgtaacccttgagcacacg |
| DMB- | gggccatc | BIP | actggt |
| BIPv1 | | | |
| HLA-DMB | gacaggtgctttccacatg | PD JUPv12 | caaccaggagagcaagctgat |
| LFv1 | | F3 | |
| HLA- | gctgggatccagaggagaata | PD JUPv12 | tggccacatctgagaggtt |
| DMB- | | B3 | |
| LBv1 | | | |
| HLA- | tgaactcccggcatctttac | PD JUPv12 | cagagcagcttttcataactgtagt |
| DMB- | | FL | |
| F3v1 | | | |
| HLA- | cgccaagctattcagcac | PD JUPv12 | agcacctgaccagcaaca |
| DMB- | | BL | |
| B3v1 | | | |
| HLA- | aatcctttggagtcccagcatcacattcctgccgctg | PD JUPv12 | ttgagcacacgactggtggcctcgtgcagatca |
| DMB- | | FIP | tgcgta |
| FIPv2 | | | |
| HLA- | tactgcatctccttcaacaaggaattcgcaaggggcc | PD JUPv12 | gcaataagcctgccattgtgacaggcagttctg |
| DMB- | atc | BIP | cacca |
| BIPv2 | | | |
| HLA- | gacaggtgctttccacatg | PD | gctcctcttccggtttccat |
| DMB- | | ZDHHC19- | |
| LFv1 | | 1v8 F3 | |
| HLA- | gctgggatccagaggagaata | PD | gacattggagggtgcagattc |
| DMB- | | ZDHHC19- | |
| LBv1 | | 1v8 B3 | |
| HLA- | tggcgaatgtcctctcac | PD | tcgaaggggttgtatccctg |
| DMB- | | ZDHHC19- | |
| F3v3 | | 1v8 FL | |
| HLA- | gatagtcacttctgctggatagaa | PD | cccaagtacatggctgaagc |
| DMB- | | ZDHHC19- | |
| B3v3 | | 1v8 BL | |
| HLA- | cctgttggtcagtgatcccaagacaccctgatgcagc | PD | aaataccagttgctggcacagcgaccagtgca |
| DMB- | | ZDHHC19- | gacacctt |
| FIPv3 | | 1v8 FIP | |
| HLA- | caccatctgtgcaagtagcatagcaggccagcatca | PD | caatttgtgcaccactgggaggcatggatgtcc |
| DMB- | c | ZDHHC19- | agtcag |
| BIPv3 | | 1v8 BIP | |
| HLA- | acaattctgaagcccattgc | PD | cagggatacaaccccttcga |
| DMB- | | ZDHHC19- | |
| LFv3 | | 1v9 F3 | |
| HLA- | accactccttttaacacgagg | PD | cgtgtggaactcctggaga |
| DMB- | | ZDHHC19- | |
| LBv3 | | 1v9 B3 | |
| HLA- | tggcgaatgtcctctcac | PD | gtcccagtggtgcacaaatt |
| DMB- | | ZDHHC19- | |
| F3v3 | | 1v9 FL | |
| HLA- | gatagtcacttctgctggatagaa | PD | atctgcaccctccaatgtcc |
| DMB- | | ZDHHC19- | |
| B3v3 | | 1v9 BL | |
| HLA- | cctgttggtcagtgatccctttttaagacaccctgat | PD | tggacagcttcagccatgtctgtgccagcaact |
| DMB- | gcagc | ZDHHC19- | ggtattt |
| FIPv4 | | 1v9 FIP | |
| HLA- | caccatctgtgcaagtagctttttatagcaggccagc | PD | tgactggacatccatgccgctttgtagggaccc |
| DMB- | atcac | ZDHHC19- | agaggtt |
| BIPv4 | | 1v9 BIP | |
| HLA- | acaattctgaagcccattgc | PD | tacaaccccttcgaccagg |
| DMB- | | ZDHHC19- | |
| LFv3 | | 1v10 F3 | |
| HLA- | accactccttttaacacgagg | PD | tgtggaactcctggagagct |
| DMB- | | ZDHHC19- | |
| LBv3 | | 1v10 B3 | |
| HLA- | aagacaccctgatgcagc | PD | tcccagtggtgcacaaattg |
| DMB- | | ZDHHC19- | |
| F3v5 | | 1v10 FL | |
| HLA- | catgacaagcttcccgttc | PD | atctgcaccctccaatgtcc |
| DMB- | | ZDHHC19- | |
| B3v5 | | 1v10 BL | |
| HLA- | cctcgtgttaaaaggagtggtgggatcactgaccaac | PD | gcttcagccatgtacttgggctgtgccagcaac |
| DMB- | agg | ZDHHC19- | tggtattt |
| FIPv5 | | 1v10 FIP | |
| HLA- | gtgatgctggcctgctatcctccacgtgatagtcact | PD | ctgactggacatccatgcctttgtagggaccca |
| DMB- | tc | ZDHHC19- | gaggttg |
| BIPv5 | | 1v10 BIP | |
| HLA- | gctacttgcacagatggtg | PD | ccaggaaatattaggaagctgtgattt |
| DMB- | | NMRK1v4 F3 | |
| LFv5 | | | |
| HLA- | gtgtggggcttctatccag | PD | ccatccagcaggaaatggct |
| DMB- | | NMRK1v4 B3 | |
| LBv5 | | | |
| HLA- | gtgactatcacgtggaggaa | PD | gtttctgcaaattcttagccagtgt |
| DMB- | | NMRK1v4 FL | |
| F3v6 | | | |
| HLA- | gaaaccttcagggtctgcat | PD | ggatttttgcagtacgatgtgctt |
| DMB- | | NMRK1v4 BL | |
| B3v6 | | | |
| HLA- | ggtctggtatgtccagtctcgaacgggaagcttgtca | PD | catcctgagatatgacactgcaattaacattta |
| DMB- | tg | NMRK1v4 | tcattggaatcagtggtgt |
| FIPv6 | | FIP | |
| HLA- | ctcccatttagccttaacccctgtccagtcccgaagg | PD | cttcaagccagagtctgagatagaacatcattt |
| DMB- | at | NMRK1v4 | tttccatgttaagtgcttc |
| BIPv6 | | BIP | |
| HLA- | attgggctgggcagtctt | PD | aacatttatcattggaatcagtggtgt |
| DMB- | | NMRK1v5 F3 | |
| LFv6 | | | |
| HLA- | ggggacacttacacctgtg | PD | gtctgttgataccacagagtgtctt |
| DMB- | | NMRK1v5 B3 | |
| LBv6 | | | |
| HLA- | aagacaccctgatgcagc | PD | gagatatgacactgcaatttgggag |
| DMB- | | NMRK1v5 FL | |
| F3v5 | | | |
| HLA- | catgacaagcttcccgttc | PD | cttaacatggaaaaaatgatgtcagcc |
| DMB- | | NMRK1v5 BL | |
| B3v5 | | | |
| HLA- | cctcgtgttaaaaggagtggttttttgggatcactga | PD | agactctggcttgaagaaatcatccacaacact |
| DMB- | ccaacagg | NMRK1v5 | ggctaagaatttgca |
| FIPv5 + | | FIP | |
| t | | | |
| HLA- | gtgatgctggcctgctattttttcctccacgtgatag | PD | ggatttttgcagtacgatgtgccgctttccatc |
| DMB- | tcacttc | NMRK1v5 | cagcaggaa |
| BIPv5 + | | BIP | |
| t | | | |
| HLA- | gctacttgcacagatggtg | PD | acaacactggctaagaatttgca |
| DMB- | | NMRK1v6 F3 | |
| LFv5 | | | |
| HLA- | gtgtggggcttctatccag | PD | tcgatgattaaaatgggaatttcctcag |
| DMB- | | NMRK1v6 B3 | |
| LBv5 | | | |
| HLA- | gtgactatcacgtggaggaa | PD | tggcttgaagaaatcatcctgagata |
| DMB- | | NMRK1v6 FL | |
| F3v6 | | | |
| HLA- | gaaaccttcagggtctgcat | PD | atggaaagcgcaagacactc |
| DMB- | | NMRK1v6 BL | |
| B3v6 | | | |
| HLA- | ggtctggtatgtccagtctctttttgaacgggaagct | PD | tttccatgttaagtgcttcaagcgaaacacctc |
| DMB- | tgtcatg | NMRK1v6 | ccaaattgcagt |
| FIPv6 + | | FIP | |
| t | | | |
| HLA- | ctcccatttagccttaaccccttttttgtccagtccc | PD | aatgatgtcagccatttcctgccactttcctgg |
| DMB- | gaaggat | NMRK1v6 | tctgttgatacca |
| BIPv6 + | | BIP | |
| t | | | |
| HLA- | attgggctgggcagtctt | PD | tgatttacgagatatgccagccaa |
| DMB- | | KIAA1370v3 | |
| LFv6 | | F3 | |
| HLA- | ggggacacttacacctgtg | PD | tcagtgtaagatttgagttcatatgcag |
| DMB- | | KIAA1370v3 | |
| LBv6 | | B3 | |
| HLA- | gagcatgatcacattcctgc | PD | tctgtgtgtcggatgttctctttat |
| DMB- | | KIAA1370v3 | |
| F3v7 | | FL | |
| HLA- | gacattcgccaagctattcag | PD | gtacggctcctgttctctagaa |
| DMB- | | KIAA1370v3 | |
| B3v7 | | BL | |
| HLA- | gttgaaggagatgcagtatgtgcatgtggaaagcac | PD | tatgaggtagcgtaataaccgttctacgacaaa |
| DMB- | ctgtc | KIAA1370v3 | gaactttttctgtacctg |
| FIPv7 | | FIP | |
| HLA- | aaggatctgctgacctgccaccccaaattcgcaagg | PD | cagagttctaaatctggaaagatctaccaccgc |
| DMB- | | KIAA1370v3 | tatcaacctccattga |
| BIPv7 | | BIP | |
| HLA- | aatcctttggagtcccagc | PD | gaactttttctgtacctgttaaacaaga |
| DMB- | | KIAA1370v5 | |
| LFv7 | | F3 | |
| HLA- | ggatccagaggagaataagatgg | PD | ggtttgttggtgattcagtgtaaga |
| DMB- | | KIAA1370v5 | |
| LBv7 | | B3 | |
| HLA- | atcacattcctgccgctg | PD | ggtagcgtaataaccgttcttctg |
| DMB- | | KIAA13705 | |
| F3v8 | | FL | |
| HLA- | cgccaagctattcagcac | PD | ctcctgttctctagaaagtcaatgga |
| DMB- | | KIAA1370v5 | |
| B3v8 | | BL | |
| HLA- | ggagatgcagtatgtgaaatccgtggaaagcacctg | PD | atttagaactctggaacctcagatgtagaagtg |
| DMB- | tctgtt | KIAA1370v5 | ttaataaagagaacatccgac |
| FIPv8 | | FIP | |
| HLA- | ttcaacaaggatctgctgaccaattcgcaaggggcca | PD | tggaaagatctacctccatagagacgatgcag |
| DMB- | tc | KIAA1370v5 | caccgctatcaac |
| BIPv8 | | BIP | |
| HLA- | ttggagtcccagcatcatc | PD | cacagaagaacggttattacgct |
| DMB- | | KIAA1370v7 | |
| LFv8 | | F3 | |
| HLA- | gctgggatccagaggagaata | PD | tgcaaacttgggtactgagtaaatac |
| DMB- | | KIAA1370v7 | |
| LBv8 | | B3 | |
| HLA- | tttggggtgctgaatagctt | PD | ctctatggaggtagatctttccagatt |
| DMB- | | KIAA1370v7 | |
| F3v9 | | FL | |
| HLA- | atagcaggccagcatcac | PD | gctgcatatgaactcaaatcttacactg |
| DMB- | | KIAA1370v7 | |
| B3v9 | | BL | |
| HLA- | gcacaattctgaagcccattcgaatgtcctctcacag | PD | ttctagagaacaggagccgtaccctcatacatc |
| DMB- | c | KIAA1370v7 | tgaggttccaga |
| FIPv9 | | FIP | |
| HLA- | ccttctggggatcactgacctcgtgttaaaaggagtg | PD | tcaatggaggttgatagcggtcatcttggtgaa |
| DMB- | gttt | KIAA1370v7 | aactgagggttt |
| BIPv9 | | BIP | |
| HLA- | gctgcatcagggtgtcttt | PD DEFA4v6 | cctagcttgaggatctgtcacc |
| DMB- | | F3 | |
| LFv9 | | | |
| HLA- | gccaccatctgtgcaagta | PD DEFA4v6 | ccggcagaatactaatctgcaaga |
| DMB- | | B3 | |
| LBv9 | | | |
| HLA- | cttgtcatgcctcacagc | PD DEFA4v6 | gctaccaagagaatagcagcga |
| DMB- | | FL | |
| F3v10 | | | |
| HLA- | ccagctgatcacaccaag | PD DEFA4v6 | ggataaaagctctgctcttcaggt |
| DMB- | | BL | |
| B3v10 | | | |
| HLA- | cacaggtgtaagtgtcccccaatggagactggacat | PD DEFA4v6 | atcacctcttgcctggagtggccatgaggatta |
| DMB- | accag | FIP | tcgccct |
| FIPv10 | | | |
| HLA- | atccttcgggactggacaagaagatgatgaggccca | PD DEFA4v6 | gaccaggacatatctatttcctttgcgaccatg |
| DMB- | g | BIP | ccccttgttgag |
| BIPv10 | | | |
| HLA- | ggggttaaggctaaatgggag | PD DEFA4v8 | ttctcttggtagccctccagg |
| DMB- | | F3 | |
| LFv10 | | | |
| HLA- | tgcagaccctgaaggtttc | PD DEFA4v8 | cagaacgttaatcgacacgc |
| DMB- | | B3 | |
| LBv10 | | | |
| HLA- | caatggagactggacataccag | PD DEFA4v8 | gcaaaggaaatagatatgtcctggtc |
| DMB- | | FL | |
| F3v11 | | | |
| HLA- | ccagctgatcacaccaag | PD DEFA4v8 | ggcgaacagaacttcgtgtt |
| DMB- | | BL | |
| B3v11 | | | |
| HLA- | ccagtgtgctctaccacacctctcccatttagcctta | PD DEFA4v8 | acctgaagagcagagcttttatcccactccagg |
| DMB- | acc | FIP | caagaggtgat |
| FIPv11 | | | |
| HLA- | atccttcgggactggacaagaagatgatgaggccca | PD DEFA4v8 | ctcaacaaggggcatggtcccaccaatgaggc |
| DMB- | g | BIP | agttcc |
| BIPv11 | | | |
| HLA- | gtaagtgtccccgtaagagg | PD DEFA4v9 | aagaggtgatgaggctccag |
| DMB- | | F3 | |
| LFv11 | | | |
| HLA- | tgcagaccctgaaggtttc | PD DEFA4v9 | ccagcatgacattctcttggaca |
| DMB- | | B3 | |
| LBv11 | | | |
| ISG15- | gcgaactcatctttgccagtac | PD DEFA4v9 | gagcttttatcccatgcaaagga |
| F3v1 | | FL | |
| ISG15- | cgccgatcttctgggtgat | PD DEFA4v9 | cttcgtgttgggaactgcc |
| B3v1 | | BL | |
| ISG15- | cgccagcatcttcaccgtttttaggagcttgtgccgt | PD DEFA4v9 | tgttgagcctgaaacctgaagagccagaagac |
| FIPv1 | gg | FIP | caggacatatctatt |
| ISG15- | ggcaacgaattccaggtgtctttttgcgccttcagct | PD DEFA4v9 | gtattctgccggcgaacagcagaacgttaatc |
| BIPv1 | ctgac | BIP | gacacgc |
| ISG15- | tcaggtcccagcccatg | PD CD163v6 | tgcagacaaagggaaaatcaacc |
| LFv1 | | F3 | |
| ISG15- | gagcagctccatgtcggt | PD CD163v6 | agatctccacacgtccagaac |
| LBv1 | | B3 | |
| ISG15- | gcgaactcatctttgccagtac | PD CD163v6 | ttgtccacccacatgggaat |
| F3v1 | | FL | |
| ISG15- | cgccgatcttctgggtgat | PD CD163v6 | cggaggagacctggatcaca |
| B3v1 | | BL | |
| ISG15- | cgccagcatcttcaccgaggagcttgtgccgtgg | PD CD163v6 | caggtccttttggacactgaacgcatctttaga |
| FIPv2 | | FIP | caaggccatgtc |
| ISG15- | ggcaacgaattccaggtgtcgcgccttcagctctgac | PD CD163v6 | ctccatgggagaagagactgggaagtgggtcc |
| BIPv2 | | BIP | ttcctgaag |
| ISG15- | tcaggtcccagcccatg | PD CD163v7 | gcatctttagacaaggccatgtc |
| LFv1 | | F3 | |
| ISG15- | gagcagctccatgtcggt | PD CD163v7 | ccacctgagcatcgtccaa |
| LBv1 | | B3 | |
| ISG15- | tctttgccagtacaggagctt | PD CD163v7 | cgtgtcaggtccttttggaca |
| F3v3 | | FL | |
| ISG15- | aagggggaccctgtcct | PD CD163v7 | cacttcctgttctggacgtgt |
| B3v3 | | BL | |
| ISG15- | accgacatggagctgctccatgggctgggacctga | PD CD163v7 | tgtgatccaggtctcctccgccatgtgggtgga |
| FIPv3 | | FIP | caatgttc |
| ISG15- | gtcagagctgaaggcgcagacgctgctggaaggc | PD CD163v7 | aacaagataagacttcaggaaggaccggaac |
| BIPv3 | | BIP | ctccatgccagatct |
| ISG15- | gacacctggaattcgttgcc | PD CD163v12 | gacgtgtggagatctggcat |
| LFv3 | | F3 | |
| ISG15- | acccagaagatcggcgtg | PD CD163v12 | tgtgcccacactcactatgg |
| LBv3 | | B3 | |
| ISG15- | tctttgccagtacaggagctt | PD CD163v12 | cacctgagcatcgtccaag |
| F3v3 | | FL | |
| ISG15- | aagggggaccctgtcct | PD CD163v12 | gtgcaaagggaatgagtcttcc |
| B3v3 | | BL | |
| ISG15- | accgacatggagctgctctttttcatgggctgggacc | PD CD163v12 | caaactctgcttctttgaatgctcagtgtgtga |
| FIPv4 | tga | FIP | tgactcttggg |
| ISG15- | gtcagagctgaaggcgctttttagacgctgctggaag | PD CD163v12 | gtcaggggactggaccgatatagcgtctggca |
| BIPv4 | gc | BIP | ggacaat |
| ISG15- | gacacctggaattcgttgcc | PD | tgctcgaagcgttcctg |
| LFv3 | | CEACAM1v8 | |
| | | F3 | |
| ISG15- | acccagaagatcggcgtg | PD | ggggcagattgtggacaag |
| LBv3 | | CEACAM1v8 | |
| | | B3 | |
| JUP- | accacgccgagctcagtt | PD | cacgcactctgtgaagtgg |
| F3v1 | | CEACAM1v8 | |
| | | FL | |
| JUP- | ggttttcttgagcgtgtactgg | PD | gcccagctcactactgaatcc |
| B3v1 | | CEACAM1v8 | |
| | | BL | |
| JUP- | ttgataggctgctccatcaggttttttcggtcaggcc | PD | tgtgagcagaagcccctaagctctcctccacag |
| FIPv1 | ccatactca | CEACAM1v8 | gtga |
| | | FIP | |
| JUP- | gtgactgagtggcagcagatttttcctccatgatgcc | PD | taaccttctggaacccgccccttcccctctgcaa |
| BIPv1 | cttgct | CEACAM1v8 | cattga |
| | | BIP | |
| JUP- | catcacctccatcgtggctac | PD | gcctcacttctaaccttctggaa |
| LFv1 | | CEACAM1v9 | |
| | | F3 | |
| JUP- | catacacctacgactcgggtatc | PD | gtctctcgaccgctgtttg |
| LBv1 | | CEACAM1v9 | |
| | | B3 | |
| JUP- | accacgccgagctcagtt | PD | ccttcccctctgcaacattga |
| F3v1 | | CEACAM1v9 | |
| | | FL | |
| JUP- | ggttttcttgagcgtgtactgg | PD | ggaaagagtggatggcaacc |
| B3v1 | | CEACAM1v9 | |
| | | BL | |
| JUP- | ttgataggctgctccatcaggtcggtcaggccccata | PD | attgtggacaaggagaagaacctgcccagctc |
| FIPv2 | ctca | CEACAM1v9 | actactgaatcc |
| | | FIP | |
| JUP- | gtgactgagtggcagcagacctccatgatgcccttgc | PD | ccagcaactttttggctacagagttcctattgc |
| BIPv2 | t | CEACAM1v9 | atatcctacaatttga |
| | | BIP | |
| JUP- | catcacctccatcgtggctac | PD | gcccagctcactactgaatcc |
| LFv1 | | CEACAM1v10 | |
| | | F3 | |
| JUP- | catacacctacgactcgggtatc | PD | gcttcttcattcacaagatctgacttta |
| LBv1 | | CEACAM1v10 | |
| | | B3 | |
| JUP- | cgagctcagttcgctgtc | PD | ctgtagccaaaaagttgctgg |
| F3v3 | | CEACAM1v10 | |
| | | FL | |
| JUP- | gtggttttcttgagcgtgtac | PD | aacagcggtcgagagacaata |
| B3v3 | | CEACAM1v10 | |
| | | BL | |
| JUP- | gtcaccttgataggctgctcaggccccatactcagta | PD | ggttgccatccactctttccaggttcttctcct |
| FIPv3 | gc | CEACAM1v10 | tgtccacaat |
| | | FIP | |
| JUP- | gagtggcagcagacatacaccctccatgatgcccttg | PD | tcaaattgtaggatatgcaataggaacttgacg |
| BIPv3 | c | CEACAM1v10 | ttctggatcagcagg |
| | | BIP | |
| JUP- | catcaggttcatcacctccatc | PD LY86v5 | tctgcagctggacaattcact |
| LFv3 | | F3 | |
| JUP- | acgactcgggtatccactc | PD LY86v5 | aggctgtaaacttgggaaagaca |
| LBv3 | | B3 | |
| JUP- | cgagctcagttcgctgtc | PD LY86v5 | catttggaatctggaatgaggcttt |
| F3v3 | | FL | |
| JUP- | gtggttttcttgagcgtgtac | PD LY86v5 | tcaagagcctgcagagactc |
| B3v3 | | BL | |
| JUP- | gtcaccttgataggctgctctttttaggccccatact | PD LY86v5 | agttgatagcacgagggctacccagaggaga |
| FIPv4 | cagtagc | FIP | ggaaagtgacaata |
| JUP- | gagtggcagcagacatacactttttcctccatgatgc | PD LY86v5 | tgggtccatacacagcctgtctgtttcccatct |
| BIPv4 | ccttgc | BIP | ccagcc |
| JUP- | catcaggttcatcacctccatc | PD LY86v6 | ccagaggagaggaaagtgacaata |
| LFv3 | | F3 | |
| JUP- | acgactcgggtatccactc | PD LY86v6 | ggctgtgaaacccttcatggt |
| LBv3 | | B3 | |
| JUP- | ccatactcagtagccacgatg | PD LY86v6 | acgagggctacagaatgacattt |
| F3v5 | | FL | |
| JUP- | ggctgttgtggacatctgg | PD LY86v6 | gggctggagatgggaaaca |
| B3v5 | | BL | |
| JUP- | agtggatacccgagtcgtagagcagcctatcaaggt | PD LY86v6 | gtgtatggacccagggacctccaaagcctcatt |
| FIPv5 | gact | FIP | ccagattcc |
| JUP- | gcatcatggaggaggatgagctccagatcaccttgg | PD LY86v6 | tgcatcaagagcctgcagaaggctgtaaactt |
| BIPv5 | ctg | BIP | gggaaagaca |
| JUP- | gtgtatgtctgctgccactc | PD LY86v9 | cttgacctagctctcatgtctcaa |
| LFv5 | | F3 | |
| JUP- | gccagtacacgctcaagaaa | PD LY86v9 | cacatgatagtagcattggcaca |
| LBv5 | | B3 | |
| JUP- | ccatactcagtagccacgatg | PD LY86v9 | ccacagaaagaaaacttgggca |
| F3v5 | | FL | |
| JUP- | ggctgttgtggacatctgg | PD LY86v9 | cctcagggagaataccaggttt |
| B3v5 | | BL | |
| JUP- | agtggatacccgagtcgtagtttttagcagcctatca | PD LY86v9 | gcatagtaaatctgctctcctttccggctcatc |
| FIPv6 | aggtgact | FIP | tgttttgaatttctccta |
| JUP- | gcatcatggaggaggatgagtttttctccagatcacc | PD LY86v9 | ggcctgtcaataatcctgaatttactggtggac |
| BIPv6 | ttggctg | BIP | cgtttttcagtgtac |
| JUP- | gtgtatgtctgctgccactc | PD KCNJ2v7 | aaccactggatcttacatgcct |
| LFv5 | | F3 | |
| JUP- | gccagtacacgctcaagaaa | PD KCNJ2v7 | ctgctttggaaaacagtctgagtt |
| LBv5 | | B3 | |
| JUP- | cgagctcagttcgctgtc | PD KCNJ2v7 | gttactttaatgactcagctgacatcc |
| F3v7 | | FL | |
| JUP- | gtggttttcttgagcgtgtac | PD KCNJ2v7 | gtgtgtcttcaccgaacattcaaa |
| B3v7 | | BL | |
| JUP- | gtcaccttgataggctgctctttttaggccccatact | PD KCNJ2v7 | accaaggtctgtctactgacatgccagcaaca |
| FIPv7 | cagtagc | FIP | ggacatgttctc |
| JUP- | gagtggcagcagacatacactttttcctccatgatgc | PD KCNJ2v7 | aaccacaaggctcccagagtgcaaaacgcttt |
| BIPv7 | ccttgc | BIP | ggagaaaca |
| JUP- | catcaggttcatcacctccatc | PD KCNJ2v8 | ctgcatgtcagtagacagacctt |
| LFv7 | | F3 | |
| JUP- | acgactcgggtatccactc | PD KCNJ2v8 | ccatttgcaactgccatggt |
| LBv7 | | B3 | |
| OASL- | gcactgatgcaggaactgtata | PD KCNJ2v8 | tgcaaaacgctttggagaaaca |
| F3v1 | | FL | |
| OASL- | agctcagaaacgccacca | PD KCNJ2v8 | tgtgcgaaccaaccgcta |
| B3v1 | | BL | |
| OASL- | ctgcctcagaaactcctccatttttactccttcgtgg | PD KCNJ2v8 | tctgctttggaaaacagtctgaggtgtgtcttc |
| FIPv1 | ctcagt | FIP | accgaacattcaaa |
| OASL- | gagcatttccaggggaagctttttctgagaaccgtgc | PD KCNJ2v8 | agcagaagcgatgggcaggccaacttcatacc |
| BIPv1 | cattcc | BIP | gtcttc |
| OASL- | cagcgtctagcacctcttc | PD KCNJ2v9 | gtgtgtcttcaccgaacattcaaa |
| LFv1 | | F3 | |
| OASL- | cgggtgctgaaggtagtca | PD KCNJ2v9 | gtgtggactttactcttcccgt |
| LBv1 | | B3 | |
| OASL- | gcactgatgcaggaactgtata | PD KCNJ2v9 | ggactccagtgcttctgct |
| F3v1 | | FL | |
| OASL- | agctcagaaacgccacca | PD KCNJ2v9 | cggtatgaagttggccacc |
| B3v1 | | BL | |
| OASL- | ctgcctcagaaactcctccaactccttcgtggctcag | PD KCNJ2v9 | gttcgcacactgcccattgtttctccaaagcgt |
| FIPv2 | t | FIP | tttgca |
| OASL- | gagcatttccaggggaagcctgagaaccgtgccattc | PD KCNJ2v9 | gctacagcatcgtctcttcagcaaagccatttg |
| BIPv2 | c | BIP | caactgcc |
| OASL- | cagcgtctagcacctcttc | PD | atccaggcactgtccgtga |
| LFv1 | | ZDHHC19- | |
| | | 3v8 F3 | |
| OASL- | cgggtgctgaaggtagtca | PD | gacattggagggtgcagattc |
| LBv1 | | ZDHHC19- | |
| | | 3v8 B3 | |
| OLFM4- | tctgtttccctgccagac | PD | tggtcgaaggggttgtatcc |
| F3v1 | | ZDHHC19- | |
| | | 3v8 FL | |
| OLFM4- | tccttcacttctaccttgatcag | PD | cccaagtacatggctgaagc |
| B3v1 | | ZDHHC19- | |
| | | 3v8 BL | |
| OLFM4- | ctcactttggaaagttctttctcaggacagagtggaa | PD | aaataccagttgctggcacaggcaagtgcaga |
| FIPv1 | cgctt | ZDHHC19- | caccttcag |
| | | 3v8 FIP | |
| OLFM4- | gttaaacctaactgtccgaattgactcgaagtccagt | PD | caatttgtgcaccactgggaggcatggatgtcc |
| BIPv1 | tcagtgtaa | ZDHHC19- | agtcag |
| | | 3v8 BIP | |
| OLFM4- | aagaacatgagctgtgaattcc | PD | tacaaccccttcgaccagg |
| LFv1 | | ZDHHC19- | |
| | | 3v9 F3 | |
| OLFM4- | catcatggagaaggataccatttc | PD | tgtggaactcctggagagct |
| LBv1 | | ZDHHC19- | |
| | | 3v9 B3 | |
| PDE4B- | agtgagatggcttctaacaagttc | PD | tcccagtggtgcacaaattg |
| F3v1 | | ZDHHC19- | |
| | | 3v9 FL | |
| PDE4B- | gttgactccaaagcgtgagat | PD | atctgcaccctccaatgtcc |
| B3v1 | | ZDHHC19- | |
| | | 3v9 BL | |
| PDE4B- | ggatctccacatcattctgcttagctgacacacctct | PD | gcttcagccatgtacttgggctgtgccagcaac |
| FIPv1 | cagag | ZDHHC19- | tggtattt |
| | | 3v9 FIP | |
| PDE4B- | ctacccagaaagacagggagtgtattgtttaggcttg | PD | ctgactggacatccatgcctttgtagggaccca |
| BIPv1 | aactatgc | ZDHHC19- | gaggttg |
| | | 3v9 BIP | |
| PDE4B- | ggttccctgatcggctcat | PD NMRK1v7 | ggatgatttcttcaagccagagtct |
| LFv1 | | F3 | |
| PDE4B- | aagcagcagctcatgacc | PD NMRK1v7 | catatggaatagtcaggaaatagcttct |
| LBv1 | | B3 | |
| PDE4B- | tgtttagaccagctagagaccata | PD NMRK1v7 | acatcattttttccatgttaagtgcttc |
| F3v2 | | FL | |
| PDE4B- | cttatctgggtcatgagctgc | PD NMRK1v7 | ctgaggaaattcccattttaatcatcga |
| B3v2 | | BL | |
| PDE4B- | ctctgagaggtgtgtcagctcggtctgtcagtgagat | PD NMRK1v7 | tgcgctttccatccagcaggatttttgcagtac |
| FIPv2 | gg | FIP | gatgtgctt |
| PDE4B- | gatcagggaaccaggtgtctctccctgtctttctggg | PD NMRK1v7 | cactctgtggtatcaacagaccaattccatata |
| BIPv2 | tag | BIP | gtgtcaaggggcttataa |
| PDE4B- | cccggttcagcattcttttg | PD | ggatttttgcagtacgatgtgctt |
| LFv2 | | NMRK1v8 F3 | |
| PDE4B- | cagaatgatgtggagatcccat | PD | ccatcaaagtatcccggagagt |
| LBv2 | | NMRK1v8 B3 | |
| PDE4B- | gaggaattagactggtgtttagacc | PD | tggtctgttgataccacagagtg |
| F3v3 | | NMRK1v8 FL | |
| PDE4B- | ctccctgtctttctgggtag | PD | agaagctatttcctgactattccatatg |
| B3v3 | | NMRK1v8 BL | |
| PDE4B- | ctcccggttcagcattctttaccatacagacctaccg | PD | aaaccttcgatgattaaaatgggaattttgctg |
| FIPv3 | gtc | NMRK1v8 | gatggaaagcgca |
| | | FIP | |
| PDE4B- | ctctcagagatgagccgatcaacatcattctgcttgt | PD | ttataagccccttgacactatatggaatctgat |
| BIPv3 | ctaagaaag | NMRK1v8 | agacccttgtactcctcc |
| | | BIP | |
| PDE4B- | tgaacttgttagaagccatctcac | PD DEFA4v5 | gctcttgctacataagacctgga |
| LFv3 | | F3 | |
| PDE4B- | ggaaccaggtgtctgaatacatt | PD DEFA4v5 | gcctgaaacctgaagagca |
| LBv3 | | B3 | |
| PDE4B- | ggtagcggtgactctgctat | PD DEFA4v5 | cgaggagggcgataatcct |
| F3v4 | | FL | |
| PDE4B- | aatgtattcagacacctggttcc | PD DEFA4v5 | gccagaagaccaggacatatctatt |
| B3v4 | | BL | |
| PDE4B- | gctggtctaaacaccagtctaatcagcctaactacat | PD DEFA4v5 | agggctaccaagagaatagcagcctagcttga |
| FIPv4 | gcctgtg | FIP | ggatctgtcacc |
| PDE4B- | cctaccggtctgtcagtgagtgatcggctcatctctg | PD DEFA4v5 | caagaggtgatgaggctccagagcttttatccc |
| BIPv4 | agag | BIP | atgcaaagga |
| PDE4B- | tccagcgtttccattgctaat | PD DEFA4v7 | gccatgaggattatcgccct |
| LFv4 | | F3 | |
| PDE4B- | acaagttcaaaagaatgctgaacc | PD DEFA4v7 | gcagttcccaacacgaagtt |
| LBv4 | | B3 | |
| PDE4B- | cagcctaactacatgcctgtg | PD DEFA4v7 | catcacctcttgcctggagt |
| F3v5 | | FL | |
| PDE4B- | atttgaaatgtattcagacacctgg | PD DEFA4v7 | aaggggcatggtctgctct |
| B3v5 | | BL | |
| PDE4B- | gaccggtaggtctgtatggtaaattagcaatggaaac | PD DEFA4v7 | ccatgcaaaggaaatagatatgtcctgttctct |
| FIPv5 | gctgg | FIP | tggtagccctccagg |
| PDE4B- | cagtgagatggcttctaacaagtttccctgatcggct | PD DEFA4v7 | tgctcttcaggtttcaggctgttcgccggcaga |
| BIPv5 | catctc | BIP | atactaa |
| PDE4B- | tggtctaaacaccagtctaattcc | PD | caggacatatctatttcctttgcatgg |
| LFv5 | | DEFA4v10 | |
| | | F3 | |
| PDE4B- | aaagaatgctgaaccgggag | PD | catttttctctattctgcaagctcag |
| LBv5 | | DEFA4v10 | |
| | | B3 | |
| PDE4B- | caatggaaacgctggaggaatta | PD | gaccatgccccttgttgag |
| F3v6 | | DEFA4v10 | |
| | | FL | |
| PDE4B- | ctccctgtctttctgggtag | PD | cgtgtcgattaacgttctgct |
| B3v6 | | DEFA4v10 | |
| | | BL | |
| PDE4B- | ggttcagcattcttttgaacttgtggtgtttagacca | PD | aacacgaagttctgttcgccaaaagctctgctc |
| FIPv6 | gctagagac | DEFA4v10 | ttcaggtttc |
| | | FIP | |
| PDE4B- | ctctcagagatgagccgatcaacatcattctgcttgt | PD | ggaactgcctcattggtggcgttcccagcatga |
| BIPv6 | tcaagaaag | DEFA4v10 | cattct |
| | | BIP | |
| PDE4B- | tctcactgacagaccggtag | PD CD163v5 | gagactgaccagtgaagcca |
| LFv6 | | F3 | |
| PDE4B- | ggaaccaggtgtctgaatacatt | PD CD163v5 | cagcgtgtcaggtccttttg |
| LBv6 | | B3 | |
| PSMB9- | gagtgtttgacaagctgtcc | PD CD163v5 | acatgctactcttgccaacagt |
| F3v1 | | FL | |
| PSMB9- | ctgacctccttcacgttgg | PD CD163v5 | caaggccatgtccattccc |
| B3v1 | | BL | |
| PSMB9- | ccaaaacaagtggaggttcctcacgagcgcatctact | PD CD163v5 | tttgtctgcacagcccagcagggcgtctggaag |
| FIPv1 | gtg | FIP | tttt |
| PSMB9- | ctgcaaatgtggtgagaaatatcagcagccagctacc | PD CD163v5 | ggaaaatcaaccctgcatctttagagacactga |
| BIPv1 | atgagatg | BIP | acattgtccaccc |
| PSMB9- | cagttctatcccatggagctc | PD CD163v9 | tgtggcagtgcccatca |
| LFv1 | | F3 | |
| PSMB9- | aaatatcgagaggacttgtctgc | PD CD163v9 | ctgaccaaactctgcttctttgaat |
| LBv1 | | B3 | |
| PSMB9- | gaaccgagtgtttgacaagc | PD CD163v9 | ggtccttcctgaagtcttatcttgtt |
| F3v2 | | FL | |
| PSMB9- | gacctccttcacgttggtc | PD CD163v9 | gggacttggacgatgctca |
| B3v2 | | BL | |
| PSMB9- | caaaacaagtggaggttcctccacgagcgcatctact | PD CD163v9 | acacgtccagaacaggaagtgcggaggagac |
| FIPv2 | gtg | FIP | ctggatcaca |
| PSMB9- | gctgcaaatgtggtgagaaatatccagccagctacca | PD CD163v9 | agatctggcatggaggttccaccacagccaag |
| BIPv2 | tgagatg | BIP | ttgttgac |
| PSMB9- | cagttctatcccatggagctc | PD CD163v11 | acccacttcctgttctggac |
| LFv2 | | F3 | |
| PSMB9- | gctataaatatcgagaggacttgtc | PD CD163v11 | agcgtctggcaggacaat |
| LBv2 | | B3 | |
| PSMB9- | gatgggttctgattcccgag | PD CD163v11 | ctgagcatcgtccaagtcc |
| F3v3 | | FL | |
| PSMB9- | cagccagctaccatgagatg | PD CD163v11 | gtcaggggactggaccgata |
| B3v3 | | BL | |
| PSMB9- | ccagttctatcccatggagctgagtgtttgacaagct | PD CD163v11 | cagccaagttgttgacacaccgatctggcatgg |
| FIPv3 | gtcc | FIP | aggttcct |
| PSMB9- | ctccacttgttttggctgctgcagacaagtcctctcg | PD CD163v11 | tgaaagcattcaaagaagcagagtttccacaa |
| BIPv3 | taatt | BIP | ggaagactcattccct |
| PSMB9- | tgcacagtagatgcgctc | PD | caatctgccccagcaacttt |
| LFv3 | | CEACAM1v11 | |
| | | F3 | |
| PSMB9- | caaatgtggtgagaaatatcagcta | PD | cgggtatacatggaactgtcca |
| LBv3 | | CEACAM1v11 | |
| | | B3 | |
| PSMB9- | agcgcatctactgtgcac | PD | agttcctattgcatatcctacaatttga |
| F3v4 | | CEACAM1v11 | |
| | | FL | |
| PSMB9- | ccaatggcaaaaggctgtc | PD | caggattctacaccctacaagtcata |
| B3v4 | | CEACAM1v11 | |
| | | BL | |
| PSMB9- | tagctgatatttctcaccacatttgagctccatggga | PD | tattgtctctcgaccgctgttggaaagagtgga |
| FIPv4 | tagaactgg | CEACAM1v11 | tggcaacc |
| | | FIP | |
| PSMB9- | aatatcgagaggacttgtctgcagagtcagcattcct | PD | cctgctgatccagaacgtcagttgcttcttcat |
| BIPv4 | cccag | CEACAM1v11 | tcacaagatctga |
| | | BIP | |
| PSMB9- | gcagccaaaacaagtggag | PD | aacagcggtcgagagacaata |
| LFv4 | | CEACAM1v12 | |
| | | F3 | |
| PSMB9- | aacgtgaaggaggtcaggtat | PD | gaggctctgattgtttatccacc |
| LBv4 | | CEACAM1v12 | |
| | | B3 | |
| PSMB9- | tgcactctctggttcagc | PD | ccagttgcttcttcattcacaagatc |
| F3v5 | | CEACAM1v12 | |
| | | FL | |
| PSMB9- | ccaatggcaaaaggctgtc | PD | gtggccttcacctgtgaac |
| B3v5 | | CEACAM1v12 | |
| | | BL | |
| PSMB9- | gcagacaagtcctctcgatatttagctccatgggata | PD | tgttgctggagatggaggggattctacacccta |
| FIPv5 | gaactgg | CEACAM1v12 | caagtcataaagtc |
| | | FIP | |
| PSMB9- | catctcatggtagctggctggagtcagcattcctccc | PD | tccaaccctgtggaggacaagtaggttgtgtcc |
| BIPv5 | ga | CEACAM1v12 | tgagtctca |
| | | BIP | |
| PSMB9- | ctgatatttctcaccacatttgcag | PD | ccctacaagtcataaagtcagatcttgt |
| LFv5 | | CEACAM1v13 | |
| | | F3 | |
| PSMB9- | aacgtgaaggaggtcaggtat | PD | ctgttgccattggacagct |
| LBv5 | | CEACAM1v13 | |
| | | B3 | |
| PSMB9- | gacttgtctgcacatctcatggta | PD | ggcagctccgggtatacat |
| F3v6 | | CEACAM1v13 | |
| | | FL | |
| PSMB9- | taatagtgaccaggtagatgacacc | PD | cacctgtgaacctgagactca |
| B3v6 | | CEACAM1v13 | |
| | | BL | |
| PSMB9- | ggagccaccaatggcaaaagccaacgtgaaggagg | PD | tccacagggttggagttgttggaatgaagaag |
| FIPv6 | tcaggtat | CEACAM1v13 | caactggacagt |
| | | FIP | |
| PSMB9- | gcagcacctttatctatggttatgtcaatagcgtctg | PD | gacaaggatgctgtggccttgaggctctgattg |
| BIPv6 | tggtgaagc | CEACAM1v13 | tttatccacc |
| | | BIP | |
| PSMB9- | cagcattcctcccagggttc | PD | ccaaccgctacagcatcgt |
| LFv6 | | KCNJ2v10 F3 | |
| PSMB9- | agcatataagccaggcatgtctc | PD | ggcagaagataaccagcatcc |
| LBv6 | | KCNJ2v10 | |
| | | B3 | |
| PSMB9- | caaatgtggtgagaaatatcagcta | PD | caaagccatttgcaactgcc |
| F3v7 | | KCNJ2v10 | |
| | | FL | |
| PSMB9- | tgcactcctcgggagacatg | PD | caacggtacctcgcagacat |
| B3v7 | | KCNJ2v10 | |
| | | BL | |
| PSMB9- | gtcagcattcctcccagggtggacttgtctgcacatc | PD | gtgtggactttactcttcccgtcggtatgaagt |
| FIPv7 | ctatgg | KCNJ2v10 | tggccacc |
| | | FIP | |
| PSMB9- | cgacagccttttgccattggtggcttatatgctgcat | PD | aaagatggccactgtaatgttcagccagcgaa |
| BIPv7 | ccacat | KCNJ2v10 | tgtccacacac |
| | | BIP | |
| PSMB9- | catatacctgacctccttcacgttg | PD | caggagccgctttgtgaa |
| LFv7 | | KCNJ2v11 F3 | |
| PSMB9- | gctccggcagcacctttat | PD | ctttgccctctttggatgca |
| LBv7 | | KCNJ2v11 B3 | |
| PSMB9- | tgcaaatgtggtgagaaatatcagc | PD | gatgtctgcgaggtaccgtt |
| F3v8 | | KCNJ2v11 FL | |
| PSMB9- | ggagacatgcctggcttatatgc | PD | gctttcgtcctgtcatggc |
| B3v8 | | KCNJ2v11 BL | |
| PSMB9- | ccagggttccatatacctgacctataaatatcgagag | PD | agcgaatgtccacacacgtgatggccactgta |
| FIPv8 | gacttgtctgc | KCNJ2v11 | atgttcagtt |
| | | FIP | |
| PSMB9- | gaggaatgctgactcgacagctccacataaccatag | PD | ggatgctggttatcttctgcccatggagcagag |
| BIPv8 | ataaaggtgc | KCNJ2v11 | ctatcaacca |
| | | BIP | |
| PSMB9- | cccagccagctaccatgagat | PD | cctggctttcgtcctgtcat |
| LFv8 | | KCNJ2v12 F3 | |
| PSMB9- | cttttgccattggtggctcc | PD | cacgattgactggaacaccac |
| LBv8 | | KCNJ2v12 B3 | |
| RAPGEF- | gctactttaagaccattgtggacaa | PD | ctttgccctctttggatgca |
| F3v1 | | KCNJ2v12 FL | |
| RAPGEF- | tgtcttctgagttcacgcctt | PD | ggctatggtttcagatgtgtcac |
| B3v1 | | KCNJ2v12 | |
| | | BL | |
| RAPGEF- | gtgctgaattcgaggatcgttcagaaggtactggaga | PD | gttgacctcggacacacaaggtgttttggttga |
| FIPv1 | tgcttcc | KCNJ2v12 | tagctctgct |
| | | FIP | |
| RAPGEF- | ttcctgctatagccgagtgtaccagcatcacttggtc | PD | ttctccattgagacccagacaaccatgaaaac |
| BIPv1 | agacc | KCNJ2v12 | agcaattgggcattc |
| | | BIP | |
| RAPGEF- | aggggtaagatggcctccag | PE C3AR1v1- | AGATAGTGGTCTAGAGCATAAG |
| LFv1 | | 1 F3 | ACT |
| RAPGEF- | agcctcgccaacctcatt | PE C3AR1v1- | AGAGTAGGTCAGTTGAATTGGT |
| LBv1 | | 1 B3 | C |
| RAPGEF- | agaaggtactggagatgcttcc | PE C3AR1v1- | ACACTTAGCCACAGTGAAGTCA |
| F3v2 | | 1 FIP | TGACGGAAACAGAGAGAGAACA |
| | | | GAA |
| RAPGEF- | cagcacagccttgatgacc | PE C3AR1v1- | CGTGGAGACATCCAGGTGCTGC |
| B3v2 | | 1 BIP | AGCAGAGAAAGACGCCAT |
| RAPGEF- | gtacactcggctatagcaggaagtgctggaggccat | PE C3AR1v1- | GGCAAGAAAATTTGCTGAGCTT |
| FIPv2 | cttac | 1 FL | TC |
| RAPGEF- | aacctcattcgctggtctgaagtcgtcaccatctcct | PE C3AR1v1- | AAGCCTTCAGCTACTGTCTCA |
| BIPv2 | tgtc | 1 BL | |
| RAPGEF- | gaggatcgttctgcaccag | PE C3AR1v5- | AGAGAGAACAGAAGAAGAGAAA |
| LFv2 | | 1 F3 | GC |
| RAPGEF- | tggaaggcgtgaactcagaa | PE C3AR1v5- | CTGGGGGCTCATTCCATG |
| LBv2 | | 1 B3 | |
| RAPGEF- | gtgctggaggccatcttac | PE C3AR1v5- | TCCACGAGTCCTGTCTGGTCCG |
| F3v3 | | 1 FIP | CAAATTTTCTTGCCATACTTCAT |
| | | | G |
| RAPGEF- | cctgaccagctccttcactc | PE C3AR1v5- | GAAGCCTTCAGCTACTGTCTCA |
| B3v3 | | 1 BIP | GTTGAGAGTAGGTCAGTTGAAT |
| | | | TGGT |
| RAPGEF- | cactcggctatagcaggaagactggtgcagaacgat | PE C3AR1v5- | CCACACTTAGCCACAGTGAAGT |
| FIPv3 | cctc | 1 FL | |
| RAPGEF- | aacctcattcgctggtctgaagtcgtcaccatctcct | PE C3AR1v5- | TTGAAGTTTAGCAATGGCGTCT |
| BIPv3 | tgtc | 1 BL | |
| RAPGEF- | ggctgagctgtgctgaattc | PE C3AR1v7- | AGAGAAAGCTCAGCAAATTTTC |
| LFv3 | | 1 F3 | TTG |
| RAPGEF- | gatgctggaaggcgtgaac | PE C3AR1v7- | AATTACTGGGGGCTCATTCC |
| LBv3 | | 1 B3 | |
| RAPGEF1- | gctgctattctgccctttcag | PE C3AR1v7- | GCACCTGGATGTCTCCACGAGT |
| F3v4 | | 1 FIP | TCATGACTTCACTGTGGCTAAG |
| RAPGEF1- | ctccatgaggagcttgttcttc | PE C3AR1v7- | GAAGCCTTCAGCTACTGTCTCA |
| B3v4 | | 1 BIP | GTTGAGAGTAGGTCAGTTGAAT |
| | | | TGGT |
| RAPGEF1- | ctgcatgtaggccagcatgttcctcagcccctgtcga | PE C3AR1v7- | CCTGTCTGGTCCCCACA |
| FIPv4 | at | 1 FL | |
| RAPGEF1- | ttgctggaggactactcggatgctggtagatgtgctc | PE C3AR1v7- | TTTTGAAGTTTAGCAATGGCGT |
| BIPv4 | gtt | 1 BL | |
| RAPGEF1- | gactcaggagcagtaaaatcacc | PE | GGACCAGACAGGACTCGTG |
| LFv4 | | C3AR1v17-1 | |
| | | F3 | |
| RAPGEF1- | ccgcagccctctatgttctac | PE | GGCCAGCCACCCACA |
| LBv4 | | C3AR1v17-1 | |
| | | B3 | |
| RAPGEF1- | gccctttcagcatggaggtt | PE | ATTGGTCTCAGCAGAGAAAGAC |
| F3v5 | | C3AR1v17-1 | GCTCCAGGTGCTGAAGCCT |
| | | FIP | |
| RAPGEF1- | ggagtcgctgaagccgtata | PE | GACCTACTCTCACAGCCATGGA |
| B3v5 | | C3AR1v17-1 | ATGGCAATCCCAGTAAAAAAGT |
| | | BIP | AAGGC |
| RAPGEF1- | ccagcaactgcatgtaggcgtcgaatttgtgggtgat | PE | CTTCAAAAAACTGAGACAGTAG |
| FIPv5 | tttactg | C3AR1v17-1 | CTG |
| | | FL | |
| RAPGEF1- | gcagccctctatgttctaccactccatgaggagcttg | PE | AGCCCCCAGTAATTCTCTCC |
| BIPv5 | ttcttc | C3AR1v17-1 | |
| | | BL | |
| RAPGEF1- | gtcaccggttgactcaggag | PE | GACATCCAGGTGCTGAAGC |
| LFv5 | | C3AR1v19-1 | |
| | | F3 | |
| RAPGEF1- | gccacagaacgagcacatcta | PE | AATTGTGTTCACTGTCCGCT |
| LBv5 | | C3AR1v19-1 | |
| | | B3 | |
| RAPGEF1- | ccctttcagcatggaggttc | PE | ATGGCTGTGAGAGTAGGTCAGT |
| F3v6 | | C3AR1v19-1 | TGAGCTACTGTCTCAGTTTTTTG |
| | | FIP | AAGTT |
| RAPGEF1- | ctccatgaggagcttgttcttc | PE | GCCCCCAGTAATTCTCTCCATGG |
| B3v6 | | C3AR1v19-1 | CACAGCACCAGCCCATT |
| | | BIP | |
| RAPGEF1- | ctgcatgtaggccagcatgctcagcccctgtcgaatt | PE | GCAGAGAAAGACGCCATTGCTA |
| FIPv6 | tg | C3AR1v19-1 | |
| | | FL | |
| RAPGEF1- | ttgctggaggactactcggagctggtagatgtgctcg | PE | TTCTCAGCCTTACTTTTTTACTG |
| BIPv6 | ttc | C3AR1v19-1 | GG |
| | | BL | |
| RAPGEF1- | gactcaggagcagtaaaatcacc | PE DEFA4v1- | CATGGCTGCTCTTGCTACA |
| LFv6 | | 1 F3 | |
| RAPGEF1- | ccgcagccctctatgttctac | PE DEFA4v1- | TGTCCTGGTCTTCTGGCC |
| LBv6 | | 1 B3 | |
| RAPGEF1- | gtcgaatttgtgggtgattttactg | PE DEFA4v1- | TGGGGTGACAGATCCTCAAGCT |
| F3v7 | | 1 FIP | AGACCTGGAACACAGGACTG |
| RAPGEF1- | ggagtcgctgaagccgtata | PE DEFA4v1- | TTATCGCCCTCCTCGCTGCTGCC |
| B3v7 | | 1 BIP | TCATCACCTCTTGCC |
| RAPGEF1- | cgagtagtcctccagcaactctcctgagtcaaccggt | PE DEFA4v1- | GCGAGCAGAGAGGGCA |
| FIPv7 | gac | 1 FL | |
| RAPGEF1- | gcagccctctatgttctaccactccatgaggagcttg | PE DEFA4v1- | CCTCCAGGTCCGGGCA |
| BIPv7 | ttcttc | 1 BL | |
| RAPGEF1- | catgtaggccagcatgtgttt | PE | CATGGCTGCTCTTGCTACA |
| LFv7 | | DEFA4v4-1 | |
| | | F3 | |
| RAPGEF1- | gccacagaacgagcacatcta | PE | TGTCCTGGTCTTCTGGCC |
| LBv7 | | DEFA4v4-1 | |
| | | B3 | |
| RAPGEF1- | cccaaagtcaccagatgctc | PE | CTCATGGCTGGGGTGACAGATC |
| F3v8 | | DEFA4v4-1 | GACTGCTGTCTGCCCTCT |
| | | FIP | |
| RAPGEF1- | gtactgcagcttcttgatgagc | PE | TTATCGCCCTCCTCGCTGCTGCC |
| B3v8 | | DEFA4v4-1 | TCATCACCTCTTGCC |
| | | BIP | |
| RAPGEF1- | cagtaagatgtccccagatcctcggacgagctgtccc | PE | CTAGGCAGGGCGAGCA |
| FIPv8 | tcatt | DEFA4v4-1 | |
| | | FL | |
| RAPGEF1- | gtccatgctactgagactgacacctgtaggtggtcag | PE | ATTCTCTTGGTAGCCCTCCA |
| BIPv8 | gaatgc | DEFA4v4-1 | |
| | | BL | |
| RAPGEF1- | gcgtcagcctggacataatttc | PE | GCCTAGCTTGAGGATCTGTC |
| LFv8 | | DEFA4v8-1 | |
| | | F3 | |
| RAPGEF1- | agatttggtgttgtactgcgag | PE | CATGCCCCTTGTTGAGCC |
| LBv8 | | DEFA4v8-1 | |
| | | B3 | |
| RAPGEF1- | ccaaagtcaccagatgctctg | PE | CACCTCTTGCCTGGAGTGGGTG |
| F3v9 | | DEFA4v8-1 | AGGATTATCGCCCTCCT |
| | | FIP | |
| RAPGEF1- | gtactgcagcttcttgatgagc | PE | CGTGGGCCAGAAGACCAGGATG |
| B3v9 | | DEFA4v8-1 | AAACCTGAAGAGCAGAGC |
| | | BIP | |
| RAPGEF1- | cagtagcatggaccagtaagatgtgaggaagtggac | PE | GCTACCAAGAGAATAGCAGCG |
| FIPv9 | gagctgtc | DEFA4v8-1 | |
| | | FL | |
| RAPGEF1- | gactgacaggaaagatttggtgttggggagatgaag | PE | CATATCTATTTCCTTTGCATGGG |
| BIPv9 | gtcctgtag | DEFA4v8-1 | AT |
| | | BL | |
| RAPGEF1- | gagcgtcagcctggacataa | PE KCNJ2v1- | CAGCGCGCAGCCTTC |
| LFv9 | | 1 F3 | |
| RAPGEF1- | gtactgcgaggcattcctg | PE KCNJ2v1- | TGACATGCAGAGTTACTTTAAT |
| LBv9 | | 1 B3 | GAC |
| S100A12- | cttggctcagtgcccttcac | PE KCNJ2v1- | TGTAAGATCCAGTGGTTTGTAA |
| F3v1 | | 1 FIP | AAAGCGGGCGGGCTGGGTCTT |
| S100A12- | tgtttgcaagctcctttgtaagc | PE KCNJ2v1- | TGCCTCTGTACCCCCCACTTCTC |
| B3v1 | | 1 BIP | AGCTGACATCCAGAGAACA |
| S100A12- | cctccagatgctcttcaagttttggcgctgtagctcc | PE KCNJ2v1- | GCCAAAGCAAACCAGAATTCCC |
| FIPv1 | acattc | 1 FL | |
| S100A12- | cttccaccaatactcagttcggagctgcttcagctca | PE KCNJ2v1- | ACTCCATGTCCCCATGCTCCT |
| BIPv1 | ccctta | 1 BL | |
| S100A12- | cccagcctaatgttaacccctc | PE KCNJ2v4- | GGCGGGCTGGGTCTT |
| LFv1 | | 2 F3 | |
| S100A12- | aggggcattttgacaccctc | PE KCNJ2v4- | GTTCTACCAAGGTCTGTCTACTG |
| LBv1 | | 2 B3 | |
| S100A12- | ggctttttgctgtagctccacat | PE KCNJ2v4- | TGGGGGGTACAGAGGCATGTAA |
| F3v2 | | 2 FIP | GTCTGGTTTGCTTTGGCTCAC |
| S100A12- | tgtttgcaagctcctttgtaagc | PE KCNJ2v4- | CCATGTCCCCATGCTCCTGCCAT |
| B3v2 | | 2 BIP | GCAGAGTTACTTTAATGACTCA |
| S100A12- | gacaattccctccagatgctctttcctgtgcattgag | PE KCNJ2v4- | TCCAGTGGTTTGTAAAAAGCGA |
| FIPv2 | gggttaac | 2 FL | |
| S100A12- | cttccaccaatactcagttcggagctgcttcagctca | PE KCNJ2v4- | GCCAGCAACAGGACATGTTC |
| BIPv2 | ccctta | 2 BL | |
| S100A12- | agttttgtcatcttcccagcctaat | PE KCNJ2v9- | GGGCTGGGTCTTGGGAAT |
| LFv2 | | 1 F3 | |
| S100A12- | aggggcattttgacaccctc | PE KCNJ2v9- | GGAGCCTTGTGGTTCTACC |
| LBv2 | | 1 B3 | |
| S100A12- | gctgtagctccacattcctg | PE KCNJ2v9- | AGCATGGGGACATGGAGTGGAA |
| F3v3 | | 1 FIP | GGCTCACTCGCTTTTTACAAAC |
| S100A12- | tgcaagctcctttgtaagcag | PE KCNJ2v9- | CCTGCGCCAGCAACAGGACTGT |
| B3v3 | | 1 BIP | CTACTGACATGCAGAGTT |
| S100A12- | attgacaattccctccagatgctgcattgaggggtta | PE KCNJ2v9- | TGGGGGGTACAGAGGCAT |
| FIPv3 | acattagg | 1 FL | |
| S100A12- | cttccaccaatactcagttcggctgcttcagctcacc | PE KCNJ2v9- | CATGTTCTCTGGATGTCAGCTG |
| BIPv3 | ctta | 1 BL | AGT |
| S100A12- | cttcaagttttgtcatcttcccag | PE | GCTTTTTACAAACCACTGGATCT |
| LFv3 | | KCNJ2v16-1 | |
| | | F3 | |
| S100A12- | ggggcattttgacaccctc | PE | TGAATGTTCGGTGAAGACACAC |
| LBv3 | | KCNJ2v16-1 | |
| | | B3 | |
| S100A12- | cttggctcagtgcccttcac | PE | AGAACATGTCCTGTTGCTGGCG |
| F3v1 | | KCNJ2v16-1 | CCTCTGTACCCCCCACTTC |
| | | FIP | |
| S100A12- | tgtttgcaagctcctttgtaagc | PE | AGTAACTCTGCATGTCAGTAGA |
| B3v1 | | KCNJ2v16-1 | CAGACCACACCAAAAAAATGAG |
| | | BIP | GAGAGATG |
| FURIN- | ttggtgaagaccttctggctttttgcaacaggaacct | PE | AGCATGGGGACATGGAGTG |
| FIPv1 + | tggtcc | KCNJ2v16-1 | |
| t | | FL | |
| FURIN- | caacagtgtggcacggaatttttggtaatagtccccg | PE | TTGGTAGAACCACAAGGCTCC |
| BIPv1 + | aagatctgg | KCNJ2v16-1 | |
| t | | BL | |
| S100A12- | cccagcctaatgttaacccctc | PE | TCCACTCCATGTCCCCATG |
| LFv1 | | KCNJ2v21-2 | |
| | | F3 | |
| S100A12- | aggggcattttgacaccctc | PE | TGGAAAACAGTCTGAGTTTTTG |
| LBv1 | | KCNJ2v21-2 | C |
| | | B3 | |
| S100A12- | ggctttttgctgtagctccacat | PE | GTCTGTCTACTGACATGCAGAG |
| F3v2 | | KCNJ2v21-2 | TTACTTCTCCTGCGCCAGCAAC |
| | | FIP | |
| S100A12- | tgtttgcaagctcctttgtaagc | PE | GTAGAACCACAAGGCTCCCAGA |
| B3v2 | | KCNJ2v21-2 | GAAACAGTTTTGAATGTTCGGT |
| | | BIP | GA |
| S100A12- | gacaattccctccagatgctcttttttttcctgtgca | PE | TGACATCCAGAGAACATGTCCT |
| FIPv2 + | ttgaggggttaac | KCNJ2v21-2 | |
| t | | FL | |
| S100A12- | cttccaccaatactcagttcggatttttgctgcttca | PE | CACCCATCTCTCCTCATTTTTTT |
| BIPv2 + | gctcaccctta | KCNJ2v21-2 | GG |
| t | | BL | |
| S100A12- | agttttgtcatcttcccagcctaat | PE | CCACTGGGACCCAAGTACA |
| LFv2 | | ZDHHC19v27- | |
| | | 8 F3 | |
| S100A12- | aggggcattttgacaccctc | PE | GCCCGTGTGGAACTCCT |
| LBv2 | | ZDHHC19v27- | |
| | | 8 B3 | |
| S100A12- | gctgtagctccacattcctg | PE | AGGGTGCAGATTCGGCATGGAA |
| F3v3 | | ZDHHC19v27- | GCTGTCCAGCTGCAGAGA |
| | | 8 FIP | |
| S100A12- | tgcaagctcctttgtaagcag | PE | CTCAACCCCCCAGCCCCAACGG |
| B3v3 | | ZDHHC19v27- | AGAGCTGCAGCCTCAC |
| | | 8 BIP | |
| S100A12- | attgacaattccctccagatgcttttttgcattgagg | PE | TGTCCAGTCAGGCCCCA |
| FIPv3 + | ggttaacattagg | ZDHHC19v27- | |
| t | | 8 FL | |
| S100A12- | cttccaccaatactcagttcggtttttctgcttcagc | PE | GGGTCCCTACAAAGCAGGGAAG |
| BIPv3 + | tcaccctta | ZDHHC19v27- | |
| t | | 8 BL | |
| S100A12- | cttcaagttttgtcatcttcccag | PE | GGGACCCAAGTACATGGCT |
| LFv3 | | ZDHHC19v34- | |
| | | 1 F3 | |
| S100A12- | ggggcattttgacaccctc | PE | GCTCCTGCAGCAGCAG |
| LBv3 | | ZDHHC19v34- | |
| | | 1 B3 | |
| S100A12- | ttccttggctcagtgccctt | PE | AGAGGGGGACATTGGAGGGTG |
| F3v4 | | ZDHHC19v34- | GAGAGTGGTGGGGCCTGA |
| | | 1 FIP | |
| S100A12- | ctgcttcagctcacccttagag | PE | ACCTCTGGGTCCCTACAAAGCA |
| B3v4 | | ZDHHC19v34- | GTGGAACTCCTGGAGAGCT |
| | | 1 BIP | |
| S100A12- | aagttttgtcatcttcccagcctggctttttgctgta | PE | ATTCGGCATGGATGTCCAG |
| FIPv4 | gctccacat | ZDHHC19v34- | |
| | | 1 FL | |
| S100A12- | gaagagcatctggagggaattgtagggtgtcaaaat | PE | GGGGCGTGGTGAGGCT |
| BIPv4 | gccccttc | ZDHHC19v34- | |
| | | 1 BL | |
| S100A12- | tgttaacccctcaatgcacagg | PE | CAGCTGCAGAGAGTGGTG |
| LFv4 | | ZDHHC19v45- | |
| | | 1 F3 | |
| S100A12- | atatcttccaccaatactcagttcg | PE | GCCCGTGTGGAACTCCT |
| LBv4 | | ZDHHC19v45- | |
| | | 1 B3 | |
| S100A12- | aggctgggaagatgacaaaactt | PE | GGTTGAGAGCAGAGGGGGACA |
| F3v5 | | ZDHHC19v45- | GGGCCTGACTGGACATCC |
| | | 1 FIP | |
| S100A12- | gcaatggctaccagggatatgaa | PE | CCAGCCCCAACCTCTGGGTGGA |
| B3v5 | | ZDHHC19v45- | GAGCTGCAGCCTCAC |
| | | 1 BIP | |
| S100A12- | gagggtgtcaaaatgcccctaagagcatctggaggg | PE | AGGGTGCAGATTCGGCAT |
| FIPv5 | aattgtc | ZDHHC19v45- | |
| | | 1 FL | |
| S100A12- | gcttacaaaggagcttgcaaacaagtcgacctgttca | PE | CCCTACAAAGCAGGGAAGGG |
| BIPv5 | tcttgattagc | ZDHHC19v45- | |
| | | 1 BL | |
| S100A12- | tccgaactgagtattggtggaag | PE | ACATCCATGCCGAATCTGC |
| LFv5 | | ZDHHC19v66- | |
| | | 1 F3 | |
| S100A12- | cattgatgaaatattccaaggcctg | PE | GGCATCTCCGGGTGCA |
| LBv5 | | ZDHHC19v66- | |
| | | 1 B3 | |
| S100A12- | gagggaattgtcaatatcttccacc | PE | TTCCCTGCTTTGTAGGGACCCAT |
| F3v6 | | ZDHHC19v66- | CCAATGTCCCCCTCTGCT |
| | | 1 FIP | |
| S100A12- | cagagagctacctactctttgtgg | PE | CAGCTCTCCAGGAGTTCCACAC |
| B3v6 | | ZDHHC19v6 | AGGAAGGCCGAACCTCG |
| | | 6-1 BIP | |
| S100A12- | caagctcctttgtaagcagctctcagttcggaagggg | PE | GGGCTGGGGGGTTGAG |
| FIPv6 | cattt | ZDHHC19v6 | |
| | | 6-1 FL | |
| S100A12- | aatattccaaggcctggatgctaatttcagcgcaatg | PE | CCCCTGCTGCTGCAGGAG |
| BIPv6 | gctaccag | ZDHHC19v6 | |
| | | 6-1 BL | |
| S100A12- | gcttcagctcacccttagagag | PE | AAACAGCAGAGGTGACAGAG |
| LFv6 | | CEACAM1v1- | |
| | | 1 F3 | |
| S100A12- | caagatgaacaggtcgactttcaag | PE | GCGGGTTCCAGAAGGTTAG |
| LBv6 | | CEACAM1v1- | |
| | | 1 B3 | |
| S100A12- | cggaaggggcattttgacac | PE | TCCTGCTGGCCCTGTCTTCA- |
| F3v7 | | CEACAM1v1- | GTGCTCGAAGCGTTCCTG |
| | | 1 FIP | |
| S100A12- | ggacattgctgggtaaaaagcctt | PE | GACACCATGGGGCACCTCTC- |
| B3v7 | | CEACAM1v1- | AGTGAGGCTGTGAGCAGAA |
| | | 1 BIP | |
| S100A12- | attcttgaaagtcgacctgttcatcacaaaggagctt | PE | TGTGGAGGAGAGCTTGGG |
| FIPv7 | gcaaacacc | CEACAM1v1- | |
| | | 1 FL | |
| S100A12- | tatccctggtagccattgcgcagagagctacctactc | PE | AGCCCCACTTCACAGAGTG |
| BIPv7 | tttgtgg | CEACAM1v1- | |
| | | 1 BL | |
| S100A12- | attagcatccaggccttggaatatt | PE | CTTCACAGAGTGCGTGTACC |
| LFv7 | | CEACAM1v2- | |
| | | 1 F3 | |
| S100A12- | ctgaaggctgcccattacc | PE | GGTTGCCATCCACTCTTTCC |
| LBv7 | | CEACAM1v2- | |
| | | 1 B3 | |
| S100A12- | cttggctcagtgcccttcac | PE | TTCAGTAGTGAGCTGGGCAGTG |
| F3v1 | | CEACAM1v2- | -TGCTCACAGCCTCACTTCT |
| | | 1 FIP | |
| S100A12- | tgtttgcaagctcctttgtaagc | PE | ATGTTGCAGAGGGGAAGGAGGT |
| B3v1 | | CEACAM1v2- | -CTTTGTACCAGCTGTAGCCA |
| | | 1 BIP | |
| S100A12- | cctccagatgctcttcaagttttggcgctgtagctcc | PE | GGCGGGTTCCAGAAGGTT |
| FIPv1 | caattc | CEACAM1v2- | |
| | | 1 FL | |
| S100A12- | cttccaccaatactcagttcggagctgcttcagctca | PE | TCTTCTCCTTGTCCACAATCTGC |
| BIPv1 | ccctta | CEACAM1v2- | |
| | | 1 BL | |
| S100A12- | cccagcctaatgttaacccctc | PE | CCCAGCTCACTACTGAATCC |
| LFv1 | | CEACAM1v3- | |
| | | 1 F3 | |
| S100A12- | aggggcattttgacaccctc | PE | GTCTCTCGACCGCTGTTTG |
| LBv1 | | CEACAM1v3- | |
| | | 1 B3 | |
| PD | gtacctggtttgcatagatgattggca | PE | GCCAAAAAGTTGCTGGGGCAG- |
| CTSBv6 | | CEACAM1v3- | |
| F3 | | 1 FIP | TCAATGTTGCAGAGGGGAAG |
| PD | cacccaggaaggtaccacat | PE | AAGGGGAAAGAGTGGATGGCA |
| CTSBv6 | | CEACAM1v3- | |
| B3 | | 1 BIP | A-TGGGGTAGCTTGTTGAGTTC |
| PD | ctccgggcattggccaaca | PE | TGTGGACAAGGAGAAGAACCTC |
| CTSBv6 | | CEACAM1v3- | |
| FL | | 1 FL | |
| PD | caggccgggcacaacttcta | PE | CCGTCAAATTGTAGGATATGCA |
| CTSBv6 | | CEACAM1v3- | ATAG |
| BL | | 1 BL | |
| PD | acaggggatggaaagagggccatctaggatccggct | PE | GGGGAAGGAGGTTCTTCTCC |
| CTSBv6 | tccaacatgt | CEACAM1v10- | |
| FIP | | 1 F3 | |
| PD | tggtcaactatgtcaacaaacggaataccacctcttc | PE | CTGTGTCATTCTGGGTGACG |
| CTSBv6 | aagtagctcatgtccacgtt | CEACAM1v10- | |
| BIP | | 1 B3 | |
| PD | gcctgcagtacctggtttgcatagat | PE | GGTTGCCATCCACTCTTTCCCC- |
| CTSBv7 | | CEACAM1v10- | CAATCTGCCCCAGCAACTT |
| F3 | | 1 FIP | |
| PD | cacccaggaaggtaccacat | PE | AACTCAACAAGCTACCCCAGGG- |
| CTSBv7 | | CEACAM1v10- | TGGATCAGCAGGGATGCA |
| B3 | | 1 BIP | |
| PD | ctccgggcattggccaaca | PE | TTTGTACCAGCTGTAGCCAAA |
| CTSBv7 | | CEACAM1v10- | |
| FL | | 1 FL | |
| PD | caggccgggcacaacttcta | PE | CCCGCAAACAGCGGTC |
| CTSBv7 | | CEACAM1v10- | |
| BL | | 1 BL | |
| PD | acaggggatggaaagagggccatctaggatccggct | PE ZDHH19- | TCCTCCCTCCAGTGAGTG |
| CTSBv7 | tccaacatgt | 207v19-1 F3 | |
| FIP | | | |
| PD | tgagctggtcaactatgtcaacaaacggcctcttcaa | PE ZDHH19- | GGGTTGTATCCCTGAAGGTG |
| CTSBv7 | gtagctcatgtccac | 207v19-1 B3 | |
| BIP | | | |
| PD JUPv4 | CCCTCCGTCAGCAGCAA | PE ZDHH19- | GTGTCCCATGTCCCCGCTCTGG |
| F3 | | 207v19-1 FIP | AGTCCTGGTTCCTTTGG |
| PD JUPv4 | CACACCAGGGCACATGG | PE ZDHH19- | GGAGTTGGTGGTGGGCAGAGAC |
| B3 | | 207v19-1 BIP | TTCAAGAGCCTTTGCTGT |
| PD JUPv4 | ACCCCCTGGGTGTAAGTGGTGGGGC | PE ZDHH19- | CTGAGATTTTCAACACCATTCTC |
| FIP | ATCATGGAGGAGGAT | 207v19-1 FL | CA |
| PD JUPv4 | GCCCCCCAGCCAAGGTGATCTCCCG | PE ZDHH19- | ATGCTGCTGGGCCACT |
| BIP | CACCCGTTTG | 207v19-1 BL | |
| PD JUPv1 | ATGAACCTGATGGAGCAGC | PE ZDHH19- | TGGTGGTGGGCAGAGATG |
| F3 | | 207v25-1 F3 | |
| PD JUPv1 | GCACCCCCTGGGTGTA | PE ZDHH19- | ACTCTCTGCAGCTGGACA |
| B3 | | 207v25-1 B3 | |
| PD JUPv1 | TTGGCGCCCGAGTGGATACCCAAGG | PE ZDHH19- | TCCCTGAAGGTGTCTGCACTGA |
| FIP | TGACTGAGTGGCAGC | 207v25-1 FIP | CTGGGCCACTTTCAGTGAC |
| PD JUPv1 | GCCCTCCGTCAGCAGCAAGGTGGTT | PE ZDHH19- | CAGGGCTGTGCCAGCAACTGCA |
| BIP | TTCTTGAGCGTGT | 207v25-1 BIP | GCCATGTACTTGGGTCC |
| PD JUPv2 | ccgagctcagttcgctgtcc | PE ZDHH19- | AGAGCCTTTGCTGTCATGTTTT |
| F3 | | 207v25-1 FL | |
| PD JUPv2 | tcctccatgatgcccttgct | PE ZDHH19- | GTATTTAACAATTTGTGCACCAC |
| B3 | | 207v25-1 BL | T |
| PD JUPv2 | aggttcatcacctccatcgtggccggtcaggccccat | PE DEFA4 | TTGGTAGCCCTCCAGGTC |
| FIP | actcagta | IIv15-1 F3 | |
| PD JUPv2 | tggagcagcctatcaaggtgactgtacccgagtcgta | PE DEFA4 | CAATGAGGCAGTTCCCAACA |
| BIP | ggtgtatgtct | IIv15-1 B3 | |
| PD | gacctggggagacatgagtcgag | PE DEFA4 | ATGTCCTGGTCTTCTGGCCCACC |
| LAX1v7 | | IIv15-1 FIP | ACTCCAGGCAAGAGGT |
| F3 | | | |
| PD | ggggacattgacataatcatgcgaa | PE DEFA4 | GGTTTCAGGCTCAACAAGGGGC |
| LAX1v7 | | IIv15-1 BIP | CGAAGTTCTGTTCGCCGG |
| FL | | | |
| PD | gctgtatgctcctggaaggcattg | PE DEFA4 | TGGCCTGGAGCCTCATC |
| LAX1v7 | | IIv15-1 FL | |
| BL | | | |
| PD | cggcacactgcatcaatgtcagag | PE DEFA4 | ATGGTCTGCTCTTGCAGATTAG |
| LAX1v7 | | IIv15-1 BL | T |
| B3 | | | |
| PD | gtcatagatacccacegcgtactctcatgtgccctcc | PE DEFA4 | AGCTCTGCTCTTCAGGTTTC |
| LAX1v7 | caagcagg | IIv24-1 F3 | |
| FIP | | | |
| PD | ccagatgtgtgggaacctcactccgaaatgcttgcgc | PE DEFA4 | GAAGCTAACACCACCGATGA |
| LAX1v7 | agtctctggaa | IIv24-1 B3 | |
| BIP | | | |
| PD | cacactgcatcaatgtcagagcttc | PE DEFA4 | AGTTCTGTTCGCCGGCAGAAAG |
| LAX1v8 | | IIv24-1 FIP | GCTCAACAAGGGGCAT |
| F3 | | | |
| PD | gtgcagtcaccagcatctccac | PE DEFA4 | CACATACTGCTGCACGCGTGTG |
| LAX1v8 | | IIv24-1 BIP | CGTTCCCAGCATGACATT |
| FL | | | |
| PD | tgtggggacattgacataatcatgc | PE DEFA4 | CGATTAACGTTCTGCTGTCCA |
| LAX1v8 | | IIv24-1 FL | |
| BL | | | |
| PD | tctttgttcttcccagtacccagaa | PE DEFA4 | AATCTGCAAGAGCAGACCA |
| LAX1v8 | | IIv24-1 BL | |
| B3 | | | |
| PD | gctagagtctcagcaatctcttctggcgcaagcattt | PE | GGGGAAGGAGGTTCTTCTCC |
| LAX1v8 | cttcagaggat | CEACAM1 | |
| FIP | | IIv1-1 F3 | |
| PD | ttctaccaaaagcccttccagaaatatctctttcctc | PE | CTGTGTCATTCTGGGTGACG |
| LAX1v8 | agtaaactccagc | CEACAM1 | |
| BIP | | IIv1-1 B3 | |
| PD | tgaacggacgatgcggtcagta | PE | TTGACGGTTGCCATCCACTCTTT |
| GPAA1- | | CEACAM1 | GCCCCAGCAACTTTTTGG |
| 4v4 F3 | | IIv1-1 FIP | |
| PD | gggaagggcagtttccggga | PE | CTCAACAAGCTACCCCAGGGCT |
| GPAA1- | | CEACAM1 | GGATCAGCAGGGATGCA |
| 4v4 FL | | IIv1-1 BIP | |
| PD | ccgagtcgcttgtgctcacc | PE | TCCCCTTTGTACCAGCTGTAG |
| GPAA1- | | CEACAM1 | |
| 4v4 BL | | IIv1-1 FL | |
| PD | agacgatatctttggcccaataaatctgc | PE | CCGCAAACAGCGGTCGA |
| GPAA1- | | CEACAM1 | |
| 4v4 B3 | | IIv1-1 BL | |
| PD | gacaccatatagcgctcgtggggctggaggtctacac | PE | CCATCTCCAGCAACAACTCC |
| GPAA1- | gcaga | CEACAM1 | |
| 4v4 FIP | | IIv3-1 F3 | |
| PD | accaacgtgtacggcatcctgagcctggctgttggta | PE | GTGCTTTGCTGGAATGTTCC |
| GPAA1- | gagtca | CEACAM1 | |
| 4v4 BIP | | IIv3-1 B3 | |
| PD | cacggctcccatggcctctt | PE | GGAAATGGTGGGGGTGTCCGCC |
| GPAA1- | | CEACAM1 | CTGTGGAGGACAAGGAT |
| 4v5 F3 | | IIv3-1 FIP | |
| PD | ccgacagcgggcatgtaga | PE | ACACCTATTACCGTCCAGGGGC |
| GPAA1- | | CEACAM1 | AGTACTGTGCAGGTGGGTTA |
| 4v5 FL | | IIv3-1 BIP | |
| PD | ccaggtcatacttgtactggcggaa | PE | TCACAGGTGAAGGCCACA |
| GPAA1- | | CEACAM1 | |
| 4v5 BL | | IIv3-1 FL | |
| PD | cgcctgcaccagtcctt | PE | AAACCTCAGCCTCTCCTGCTA |
| GPAA1- | | CEACAM1 | |
| 4v5 B3 | | IIv3-1 BL | |
| PD | tccaaagccttgcccactgcaccgtgtggaggcccta | PE | ACCTGACCTGCTCCACAA |
| GPAA1- | acc | CEACAM1 | |
| 4v5 FIP | | IIv6-1 F3 | |
| PD | atgttccgcaagctcaaccaccgccggggagcaagt | PE | ATGATGGGGTCGCTTTGG |
| GPAA1- | aga | CEACAM1 | |
| 4v5 BIP | | IIv6-1 B3 | |
| PD | cagctgcccaaccttgacc | PE | TCCTCTCCGAGGACGGGAGAGA |
| GPAA1- | | CEACAM1 | CACTGGAATCTCCATCCG |
| 4v6 F3 | | IIv6-1 FIP | |
| PD | tggcggaagctattgatgccac | PE | ACCACCCTCAGCATAAACCCTGG |
| GPAA1- | | CEACAM1 | GTTGAAGACCTCACACCAA |
| 4v6 FL | | IIv6-1 BIP | |
| PD | ggtccatccaatgatgtccagtcctc | PE | CTCTGGTTTTTGAAGAACCAAC |
| GPAA1- | | CEACAM1 | |
| 4v6 BL | | IIv6-1 FL | |
| PD | cacggctcccatggcctctt | PE | TCAAGAGGGAGGATGCTGG |
| GPAA1- | | CEACAM1 | |
| 4v6 B3 | | IIv6-1 BL | |
| PD | agtgtctgcaggccctgcaatctcttccagaccttct | PE | TTGGTGTGAGGTCTTCAACC |
| GPAA1- | gccaga | CEACAM1 | |
| 4v6 FIP | | IIv12-1 F3 | |
| PD | gctcatggttctgcggcaggggttagggcctccacac | PE | GTGCTCTGTGAGATCACGC |
| GPAA1- | ggta | CEACAM1 | |
| 4v6 BIP | | IIv12-1 B3 | |
| PD | ctctccagggacagtggcccta | PE | GCAATGGCCCCAGGTGAGAGAA |
| C3AR1v5 | | CEACAM1 | AGCGACCCCATCATGC |
| F3 | | IIv12-1 FIP | |
| PD | gcaggccatccctacattgcgat | PE | GTGATTGGAGTAGTGGCCCTGG |
| C3AR1v5 | | CEACAM1 | GGTCTTCCCGAAATGCAGAA |
| B3 | | IIv12-1 BIP | |
| PD | actggcaaacatgttgaggacaatgatggcggcagg | PE | GTGGTAGAGCATTATAGTTTAC |
| C3AR1v5 | ttcctatgcaagctca | CEACAM1 | GTTCA |
| FIP | | IIv12-1 FL | |
| PD | ttcctgcttactgccattagcctggagacaccagatt | PE | TTGCTCTGATAGCAGTAGCCC |
| C3AR1v5 | ggcttgaataccacaagac | CEACAM1 | |
| BIP | | IIv12-1 BL | |
| PD | ctcacttggctctccagggacagt | PE | AGAGCACAAACCCTCAGTCT |
| C3AR1v4 | | CEACAM1 | |
| F3 | | IIv17-1 F3 | |
| PD | gcaggccatccctacattgcgat | PE | AGTGATGAGGGTGAGAGACT |
| C3AR1v4 | | CEACAM1 | |
| B3 | | IIv17-1 B3 | |
| PD | actggcaaacatgttgaggacaatgatggcctacgg | PE | TGTGGGTTGCTGGGCTTCAAAG |
| C3AR1v4 | caggttcctatgcaagc | CEACAM1 | TCAGGACCACTCCAATGACC |
| FIP | | IIv17-1 FIP | |
| PD | ttcctgcttactgccattagcctggagacaccagatt | PE | GCCTCCCCATCCCTAACAGCAGT |
| C3AR1v4 | ggcttgaataccacaagac | CEACAM1 | GAGCAGGACAGGTTTCA |
| BIP | | IIv17-1 BIP | |
| PD | gaagcctggtggctctgacctc | PE | AATAAGTAACTTCATTCATCTTG |
| ZDHHC19- | | CEACAM1 | TTAGGTG |
| 4v4 F3 | | IIv17-1 FL | |
| PD | gggtctgagaagttgagtgaaacaagactg | PE | CAGAAATAATTTATTCAGAAGT |
| ZDHHC19- | | CEACAM1 | AAAAAAGC |
| 4v4 FL | | IIv17-1 BL | |
| PD | gggatggggctccttcacca | PE | CTCCTCTTCCGGTTTCCATC |
| ZDHHC19- | | ZDHHC19- | |
| 4v4 BL | | 204 IIv1-1 | |
| | | F3 | |
| PD | cttgcaggtggctggctcagaa | PE | AGGGTGCAGATTCGGCAT |
| ZDHHC19- | | ZDHHC19- | |
| 4v4 B3 | | 204 IIv1-1 | |
| | | B3 | |
| PD | agcaaagaggctagggaggaaccagcagccatgac | PE | TTGCTGGCACAGCCCTGGTCCC |
| ZDHHC19- | actcttaacggatgcc | ZDHHC19- | GACCAGTGCAGACA |
| 4v4 FIP | | 204 IIv1-1 | |
| | | FIP | |
| PD | ccttcaatgtggtgctgctggtctctgtgataacagg | PE | CAATTTGTGCACCACTGGGACC |
| ZDHHC19- | aaaggcccactc | ZDHHC19- | AGGCCCCACCACTCTCTG |
| 4v4 BIP | | 204 IIv1-1 | |
| | | BIP | |
| PD | gctggtgaaggagccccat | PE | AGGGGTTGTATCCCTGAAGG |
| ZDHHC19- | | ZDHHC19- | |
| 4v5 F3 | | 204 IIv1-1 | |
| | | FL | |
| PD | cccgtggttcacccaca | PE | CAAGTACATGGCTGAAGCTGTC |
| ZDHHC19- | | ZDHHC19- | C |
| 4v5 FL | | 204 IIv1-1 | |
| | | BL | |
| PD | gcagcaccacattgaaggcagc | PE | GGATGCGTATTGCCCTATGG |
| ZDHHC19- | | ZDHHC19- | |
| 4v5 BL | | 207v1-1 F3 | |
| PD | tccttaccttcttcagtcttgtttcactcaac | PE | CTGAAAGTGGCCCAGCAG |
| ZDHHC19- | | ZDHHC19- | |
| 4v5 B3 | | 207v1-1 B3 | |
| PD | cgttctgagccagccacctggtccctggttcctccct | PE | GCACTCACTGGAGGGAGGAAAA |
| ZDHHC19- | agc | ZDHHC19- | CAACAGTGAGTGGCGACA |
| 4v5 FIP | | 207v1-1 FIP | |
| PD | gagtgggcctttcctgttatcacagccttgatgtaag | PE | GGAGTCCTGGTTCCTTTGGTGG |
| ZDHHC19- | atgccagggtctgagaa | ZDHHC19- | TCCCCTGTGTCCCATGTC |
| 4v5 BIP | | 207v1-1 BIP | |
| PD | gtccctggttcctccctagc | PE | GGACAGCTCCATAACAGCTCA |
| ZDHHC19- | | ZDHHC19- | |
| 4v6 F3 | | 207v1-1 FL | |
| PD | cccgtggttcacccaca | PE | AGAATGGTGTTGAAAATCTCAG |
| ZDHHC19- | | ZDHHC19- | CC |
| 4v6 FL | | 207v1-1 BL | |
| PD | gcgaagaagaggccactgaaaaagacc | PE | GGAGCTGTCCAGACTTTTCC |
| ZDHHC19- | | ZDHHC19- | |
| 4v6 BL | | 207v8-1 F3 | |
| PD | tccttaccttcttcagtcttgtttcactcaac | PE | AAGGTGTCTGCACTGACTTC |
| ZDHHC19- | | ZDHHC19- | |
| 4v6 B3 | | 207v8-1 B3 | |
| PD | ttctgagccagccacctgcaagctctttgctgccttc | PE | CGCCGCAAAGGCTGAGATTTTC |
| ZDHHC19- | aatgtggtgc | ZDHHC19- | GTGAGTGCCAGCTGTCCT |
| 4v6 FIP | | 207v8-1 FIP | |
| PD | gagtgggcctttcctgttatcacagccttgatgtaag | PE | GGACACAGGGGAGTTGGTGGAA |
| ZDHHC19- | atgccagggtctgagaa | ZDHHC19- | GAGCCTTTGCTGTCATGT |
| 4v6 BIP | | 207v8-1 BIP | |
| PD | ctgctggtctttttcagtggcctct | PE | GGAACCAGGACTCCATGCA |
| ZDHHC19- | | ZDHHC19- | |
| 4v7 F3 | | 207v8-1 FL | |
| PD | aggggcagtggtaagtccgg | PE | TGGGCAGAGATGCTGCT |
| ZDHHC19- | | ZDHHC19- | |
| 4v7 FL | | 207v8-1 BL | |
| PD | aggacaaagagggagcctgtgataacag | PE | TCCTCCCTCCAGTGAGTG |
| ZDHHC19- | | ZDHHC19- | |
| 4v7 BL | | 207v11-1 F3 | |
| PD | gccttccgcctgcaatggtg | PE | AGGTGTCTGCACTGACTTCA |
| ZDHHC19- | | ZDHHC19- | |
| 4v7 B3 | | 207v11-1 B3 | |
| PD | gatgtaagatgccagggtctgagaagttgatcagaa | PE | CGCCGCAAAGGCTGAGATTTTC |
| ZDHHC19- | cggggagtgggccttt | ZDHHC19- | GCTGTCCTTGCATGGAGTC |
| 4v7 FIP | | 207v11-1 | |
| | | FIP | |
| PD | tgtgggtgaaccacggggcggtggaagcagcacttt | PE | CATGGGACACAGGGGAGTTGGA |
| ZDHHC19- | gga | ZDHHC19- | GAGCCTTTGCTGTCATGTT |
| 4v7 BIP | | 207v11-1 | |
| | | BIP | |
| PD | acacatccaagcttaagacggtga | PE | CATTCTCCACCAAAGGAACCAG |
| IFI27v1 | | ZDHHC19- | |
| F3 | | 207v11-1 FL | |
| PD | caactccacccccattggcaa | PE | TGGTGGGCAGAGATGCTG |
| IFI27v1 | | ZDHHC19- | |
| B3 | | 207v11-1 BL | |
| PD | aactgtagcaatcctggccaaagagcttcacattctc | RREB1_204- | GTGTCTGTGTTGTGCTGGCGAA |
| IFI27v1 | aggaactctcc | 11_FIP | TAACTGCCCCCTGTGTGAG |
| FIP | | | |
| PD | tcagtgccatgggcttcactatcatcttggctgctat | RREB1_204- | CAGAAGGAACCAGGAAACGA |
| IFI27v1 | ggaggac | 11_F3 | |
| BIP | | | |
| PD | TCGCCTCGTCCTCCATAG | RREB1_204- | GAGCCGACCACTCATGCAGCCA |
| IFI27v2 | | 11_BIP | CCAGCATGTGGCGATC |
| F3 | | | |
| PD | GCATGGTTCTCTTCTCTGCA | RREB1_204- | GCACTTGTAAGGCCTCTCG |
| IFI27v2 | | 11_B3 | |
| B3 | | | |
| PD | GAGTAGCCACAAGGCTGCCCCAGCC | RREB1_204- | GTGCTGGGTAGTGCAAATCTT |
| IFI27v2 | AAGATGATGTCCGC | 11_LF1 | |
| FIP | | | |
| PD | TGACCAAGTTCATCCTGGGCTCAGG | RREB1_204- | ATCTGCGGAAAGTCACTGAGC |
| IFI27v2 | GAGCTAGTAGAACCTCG | 11_LB1 | |
| BIP | | | |
| PD | GTGCCATGGGCTTCACTG | RREB1_204- | TCGTGGAAGCCAGCAGGAATTG |
| IFI27v3 | | 14_FIP | AACTTCCCAGGGATGCAATG |
| F3 | | | |
| PD | CGCAATGACAGCCGCAAT | RREB1_204- | ACGCGCTTGTCCACAAAC |
| IFI27v3 | | 14_F3 | |
| B3 | | | |
| PD | TCCACCCCCATTGGCAATGGAATCG | RREB1_204- | CTCGCATTTCTCAGGCCTGGTGT |
| IFI27v3 | CCTCGTCCTCCATAG | 14_BIP | CGCAGACAAAACGGTGT |
| FIP | | | |
| PD | GGCAGCCTTGTGGCTACTCTCCCAG | RREB1_204- | GGGAACGCCTTGTCACAG |
| IFI27v3 | GATGAACTTGGTCAA | 14_B3 | |
| BIP | | | |
| PD | ggtgatgaaatcctggttagcgga | RREB1_204- | GGGTTGTTCTGTATGAAAGGTC |
| TGFBIv4 | | 14_LF1 | T |
| F3 | | | |
| PD | cgctgatgcttgtttgaagatctc | RREB1_204- | GCGAAACAAACCTGCGGAG |
| TGFBIv4 | | 14_LB1 | |
| FL | | | |
| PD | gctgacttccagcttgtcacct | RREB1_204- | CTGTGCATGTTCCCATTGGTGG |
| TGFBIv4 | | 27_FIP | ACATGCTGGTGCACTCTG |
| BL | | | |
| PD | ctccagccaacagacctcaggaa | RREB1_204- | AAGTCACTGAGCTCGGCC |
| TGFBIv4 | | 27_F3 | |
| B3 | | | |
| PD | aggctccttgttgacactcaccacgccctggtgcggc | RREB1_204- | AAGGACCCTAACAGTGCCACAG |
| TGFBIv4 | taaagtct | 27_BIP | TCTTGGAGGACAATCGCCT |
| FIP | | | |
| PD | tgacatcatggccacaaatggcgtcagagtctgcaa | RREB1_204- | GGCATCGTGACTCAGTTTCC |
| TGFBIv4 | gttcatcccct | 27_B3 | |
| BIP | | | |
| PD | tgacatcatggccacaaatggcgt | RREB1_204- | CACAGTGCACTTGTAAGGCC |
| TGFBIv5 | | 27_LF1 | |
| F3 | | | |
| PD | tgccgtggtgcattcctcctgta | RREB1_204- | CCACAGCCCCTCCATCTCCT |
| TGFBIv5 | | 27_LB1 | |
| FL | | | |
| PD | gttcatcccctctttcctgaggtc | RREB1_204- | AATCTTCTCACACAGGGGGCAG |
| TGFBIv5 | | 109_FIP | GGATTGGCAGAAGGAACCA |
| BL | | | |
| PD | gtgcgactagcccctgtctatcaa | RREB1_204- | CGAAGCCTCCAGGACCAA |
| TGFBIv5 | | 109_F3 | |
| B3 | | | |
| PD | agatctcaagcgcagagtctggtcatcaccaatgttc | RREB1_204- | CACATTCGCCAGCACAACACAG |
| TGFBIv5 | tgcagcct | 109_BIP | GAGCTCAGTGACTTTCCGC |
| FIP | | | |
| PD | agcgttttccagggcttcccagagcttcaagctaatg | RREB1_204- | ATGTGGCGATCGAGGGAG |
| TGFBIv5 | cttcatcctc | 109_B3 | |
| BIP | | | |
| PD | ctacataagacctggaacacaggactgct | RREB1_204- | AAGACTTCTCCTCTTTCGTTTCC |
| DEFA4v4 | | 109_LF1 | |
| F3 | | | |
| PD | atgccccttgttgagcctgaaacc | RREB1_204- | ACACTGGAGGAGCCGACCA |
| DEFA4v4 | | 109_LB1 | |
| FL | | | |
| PD | aggagggcgataatcctcatggct | RREB1_204- | TTCAGGGAGAGACCGAGGAGAG |
| DEFA4v4 | | 116_FIP | CTTTGCACATCCAGAAACGC |
| BL | | | |
| PD | cgtgggccagaagaccaggacatatcta | RREB1_204- | GGAGGAGGGGAAGTGACT |
| DEFA4v4 | | 116_F3 | |
| B3 | | | |
| PD | gagggctaccaagagaatagcagegccctgcctagc | RREB1_204- | CCGGTCCCCAACGCATCATGCTC |
| DEFA4v4 | ttgaggatctgtcac | 116_BIP | CTGGAACACACAGTCG |
| FIP | | | |
| PD | actccaggcaagaggtgatgaggcgagcagagcttt | RREB1_204- | CACACCTCAGAGCCACCA |
| DEFA4v4 | tatcccatgcaaaggaa | 116_B3 | |
| BIP | | | |
| PD | gggcactgttggcaagagtagcat | RREB1_204- | TGCAAACTTGTTCCGGAAAAGG |
| CD163v4 | | 116_LF12 | |
| F3 | | | |
| PD | caggaacctccatgccagatctcca | RREB1_204- | AGGGAGCCAAGCGTCCT |
| CD163v4 | | 116_LB12 | |
| FL | | | |
| PD | actgaacattgtccacccacatgggaa | RREB1_204- | AATCTTCTCACACAGGGGGCAG |
| CD163v4 | | 231_FIP | GGATTGGCAGAAGGAACCAG |
| BL | | | |
| PD | ccctcggaggagacctggatca | RREB1_204- | CGAAGCCTCCAGGACCAA |
| CD163v4 | | 231_F3 | |
| B3 | | | |
| PD | atgatgggcactgccacagcgtcaaccctgcatcttt | RREB1_204- | CACATTCGCCAGCACAACACAG |
| CD163v4 | agacaaggccat | 231_BIP | GAGCTCAGTGACTTTCCGC |
| FIP | | | |
| PD | tccatgggagaagagactggcccgtccagaacagga | RREB1_204- | GAGTGCACCAGCATGTGG |
| CD163v4 | agtgggtcctt | 231_B3 | |
| BIP | | | |
| PD | gcccaagctctcctccacaggt | RREB1_204- | GGAAGACTTCTCCTCTTTCGTTT |
| CEACAM | | 231_LF1 | C |
| 1v4 F3 | | | |
| PD | cctacaatttgacggttgccatccact | RREB1_204- | ACACTGGAGGAGCCGACCA |
| CEACAM | | 231_LB1 | |
| 1v4 FL | | | |
| PD | gtgggcgggttccagaaggtt | YWHAB_201_ | TGCATGATCAGAGTGCTGTCTT |
| CEACAM | | 145_FIP | TATAAAACGGCATTTGATGAAG |
| 1v4 BL | | | C |
| PD | gaggttcttctccttgtccacaatctgc | YWHAB_201_ | CTGTGGACATCGGAAAACCAGT |
| CEACAM | | 145_BIP | CACAAAGCACGAGAAACA |
| 1v4 B3 | | | |
| PD | ggattcagtagtgagctgggcagtgtctcagccccac | YWHAB_201_ | CTGAAAAGGCCTGTAGCC |
| CEACAM | ttcacagagt | 145_F3 | |
| 1v4 FIP | | | |
| PD | atgccattcaatgttgcagaggggaagtaccagctgt | YWHAB_201_ | CAGAGTGACACTGAACAGA |
| CEACAM | agccaaaaagttgc | 145_B3 | |
| 1v4 BIP | | | |
| PD | ctcacagcctcacttctaaccttctggaa | YWHAB_201_ | TCAGCGTATCCAATTCAGCAAT |
| CEACAM | | 145_LF1 | |
| 1v5 F3 | | | |
| PD | ggatgcattggggtatattgtctctcgacc | YWHAB_201_ | GAGACGAAGGAGACGCTGGG |
| CEACAM | | 145_LB1 | |
| 1v5 FL | | | |
| PD | cttcccctctgcaacattgaatggcat | CTSB_204_5_ | GCAGGCCCTCTTTCCATC |
| CEACAM | | F3 | |
| 1v5 BL | | | |
| PD | gaaagagtggatggcaaccgtcaaattgta | CTSB_204_5_ | TGTTCCCGTGCATCGAAG |
| CEACAM | | _B3 | |
| 1v5 B3 | | | |
| PD | gcagattgtggacaaggagaagaacctccactgccc | CTSB_204_5_ | TAGAAGTTGTGCCCGGCCTGCT |
| CEACAM | agctcactactgaatcc | FIP | GTCGGATGAGCTGGTCA |
| 1v5 FIP | | | |
| PD | gcaactttttggctacagctggtaggggtagcttgtt | CTSB_204_5_ | TGTGGTACCTTCCTGGGTGGGC |
| CEACAM | gagttcctattgcatatcc | BIP | AGCTTCAGGTCCTCGGTA |
| 1v5 BIP | | | |
| PD | tctcctgctatgcagcctctaacc | CTSB_204_ | TCTGTGAGCCTGGCTACAG |
| CEACAM | | 27_F3 | |
| 1v6 F3 | | | |
| PD | ggtcaggttcacagagtccttatctcctgt | CTSB_204_ | TGGCCACCCATCATCTCTC |
| CEACAM | | 72_B3 | |
| 1v6 FL | | | |
| PD | tgtgtgctttgctggaatgttcc | CTSB_204_ | CGGCCATGATGTCCTTCTCGCAA |
| CEACAM | | 27_FIP | CAGGACAAGCACTACGGA |
| 1v6 BL | | | |
| PD | agtcactggctgcaacaggacc | CTSB_204_ | CCGTGGAGGGAGCTTTCTCTGG |
| CEACAM | | 27_BIP | GTGACGTGTTGGTACACTC |
| 1v6 B3 | | | |
| PD | tcacagtgatgttagggataaagagctccacctgcac | CTSB_204_ | GGCTGTGGAAGCCATCTC |
| CEACAM | agtactcctggcttatcaa | 41_F3 | |
| 1v6 FIP | | | |
| PD | tagtggatcctatacctgccacgcccttagctcagtg | CTSB_204_ | GGATTCATAGAGGCCACCAG |
| CEACAM | actatgatcgtcttgactgt | 41_B3 | |
| 1v6 BIP | | | |
| PD | ctcattccagattccaaatgtcattctgtagc | CTSB_204_ | ACAGCATGTGAGCAGGTCCTCT |
| LY86v3 | | 41_FIP | GACCGGATCTGCATCCA |
| F3 | | | |
| PD | gtgtgggccaggctttccca | CTSB_204_ | TGTGTGGGGACGGCTGTAATGC |
| LY86v3 | | 41_BIP | AGGCCTTTTCTTGTCCAGA |
| FL | | | |
| PD | caccatctgtttcccatctccagcc | CTSB_204_ | GCAAGATCTGTGAGCCTGG |
| LY86v3 | | 715_F3 | |
| BL | | | |
| PD | cccaagaggcccccaccatgaa | CTSB_204_ | CATGGCCACCCATCATCTC |
| LY86v3 | | 715_B3 | |
| B3 | | | |
| PD | ggtgaggctgtaaacttgggaaagacaaagggtccc | CTSB_204_ | CGGCCATGATGTCCTTCTCGCG |
| LY86v3 | tgggtccatacacagc | 715_FIP | CCCGACCTACAAACAGG |
| FIP | | | |
| PD | gtggaggctgttctgaggggccagaggaagagagtg | CTSB_204_ | ACAAAAACGGCCCCGTGGAGAC |
| LY86v3 | gctgtgaaac | 715_BIP | GTGTTGGTACACTCCTGA |
| BIP | | | |
| PD | ggtccctgggtccatacacagc | CTSB_204_ | ACCCCCAAGTGTAGCAAGA |
| LY86v4 | | 709_F3 | |
| F3 | | | |
| PD | gctgtcgctacagaccacgtgt | CTSB_204_ | TCCGGTGACGTGTTGGTA |
| LY86v4 | | 709_B3 | |
| FL | | | |
| PD | caccatctgtttcccatctccagcc | CTSB_204_ | CGGCCATGATGTCCTTCTCGCG |
| LY86v4 | | 709_FIP | CCCGACCTACAAACAGG |
| BL | | | |
| PD | ccaagaggcccccaccatgaag | CTSB_204_ | ATCTACAAAAACGGCCCCGTGG |
| LY86v4 | | 709_BIP | TTGTAGAGCAGGAAGTCCGA |
| B3 | | | |
| PD | gctgtaaacttgggaaagacaaagcgctgcatcaag | CTSB_204_ | ACCCCCAAGTGTAGCAAGA |
| LY86v4 | agcctgcagagactc | 712_F3 | |
| FIP | | | |
| PD | gtggaggctgttctgaggggccagaggaagagagtg | CTSB_204_ | TCCGGTGACGTGTTGGTA |
| LY86v4 | gctgtgaaac | 712_B3 | |
| BIP | | | |
| PD | ccgaacattcaaaactgtttctccaaagcgt | CTSB_204_ | CGGCCATGATGTCCTTCTCGCG |
| KCNJ2v4 | | 712_FIP | CCCGACCTACAAACAGG |
| F3 | | | |
| PD | tgatgaactgaacattacagtggccat | CTSB_204_ | ATCTACAAAAACGGCCCCGTGG |
| KCNJ2v4 | | 712_BIP | CTTGTAGAGCAGGAAGTCCG |
| FL | | | |
| PD | cgcacactgcccatcgcttct | CTSB_204_ | GCAAGATCTGTGAGCCTGG |
| KCNJ2v4 | | 718_F3 | |
| BL | | | |
| PD | ctttgggaacgggaagagtaaagtccac | CTSB_204_ | CATGGCCACCCATCATCTC |
| KCNJ2v4 | | 718_B3 | |
| B3 | | | |
| PD | gagacgatgctgtagcggttggttgactgttttccaa | CTSB_204_ | CGGCCATGATGTCCTTCTCGCG |
| KCNJ2v4 | agcagaagcactgga | 718_FIP | CCCGACCTACAAACAGG |
| FIP | | | |
| PD | agacggtatgaagttggccacgcggctcctgcactgt | CTSB_204_ | CAAAAACGGCCCCGTGGAGGAC |
| KCNJ2v4 | tgtc | 718_BIP | GTGTTGGTACACTCCTGA |
| BIP | | | |
| PD | gcagtgtgcgaaccaaccgcta | CTSB_204_ | GCAAGATCTGTGAGCCTGG |
| KCNJ2v5 | | 721_F3 | |
| F3 | | | |
| PD | gccaggcagaagataaccagcatcc | CTSB_204_ | CATGGCCACCCATCATCTC |
| KCNJ2v5 | | 721_B3 | |
| FL | | | |
| PD | tcccgttcccaaagccatttgca | CTSB_204_ | CGGCCATGATGTCCTTCTCGC- |
| KCNJ2v5 | | 721_FIP | GCCCGACCTACAAACAGG |
| BL | | | |
| PD | ggtgagaaggggcaacggtacct | CTSB_204_ | CCGTGGAGGGAGCTTTCTCTG- |
| KCNJ2v5 | | 721_BIP | ACGTGTTGGTACACTCCTGA |
| B3 | | | |
| PD | gcactgttgtcgggtgtggactgtatgaagttggcca | CTSB_204_ | TACCCCCAAGTGTAGCAAGA |
| KCNJ2v5 | ccatggcagt | 724_F3 | |
| FIP | | | |
| PD | agaaagatggccactgtaatgttcagttcaccgccag | CTSB_204_ | CATGGCCACCCATCATCTC |
| KCNJ2v5 | cgaatgtccacac | 724_B3 | |
| BIP | | | |
| PD | cggtccccaacgcatcatgc | CTSB_204_ | CGGCCATGATGTCCTTCTCGC- |
| RREB1v6 | | 724_FIP | GCCCGACCTACAAACAGG |
| F3 | | | |
| PD | gacccactgatagactatgaaactttcccc | CTSB_204_ | CCGTGGAGGGAGCTTTCTCTG- |
| RREB1v6 | | 724_BIP | TCCGGTGACGTGTTGGTA |
| FL | | | |
| PD | ctcagagccaccactcctggaac | IFI27_48_ | CTGTGCCCATGGTGCTCAGTTG |
| RREB1v6 | | BIP | GAGGACGAGGCGATTC |
| BL | | | |
| PD | agaagacaagaggtcaacacagactcatt | IFI27_49_ | GCCAAAGTGGTCAGGGTG |
| RREB1v6 | | F3 | |
| B3 | | | |
| PD | tattggggggctgaacctggcggccgttgctccgact | IFI27_49_ | GGACATCATCTTGGCTGCTA |
| RREB1v6 | gtgt | B3 | |
| FIP | | | |
| PD | gggcagcttgataacacaaagaaaacagttgctgta | IFI27_49_ | CGCCATGGCCACAACTCCTCGG |
| RREB1v6 | gctatcattcacacggagtagaa | FIP | CTCTGCCGTAGTTTTGC |
| BIP | | | |
| PD | gccattttgattccttttccggaacaagt | IFI27_49_ | CTGTGCCCATGGTGCTCAGTTG |
| RREB1v7 | | BIP | GAGGACGAGGCGATTC |
| F3 | | | |
| PD | cggagtagaaaatgagtctgtgttgacctctt | IFI27_50_ | GCCAAAGTGGTCAGGGTG |
| RREB1v7 | | F3 | |
| FL | | | |
| PD | ctccctggcatgatgcgttgg | IFI27_50_ | GGACATCATCTTGGCTGCTA |
| RREB1v7 | | B3 | |
| BL | | | |
| PD | gccaggttcagccccccaata | IFI27_50_ | CGCCATGGCCACAACTCCTCGCT |
| RREB1v7 | | FIP | CTGCCGTAGTTTTGCC |
| B3 | | | |
| PD | acacagtcggagcaacggccctccteggtctctccct | IFI27_50_ | CTGTGCCCATGGTGCTCAGTTG |
| RREB1v7 | gaagc | BIP | GAGGACGAGGCGATTC |
| FIP | | | |
| PD | gttccaggagtggtggctctgagactgttttctttgt | IFI27_65_F3 | CAATGGGGGTGGAGTTGC |
| RREB1v7 | gttatcaagctgccc | | |
| BIP | | | |
| PD | gccaggttcagccccccaata | IFI27_65_B3 | GATAGTTGGCTCCTCGCTG |
| RREB1v9 | | | |
| F3 | | | |
| PD | gatggaagataggtctgaaccttccaagc | IFI27_65_ | TGGAGCCCAGGATGAACTTGGT |
| RREB1v9 | | FIP | TTGTGGCTACTCTGCAGTCA |
| FL | | | |
| PD | cggagtagaaaatgagtctgtgttgacctctt | IFI27_65_ | TGCGAGGTTCTACTAGCTCCCTT |
| RREB1v9 | | BIP | TCTCCCCTGGCATGGTT |
| BL | | | |
| PD | cttcttagaagcttaaacccctgtcccaat | IFI27_94_F3 | CAGATGGGAAGGGACTCGA |
| RREB1v9 | | | |
| B3 | | | |
| PD | atgaaactttcccctgctgtagctatcattccaaaga | IFI27_94_B3 | ATGGCACTGAGCACCATG |
| RREB1v9 | aaacagtgtcaacgagtactacca | | |
| FIP | | | |
| PD | cagtgggtcagaaaatggagttttatagcagacagc | IFI27_94_ | GCTGATGAGGTGAGAGCAGAGG |
| RREB1v9 | gggcgaacttgacgt | FIP | CTCTGGAATGCCACGGAAT |
| BIP | | | |
| PD | atccaggcagtcatagactccgga | IFI27_94_ | CTGGCTCTGCCGTAGTTTTGCCC |
| KPNA6v6 | | BIP | CACAACTCCTCCAATCACA |
| F3 | | | |
| PD | atttgagccctgttgccagcagta | IFI27_198_ | GCCAAGATGATGTCCGCG |
| KPNA6v6 | | F3 | |
| FL | | | |
| PD | cagggcaggagaagccactttgta | IFI27_198_ | TCTTCTCTGCAGGGCGAG |
| KPNA6v6 | | B3 | |
| BL | | | |
| PD | ccacttgttgagcagtcccaagga | IFI27_198_ | TGCTCCCAGTGACTGCAGAGTA |
| KPNA6v6 | | FIP | ATTGCCAATGGGGGTGGA |
| B3 | | | |
| PD | agtgacgatgttacccacggctctattggtagagctg | IFI27_198_ | GACCAAGTTCATCCTGGGCTCC |
| KPNA6v6 | ctgatgcacaa | BIP | GCAGGGAGCTAGTAGAACCT |
| FIP | | | |
| PD | tcttaactgttcagccctaccttgagtccagcaagct | IFI27_295_ | CAGATGGGAAGGGACTCGA |
| KPNA6v6 | tccttccggat | F3 | |
| BIP | | | |
| PD | tgtggagctgattaatgaagaagctgcca | IFI27_295_ | ATGGCACTGAGCACCATG |
| KPNA6v7 | | 3B | |
| F3 | | | |
| PD | cgaaccgatccaccactcttggagt | IFI27_295_ | GCTGATGAGGTGAGAGCAGAGG |
| KPNA6v7 | | FIP | CTCTGGAATGCCACGGAAT |
| FL | | | |
| PD | acactctccccagtggtagagctca | IFI27_295_ | GGCTCTGCCGTAGTTTTGCCCG |
| KPNA6v7 | | BIP | CCACAACTCCTCCAATCAC |
| BL | | | |
| PD | acacagaaattccggaaactgctctcca | IFI27_332_ | GCAGCCAAGATGATGTCCG |
| KPNA6v7 | | F3 | |
| B3 | | | |
| PD | aaagagcatctccaccatctctcttgtgattgttcga | IFI27_332_ | TCTCCCCTGGCATGGTTC |
| KPNA6v7 | tagtcttctcatggactcttatg | B3 | |
| FIP | | | |
| PD | atgattctgacctgcagttagcaacctaacttcatct | IFI27_332_ | TGCTCCCAGTGACTGCAGAGTA |
| KPNA6v7 | attggaggactaggctctt | FIP | ATTGCCAATGGGGGTGGA |
| BIP | | | |
| PD | ccagccaatcatcagacattcctacgac | IFI27_332_ | ATTGGGTCTGCCATTGCGGCTC |
| KIAA1370 | | BIP | TTCTCTGCAGGGCGAG |
| v1 F3 | | | |
| PD | aactgagggtttgttggtgattcagtgtaaga | IFI27_64_ | TGCTCCCAGTGACTGCAGAGTA |
| KIAA1370 | | FIP | ATTGCCAATGGGGGTGGA |
| v1 B3 | | | |
| PD | tttagaactctggaacctcagatgtatgaggaacatc | IFI27_64_F3 | AGCAGCCAAGATGATGTCC |
| KIAA1370 | cgacacacagaagaacggtt | | |
| v1 FIP | | | |
| PD | tctggaaagatctacctccatagagacgtacggcacc | IFI27_467_ | TGGAGCCCAGGATGAACTTGGT |
| KIAA1370 | gctatcaacctccattgactttc | FIP | TTGTGGCTACTCTGCAGTCA |
| v1 BIP | | | |
| PD | ctgtgcccatggtgctca | IFI27_467_ | ATTGCCAATGGGGGTGGA |
| IFI27v5 | | F3 | |
| F3 | | | |
| PD | ctccacccccattggcaa | IFI27_599_ | ACTGCAGAGTAGCCACAAGGCT |
| IFI27v5 | | FIP | GCCAAGATGATGTCCGCG |
| FL | | | |
| PD | ctctccggattgaccaagttca | IFI27_599_ | GCCTCGTCCTCCATAGCA |
| IFI27v5 | | F3 | |
| BL | | | |
| PD | cccctggcatggttctctt | | |
| IFI27v5 | | | |
| B3 | | | |
| PD | agtagccacaaggctgcccctccatagcagccaaga | | |
| IFI27v5 | tgat | | |
| FIP | | | |
| PD | agtcactgggagcaactggagctagtagaacctcgc | | |
| IFI27v5 | aatga | | |
| BIP | | | |
| PD | tttggccaggattgctacagtt | | |
| IFI27v6 | | | |
| F3 | | | |
| PD | ccatggcactgagcaccat | | |
| IFI27v6 | | | |
| FL | | | |
| PD | gcagccttgtggctactct | | |
| IFI27v6 | | | |
| BL | | | |
| PD | gcccaggatgaacttggtcaa | | |
| IFI27v6 | | | |
| B3 | | | |
| PD | ttggctgctatggaggacgatgattggaggagttgtg | | |
| IFI27v6 | gccat | | |
| FIP | | | |
| PD | ttgccaatgggggtggagtccagttgctcccagtgac | | |
| IFI27v6 | t | | |
| BIP | | | |
| PD | acctgaggagagtgactagcttct | | |
| HK3v4 F3 | | | |
| PD | ccgcaaccctgaagaccca | | |
| HK3v4 FL | | | |

The primers were assessed in different sets (e.g., “versions” for each target), using RNA or genomic DNA (gDNA) as a template, or in the absence of a template (NTC, no template control). The results are shown in Table 8.

TABLE 8
First set of primer screening results.

| | | | | | Passes | |
|---|---|---|---|---|---|---|
| | | | | | Criteria? | |
| Target | Version | RNA | gDNA | NTC | (Y/N) | Notes |
| IFI27 | 5 | + | + | + | N | RNA and gDNA melt curves |
| | | | | | | identical; RNA amplification too |
| | | | | | | late |
| IFI27 | 6 | + | + | − | N | RNA and gDNA melt curves |
| | | | | | | identical |
| HK3 | 4 | + | − | − | Y | |
| HK3 | 5 | + | + | − | Y | |
| HK3 | 6 | + | + | + | N | |
| JUP | 6 | + | + | − | N | RNA amplification too late |
| JUP | 7 | + | + | − | N | cDNA and gDNA melt curves |
| | | | | | | identical; RNA amplification too |
| | | | | | | late |
| JUP | 8 | + | + | + | N | |
| LAX1 | 4 | − | + | + | N | Early primer interactions |
| LAX1 | 5 | − | − | − | N | Early primer interactions |
| LAX1 | 6 | + | − | − | N | Early primer interactions; RNA |
| | | | | | | amplification too late |
| CTSB | 5 | + | + | − | Y | Meets criteria, however, high |
| | | | | | | variation among RNA Tts. RNA |
| | | | | | | amplification too late |
| PER1 | 4 | + | − | − | Y | Meets criteria, but RNA |
| | | | | | | amplification is late |
| ZDHHC19-1 | 6 | − | − | − | N | |
| ZDHHC19-1 | 7 | − | − | − | N | Early primer interactions |
| C3AR1 | 6 | + | + | − | N | |
| C3AR1 | 7 | + | + | − | N | |
| NMRK1 | 3 | − | − | − | N | |
| GNA15 | 4 | + | − | − | Y | Meets criteria, but RNA |
| | | | | | | amplification is late |
| GNA15 | 5 | + | − | − | Y | |
| BATF | 3 | + | − | − | Y | |
| BATF | 5 | − | + | − | N | |
| TGFBI | 4 | + | − | − | Y | Meets criteria, but RNA |
| | | | | | | amplification is late |
| TGFBI | 5 | − | − | − | N | Early primer interations |
| CD163 | 4 | + | + | − | N | |
| CEACAM1 | 4 | − | − | − | N | |
| CEACAM1 | 5 | − | + | − | N | |
| CEACAM1 | 6 | − | + | − | N | |
| LY86 | 3 | − | − | − | N | |
| LY86 | 4 | − | + | − | N | |
| RREB1 | 6 | − | − | − | N | |
| RREB1 | 7 | + | − | − | Y | |
| RREB1 | 9 | + | + | − | N | |
| KPNA6 | 6 | + | − | − | Y | |
| KPNA6 | 7 | + | − | − | Y | Meets criteria, but RNA |
| | | | | | | amplification is late |
| KIAA1370 | 1 | − | − | − | N | |
| KCNJ2 | 4 | − | + | − | N | |
| KCNJ2 | 5 | − | + | − | N | |
| CTSB | 6 | + | − | − | Y | |
| CTSB | 7 | + | − | − | N | |
| JUP | 1 | + | + | − | N | No loop primers |
| JUP | 2 | − | − | − | N | No loop primers |
| JUP | 4 | + | − | − | Y | No loop primers |
| C3AR1 | 4 | + | + | − | N | No loop primers |
| C3AR1 | 5 | + | + | − | N | No loop primers |
| ZDHHC19-4 | 4 | − | + | − | N | |
| ZDHHC19-4 | 5 | − | − | − | N | |
| ZDHHC19-4 | 6 | − | − | − | N | |
| ZDHHC19-4 | 7 | − | − | − | N | |
| IFI27 | 1 | − | − | − | N | No loop primers |
| IFI27 | 2 | − | − | − | N | No loop primers |
| IFI27 | 3 | − | − | − | N | No loop primers |
| IFI27 | 7 | − | − | − | N | NTC wells show consistent primer |
| | | | | | | interactions melt curve |
| IFI27 | 8 | + | − | − | N | NTC wells show consistent primer |
| | | | | | | interactions melt curve |
| JUP | 9 | + | − | − | Y | |
| JUP | 10 | + | − | − | N | NTC wells show consistent primer |
| | | | | | | interactions melt curve; RNA |
| | | | | | | amplification too late |
| JUP | 12 | + | + | − | N | NTC wells show consistent primer |
| | | | | | | interactions melt curve |
| ZDHHC19-1 | 8 | − | − | − | N | gDNA amplification interspersed |
| | | | | | | with RNA amplification |
| ZDHHC19-1 | 9 | + | + | − | N | |
| ZDHHC19-1 | 10 | − | + | − | N | |
| KIAA1370 | 3 | + | − | − | N | NTC wells show consistent primer |
| | | | | | | interactions melt curve |
| KIAA1370 | 5 | + | − | − | Y | |
| KIAA1370 | 7 | + | + | − | N | NTC wells show consistent primer |
| | | | | | | interactions melt curve |
| DEFA4 | 6 | − | + | − | N | |
| DEFA4 | 8 | − | − | − | N | NTC wells show consistent primer |
| | | | | | | interactions melt curve |
| DEFA4 | 9 | − | − | − | N | NTC wells show consistent primer |
| | | | | | | interactions melt curve |
| CD163 | 6 | + | − | + | N | NTC wells show consistent primer |
| | | | | | | interactions melt curve |
| CD163 | 7 | + | − | − | N | NTC wells show consistent primer |
| | | | | | | interactions melt curve |
| CD163 | 12 | + | + | − | N | |
| CEACAM1 | 8 | − | − | − | N | |
| CEACAM1 | 9 | − | − | − | N | |
| CEACAM1 | 10 | − | + | − | N | |
| LY86 | 5 | − | + | − | N | |
| LY86 | 6 | − | + | − | N | |
| LY86 | 9 | + | − | − | Y | |
| KCNJ2 | 7 | − | + | − | N | |
| KCNJ2 | 8 | − | + | − | N | |
| KCNJ2 | 9 | − | + | − | N | |
| ZDHHC19-3 | 9 | − | + | − | N | |
| ZDHHC19-3 | 8 | − | − | − | N | |
| DEFA4 | 5 | − | − | − | N | |
| DEFA4 | 7 | − | − | − | N | |
| DEFA4 | 10 | − | + | − | N | |
| CD163 | 5 | + | + | − | N | |
| CD163 | 9 | + | − | − | Y | |
| CD163 | 10 | + | + | − | N | |
| CEACAM1 | 11 | − | − | − | N | |
| CEACAM1 | 12 | − | + | − | N | |
| CEACAM1 | 13 | + | − | − | Y | Meets criteria, but RNA |
| | | | | | | amplification is late |
| KCNJ2 | 10 | + | + | − | N | |
| KCNJ2 | 11 | − | + | − | N | |
| KCNJ2 | 12 | − | + | − | N | |
| CEACAM1 | 13 | + | − | − | Y | |
| CEACAM1 | 13 | + | − | − | Y | |
| RREB1 | 7 + F3_DR | + | − | − | Y | |
| DEFA4 | 1 | − | − | − | N | |
| DEFA4 | 4 | − | − | − | N | |
| DEFA4 | 8 | − | + | − | N | |
| KCNJ2 | 1 | − | − | − | N | |
| KCNJ2 | 4 | − | − | − | N | |
| KCNJ2 | 9 | − | + | − | N | |
| C3AR1 | 1 | + | − | − | Y | |
| C3AR1 | 5 | + | + | + | N | |
| C3AR1 | 7 | + | + | − | N | |
| C3AR1 | 17 | + | − | − | Y | |
| C3AR1 | 19 | + | + | − | N | |
| ZDHHC19 | 27 | + | + | − | N | |
| ZDHHC19 | 34 | + | + | − | N | |
| ZDHHC19 | 45 | + | + | − | N | |
| ZDHHC19 | 66 | + | + | − | N | |
| CEACAM1 | 1 | + | + | − | N | |
| CEACAM1 | 2 | − | + | − | N | |
| CEACAM1 | 3 | − | + | − | N | |
| CEACAM1 | 10 | + | + | − | N | |
| KCNJ2 | 1 | − | − | − | N | |
| KCNJ2 | 4 | − | − | − | N | |
| KCNJ2 | 9 | + | − | − | N | |
| KCNJ2 | 16 | − | − | − | N | |
| KCNJ2 | 21 | − | − | − | N | |
| CEACAM1 | 13 | − | − | − | N | |
| DEFA4 | II 15 | + | − | − | Y | |
| DEFA4 | II 24 | − | + | − | N | |
| ZDHHC19 | 204 II 1 | − | + | − | N | |
| ZDHHC19 | 207 1 | + | + | − | N | |
| ZDHHC19 | 207 8 | − | + | − | N | |
| ZDHHC19 | 207 11 | − | + | − | N | |
| ZDHHC19 | 207 19 | − | + | − | N | |
| ZDHHC19 | 207 25 | − | − | − | N | |
| RREB1 | 11 | − | − | − | N | |
| RREB1 | 14 | + | − | + | N | |
| RREB1 | 27 | + | − | − | N | RNA amplification too late |
| RREB1 | 109 | + | − | − | N | RNA amplification too late |
| RREB1 | 116 | + | + | − | N | RNA amplification too late |
| RREB1 | 231 | + | − | − | Y | |
| YWHAB | 145 | + | − | − | Y | |

Different sets of primers were then assessed and determined to satisfy (pass) or not satisfy (fail) the criteria. The results are shown in Table 9.

TABLE 9
Second screen of primer combinations.

| Markers | Result | Primer mix | Speed | NTC | RNA | gDNA |
|---|---|---|---|---|---|---|
| ARG1 | pass | v1 | in range | − | + | − |
| | | ARG1-F3v1 | | | | |
| | | ARG1-B3v1 | | | | |
| | | ARG1-FIPv1 | | | | |
| | | ARG1-BIPv1 | | | | |
| | | ARG1-LFv1 | | | | |
| | | ARG1-LBv1 | | | | |
| | pass | v2 | in range | − | | − |
| | | ARG1-F3v1 | | | | |
| | | ARG1-B3v1 | | | | |
| | | ARG1-FIP2 | | | | |
| | | ARG1-BIPv2 | | | | |
| | | ARG1-LFv1 | | | | |
| | | ARG1-LBv1 | | | | |
| C3AR1 | fail | v1 | too slow | − | + | − |
| | | C3AR1-F3v1 | | | | |
| | | C3AR1-B3v1 | | | | |
| | | C3AR1-FIPv1 | | | | |
| | | C3AR1-BIPv1 | | | | |
| | | C3AR1-LFv1 | | | | |
| | | C3AR1-LBv1 | | | | |
| | pass | v2 | in range | − | + | − |
| | | C3AR1-F3v1 | | | | |
| | | C3AR1-B3v1 | | | | |
| | | C3AR1-FIPv2 | | | | |
| | | C3AR1-BIPv2 | | | | |
| | | C3AR1-LFv1 | | | | |
| | | C3AR1-LBv1 | | | | |
| CTSL1 | pass | v1 | in range | − | + | − |
| | | CTSL1-F3v1 | | | | |
| | | CTSL1-B3v1 | | | | |
| | | CTSL1-FIPv1 | | | | |
| | | CTSL1-BIPv1 | | | | |
| | | CTSL1-LFv1 | | | | |
| | | CTSL1-LBv1 | | | | |
| | fail | v2 | too slow | − | | − |
| | | CTSL1-F3v1 | | | | |
| | | CTSL1-B3v1 | | | | |
| | | CTSL1-FIPv2 | | | | |
| | | CTSL1-BIPv2 | | | | |
| | | CTSL1-LFv1 | | | | |
| | | CTSL1-LBv1 | | | | |
| FURIN | fail | v1 | too fast | − | | |
| | | FURIN-F3v1 | | | | |
| | | FURIN-B3v1 | | | | |
| | | FURIN-FIPv1 | | | | |
| | | FURIN-BIPv1 | | | | |
| | | FURIN-LFv1 | | | | |
| | | FURIN-LBv1 | | | | |
| | fail | v2 | too fast | − | | |
| | | FURIN-F3v2 | | | | |
| | | FURIN-B3v2 | | | | |
| | | FURIN-FIPv2 | | | | |
| | | FURIN-BIPv2 | | | | |
| | | FURIN-LFv2 | | | | |
| | | FURIN-LBv2 | | | | |
| | fail | v3 | too fast | + | | |
| | | FURIN-F3v3 | | | | |
| | | FURIN-B3v3 | | | | |
| | | FURIN-FIPv3 | | | | |
| | | FURIN-BIPv3 | | | | |
| | | FURIN-LFv3 | | | | |
| | | FURIN-LBv3 | | | | |
| | fail | v1 + t | too fast | − | | |
| | | FURIN-F3v1 | | | | |
| | | FURIN-B3v1 | | | | |
| | | FURIN- | | | | |
| | | FIPv1 + t | | | | |
| | | FURIN- | | | | |
| | | BIPv1 + t | | | | |
| | | FURIN-LFv1 | | | | |
| | | FURIN-LBv1 | | | | |
| | fail | v2 + t | too fast | − | | |
| | | FURIN-F3v2 | | | | |
| | | FURIN-B3v2 | | | | |
| | | FURIN- | | | | |
| | | FIPv2 + t | | | | |
| | | FURIN- | | | | |
| | | BIPv2 + t | | | | |
| | | FURIN-LFv2 | | | | |
| | | FURIN-LBv2 | | | | |
| | fail | v3 + t | too fast | − | | |
| | | FURIN-F3v3 | | | | |
| | | FURIN-B3v3 | | | | |
| | | FURIN- | | | | |
| | | FIPv3 + t | | | | |
| | | FURIN- | | | | |
| | | BIPv3 + t | | | | |
| | | FURIN-LFv3 | | | | |
| | | FURIN-LBv3 | | | | |
| | fail | v4 | too slow | − | | |
| | | FURIN-F3v4 | | | | |
| | | FURIN-B3v4 | | | | |
| | | FURIN-FIPv4 | | | | |
| | | FURIN-BIPv4 | | | | |
| | | FURIN-LFv4 | | | | |
| | | FURIN-LBv4 | | | | |
| | fail | v5 | too fast | − | | |
| | | FURIN-F3v5 | | | | |
| | | FURIN-B3v5 | | | | |
| | | FURIN-FIPv5 | | | | |
| | | FURIN-BIPv5 | | | | |
| | | FURIN-LFv5 | | | | |
| | | FURIN-LBv5 | | | | |
| | fail | v6 | too fast | − | | |
| | | FURIN-F3v6 | | | | |
| | | FURIN-B3v6 | | | | |
| | | FURIN-FIPv6 | | | | |
| | | FURIN-BIPv6 | | | | |
| | | FURIN-LFv6 | | | | |
| | | FURIN-LBv6 | | | | |
| | fail | v7 | too fast | − | | |
| | | FURIN-F3v7 | | | | |
| | | FURIN-B3v7 | | | | |
| | | FURIN-FIPv7 | | | | |
| | | FURIN-BIPv7 | | | | |
| | | FURIN-LFv7 | | | | |
| | | FURIN-LBv7 | | | | |
| | pass | v1(50%) | in range | − | + | − |
| | | CTSL1-F3v1 | | | | |
| | | CTSL1-B3v1 | | | | |
| | | CTSL1-FIPv1 | | | | |
| | | CTSL1-BIPv1 | | | | |
| | | CTSL1-LFv1 | | | | |
| | | CTSL1-LBv1 | | | | |
| GADD45A | fail | v1 | too slow | − | | |
| | | GADD45A- | | | | |
| | | F3v1 | | | | |
| | | GADD45A- | | | | |
| | | B3v1 | | | | |
| | | GADD45A- | | | | |
| | | FIPv1 | | | | |
| | | GADD45A- | | | | |
| | | BIPv1 | | | | |
| | | GADD45A- | | | | |
| | | LFv1 | | | | |
| | | GADD45A- | | | | |
| | | LBv1 | | | | |
| | pass | v2 | in range | − | + | − |
| | | GADD45A- | | | | |
| | | F3v1 | | | | |
| | | GADD45A- | | | | |
| | | B3v1 | | | | |
| | | GADD45A- | | | | |
| | | FIPv2 | | | | |
| | | GADD45A- | | | | |
| | | BIPv2 | | | | |
| | | GADD45A- | | | | |
| | | LFv1 | | | | |
| | | GADD45A- | | | | |
| | | LBv1 | | | | |
| | fail | v3 | too slow | − | | |
| | | GADD45A- | | | | |
| | | F3v3 | | | | |
| | | GADD45A- | | | | |
| | | B3v3 | | | | |
| | | GADD45A- | | | | |
| | | FIPv3 | | | | |
| | | GADD45A- | | | | |
| | | BIPv3 | | | | |
| | | GADD45A- | | | | |
| | | LFv3 | | | | |
| | | GADD45A- | | | | |
| | | LBv3 | | | | |
| HLA-DMB | fail | v1 | too slow | − | | |
| | | HLA-DMB- | | | | |
| | | F3v1 | | | | |
| | | HLA-DMB- | | | | |
| | | B3v1 | | | | |
| | | HLA-DMB- | | | | |
| | | FIPv1 | | | | |
| | | HLA-DMB- | | | | |
| | | BIPv1 | | | | |
| | | HLA-DMB- | | | | |
| | | LFv1 | | | | |
| | | HLA-DMB- | | | | |
| | | LBv1 | | | | |
| | fail | v2 | too slow | − | | |
| | | HLA-DMB- | | | | |
| | | F3v1 | | | | |
| | | HLA-DMB- | | | | |
| | | B3v1 | | | | |
| | | HLA-DMB- | | | | |
| | | FIPv2 | | | | |
| | | HLA-DMB- | | | | |
| | | BIPv2 | | | | |
| | | HLA-DMB- | | | | |
| | | LFv1 | | | | |
| | | HLA-DMB- | | | | |
| | | LBv1 | | | | |
| | fail | v3 | too fast | − | | |
| | | HLA-DMB- | | | | |
| | | F3v3 | | | | |
| | | HLA-DMB- | | | | |
| | | B3v3 | | | | |
| | | HLA-DMB- | | | | |
| | | FIPv3 | | | | |
| | | HLA-DMB- | | | | |
| | | BIPv3 | | | | |
| | | HLA-DMB- | | | | |
| | | LFv3 | | | | |
| | | HLA-DMB- | | | | |
| | | LBv3 | | | | |
| | fail | v4 | too fast | + | | |
| | | HLA-DMB- | | | | |
| | | F3v3 | | | | |
| | | HLA-DMB- | | | | |
| | | B3v3 | | | | |
| | | HLA-DMB- | | | | |
| | | FIPv4 | | | | |
| | | HLA-DMB- | | | | |
| | | BIPv4 | | | | |
| | | HLA-DMB- | | | | |
| | | LFv3 | | | | |
| | | HLA-DMB- | | | | |
| | | LBv3 | | | | |
| | fail | v5 | too fast | − | | |
| | | HLA-DMB- | | | | |
| | | F3v5 | | | | |
| | | HLA-DMB- | | | | |
| | | B3v5 | | | | |
| | | HLA-DMB- | | | | |
| | | FIPv5 | | | | |
| | | HLA-DMB- | | | | |
| | | BIPv5 | | | | |
| | | HLA-DMB- | | | | |
| | | LFv5 | | | | |
| | | HLA-DMB- | | | | |
| | | LBv5 | | | | |
| | fail | v6 | too fast | − | | |
| | | HLA-DMB- | | | | |
| | | F3v6 | | | | |
| | | HLA-DMB- | | | | |
| | | B3v6 | | | | |
| | | HLA-DMB- | | | | |
| | | FIPv6 | | | | |
| | | HLA-DMB- | | | | |
| | | BIPv6 | | | | |
| | | HLA-DMB- | | | | |
| | | LFv6 | | | | |
| | | HLA-DMB- | | | | |
| | | LBv6 | | | | |
| | fail | v5 + t | too fast | − | | |
| | | HLA-DMB- | | | | |
| | | F3v5 | | | | |
| | | HLA-DMB- | | | | |
| | | B3v5 | | | | |
| | | HLA-DMB- | | | | |
| | | FIPv5 + t | | | | |
| | | HLA-DMB- | | | | |
| | | BIPv5 + t | | | | |
| | | HLA-DMB- | | | | |
| | | LFv5 | | | | |
| | | HLA-DMB- | | | | |
| | | LBv5 | | | | |
| | fail | v6 + t | too fast | − | | |
| | | HLA-DMB- | | | | |
| | | F3v6 | | | | |
| | | HLA-DMB- | | | | |
| | | B3v6 | | | | |
| | | HLA-DMB- | | | | |
| | | FIPv6 + t | | | | |
| | | HLA-DMB- | | | | |
| | | BIPv6 + t | | | | |
| | | HLA-DMB- | | | | |
| | | LFv6 | | | | |
| | | HLA-DMB- | | | | |
| | | LBv6 | | | | |
| | fail | v5 (50%) | too fast | − | | |
| | fail | v6 (50%) | too fast | − | | |
| | fail | v7 | too fast | − | | |
| | | HLA-DMB- | | | | |
| | | F3v7 | | | | |
| | | HLA-DMB- | | | | |
| | | B3v7 | | | | |
| | | HLA-DMB- | | | | |
| | | FIPv7 | | | | |
| | | HLA-DMB- | | | | |
| | | BIPv7 | | | | |
| | | HLA-DMB- | | | | |
| | | LFv7 | | | | |
| | | HLA-DMB- | | | | |
| | | LBv7 | | | | |
| | pass | v8 | in range | − | + | − |
| | | HLA-DMB- | | | | |
| | | F3v8 | | | | |
| | | HLA-DMB- | | | | |
| | | B3v8 | | | | |
| | | HLA-DMB- | | | | |
| | | FIPv8 | | | | |
| | | HLA-DMB- | | | | |
| | | BIPv8 | | | | |
| | | HLA-DMB- | | | | |
| | | LFv8 | | | | |
| | | HLA-DMB- | | | | |
| | | LBv8 | | | | |
| | fail | v9 | too fast | − | | |
| | | HLA-DMB- | | | | |
| | | F3v9 | | | | |
| | | HLA-DMB- | | | | |
| | | B3v9 | | | | |
| | | HLA-DMB- | | | | |
| | | FIPv9 | | | | |
| | | HLA-DMB- | | | | |
| | | BIPv9 | | | | |
| | | HLA-DMB- | | | | |
| | | LFv9 | | | | |
| | | HLA-DMB- | | | | |
| | | LBv9 | | | | |
| | fail | v10 | in range | − | + | − |
| | (irregular | HLA-DMB- | | | | |
| | amp curve) | F3v10 | | | | |
| | | HLA-DMB- | | | | |
| | | B3v10 | | | | |
| | | HLA-DMB- | | | | |
| | | FIPv10 | | | | |
| | | HLA-DMB- | | | | |
| | | BIPv10 | | | | |
| | | HLA-DMB- | | | | |
| | | LFv10 | | | | |
| | | HLA-DMB- | | | | |
| | | LBv10 | | | | |
| | fail | v11 | too slow | + | + | − |
| | | HLA-DMB- | | | | |
| | | F3v11 | | | | |
| | | HLA-DMB- | | | | |
| | | B3v11 | | | | |
| | | HLA-DMB- | | | | |
| | | FIPv11 | | | | |
| | | HLA-DMB- | | | | |
| | | BIPv11 | | | | |
| | | HLA-DMB- | | | | |
| | | LFv11 | | | | |
| | | HLA-DMB- | | | | |
| | | LBv11 | | | | |
| ISG15 | pass | v1 | in range | − | + | − |
| | | ISG15-F3v1 | | | | |
| | | ISG15-B3v1 | | | | |
| | | ISG15-FIPv1 | | | | |
| | | ISG15-BIPv1 | | | | |
| | | ISG15-LFv1 | | | | |
| | | ISG15-LBv1 | | | | |
| | pass | v2 | in range | − | + | − |
| | | ISG15-F3v1 | | | | |
| | | ISG15-B3v1 | | | | |
| | | ISG15-FIPv2 | | | | |
| | | ISG15-BIPv2 | | | | |
| | | ISG15-LFv1 | | | | |
| | | ISG15-LBv1 | | | | |
| | fail | v3 | too fast | − | | |
| | | ISG15-F3v3 | | | | |
| | | ISG15-B3v3 | | | | |
| | | ISG15-FIPv3 | | | | |
| | | ISG15-BIPv3 | | | | |
| | | ISG15-LFv3 | | | | |
| | | ISG15-LBv3 | | | | |
| | fail | v4 | in range | − | + | + |
| | | ISG15-F3v3 | | | | |
| | | ISG15-B3v3 | | | | |
| | | ISG15-FIPv4 | | | | |
| | | ISG15-BIPv4 | | | | |
| | | ISG15-LFv3 | | | | |
| | | ISG15-LBv3 | | | | |
| JUP | fail | v1 | too fast | − | | − |
| | | JUP-F3v1 | | | | |
| | | JUP-B3v1 | | | | |
| | | JUP-FIPv1 | | | | |
| | | JUP-BIPv1 | | | | |
| | | JUP-LFv1 | | | | |
| | | JUP-LBv1 | | | | |
| | fail | v2 | too fast | − | + | |
| | | JUP-F3v1 | | | | |
| | | JUP-B3v1 | | | | |
| | | JUP-FIPv2 | | | | |
| | | JUP-BIPv2 | | | | |
| | | JUP-LFv1 | | | | |
| | | JUP-LBv1 | | | | |
| | fail | v3 | too slow | − | − | |
| | | JUP-F3v3 | | | | |
| | | JUP-B3v3 | | | | |
| | | JUP-FIPv3 | | | | |
| | | JUP-BIPv3 | | | | |
| | | JUP-LFv3 | | | | |
| | | JUP-LBv3 | | | | |
| | fail | v4 | too slow | − | + | − |
| | | JUP-F3v3 | | | | |
| | | JUP-B3v3 | | | | |
| | | JUP-FIPv4 | | | | |
| | | JUP-BIPv4 | | | | |
| | | JUP-LFv3 | | | | |
| | | JUP-LBv3 | | | | |
| | fail | v5 | too slow | − | + | − |
| | | JUP-F3v5 | | | | |
| | | JUP-B3v5 | | | | |
| | | JUP-FIPv5 | | | | |
| | | JUP-BIPv5 | | | | |
| | | JUP-LFv5 | | | | |
| | | JUP-LBv5 | | | | |
| | fail | v6 | too slow | − | − | |
| | | JUP-F3v5 | | | | |
| | | JUP-B3v5 | | | | |
| | | JUP-FIPv6 | | | | |
| | | JUP-BIPv6 | | | | |
| | | JUP-LFv5 | | | | |
| | | JUP-LBv5 | | | | |
| | pass | v7 | in range | − | + | − |
| | | JUP-F3v7 | | | | |
| | | JUP-B3v7 | | | | |
| | | JUP-FIPv7 | | | | |
| | | JUP-BIPv7 | | | | |
| | | JUP-LFv7 | | | | |
| | | JUP-LBv7 | | | | |
| OASL | fail | v1 | too slow | − | | |
| | | OASL-F3v1 | | | | |
| | | OASL-B3v1 | | | | |
| | | OASL-FIPv1 | | | | |
| | | OASL-BIPv1 | | | | |
| | | OASL-LFv1 | | | | |
| | | OASL-LBv1 | | | | |
| | pass | v2 | in range | − | + | − |
| | | OASL-F3v1 | | | | |
| | | OASL-B3v1 | | | | |
| | | OASL-FIPv2 | | | | |
| | | OASL-BIPv2 | | | | |
| | | OASL-LFv1 | | | | |
| | | OASL-LBv1 | | | | |
| OLFM4 | pass | v1 | in range | − | + | − |
| | | OLFM4-F3v1 | | | | |
| | | OLFM4-B3v1 | | | | |
| | | OLFM4-FIPv1 | | | | |
| | | OLFM4-BIPv1 | | | | |
| | | OLFM4-LFv1 | | | | |
| | | OLFM4-LBv1 | | | | |
| PDE4B | fail | v1 | too slow | − | | − |
| | | PDE4B-F3v1 | | | | |
| | | PDE4B-B3v1 | | | | |
| | | PDE4B-FIPv1 | | | | |
| | | PDE4B-BIPv1 | | | | |
| | | PDE4B-LFv1 | | | | |
| | | PDE4B-LBv1 | | | | |
| | fail | v2 | too slow | − | | |
| | | PDE4B-F3v2 | | | | |
| | | PDE4B-B3v2 | | | | |
| | | PDE4B-FIPv2 | | | | |
| | | PDE4B-BIPv2 | | | | |
| | | PDE4B-LFv2 | | | | |
| | | PDE4B-LBv2 | | | | |
| | fail | v3 | too slow | − | | |
| | | PDE4B-F3v3 | | | | |
| | | PDE4B-B3v3 | | | | |
| | | PDE4B-FIPv3 | | | | |
| | | PDE4B-BIPv3 | | | | |
| | | PDE4B-LFv3 | | | | |
| | | PDE4B-LBv3 | | | | |
| | fail | v4 | too slow | − | | |
| | | PDE4B-F3v4 | | | | |
| | | PDE4B-B3v4 | | | | |
| | | PDE4B-FIPv4 | | | | |
| | | PDE4B-BIPv4 | | | | |
| | | PDE4B-LFv4 | | | | |
| | | PDE4B-LBv4 | | | | |
| | pass | v5 | in range | − | + | − |
| | | PDE4B-F3v5 | | | | |
| | | PDE4B-B3v5 | | | | |
| | | PDE4B-FIPv5 | | | | |
| | | PDE4B-BIPv5 | | | | |
| | | PDE4B-LFv5 | | | | |
| | | PDE4B-LBv5 | | | | |
| | fail | v6 | too fast | − | | |
| | | PDE4B-F3v6 | | | | |
| | | PDE4B-B3v6 | | | | |
| | | PDE4B-FIPv6 | | | | |
| | | PDE4B-BIPv6 | | | | |
| | | PDE4B-LFv6 | | | | |
| | | PDE4B-LBv6 | | | | |
| PSMB9 | fail | v1 | too fast | + | | |
| | | PSMB9-F3v1 | | | | |
| | | PSMB9-B3v1 | | | | |
| | | PSMB9-FIPv1 | | | | |
| | | PSMB9-BIPv1 | | | | |
| | | PSMB9-LFv1 | | | | |
| | | PSMB9-LBv1 | | | | |
| | fail | v2 | in range | − | + | + |
| | | PSMB9-F3v2 | | | | |
| | | PSMB9-B3v2 | | | | |
| | | PSMB9-FIPv2 | | | | |
| | | PSMB9-BIPv2 | | | | |
| | | PSMB9-LFv2 | | | | |
| | | PSMB9-LBv2 | | | | |
| | fail | v3 | too fast | + | | |
| | | PSMB9-F3v3 | | | | |
| | | PSMB9-B3v3 | | | | |
| | | PSMB9-FIPv3 | | | | |
| | | PSMB9-BIPv3 | | | | |
| | | PSMB9-LFv3 | | | | |
| | | PSMB9-LBv3 | | | | |
| | fail | v4 | too slow | − | | |
| | | PSMB9-F3v4 | | | | |
| | | PSMB9-B3v4 | | | | |
| | | PSMB9-FIPv4 | | | | |
| | | PSMB9-BIPv4 | | | | |
| | | PSMB9-LFv4 | | | | |
| | | PSMB9-LBv4 | | | | |
| | fail | v5 | too slow | − | | |
| | | PSMB9-F3v5 | | | | |
| | | PSMB9-B3v5 | | | | |
| | | PSMB9-FIPv5 | | | | |
| | | PSMB9-BIPv5 | | | | |
| | | PSMB9-LFv5 | | | | |
| | | PSMB9-LBv5 | | | | |
| | fail | v6 | too fast | − | | |
| | | PSMB9-F3v6 | | | | |
| | | PSMB9-B3v6 | | | | |
| | | PSMB9-FIPv6 | | | | |
| | | PSMB9-BIPv6 | | | | |
| | | PSMB9-LFv6 | | | | |
| | | PSMB9-LBv6 | | | | |
| | pass | v7 | in range | − | + | − |
| | | PSMB9-F3v7 | | | | |
| | | PSMB9-B3v7 | | | | |
| | | PSMB9-FIPv7 | | | | |
| | | PSMB9-BIPv7 | | | | |
| | | PSMB9-LFv7 | | | | |
| | | PSMB9-LBv7 | | | | |
| | fail | v8 | too fast | − | | |
| | | PSMB9-F3v8 | | | | |
| | | PSMB9-B3v8 | | | | |
| | | PSMB9-FIPv8 | | | | |
| | | PSMB9-BIPv8 | | | | |
| | | PSMB9-LFv8 | | | | |
| | | PSMB9-LBv8 | | | | |
| RAPGEF1 | pass | v1 | in range | − | + | − |
| | | RAPGEF-F3v1 | | | | |
| | | RAPGEF-B3v1 | | | | |
| | | RAPGEF- | | | | |
| | | FIPv1 | | | | |
| | | RAPGEF- | | | | |
| | | BIPv1 | | | | |
| | | RAPGEF-LFv1 | | | | |
| | | RAPGEF-LBv1 | | | | |
| | fail | v2 | too fast | + | | |
| | | RAPGEF-F3v2 | | | | |
| | | RAPGEF-B3v2 | | | | |
| | | RAPGEF- | | | | |
| | | FIPv2 | | | | |
| | | RAPGEF- | | | | |
| | | BIPv2 | | | | |
| | | RAPGEF-LFv2 | | | | |
| | | RAPGEF-LBv2 | | | | |
| | fail | v3 | too fast | − | | |
| | | RAPGEF-F3v3 | | | | |
| | | RAPGEF-B3v3 | | | | |
| | | RAPGEF- | | | | |
| | | FIPv3 | | | | |
| | | RAPGEF- | | | | |
| | | BIPv3 | | | | |
| | | RAPGEF-LFv3 | | | | |
| | | RAPGEF-LBv3 | | | | |
| | fail | v4 | too fast | − | | |
| | | RAPGEF1- | | | | |
| | | F3v4 | | | | |
| | | RAPGEF1- | | | | |
| | | B3v4 | | | | |
| | | RAPGEF1- | | | | |
| | | FIPv4 | | | | |
| | | RAPGEF1- | | | | |
| | | BIPv4 | | | | |
| | | RAPGEF1- | | | | |
| | | LFv4 | | | | |
| | | RAPGEF1- | | | | |
| | | LBv4 | | | | |
| | fail | v5 | in range | + | | |
| | | RAPGEF1- | | | | |
| | | F3v5 | | | | |
| | | RAPGEF1- | | | | |
| | | B3v5 | | | | |
| | | RAPGEF1- | | | | |
| | | FIPv5 | | | | |
| | | RAPGEF1- | | | | |
| | | BIPv5 | | | | |
| | | RAPGEF1- | | | | |
| | | LFv5 | | | | |
| | | RAPGEF1- | | | | |
| | | LBv5 | | | | |
| | fail | v6 | too fast | − | | |
| | | RAPGEF1- | | | | |
| | | F3v6 | | | | |
| | | RAPGEF1- | | | | |
| | | B3v6 | | | | |
| | | RAPGEF1- | | | | |
| | | FIPv6 | | | | |
| | | RAPGEF1- | | | | |
| | | BIPv6 | | | | |
| | | RAPGEF1- | | | | |
| | | LFv6 | | | | |
| | | RAPGEF1- | | | | |
| | | LBv6 | | | | |
| | pass | v7 | in range | − | + | − |
| | | RAPGEF1- | | | | |
| | | F3v7 | | | | |
| | | RAPGEF1- | | | | |
| | | B3v7 | | | | |
| | | RAPGEF1- | | | | |
| | | FIPv7 | | | | |
| | | RAPGEF1- | | | | |
| | | BIPv7 | | | | |
| | | RAPGEF1- | | | | |
| | | LFv7 | | | | |
| | | RAPGEF1- | | | | |
| | | LBv7 | | | | |
| | fail | v8 | too fast | − | | |
| | | RAPGEF1- | | | | |
| | | F3v8 | | | | |
| | | RAPGEF1- | | | | |
| | | B3v8 | | | | |
| | | RAPGEF1- | | | | |
| | | FIPv8 | | | | |
| | | RAPGEF1- | | | | |
| | | BIPv8 | | | | |
| | | RAPGEF1- | | | | |
| | | LFv8 | | | | |
| | | RAPGEF1- | | | | |
| | | LBv8 | | | | |
| | fail | v9 | too fast | − | | |
| | | RAPGEF1- | | | | |
| | | F3v9 | | | | |
| | | RAPGEF1- | | | | |
| | | B3v9 | | | | |
| | | RAPGEF1- | | | | |
| | | FIPv9 | | | | |
| | | RAPGEF1- | | | | |
| | | BIPv9 | | | | |
| | | RAPGEF1- | | | | |
| | | LFv9 | | | | |
| | | RAPGEF1- | | | | |
| | | LBv9 | | | | |
| S100A12 | fail | v1 | too fast | − | | |
| | | S100A12-F3v1 | | | | |
| | | S100A12-B3v1 | | | | |
| | | S100A12-FIPv1 | | | | |
| | | S100A12- | | | | |
| | | BIPv1 | | | | |
| | | S100A12-LFv1 | | | | |
| | | S100A12-LBv1 | | | | |
| | fail | v2 | too fast | − | | |
| | | S100A12-F3v2 | | | | |
| | | S100A12-B3v2 | | | | |
| | | S100A12- | | | | |
| | | FIPv2 | | | | |
| | | S100A12- | | | | |
| | | BIPv2 | | | | |
| | | S100A12-LFv2 | | | | |
| | | S100A12-LBv2 | | | | |
| | fail | v3 | too fast | − | | |
| | | S100A12-F3v3 | | | | |
| | | S100A12-B3v3 | | | | |
| | | S100A12- | | | | |
| | | FIPv3 | | | | |
| | | S100A12- | | | | |
| | | BIPv3 | | | | |
| | | S100A12-LFv3 | | | | |
| | | S100A12-LBv3 | | | | |
| | fail | v1 + t | too fast | + | | |
| | | S100A12-F3v1 | | | | |
| | | S100A12-B3v1 | | | | |
| | | FURIN- | | | | |
| | | FIPv1 + t | | | | |
| | | FURIN- | | | | |
| | | BIPv1 + t | | | | |
| | | S100A12-LFv1 | | | | |
| | | S100A12-LBv1 | | | | |
| | fail | v2 + t | too fast | − | | |
| | | S100A12-F3v2 | | | | |
| | | S100A12-B3v2 | | | | |
| | | S100A12- | | | | |
| | | FIPv2 + t | | | | |
| | | S100A12- | | | | |
| | | BIPv2 + t | | | | |
| | | S100A12-LFv2 | | | | |
| | | S100A12-LBv2 | | | | |
| | fail | v3 + t | too fast | − | | |
| | | S100A12-F3v3 | | | | |
| | | S100A12-B3v3 | | | | |
| | | S100A12- | | | | |
| | | FIPv3 + t | | | | |
| | | S100A12- | | | | |
| | | BIPv3 + t | | | | |
| | | S100A12-LFv3 | | | | |
| | | S100A12-LBv3 | | | | |
| | fail | v4 | too fast | − | | |
| | | S100A12-F3v4 | | | | |
| | | S100A12-B3v4 | | | | |
| | | S100A12- | | | | |
| | | FIPv4 | | | | |
| | | S100A12- | | | | |
| | | BIPv4 | | | | |
| | | S100A12-LFv4 | | | | |
| | | S100A12-LBv4 | | | | |
| | fail | v5 | too fast | − | | |
| | | S100A12-F3v5 | | | | |
| | | S100A12-B3v5 | | | | |
| | | S100A12- | | | | |
| | | FIPv5 | | | | |
| | | S100A12- | | | | |
| | | BIPv5 | | | | |
| | | S100A12-LFv5 | | | | |
| | | S100A12-LBv5 | | | | |
| | fail | v6 | too slow | − | | |
| | | S100A12-F3v6 | | | | |
| | | S100A12-B3v6 | | | | |
| | | S100A12- | | | | |
| | | FIPv6 | | | | |
| | | S100A12- | | | | |
| | | BIPv6 | | | | |
| | | S100A12-LFv6 | | | | |
| | | S100A12-LBv6 | | | | |
| | fail | v7 | too fast | − | | |
| | | S100A12-F3v7 | | | | |
| | | S100A12-B3v7 | | | | |
| | | S100A12- | | | | |
| | | FIPv7 | | | | |
| | | S100A12- | | | | |
| | | BIPv7 | | | | |
| | | S100A12-LFv7 | | | | |
| | | S100A12-LBv7 | | | | |
| | pass | v1 (50%) | in range | − | + | − |
| | | S100A12-F3v1 | | | | |
| | | S100A12-B3v1 | | | | |
| | | S100A12-FIPv1 | | | | |
| | | S100A12- | | | | |
| | | BIPv1 | | | | |
| | | S100A12-LFv1 | | | | |
| | | S100A12-LBv1 | | | | |

Validated primer sets having satisfied all the criteria, as indicated with a “pass” designation in Table 9, were selected and are presented in Table 10. It will be appreciated that certain primer sets passed the criteria as shown in Table 9, but are not present in Table 10, are entirely suitable for use in the herein-described methods.

TABLE 10
Validated primer sets that can be used for isothermal amplification of biomarkers
related to acute infection.

| Target and | | |
|---|---|---|
| Version | Oligo ID | Sequences |
| ARG1 | ARG1-F3v1 | ACTGAGGGTTGACTGACTGG |
| | ARG1-B3V1 | CACATCACACTCTTGTTCTTTAAGTTTCTCA |
| | ARG1-FIPv1 | CTCCAATAATCCCTATGGTTCTGGACTTTTTTAGAGCTCAAGTGCAG |
| | | CAAAGAG |
| | ARG1-BIPv1 | CTTTCTCAAAGGGACAGCCACGTTTTTAGCAGACCAGCCTTTCTCAA |
| | | TACT |
| | ARG1-LFv1 | GCGCTCATGCTCTGACACTT |
| | ARG1-LBv1 | AGGGGTGGAAGAAGGCCCTA |
| BATF | PD BATFv3 | CTGAGTGTGAGAGCCCGGAAGATTT |
| | F3 | |
| | PD BATFv3 | TGTTCAGCACCGACGTGAAGTACTT |
| | B3 | |
| | PD BATFv3 | TACGATTTTTCTCCCTCCTCTGAACTCTTCAGCAGTGACTCCAGCTT |
| | FIP | CAGC |
| | PD BATFv3 | GAAGAGCCGACAGAGGCAGTGCTTGATCTCCTTGCGTAGAGCC |
| | BIP | |
| | PD BATFv3 | CATCAGATGAGTCCTGTTTGCCAGG |
| | LF | |
| | PD BATFv3 | GCACCTGGAGAGCGAAGACCT |
| | LB | |
| C3AR1 | C3AR1-F3v1 | GGACCAGACAGGACTCGTG |
| | C3AR1-B3v1 | CGCTGCATCTTCAGGCCA |
| | C3AR1-FIPv2 | TGAGAGTAGGTCAGTTGAATTGGTCTCCAGGTGCTGAAGCCTTC |
| | C3AR1-BIPv2 | ATGGAATGAGCCCCCAGTAATTCTGCCTGGCAATCCCAGTA |
| | C3AR1-LFv1 | GAAAGACGCCATTGCTAAACTTCA |
| | C3AR1-LBv1 | CCATGGTCATTCTCAGCCTTACT |
| Cgorf95/ | PD NMRK1v7 | GGATGATTTCTTCAAGCCAGAGTCT |
| NMRK1 | F3 | |
| | PD NMRK1v7 | CATATGGAATAGTCAGGAAATAGCTTCT |
| | B3 | |
| | PD NMRK1v7 | TGCGCTTTCCATCCAGCAGGATTTTTGCAGTACGATGTGCTT |
| | FIP | |
| | PD NMRK1v7 | CACTCTGTGGTATCAACAGACCAATTCCATATAGTGTCAAGGGGCTT |
| | BIP | ATAA |
| | PD NMRK1v7 | ACATCATTTTTTCCATGTTAAGTGCTTC |
| | FL | |
| | PD NMRK1v7 | CTGAGGAAATTCCCATTTTAATCATCGA |
| | BL | |
| CD163 | PD CD163v9 | TGTGGCAGTGCCCATCA |
| | F3 | |
| | PD CD163v9 | CTGACCAAACTCTGCTTCTTTGAAT |
| | B3 | |
| | PD CD163v9 | ACACGTCCAGAACAGGAAGTGCGGAGGAGACCTGGATCACA |
| | FIP | |
| | PD CD163v9 | AGATCTGGCATGGAGGTTCCACCACAGCCAAGTIGTTGAC |
| | BIP | |
| | PD CD163v9 | GGTCCTTCCTGAAGTCTTATCTTGTT |
| | FL | |
| | PD CD163v9 | GGGACTTGGACGATGCTCA |
| | BL | |
| CEACAM1 | PD | CCCTACAAGTCATAAAGTCAGATCTTGT |
| | CEACAM1v13 | |
| | F3 | |
| | PD | CTGTTGCCATTGGACAGCT |
| | CEACAM1v13 | |
| | B3 | |
| | PD | TCCACAGGGTTGGAGTTGTTGGAATGAAGAAGCAACTGGACAGT |
| | CEACAM1v13 | |
| | FIP | |
| | PD | GACAAGGATGCTGTGGCCTTGAGGCTCTGATTGTTTATCCACC |
| | CEACAM1v13 | |
| | BIP | |
| | PD | GGCAGCTCCGGGTATACAT |
| | CEACAM1v13 | |
| | FL | |
| | PD | CACCTGTGAACCTGAGACTCA |
| | CEACAM1v13 | |
| | BL | |
| CTSB | CTSB_27_F3 | TCTGTGAGCCTGGCTACAG |
| | CTSB_715_ | CATGGCCACCCATCATCTC |
| | B3 | |
| | CTSB_27_ | CGGCCATGATGTCCTTCTCGCAACAGGACAAGCACTACGGA |
| | FIP | |
| | CTSB_715_ | ACAAAAACGGCCCCGTGGAGACGTGTTGGTACACTCCTGA |
| | BIP | |
| | CTSB_LF_27- | ATGTTAAGGATGTCGCAGAGGT |
| | 1 | |
| | CTSB_LB_715- | GGAGCTTTCTCTGTGTATTCGG |
| | 1 | |
| CTSL1 | CTSL1-F3v1 | CGACCTCCGCAACCTTGAG |
| | CTSL1-B3v1 | CTTGGTCCACTGTGCCTCTAAAC |
| | CTSL1-FIPv1 | AAACCTACTCGACCGCGTCCTTTTTCTACGACCGCAGCAGGAA |
| | CTSL1-BIPv1 | TAAAACATGAATCCTACACTCATCCTTTTTTTTGTGATCAAATGTTA |
| | | GAGTAGCTGAGG |
| | CTSL1-LFv1 | CTCTTCCAGTCCCTGTCCGG |
| | CTSL1-LBv1 | GCCTTTTGCCTGGGAATTG |
| DEFA4 | PE DEFA4 | AGGTGATGAGGCTCCAGG |
| | i2v4-12 F3 | |
| | PE DEFA4 | TGAAACTCACACCACCAATGA |
| | i2v4-12 B3 | |
| | PE DEFA4 | ACCTGAAGAGCAGAGCTTTTATCCCAGCGTGGGCCAGAAGAC |
| | i2v4-12 FIP | |
| | PE DEFA4 | TCAGGCTCAACAAGGGGCATGGCAGTTCCCAACACGAAGTT |
| | i2v4-12 BIP | |
| | PE DEFA4 | GCTCTTGCAGATTAGTATTCTGCCGG |
| | i2v4-12 FL | |
| | PE DEFA4 | GTCCTGTATAGATAAAGGAAACGTA |
| | i2v4-12 BL | |
| FURIN | FURIN-F3v1 | TGAGGCCCTGGTTGCTAT |
| | FURIN-B3v1 | TCACTCCTCGATGCCAGAA |
| | FURIN-FIPv1 | TTGGTGAAGACCTTCTGGCGCAACAGGAACCTTGGTCC |
| | FURIN-BIPv1 | CAACAGTGTGGCACGGAAGGTAATAGTCCCCGAAGATCTGG |
| | FURIN-LFv1 | CCTGAGCATCAGCTGCTAG |
| | FURIN-LBv1 | GCATGGGTTCCTCAACCTG |
| GADD45A | GADD45A- | CCTGTGAGTGAGTGCAGAAA |
| | F3v1 | |
| | GADD45A- | ACCCCGACAGTGATCGTG |
| | B3v1 | |
| | GADD45A- | TTCGGTCTTCTGCTCTCCAGCGACCTGCAGTTTGCAATA |
| | FIPv2 | |
| | GADD45A- | GGATGGATAAGGTGGGGGAGCTGACTCAGGGCTTTGC |
| | BIPv2 | |
| | GADD45A- | GCCGAGAATTCCTCCAAAGT |
| | LFv1 | |
| | GADD45A- | CCCTGGAGGAAGTGCTCA |
| | LBv1 | |
| GNA15 | PD GNA15v5 | GGCATCCGGGCCTGCTATGA |
| | F3 | |
| | PD GNA15v5 | TGCCCACCACTGGCATCAAC |
| | B3 | |
| | PD GNA15v5 | TCCTCGGTGATGCGCTCCAGCGTCGGCGGGAATTCCACCT |
| | FIP | |
| | PD GNA15v5 | CACAGCTCAGGACGTGCTCCGATCCGCAGGTTGGTTTTCTGCAC |
| | BIP | |
| | PD GNA15v5 | CCATTTCTTACGCTCTGACTTCTGGCC |
| | FL | |
| | PD GNA15v5 | GGACAGGTAGTACACGGCTGAATCGAG |
| | BL | |
| HK3 | PD HK3v4 F3 | acctgaggagagtgactagcttct |
| | PD HK3v4 B3 | gcctgctccatggaacccaaga |
| | PD HK3v4 | tcagagcaactcagggtttcttccccactgtggaagctcatggac |
| | FIP | |
| | PD HK3v4 | tcagagctggtgcaggagtgcgctggcttggatctgctgtagc |
| | BIP | |
| | PD HK3v4 FL | ccgcaaccctgaagaccca |
| | PD HK3v4 BL | gcagttcaaggtgacaagggcac |
| HLA-DMB | HLA-DMB- | ATCACATTCCTGCCGCTG |
| | F3v8 | |
| | HLA-DMB- | CGCCAAGCTATTCAGCAC |
| | B3v8 | |
| | HLA-DMB- | GGAGATGCAGTATGTGAAATCCGTGGAAAGCACCTGTCTGTT |
| | FIPv8 | |
| | HLA-DMB- | TTCAACAAGGATCTGCTGACCAATTCGCAAGGGGCCATC |
| | BIPv8 | |
| | HLA-DMB- | TTGGAGTCCCAGCATCATC |
| | LFv8 | |
| | HLA-DMB- | GCTGGGATCCAGAGGAGAATA |
| | LBv8 | |
| IFI27 | IFI27_64_F3 | AGCAGCCAAGATGATGTCC |
| | IFI27_65_B3 | GATAGTTGGCTCCTCGCTG |
| | IFI27_64_ | TGCTCCCAGTGACTGCAGAGTAATTGCCAATGGGGGTGGA |
| | FIP | |
| | IFI27_65_ | TGCGAGGTTCTACTAGCTCCCTTTCTCCCCTGGCATGGTT |
| | BIP | |
| | IFI27_64_LF- | TGGGTCTGCCATTGCGG |
| | 4 | |
| | IFI27_65_LB- | CCCTCGCCCTGCAGAGAAGA |
| | 1 | |
| ISG15 | ISG15-FIPv1 | CGCCAGCATCTTCACCGTTTTTAGGAGCTTGTGCCGTGG |
| | ISG15-BIPv1 | GGCAACGAATTCCAGGTGTCTTTTTGCGCCTTCAGCTCTGAC |
| | ISG15-F3v1 | GCGAACTCATCTTTGCCAGTAC |
| | ISG15-B3v1 | CGCCGATCTTCTGGGTGAT |
| | ISG15-LFv1 | TCAGGTCCCAGCCCATG |
| | ISG15-LBv1 | GAGCAGCTCCATGTCGGT |
| JUP | JUP-FIPv7 | GTCACCTTGATAGGCTGCTCTTTTTAGGCCCCATACTCAGTAGC |
| | JUP-BIPv7 | GAGTGGCAGCAGACATACACTTTTTCCTCCATGATGCCCTTGC |
| | JUP-F3v7 | CGAGCTCAGTTCGCTGTC |
| | JUP-B3v7 | GTGGTTTTCTTGAGCGTGTAC |
| | JUP-LFv7 | CATCAGGTTCATCACCTCCATC |
| | JUP-LBv7 | ACGACTCGGGTATCCACTC |
| KCNJ2 | PE KCNJ2v9 | GGGCTGGGTCTTGGGAAT |
| | F3 | |
| | PE KCNJ2v9 | GGAGCCTTGTGGTTCTACC |
| | B3 | |
| | PE KCNJ2v9 | AGCATGGGGACATGGAGTGGAAGGCTCACTCGCTTTTTACAAAC |
| | FIP | |
| | PE KCNJ2v9 | CCTGCGCCAGCAACAGGACTGTCTACTGACATGCAGAGTT |
| | BIP | |
| | PE KCNJ2v9- | TGGGGGGTACAGAGGCAT |
| | 1 FL | |
| | PE KCNJ2v9- | CATGTTCTCTGGATGTCAGCTGAGT |
| | 1 BL | |
| KIAA1370 | PD | GAACTTTTTCTGTACCTGTTAAACAAGA |
| | KIAA1370v5 | |
| | F3 | |
| | PD | GGTTTGTTGGTGATTCAGTGTAAGA |
| | KIAA1370v5 | |
| | B3 | |
| | PD | ATTTAGAACTCTGGAACCTCAGATGTAGAAGTGTTAATAAAGAGAA |
| | KIAA1370v5 | CATCCGAC |
| | FIP | |
| | PD | TGGAAAGATCTACCTCCATAGAGACGATGCAGCACCGCTATCAAC |
| | KIAA1370v5 | |
| | BIP | |
| | PD | GGTAGCGTAATAACCGTTCTTCTG |
| | KIAA1370v5 | |
| | FL | |
| | PD | CTCCTGTTCTCTAGAAAGTCAATGGA |
| | KIAA1370v5 | |
| | BL | |
| KPNA6 | PD KPNA6v6 | atccaggcagtcatagactccgga |
| | F3 | |
| | PD KPNA6v6 | ccacttgttgagcagtcccaagga |
| | B3 | |
| | PD KPNA6v6 | agtgacgatgttacccacggctctattggtagagctgctgatgcacaa |
| | FIP | |
| | PD KPNA6v6 | tcttaactgttcagccctaccttgagtccagcaagcttccttccggat |
| | BIP | |
| | PD KPNA6v6 | atttgagccctgttgccagcagta |
| | FL | |
| | PD KPNA6v6 | cagggcaggagaagccactttgta |
| | BL | |
| LY86 | PD LY86v9 F3 | cttgacctagctctcatgtctcaa |
| | PD LY86v9 | cacatgatagtagcattggcaca |
| | B3 | |
| | PD LY86v9 | gcatagtaaatctgctctcctttccggctcatctgttttgaatttctccta |
| | FIP | |
| | PD LY86v9 | ggcctgtcaataatcctgaatttactggtggaccgtttttcagtgtac |
| | BIP | |
| | PD LY86v9 | ccacagaaagaaaacttgggca |
| | FL | |
| | PD LY86v9 | cctcagggagaataccaggttt |
| | BL | |
| OASL | OASL-F3v1 | GCACTGATGCAGGAACTGTATA |
| | OASL-B3v1 | AGCTCAGAAACGCCACCA |
| | OASL-FIPv2 | CTGCCTCAGAAACTCCTCCAACTCCTTCGTGGCTCAGT |
| | OASL-BIPv2 | GAGCATTTCCAGGGGAAGCCTGAGAACCGTGCCATTCC |
| | OASL-LFv1 | CAGCGTCTAGCACCTCTTC |
| | OASL-LBv1 | CGGGTGCTGAAGGTAGTCA |
| OLFM4 | OLFM4-F3v1 | TCTGTTTCCCTGCCAGAC |
| | OLFM4-B3v1 | TCCTTCACTTCTACCTTGATCAG |
| | OLFM4-FIPv1 | CTCACTTTGGAAAGTTCTTTCTCAGGACAGAGTGGAACGCTT |
| | OLFM4- | GTTAAACCTAACTGTCCGAATTGACTCGAAGTCCAGTTCAGTGTAA |
| | BIPv1 | |
| | OLFM4-LFv1 | AAGAACATGAGCTGTGAATTCC |
| | OLFM4-LBv1 | CATCATGGAGAAGGATACCATTTC |
| PDE4B | PDE4B-F3v5 | CAGCCTAACTACATGCCTGTG |
| | PDE4B-B3v5 | ATTTGAAATGTATTCAGACACCTGG |
| | PDE4B-FIPv5 | GACCGGTAGGTCTGTATGGTAAATTAGCAATGGAAACGCTGG |
| | PDE4B-BIPv5 | CAGTGAGATGGCTTCTAACAAGTTTCCCTGATCGGCTCATCTC |
| | PDE4B-LFv5 | TGGTCTAAACACCAGTCTAATTCC |
| | PDE4B-LBv5 | AAAGAATGCTGAACCGGGAG |
| PER1 | PE PER1v22 | GGCAGGCGTGCGGA |
| | F3 | |
| | PE PER1v22 | AGGGGGCCACTCATGTC |
| | B3 | |
| | PE PER1v22 | AGGGAGTGAGGTGGAAGATCTAAGTGGACACTCCTGCGACCAG |
| | FIP | |
| | PE PER1v22 | AGCCAAGCCTCCAGGCCCTGGGCCATGGGGAGAAC |
| | BIP | |
| | PE PER1v22- | AGTTCGATCACAGCCAGTACC |
| | 4 FL | |
| | PE PER1v22- | CATCCGTGGTGGCCTCTCT |
| | 4 BL | |
| PSMB9 | PSMB9-F3v7 | CAAATGTGGTGAGAAATATCAGCTA |
| | PSMB9-B3v7 | TGCACTCCTCGGGAGACATG |
| | PSMB9-FIPv7 | GTCAGCATTCCTCCCAGGGTGGACTTGTCTGCACATCTCATGG |
| | PSMB9-BIPv7 | CGACAGCCTTTTGCCATTGGTGGCTTATATGCTGCATCCACAT |
| | PSMB9-LFv7 | CATATACCTGACCTCCTTCACGTTG |
| | PSMB9-LBv7 | GCTCCGGCAGCACCTTTAT |
| RAPGEF1 | RAPGEF1- | GTCGAATTTGTGGGTGATTTTACTG |
| | F3v7 | |
| | RAPGEF1- | GGAGTCGCTGAAGCCGTATA |
| | B3v7 | |
| | RAPGEF1- | CGAGTAGTCCTCCAGCAACTCTCCTGAGTCAACCGGTGAC |
| | FIPv7 | |
| | RAPGEF1- | GCAGCCCTCTATGTTCTACCACTCCATGAGGAGCTTGTTCTTC |
| | BIPv7 | |
| | RAPGEF1- | CATGTAGGCCAGCATGTGTTT |
| | LFv7 | |
| | RAPGEF1- | GCCACAGAACGAGCACATCTA |
| | LBv7 | |
| RREB1 | PD RREB1v7 | ATTCCTTTTCCGGAACAAGTTTGCATC |
| | F3 | |
| | PD RREB1v7 | cggagtagaaaatgagtctgtgttgacctctt |
| | B3 | |
| | PD RREB1v7 | acacagtcggagcaacggccctcctcggtctctccctgaagc |
| | FIP | |
| | PD RREB1v7 | gttccaggagtggtggctctgagactgttttctttgtgttatcaagctgccc |
| | BIP | |
| | PD RREB1v7 | ctccctggcatgatgcgttgg |
| | LF | |
| | PD RREB1v7 | gccaggttcagccccccaata |
| | LB | |
| S100A12 | S100A12-F3v1 | CTTGGCTCAGTGCCCTTCAC |
| | S100A12- | TGTTTGCAAGCTCCTTTGTAAGC |
| | B3v1 | |
| | S100A12- | CCTCCAGATGCTCTTCAAGTTTTGGCGCTGTAGCTCCACATTC |
| | FIPv1 | |
| | S100A12- | CTTCCACCAATACTCAGTTCGGAGCTGCTTCAGCTCACCCTTA |
| | BIPv1 | |
| | S100A12- | CCCAGCCTAATGTTAACCCCTC |
| | LFv1 | |
| | S100A12- | AGGGGCATTTTGACACCCTC |
| | LBv1 | |
| TGFBI | PD TGFBIv4 | ggtgatgaaatcctggttagcgga |
| | F3 | |
| | PD TGFBIv4 | cgctgatgcttgtttgaagatctc |
| | B3 | |
| | PD TGFBIv4 | aggctccttgttgacactcaccacgccctggtgcggctaaagtct |
| | FIP | |
| | PD TGFBIv4 | tgacatcatggccacaaatggcgtcagagtctgcaagttcatcccct |
| | BIP | |
| | PD TGFBIv4 | gctgacttccagcttgtcacct |
| | LF | |
| | PD TGFBIv4 | ctccagccaacagacctcaggaa |
| | LB | |
| YWHAB | PE | CTGAAAAGGCCTGTAGCC |
| | YWHABv145 | |
| | F3 | |
| | PE | CAGAGTGACACTGAACAGA |
| | YWHABv145 | |
| | B3 | |
| | PE | TGCATGATCAGAGTGCTGTCTTTATAAAACGGCATTTGATGAAGC |
| | YWHABv145 | |
| | FIP | |
| | PE | CTGTGGACATCGGAAAACCAGTCACAAAGCACGAGAAACA |
| | YWHABv145 | |
| | BIP | |
| | PE | TCAGCGTATCCAATTCAGCAAT |
| | YWHABv145- | |
| | 1 FL | |
| | PE | GAGACGAAGGAGACGCTGGG |
| | YWHABv145- | |
| | 1 BL | |
| ZDHHC19 | PE | TGTCCGTGAGCTCGGC |
| | ZDHHC19- | |
| | 201v15 F3 | |
| | PE | GGGGGGTTGAGAGCAGA |
| | ZDHHC19- | |
| | 201v15 B3 | |
| | PE | TTGGGTCCCAGTGGTGCACAAACTACAAGGGCAAGTGCAGAC |
| | ZDHHC19- | |
| | 201v15 FIP | |
| | PE | TACATGGCTGAAGCTGTCCAGCCATTGGAGGGTGCAGATTCG |
| | ZDHHC19- | |
| | 201v15 BIP | |
| | PE | GGGGTTGTATCCCTGAAGGT |
| | ZDHHC19- | |
| | 201v15 FL | |
| | PE | CAGAGAGTGGTGGGGCCTGA |
| | ZDHHC19- | |
| | 201v15 BL | |

The above description of example embodiments of the present disclosure has been presented for the purposes of illustration and description. It is not intended to be exhaustive or to limit the disclosure to the precise form described, and many modifications and variations are possible in light of the teaching above.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art. Where a conflict exists between the instant application and a reference provided herein, the instant application shall dominate.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “1′ or ‘2’ or ‘3’ or ‘1 and 2’ or ‘1 and 3’ or ‘2 and 3’ or ‘1, 2 and 3’”. Whenever a range is given in the specification, for example, a temperature range, a time range, or a composition range, all intermediate ranges and subranges, as well as all individual values included in the ranges given are intended to be included in the disclosure.

1. A method of treating an acute illness in a subject, comprising the steps of:
a. selecting a patient presenting clinical symptoms of an acute illness and having a biomarker gene score exceeding a threshold value indicating the presence of a bacterial or a viral infection in the patient, wherein the biomarker gene score is based on measured expression levels in blood from the patient of at least two biomarker genes selected from the group consisting of IFI27, JUP, LAX1, CTSB, GPAA1, HK3, and TNIP1;
(i) wherein the expression levels of the two or more biomarker genes are quantitatively determined by amplification and detection of subsequences of mRNAs encoding the two or more biomarker genes,
(ii) wherein the amplification of the subsequence is performed by Reverse-Transcription Loop-Mediated Amplification (RT-LAMP) using a biomarker RT-LAMP primer combination comprising a plurality of biomarker core (FIP, BIP, F3, and B3) primers;
(iii) wherein the plurality of biomarker core primers is selected from the group consisting of:
For IFI27:
(IFI27_64_FIP)
tgctcccagtgactgcagagtaattgccaatgggggtgga,
(IFI27_65_BIP)
tgcgaggttctactagctccctttctcccctggcatggtt,
(IFI27_64_F3)
agcagccaagatgatgtcc,
and
(IFI27_65_B3)
gatagttggctcctcgctg;
For JUP:
(PD JUPv9 F3)
accccaagttcctggccatc,
(PD JUPv9 B3)
tcccaccagcctccacaatg,
(PD JUPv9 FIP)
gatctgcacgagggccttgcagctcctggcctac,
PD JUPv9 BIP
atgcgtaactacagttatgaaaagctgcgcttattgctgggacacacgg
atag;
For LAX1:
(PD LAX1v9 F3)
gaaataaagaccagatcaccaacatctt,
(PD LAX1v9 B3)
gaggaggctctcagtactgaaaat,
(PD LAX1v9 FIP)
gcatgacggtaactcggagcgttgcggttttctgcatc,
and
(PD LAX19 BIP)
tgactttgccacaaaccagacactcatgtctccccaggtctt;
For CTSB:
(CTSB_27_FIP)
cggccatgatgtccttctcgcaacaggacaagcactacgga,
(CTSB_27_F3)
tctgtgagcctggctacag,
(CTSB_715_BIP)
acaaaaacggccccgtggagacgtgttggtacactcctga,
and
(CTSB_715_B3)
catggccacccatcatctc;
For GPAA1:
(GPAA1_23 F3)
gtggaggagcagtttgcg,
(GPAA1_23 B3)
ttggtgcccgacaccata,
(GPAA1_23 FIP)
gttcaagccaggccactggccttttgcccgggacttcg,
(GPAA1_23 BIP)
gatgcggtcagtagggctggacgctcgtgggtctcatct;
For HK3:
(PD HK3v4 F3)
acctgaggagagtgactagcttct,
(PD HK3v4 B3)
gcctgctccatggaacccaaga,
(PD HK3v4 FIP)
tcagagcaactcagggtttcttccccactgtggaagctcatggac,
and
(PD HK3v4 BIP)
tcagagctggtgcaggagtgcgctggcttggatctgctgtagc;
and
For TNIP1:
(PD TNIP1v21-1 F3)
ggatcagctgagcccact,
(PD TNIP1v21-1 B3)
cagcaactcattctgcgtga,
(PD TNIP1v21-1 FIP)
gtgcttcctccagggccttgacccgacagcgtgagtac,
and
(PD TNIP1Vv1-1 BIP)
ccaaaccccgccatcatctccccagctcctgtttccttagg;
including variants of the sets wherein one or more of the biomarker core primers contains 1, 2, or 3 nucleotide substitutions relative to any one of the above sequences; and
b. treating the selected patient with an agent in an amount sufficient to reduce the clinical symptoms of the acute illness.

2. The method of claim 1, wherein the biomarker RT-LAMP primer combination further comprises a pair of biomarker loop (LF and LB) primers.

3. The method of claim 2, wherein the pair of biomarker loop primers is selected from the group consisting of:
For IFI27:
(IFI27_64_LF-4)
tgggtctgccattgcgg,
(IFI27_65_LB-1)
ccctcgccctgcagagaaga;
For JUP:
(PD JUPv9 FL)
gatcagcttgctctcctggtt,
(PD JUPv9 BL)
accaccagtcgtgtgctcaag;
For LAX1:
(PD LAX1v9 FL)
gtcgcttcttccgtttattccaat,
(PD LAX1v9 BL)
agccaaaaatatttatgacatcttgcct;
For CTSB:
(CTSB_LF_27-1)
atgttaaggatgtcgcagaggt,
CTSB_LB_715-1
ggagctttctctgtgtattcgg;
For GPAA1:
(GPAA1_23-1 FL)
ccccgacttcttgcggt,
(GPAA1_23-1 BL)
gcagagtttctcccggaaac;
For HK3:
(PD HK3v4 FL)
ccgcaaccctgaagaccca,
(PD HK3v4 BL)
gcagttcaaggtgacaagggcac;
and
For TNIP1:
(PD TNIP1v21-1 FL)
ccgctggatctccttttcctg,
(PD TNIP1v21-1 BL)
caacagcatttgggagcccag,
including variants of the pairs wherein one or more of the biomarker loop primers contains 1, 2, or 3 nucleotide substitutions relative to one or more of the above sequences.

4. The method of claim 1, wherein the determination of the biomarker gene score is based on relative expression levels of the at least two biomarkers in the biological sample as compared to expression levels of one or more reference genes, and wherein the one or more reference genes comprise KPNA6, RREB1, or YWHAB.

5. (canceled)

6. The method of claim 5, wherein the expression levels of the one or more reference genes are determined by amplification and detection of one or more subsequences of one or more reference gene mRNAs encoding the one or more reference genes,
wherein the amplification of the one or more subsequences of the one or more reference gene mRNAs is performed by RT-LAMP using a reference gene RT-LAMP primer combination comprising a plurality of reference gene core (FIP, BIP, F3, B3) primers; and
wherein the plurality of reference gene core primers is selected from the group consisting of:
For KPNA6:
(PD KPNA6v6 B3)
ccacttgttgagcagtcccaagga,
(PD KPNA6v6 FIP)
agtgacgatgttacccacggctctattggtagagctgctgatgcacaa,
(PD KPNA6v6 BIP)
tcttaactgttcagccctaccttgagtccagcaagcttccttccggat,
For RREB1:
(PD RREB1v7 F3)
gccattttgattccttttccggaacaagt,
(PD RREB1v7 B3)
gccaggttcagccccccaata,
(PD RREB1v7 FIP)
acacagteggagcaacggccctcctcggtctctccctgaagc,
(PD RREB1v7 BIP)
gttccaggagtggtggctctgagactgttttctttgtgttatcaagctg
cccand,
and
For YWHAB:
(PE YWHABv145 FIP)
tgcatgatcagagtgctgtctttataaaacggcatttgatgaagc,
(PE YWHABv145 F3)
ctgaaaaggcctgtagcc,
(PE YWHABv145 BIP)
ctgtggacatcggaaaaccagtcacaaagcacgagaaaca,
(PE YWHABv145 B3)
cagagtgacactgaacaga,
including variants of the pluralities wherein one or more of the reference gene core primers contains 1, 2, or 3 nucleotide substitutions relative to one or more of the above sequences.

7. The method of claim 6, wherein the reference gene RT-LAMP primer combination further comprises a pair of reference gene loop (LF and LB) primers.

8. The method of claim 7, wherein the pair of reference gene loop primers is selected from the group consisting of:
For KPNA6:
(PD KPNA6v6 FL)
atttgagccctgttgccagcagta,
(PD KPNA6v6 BL)
cagggcaggagaagccactttgta,
For RREB1:
(PD RREB1v7 FL)
cggagtagaaaatgagtctgtgttgacctctt,
(PD RREB1v7 BL)
ctccctggcatgatgcgttgg,
and
For YWHAB:
(PE YWHABv145-1 FL)
tcagcgtatccaattcagcaat,
(PE YWHABv145-1 BL)
gagacgaaggagacgctggg
including variants of the pairs wherein one or more of the reference gene loop primers contains 1, 2, or 3 nucleotide substitutions relative to one or more of the above sequences.

9. (canceled)

10. The method of claim 1, wherein the measured expression level of one ore more of IFI27, JUP, and LAX is elevated in the biological sample relative to an expression level representative of an individual without a viral infection.

11. (canceled)

12. The method of claim 1, wherein the measured expression level of one or more of CTSB, GPAA1, HK3, and TNIP1 is elevated in the biological sample relative to an expression level representative of an individual without a bacterial infection.

13. (canceled)

14. A genetic amplification system for diagnosing an acute infection, comprising a multiplicity of reaction vessels and a blood sample from a patient presenting clinical symptoms of an acute infection,
wherein the system is configured to measure the expression levels of at least two biomarker genes by Reverse-Transcription Loop-Mediated Amplification (RT-LAMP) and detection of subsequences of mRNAs encoding the biomarker genes,
wherein a score generated from the measured expression levels is indicative of a likelihood of the presence of a bacterial or a viral infection in the patient, wherein the biomarker genes are selected from the group consisting of IFI27, JUP, LAX1, CTSB, GPAA1, HK3, and TNIP1,
wherein the reaction vessels comprise biomarker RT-LAMP primer combinations for amplification of the biomarker genes, and
wherein the biomarker RT-LAMP primer combination used to amplify the biomarker genes comprises a plurality of biomarker core primers selected from the group consisting of:
For IFI27:
(IFI27_64_FIP)
tgctcccagtgactgcagagtaattgccaatgggggtgga,
(IFI27_65_BIP)
tgcgaggttctactagctccctttctcccctggcatggtt,
(IFI27_64_F3)
agcagccaagatgatgtcc,
and
(IFI27_65_B3)
gatagttggctcctcgctg;
For JUP:
(PD JUPv9 F3)
accccaagttcctggccatc,
(PD JUPv9 B3)
tcccaccagcctccacaatg,
(PD JUPv9 FIP)
gatctgcacgagggccttgcagctcctggcctac,
PD JUPv9 BIP
atgcgtaactacagttatgaaaagctgcgcttattgctgggacacacgg
atag;
For LAX1:
(PD LAX1v9 F3)
gaaataaagaccagatcaccaacatctt,
(PD LAX1v9 B3)
gaggaggctctcagtactgaaaat,
(PD LAX1v9 FIP)
gcatgacggtaactcggagcgttgcggttttctgcatc,
and
(PD LAX1v9 BIP)
tgactttgccacaaaccagacactcatgtctccccaggtctt;
For CTSB:
(CTSB_27_FIP)
cggccatgatgtccttctcgcaacaggacaagcactacgga,
(CTSB_27_F3)
tctgtgagcctggctacag,
(CTSB_715_BIP)
acaaaaacggccccgtggagacgtgttggtacactcctga,
and
(CTSB_715_B3)
catggccacccatcatctc;
For GPAA1:
(GPAA1_23 F3)
gtggaggagcagtttgcg,
(GPAA1_23 B3)
ttggtgcccgacaccata,
(GPAA1_23 FIP)
gttcaagccaggccactggccttttgcccgggacttcg,
(GPAA1_23 BIP)
gatgcggtcagtagggctggacgctcgtgggtctcatct;
For HK3:
(PD HK3v4 F3)
acctgaggagagtgactagcttct,
(PD HK3v4 B3)
gcctgctccatggaacccaaga,
(PD HK3v4 FIP)
tcagagcaactcagggtttcttccccactgtggaagctcatggac,
and
(PD HK3v4 BIP)
tcagagctggtgcaggagtgcgctggcttggatctgctgtagc;
and
For TNIP1:
(PD TNIP1v21-1 F3)
ggatcagctgagcccact,
(PD TNIP1v21-1 B3)
cagcaactcattctgcgtga,
(PD TNIP1v21-1 FIP)
gtgcttcctccagggccttgacccgacagcgtgagtac,
and
(PD TNIP1v21-1 BIP)
ccaaaccccgccatcatctccccagctcctgtttccttagg;
including variants of the pluralities wherein one or more of the biomarker core primers within the combination contains 1, 2, or 3 nucleotide substitutions relative to any one of the above sequences.

15. The system of claim 14, wherein the biomarker RT-LAMP primer combination further comprises a pair of biomarker loop primers selected from the group consisting of:
For IFI27:
(IFI27_64_LF-4)
tgggtctgccattgcgg,
(IFI27_65_LB-1)
ccctcgccctgcagagaaga;
For JUP:
(PD JUPv9 FL)
gatcagcttgctctcctggtt,
(PD JUPv9 BL)
accaccagtcgtgtgctcaag;
For LAX1:
(PD LAX1v9 FL)
gtcgcttcttccgtttattccaat,
(PD LAX1v9 BL)
agccaaaaatatttatgacatcttgcct;
For CTSB:
(CTSB_LF_27-1)
atgttaaggatgtcgcagaggt,
CTSB_LB_715-1
ggagctttctctgtgtattcgg;
For GPAA1:
(GPAA1_23-1 FL)
ccccgacttcttgcggt,
(GPAA1_23-1 BL)
gcagagtttctcccggaaac;
For HK3:
(PD HK3v4 FL)
ccgcaaccctgaagaccca,
(PD HK3v4 BL)
gcagttcaaggtgacaagggcac;
and
For TNIP1:
(PD TNIP1v21-1 FL)
ccgctggatctccttttcctg,
(PD TNIP1v21-1 BL)
caacagcatttgggagcccag,
including variants of the pairs wherein one or more of the biomarker loop primers contains 1, 2, or 3 nucleotide substitutions relative to one or more of the above sequences.

16. The system of claim 14, wherein the reaction vessels further comprise a reference gene RT-LAMP primer combination for amplification of one or more reference genes, and
wherein the reference gene RT-LAMP primer combination comprises a plurality of reference gene core primers selected from the group consisting of:
For KPNA6:
(PD KPNA6v6 B3)
ccacttgttgagcagtcccaagga,
(PD KPNA6v6 FIP)
agtgacgatgttacccacggctctattggtagagctgctgatgcacaa,
(PD KPNA6v6 BIP)
tcttaactgttcagccctaccttgagtccagcaagcttccttccggat,
For RREB1:
(PD RREB1v7 F3)
gccattttgattccttttccggaacaagt,
(PD RREB1v7 B3)
gccaggttcagccccccaata,
(PD RREB1v7 FIP)
acacagteggagcaacggccctcctcggtctctccctgaagc,
(PD RREB1v7 BIP)
gttccaggagtggtggctctgagactgttttctttgtgttatcaagctg
cccand,
and
For YWHAB:
(PE YWHABv145 FIP)
tgcatgatcagagtgctgtctttataaaacggcatttgatgaagc,
(PE YWHABv145 F3)
ctgaaaaggcctgtagcc,
(PE YWHABv145 BIP)
ctgtggacatcggaaaaccagtcacaaagcacgagaaaca,
(PE YWHABv145 B3)
cagagtgacactgaacaga,
including variants of the pluralities wherein one or more of the reference gene core primers contains 1, 2, or 3 nucleotide substitutions relative to one or more of the above sequences.

17. The system of claim 16, wherein the reference gene RT-LAMP primer combination further comprises a pair of reference gene loop primers selected from the group consisting of:
For KPNA6:
(PD KPNA6v6 FL)
atttgagccctgttgccagcagta,
(PD KPNA6v6 BL)
cagggcaggagaagccactttgta,
For RREB1:
(PD RREB1v7 FL)
cggagtagaaaatgagtctgtgttgacctctt,
(PD RREB1v7 BL)
ctccctggcatgatgcgttgg,
and
For YWHAB:
(PE YWHABv145-1 FL)
tcagcgtatccaattcagcaat,
(PE YWHABv145-1 BL)
gagacgaaggagacgctggg
including variants of the pairs wherein one or more of the reference gene loop primers contains 1, 2, or 3 nucleotide substitutions relative to one or more of the above sequences.

18. (canceled)

19. A method of diagnosing a bacterial or viral infection in a patient with symptoms of an acute infection, comprising:
a. selecting a blood sample from a patient presenting clinical symptoms of an acute infection, and quantitatively determining a diagnostic score indicative of a bacterial or viral infection based on measured levels in the patient sample of at least two biomarker genes selected from the group consisting of IFI27, JUP, LAX1, CTSB, GPAA1, HK3, and TNIP1;
(i) where the levels of the biomarker genes are measured by the amplification and detection of subsequences of mRNAs encoding the biomarker genes and wherein the diagnostic score exceeds a threshold indicative of a bacterial or a viral infection, wherein the threshold value is generated by a quantitative comparison of biomarker gene expression level scores of at least 100 patients known to have a diagnosis of a bacterial or a viral infection, and 100 healthy controls;
(ii) wherein the amplification is performed by Reverse-Transcription Loop-Mediated Amplification (RT-LAMP) using a biomarker RT-LAMP primer combination comprising a plurality of biomarker core (FIP, BIP, F3, and B3) primers, and
(iii) wherein the plurality of biomarker core primers is selected from the group consisting of:
For IFI27:
(IFI27_64_FIP)
tgctcccagtgactgcagagtaattgccaatgggggtgga,
(IFI27_65_BIP)
tgcgaggttctactagctccctttctcccctggcatggtt,
(IFI27_64_F3)
agcagccaagatgatgtcc,
and
(IFI27_65_B3)
gatagttggctcctcgctg;
For JUP:
(PD JUPv9 F3)
accccaagttcctggccatc,
(PD JUPv9 B3)
tcccaccagcctccacaatg,
(PD JUPv9 FIP)
gatctgcacgagggccttgcagctcctggcctac,
PD JUPv9 BIP
atgcgtaactacagttatgaaaagctgcgcttattgctgggacacacgg
atag;
For LAX1:
(PD LAX1v9 F3)
gaaataaagaccagatcaccaacatctt,
(PD LAX1v9 B3)
gaggaggctctcagtactgaaaat,
(PD LAX1v9 FIP)
gcatgacggtaactcggagcgttgcggttttctgcatc,
and
(PD LAX1v9 BIP)
tgactttgccacaaaccagacactcatgtctccccaggtctt;
For CTSB:
(CTSB_27_FIP)
cggccatgatgtccttctcgcaacaggacaagcactacgga,
(CTSB_27_F3)
tctgtgagcctggctacag,
(CTSB_715_BIP)
acaaaaacggccccgtggagacgtgttggtacactcctga,
and
(CTSB_715_B3)
catggccacccatcatctc;
For GPAA1:
(GPAA1_23 F3)
gtggaggagcagtttgcg,
(GPAA1_23 B3)
ttggtgcccgacaccata,
(GPAA1_23 FIP)
gttcaagccaggccactggccttttgcccgggacttcg,
(GPAA1_23 BIP)
gatgcggtcagtagggctggacgctcgtgggtctcatct;
For HK3:
(PD HK3v4 F3)
acctgaggagagtgactagcttct,
(PD HK3v4 B3)
gcctgctccatggaacccaaga,
(PD HK3v4 FIP)
tcagagcaactcagggtttcttccccactgtggaagctcatggac,
and
(PD HK3v4 BIP)
tcagagctggtgcaggagtgcgctggcttggatctgctgtagc;
and
For TNIP1:
(PD TNIP1v21-1 F3)
ggatcagctgagcccact,
(PD TNIP1v21-1 B3)
cagcaactcattctgcgtga,
(PD TNIP1v21-1 FIP)
gtgcttcctccagggccttgacccgacagcgtgagtac,
and
(PD TNIP1v21-1 BIP)
ccaaaccccgccatcatctccccagctcctgtttccttagg;
including variants of the pluralities wherein one or more of the biomarker core primers contains 1, 2, or 3 nucleotide substitutions relative to any one of the above sequences.

20. The method of claim 19, wherein the biomarker RT-LAMP primer combination further comprises a pair of biomarker loop primers.

21. The method of claim 20, wherein the pair of biomarker loop primers is selected from the group consisting of:
For IFI27:
(IFI27_64_LF-4)
tgggtctgccattgcgg,
(IFI27_65_LB-1)
ccctcgccctgcagagaaga;
For JUP:
(PD JUPv9 FL)
gatcagcttgctctcctggtt,
(PD JUPv9 BL)
accaccagtcgtgtgctcaag;
For LAX1:
(PD LAX1v9 FL)
gtcgcttcttccgtttattccaat,
(PD LAX1v9 BL)
agccaaaaatatttatgacatcttgcct;
For CTSB:
(CTSB_LF_27-1)
atgttaaggatgtcgcagaggt,
CTSB_LB_715-1
ggagctttctctgtgtattcgg;
For GPAA1:
(GPAA1_23-1 FL)
ccccgacttcttgcggt,
(GPAA1_23-1 BL)
gcagagtttctcccggaaac;
For HK3:
(PD HK3v4 FL)
ccgcaaccctgaagaccca,
(PD HK3v4 BL)
gcagttcaaggtgacaagggcac;
and
For TNIP1:
(PD TNIP1v21-1 FL)
ccgctggatctccttttcctg,
(PD TNIP1v21-1 BL)
caacagcatttgggagcccag,
including variants of the pairs wherein one or more of the biomarker loop primers contains 1, 2, or 3 nucleotide substitutions relative to one or more of the above sequences.

22. The method claim 19, wherein the determination of the biomarker gene score is based on relative expression levels of the at least two biomarkers in the biological sample as compared to expression levels of one or more reference genes, and wherein the one or more reference genes comprise KPNA6, RREB1, or YWHAB.

23. (canceled)

24. The method of claim 23, wherein the expression levels of the one or more reference genes are determined by amplification and detection of one or more subsequences of one or more mRNAs encoding the one or more reference genes,
wherein the amplification of the one or more subsequences of one or more mRNAs is performed by RT-LAMP using a reference gene RT-LAMP primer combination comprising a plurality of reference gene core (FIP, BIP, F3, B3) primers; and
wherein the plurality of reference gene core is selected from the group consisting of:
For KPNA6:
(PD KPNA6v6 B3)
ccacttgttgagcagtcccaagga,
(PD KPNA6v6 FIP)
agtgacgatgttacccacggctctattggtagagctgctgatgcacaa,
(PD KPNA6v6 BIP)
tcttaactgttcagccctaccttgagtccagcaagcttccttccggat,
For RREB1:
(PD RREB1v7 F3)
gccattttgattccttttccggaacaagt,
(PD RREB1v7 B3)
gccaggttcagccccccaata,
(PD RREB1v7 FIP)
acacagteggagcaacggccctcctcggtctctccctgaagc,
(PD RREB1v7 BIP)
gttccaggagtggtggctctgagactgttttctttgtgttatcaagctg
cccand,
and
For YWHAB:
(PE YWHABv145 FIP)
tgcatgatcagagtgctgtctttataaaacggcatttgatgaagc,
(PE YWHABv145 F3)
ctgaaaaggcctgtagcc,
(PE YWHABv145 BIP)
ctgtggacatcggaaaaccagtcacaaagcacgagaaaca,
(PE YWHABv145 B3)
cagagtgacactgaacaga,
including variants of the pluralities wherein one or more of the reference gene core primers contains 1, 2, or 3 nucleotide substitutions relative to one or more of the above sequences.

25. The method of claim 24, wherein the reference gene RT-LAMP primer combination further comprises a pair of reference gene loop primers.

26. The method of claim 25, wherein the pair of reference gene loop primers is selected from the group consisting of:
For KPNA6:
(PD KPNA6v6 FL)
atttgagccctgttgccagcagta,
(PD KPNA6v6 BL)
cagggcaggagaagccactttgta,
For RREB1:
(PD RREB1v7 FL)
cggagtagaaaatgagtctgtgttgacctctt,
(PD RREB1v7 BL)
ctccctggcatgatgcgttgg,
and
For YWHAB:
(PE YWHABv145-1 FL)
tcagcgtatccaattcagcaat,
(PE YWHABv145-1 BL)
gagacgaaggagacgctggg
including variants of the pairs wherein one or more of the reference loop primers contains 1, 2, or 3 nucleotide substitutions relative to one or more of the above sequences.

27. (canceled)

28. (canceled)


SEQUENCE LISTING (1636 sequences)

SEQ ID NO: 1 - DNA, 41 bp, synthetic construct
  source 1..41: mol_type = other DNA
cggccatgatgtccttctcgcaacaggacaagcactacgga

SEQ ID NO: 2 - DNA, 19 bp, synthetic construct
  source 1..19: mol_type = other DNA
tctgtgagcctggctacag

SEQ ID NO: 3 - DNA, 40 bp, synthetic construct
  source 1..40: mol_type = other DNA
acaaaaacggccccgtggagacgtgttggtacactcctga

SEQ ID NO: 4 - DNA, 19 bp, synthetic construct
  source 1..19: mol_type = other DNA
catggccacccatcatctc

SEQ ID NO: 5 - DNA, 22 bp, synthetic construct
  source 1..22: mol_type = other DNA
atgttaaggatgtcgcagaggt

SEQ ID NO: 6 - DNA, 22 bp, synthetic construct
  source 1..22: mol_type = other DNA
ggagctttctctgtgtattcgg

SEQ ID NO: 7 - DNA, 17 bp, synthetic construct
  source 1..17: mol_type = other DNA
ccccgacttcttgcggt

SEQ ID NO: 8 - DNA, 20 bp, synthetic construct
  source 1..20: mol_type = other DNA
gcagagtttctcccggaaac

SEQ ID NO: 9 - DNA, 18 bp, synthetic construct
  source 1..18: mol_type = other DNA
gtggaggagcagtttgcg

SEQ ID NO: 10 - DNA, 18 bp, synthetic construct
  source 1..18: mol_type = other DNA
ttggtgcccgacaccata

SEQ ID NO: 11 - DNA, 38 bp, synthetic construct
  source 1..38: mol_type = other DNA
gttcaagccaggccactggccttttgcccgggacttcg

SEQ ID NO: 12 - DNA, 39 bp, synthetic construct
  source 1..39: mol_type = other DNA
gatgcggtcagtagggctggacgctcgtgggtctcatct

SEQ ID NO: 13 - DNA, 24 bp, synthetic construct
  source 1..24: mol_type = other DNA
acctgaggagagtgactagcttct

SEQ ID NO: 14 - DNA, 19 bp, synthetic construct
  source 1..19: mol_type = other DNA
ccgcaaccctgaagaccca

SEQ ID NO: 15 - DNA, 23 bp, synthetic construct
  source 1..23: mol_type = other DNA
gcagttcaaggtgacaagggcac

SEQ ID NO: 16 - DNA, 22 bp, synthetic construct
  source 1..22: mol_type = other DNA
gcctgctccatggaacccaaga

SEQ ID NO: 17 - DNA, 45 bp, synthetic construct
  source 1..45: mol_type = other DNA
tcagagcaactcagggtttcttccccactgtggaagctcatggac

SEQ ID NO: 18 - DNA, 43 bp, synthetic construct
  source 1..43: mol_type = other DNA
tcagagctggtgcaggagtgcgctggcttggatctgctgtagc

SEQ ID NO: 19 - DNA, 40 bp, synthetic construct
  source 1..40: mol_type = other DNA
tgctcccagtgactgcagagtaattgccaatgggggtgga

SEQ ID NO: 20 - DNA, 40 bp, synthetic construct
  source 1..40: mol_type = other DNA
tgcgaggttctactagctccctttctcccctggcatggtt

SEQ ID NO: 21 - DNA, 19 bp, synthetic construct
  source 1..19: mol_type = other DNA
agcagccaagatgatgtcc

SEQ ID NO: 22 - DNA, 19 bp, synthetic construct
  source 1..19: mol_type = other DNA
gatagttggctcctcgctg

SEQ ID NO: 23 - DNA, 17 bp, synthetic construct
  source 1..17: mol_type = other DNA
tgggtctgccattgcgg

SEQ ID NO: 24 - DNA, 20 bp, synthetic construct
  source 1..20: mol_type = other DNA
ccctcgccctgcagagaaga

SEQ ID NO: 25 - DNA, 20 bp, synthetic construct
  source 1..20: mol_type = other DNA
accccaagttcctggccatc

SEQ ID NO: 26 - DNA, 20 bp, synthetic construct
  source 1..20: mol_type = other DNA
tcccaccagcctccacaatg

SEQ ID NO: 27 - DNA, 21 bp, synthetic construct
  source 1..21: mol_type = other DNA
gatcagcttgctctcctggtt

SEQ ID NO: 28 - DNA, 21 bp, synthetic construct
  source 1..21: mol_type = other DNA
accaccagtcgtgtgctcaag

SEQ ID NO: 29 - DNA, 34 bp, synthetic construct
  source 1..34: mol_type = other DNA
gatctgcacgagggccttgcagctcctggcctac

SEQ ID NO: 30 - DNA, 53 bp, synthetic construct
  source 1..53: mol_type = other DNA
atgcgtaactacagttatgaaaagctgcgcttattgctgggacacacggatag

SEQ ID NO: 31 - DNA, 24 bp, synthetic construct
  source 1..24: mol_type = other DNA
atttgagccctgttgccagcagta

SEQ ID NO: 32 - DNA, 24 bp, synthetic construct
  source 1..24: mol_type = other DNA
cagggcaggagaagccactttgta

SEQ ID NO: 33 - DNA, 24 bp, synthetic construct
  source 1..24: mol_type = other DNA
ccacttgttgagcagtcccaagga

SEQ ID NO: 34 - DNA, 48 bp, synthetic construct
  source 1..48: mol_type = other DNA
agtgacgatgttacccacggctctattggtagagctgctgatgcacaa

SEQ ID NO: 35 - DNA, 48 bp, synthetic construct
  source 1..48: mol_type = other DNA
tcttaactgttcagccctaccttgagtccagcaagcttccttccggat

SEQ ID NO: 36 - DNA, 28 bp, synthetic construct
  source 1..28: mol_type = other DNA
gaaataaagaccagatcaccaacatctt

SEQ ID NO: 37 - DNA, 24 bp, synthetic construct
  source 1..24: mol_type = other DNA
gaggaggctctcagtactgaaaat

SEQ ID NO: 38 - DNA, 24 bp, synthetic construct
  source 1..24: mol_type = other DNA
gtcgcttcttccgtttattccaat

SEQ ID NO: 39 - DNA, 28 bp, synthetic construct
  source 1..28: mol_type = other DNA
agccaaaaatatttatgacatcttgcct

SEQ ID NO: 40 - DNA, 38 bp, synthetic construct
  source 1..38: mol_type = other DNA
gcatgacggtaactcggagcgttgcggttttctgcatc

SEQ ID NO: 41 - DNA, 42 bp, synthetic construct
  source 1..42: mol_type = other DNA
tgactttgccacaaaccagacactcatgtctccccaggtctt

SEQ ID NO: 42 - DNA, 29 bp, synthetic construct
  source 1..29: mol_type = other DNA
gccattttgattccttttccggaacaagt

SEQ ID NO: 43 - DNA, 32 bp, synthetic construct
  source 1..32: mol_type = other DNA
cggagtagaaaatgagtctgtgttgacctctt

SEQ ID NO: 44 - DNA, 21 bp, synthetic construct
  source 1..21: mol_type = other DNA
ctccctggcatgatgcgttgg

SEQ ID NO: 45 - DNA, 21 bp, synthetic construct
  source 1..21: mol_type = other DNA
gccaggttcagccccccaata

SEQ ID NO: 46 - DNA, 42 bp, synthetic construct
  source 1..42: mol_type = other DNA
acacagtcggagcaacggccctcctcggtctctccctgaagc

SEQ ID NO: 47 - DNA, 52 bp, synthetic construct
  source 1..52: mol_type = other DNA
gttccaggagtggtggctctgagactgttttctttgtgttatcaagctgccc

SEQ ID NO: 48 - DNA, 18 bp, synthetic construct
  source 1..18: mol_type = other DNA
ggatcagctgagcccact

SEQ ID NO: 49 - DNA, 21 bp, synthetic construct
  source 1..21: mol_type = other DNA
ccgctggatctccttttcctg

SEQ ID NO: 50 - DNA, 21 bp, synthetic construct
  source 1..21: mol_type = other DNA
caacagcatttgggagcccag

SEQ ID NO: 51 - DNA, 20 bp, synthetic construct
  source 1..20: mol_type = other DNA
cagcaactcattctgcgtga

SEQ ID NO: 52 - DNA, 38 bp, synthetic construct
  source 1..38: mol_type = other DNA
gtgcttcctccagggccttgacccgacagcgtgagtac

SEQ ID NO: 53 - DNA, 41 bp, synthetic construct
  source 1..41: mol_type = other DNA
ccaaaccccgccatcatctccccagctcctgtttccttagg

SEQ ID NO: 54 - DNA, 45 bp, synthetic construct
  source 1..45: mol_type = other DNA
tgcatgatcagagtgctgtctttataaaacggcatttgatgaagc

SEQ ID NO: 55 - DNA, 18 bp, synthetic construct
  source 1..18: mol_type = other DNA
ctgaaaaggcctgtagcc

SEQ ID NO: 56 - DNA, 40 bp, synthetic construct
  source 1..40: mol_type = other DNA
ctgtggacatcggaaaaccagtcacaaagcacgagaaaca

SEQ ID NO: 57 - DNA, 19 bp, synthetic construct
  source 1..19: mol_type = other DNA
cagagtgacactgaacaga

SEQ ID NO: 58 - DNA, 22 bp, synthetic construct
  source 1..22: mol_type = other DNA
tcagcgtatccaattcagcaat

SEQ ID NO: 59 - DNA, 20 bp, synthetic construct
  source 1..20: mol_type = other DNA
gagacgaaggagacgctggg

SEQ ID NO: 60 - DNA, 20 bp, synthetic construct
  source 1..20: mol_type = other DNA
actgagggttgactgactgg

SEQ ID NO: 61 - DNA, 31 bp, synthetic construct
  source 1..31: mol_type = other DNA
cacatcacactcttgttctttaagtttctca

SEQ ID NO: 62 - DNA, 54 bp, synthetic construct
  source 1..54: mol_type = other DNA
ctccaataatccctatggttctggacttttttagagctcaagtgcagcaaagag

SEQ ID NO: 63 - DNA, 51 bp, synthetic construct
  source 1..51: mol_type = other DNA
ctttctcaaagggacagccacgtttttagcagaccagcctttctcaatact

SEQ ID NO: 64 - DNA, 20 bp, synthetic construct
  source 1..20: mol_type = other DNA
gcgctcatgctctgacactt

SEQ ID NO: 65 - DNA, 20 bp, synthetic construct
  source 1..20: mol_type = other DNA
aggggtggaagaaggcccta

SEQ ID NO: 66 - DNA, 20 bp, synthetic construct
  source 1..20: mol_type = other DNA
actgagggttgactgactgg

SEQ ID NO: 67 - DNA, 31 bp, synthetic construct; identical to SEQ ID NO: 61
  source 1..31: mol_type = other DNA

SEQ ID NO: 68 - DNA, 49 bp, synthetic construct
  source 1..49: mol_type = other DNA
ctccaataatccctatggttctggactagagctcaagtgcagcaaagag

SEQ ID NO: 69 - DNA, 46 bp, synthetic construct
  source 1..46: mol_type = other DNA
ctttctcaaagggacagccacgagcagaccagcctttctcaatact

SEQ ID NO: 70 - DNA, 20 bp, synthetic construct
  source 1..20: mol_type = other DNA
gcgctcatgctctgacactt

SEQ ID NO: 71 - DNA, 20 bp, synthetic construct
  source 1..20: mol_type = other DNA
aggggtggaagaaggcccta

SEQ ID NO: 72 - DNA, 19 bp, synthetic construct
  source 1..19: mol_type = other DNA
ggaccagacaggactcgtg

SEQ ID NO: 73 - DNA, 18 bp, synthetic construct
  source 1..18: mol_type = other DNA
cgctgcatcttcaggcca

SEQ ID NO: 74 - DNA, 49 bp, synthetic construct
  source 1..49: mol_type = other DNA
tgagagtaggtcagttgaattggtcttttttccaggtgctgaagccttc

SEQ ID NO: 75 - DNA, 46 bp, synthetic construct
  source 1..46: mol_type = other DNA
atggaatgagcccccagtaattcttttttgcctggcaatcccagta

SEQ ID NO: 76 - DNA, 24 bp, synthetic construct
  source 1..24: mol_type = other DNA
gaaagacgccattgctaaacttca

SEQ ID NO: 77 - DNA, 23 bp, synthetic construct
  source 1..23: mol_type = other DNA
ccatggtcattctcagccttact

SEQ ID NO: 78 - DNA, 19 bp, synthetic construct
  source 1..19: mol_type = other DNA
ggaccagacaggactcgtg

SEQ ID NO: 79 - DNA, 18 bp, synthetic construct
  source 1..18: mol_type = other DNA
cgctgcatcttcaggcca

SEQ ID NO: 80 - DNA, 44 bp, synthetic construct
  source 1..44: mol_type = other DNA
tgagagtaggtcagttgaattggtctccaggtgctgaagccttc

SEQ ID NO: 81 - DNA, 41 bp, synthetic construct
  source 1..41: mol_type = other DNA
atggaatgagcccccagtaattctgcctggcaatcccagta

SEQ ID NO: 82 - DNA, 24 bp, synthetic construct
  source 1..24: mol_type = other DNA
gaaagacgccattgctaaacttca

SEQ ID NO: 83 - DNA, 23 bp, synthetic construct
  source 1..23: mol_type = other DNA
ccatggtcattctcagccttact

SEQ ID NO: 84 - DNA, 19 bp, synthetic construct
  source 1..19: mol_type = other DNA
cgacctccgcaaccttgag

SEQ ID NO: 85 - DNA, 23 bp, synthetic construct
  source 1..23: mol_type = other DNA
cttggtccactgtgcctctaaac

SEQ ID NO: 86 - DNA, 43 bp, synthetic construct
  source 1..43: mol_type = other DNA
aaacctactcgaccgcgtcctttttctacgaccgcagcaggaa

SEQ ID NO: 87 - DNA, 59 bp, synthetic construct
  source 1..59: mol_type = other DNA
taaaacatgaatcctacactcatccttttttttgtgatcaaatgttagagtagctgagg

SEQ ID NO: 88 - DNA, 20 bp, synthetic construct
  source 1..20: mol_type = other DNA
ctcttccagtccctgtccgg

SEQ ID NO: 89 - DNA, 19 bp, synthetic construct
  source 1..19: mol_type = other DNA
gccttttgcctgggaattg

SEQ ID NO: 90 - DNA, 19 bp, synthetic construct
  source 1..19: mol_type = other DNA
cgacctccgcaaccttgag

SEQ ID NO: 91 - DNA, 23 bp, synthetic construct
  source 1..23: mol_type = other DNA
cttggtccactgtgcctctaaac

SEQ ID NO: 92 - DNA, 38 bp, synthetic construct
  source 1..38: mol_type = other DNA
aaacctactcgaccgcgtccctacgaccgcagcaggaa

SEQ ID NO: 93 - DNA, 54 bp, synthetic construct
  source 1..54: mol_type = other DNA
taaaacatgaatcctacactcatcctttgtgatcaaatgttagagtagctgagg

SEQ ID NO: 94 - DNA, 20 bp, synthetic construct
  source 1..20: mol_type = other DNA
ctcttccagtccctgtccgg

SEQ ID NO: 95 - DNA, 19 bp, synthetic construct
  source 1..19: mol_type = other DNA
gccttttgcctgggaattg

SEQ ID NO: 96 - DNA, 18 bp, synthetic construct
  source 1..18: mol_type = other DNA
tgaggccctggttgctat

SEQ ID NO: 97 - DNA, 19 bp, synthetic construct
  source 1..19: mol_type = other DNA
tcactcctcgatgccagaa

SEQ ID NO: 98 - DNA, 38 bp, synthetic construct
  source 1..38: mol_type = other DNA
ttggtgaagaccttctggcgcaacaggaaccttggtcc

SEQ ID NO: 99 - DNA, 41 bp, synthetic construct
  source 1..41: mol_type = other DNA
caacagtgtggcacggaaggtaatagtccccgaagatctgg

SEQ ID NO: 100 - DNA, 19 bp, synthetic construct
  source 1..19: mol_type = other DNA
cctgagcatcagctgctag

SEQ ID NO: 101 - DNA, 19 bp, synthetic construct
  source 1..19: mol_type = other DNA
gcatgggttcctcaacctg

SEQ ID NO: 102 - DNA, 19 bp, synthetic construct
  source 1..19: mol_type = other DNA
aagcatgggttcctcaacc

SEQ ID NO: 103 - DNA, 21 bp, synthetic construct
  source 1..21: mol_type = other DNA
ctgagtgacaccagacaggta

SEQ ID NO: 104 - DNA, 41 bp, synthetic construct
  source 1..41: mol_type = other DNA
ctgtacttgaggctccctctgccagatcttcggggactatt

SEQ ID NO: 105 - DNA, 37 bp, synthetic construct
  source 1..37: mol_type = other DNA
ggaacagcaggtggcaaagctgaggaaacttggggtc

SEQ ID NO: 106 - DNA, 19 bp, synthetic construct
  source 1..19: mol_type = other DNA
ctcctcgatgccagaagtg

SEQ ID NO: 107 - DNA, 20 bp, synthetic construct
  source 1..20: mol_type = other DNA
ctaaacgggacgtgtaccag

SEQ ID NO: 108 - DNA, 21 bp, synthetic construct
  source 1..21: mol_type = other DNA
attaccacttctggcatcgag

SEQ ID NO: 109 - DNA, 21 bp, synthetic construct
  source 1..21: mol_type = other DNA
gtccagaatggagaccacaat

SEQ ID NO: 110 - DNA, 40 bp, synthetic construct
  source 1..40: mol_type = other DNA
ctggtacacgtcccgtttagagagggagcctcaagtacag

SEQ ID NO: 111 - DNA, 38 bp, synthetic construct
  source 1..38: mol_type = other DNA
gaccccaagtttcctcagccgccttcacattcaggtcc

SEQ ID NO: 112 - DNA, 18 bp, synthetic construct
  source 1..18: mol_type = other DNA
tttgccacctgctgttcc

SEQ ID NO: 113 - DNA, 19 bp, synthetic construct
  source 1..19: mol_type = other DNA
ctgtctggtgtcactcagc

SEQ ID NO: 114 - DNA, 18 bp, synthetic construct
  source 1..18: mol_type = other DNA
tgaggccctggttgctat

SEQ ID NO: 115 - DNA, 19 bp, synthetic construct
  source 1..19: mol_type = other DNA
tcactcctcgatgccagaa

SEQ ID NO: 116 - DNA, 43 bp, synthetic construct
  source 1..43: mol_type = other DNA
ttggtgaagaccttctggctttttgcaacaggaaccttggtcc

SEQ ID NO: 117 - DNA, 46 bp, synthetic construct
  source 1..46: mol_type = other DNA
caacagtgtggcacggaatttttggtaatagtccccgaagatctgg

SEQ ID NO: 118 - DNA, 19 bp, synthetic construct
  source 1..19: mol_type = other DNA
cctgagcatcagctgctag

SEQ ID NO: 119 - DNA, 19 bp, synthetic construct
  source 1..19: mol_type = other DNA
gcatgggttcctcaacctg

SEQ ID NO: 120 - DNA, 19 bp, synthetic construct
  source 1..19: mol_type = other DNA
aagcatgggttcctcaacc

SEQ ID NO: 121 - DNA, 21 bp, synthetic construct
  source 1..21: mol_type = other DNA
ctgagtgacaccagacaggta

SEQ ID NO: 122 - DNA, 46 bp, synthetic construct
  source 1..46: mol_type = other DNA
ctgtacttgaggctccctcttttttgccagatcttcggggactatt

SEQ ID NO: 123 - DNA, 42 bp, synthetic construct
  source 1..42: mol_type = other DNA
ggaacagcaggtggcaaatttttgctgaggaaacttggggtc

SEQ ID NO: 124 - DNA, 19 bp, synthetic construct
  source 1..19: mol_type = other DNA
ctcctcgatgccagaagtg

SEQ ID NO: 125 - DNA, 20 bp, synthetic construct
  source 1..20: mol_type = other DNA
ctaaacgggacgtgtaccag

SEQ ID NO: 126 - DNA, 21 bp, synthetic construct
  source 1..21: mol_type = other DNA
attaccacttctggcatcgag

SEQ ID NO: 127 - DNA, 21 bp, synthetic construct
  source 1..21: mol_type = other DNA
gtccagaatggagaccacaat

SEQ ID NO: 128 - DNA, 45 bp, synthetic construct
  source 1..45: mol_type = other DNA
ctggtacacgtcccgtttagtttttagagggagcctcaagtacag

SEQ ID NO: 129 - DNA, 43 bp, synthetic construct
  source 1..43: mol_type = other DNA
gaccccaagtttcctcagctttttcgccttcacattcaggtcc

SEQ ID NO: 130 - DNA, 18 bp, synthetic construct
  source 1..18: mol_type = other DNA
tttgccacctgctgttcc

SEQ ID NO: 131 - DNA, 19 bp, synthetic construct
  source 1..19: mol_type = other DNA
ctgtctggtgtcactcagc

SEQ ID NO: 132 - DNA, 20 bp, synthetic construct
  source 1..20: mol_type = other DNA
agagggagcctcaagtacag

SEQ ID NO: 133 - DNA, 22 bp, synthetic construct
  source 1..22: mol_type = other DNA
caggatcataattgcctgccaa

SEQ ID NO: 134 - DNA, 37 bp, synthetic construct
  source 1..37: mol_type = other DNA
gctgagtgacaccagacagggcaaagcgacggactaa

SEQ ID NO: 135 - DNA, 39 bp, synthetic construct
  source 1..39: mol_type = other DNA
ggacctgaatgtgaaggcgtggttcttctcgatgccatc

SEQ ID NO: 136 - DNA, 19 bp, synthetic construct
  source 1..19: mol_type = other DNA
gctgaggaaacttggggtc

SEQ ID NO: 137 - DNA, 20 bp, synthetic construct
  source 1..20: mol_type = other DNA
cggcattgtggtctccattc

SEQ ID NO: 138 - DNA, 20 bp, synthetic construct
  source 1..20: mol_type = other DNA
ctaaacgggacgtgtaccag

SEQ ID NO: 139 - DNA, 22 bp, synthetic construct
  source 1..22: mol_type = other DNA
cattcatctgtgtgtaccgagg

SEQ ID NO: 140 - DNA, 40 bp, synthetic construct
  source 1..40: mol_type = other DNA
gaatggagaccacaatgccgcacagaccccaagtttcctc

SEQ ID NO: 141 - DNA, 43 bp, synthetic construct
  source 1..43: mol_type = other DNA
gatggcatcgagaagaaccactggtcattgacatcaaaactgg

SEQ ID NO: 142 - DNA, 21 bp, synthetic construct
  source 1..21: mol_type = other DNA
ctgagtgacaccagacaggta

SEQ ID NO: 143 - DNA, 22 bp, synthetic construct
  source 1..22: mol_type = other DNA
acttggcaggcaattatgatcc

SEQ ID NO: 144 - DNA, 19 bp, synthetic construct
  source 1..19: mol_type = other DNA
gaccccaagtttcctcagc

SEQ ID NO: 145 - DNA, 22 bp, synthetic construct
  source 1..22: mol_type = other DNA
cattcatctgtgtgtaccgagg

SEQ ID NO: 146 - DNA, 39 bp, synthetic construct
  source 1..39: mol_type = other DNA
atcgtccagaatggagaccactgtctggtgtcactcagc

SEQ ID NO: 147 - DNA, 42 bp, synthetic construct
  source 1..42: mol_type = other DNA
gcatcgagaagaaccacccctggtcattgacatcaaaactgg

SEQ ID NO: 148 - DNA, 19 bp, synthetic construct
  source 1..19: mol_type = other DNA
cgccttcacattcaggtcc

SEQ ID NO: 149 - DNA, 22 bp, synthetic construct
  source 1..22: mol_type = other DNA
acttggcaggcaattatgatcc

SEQ ID NO: 150 - DNA, 18 bp, synthetic construct
  source 1..18: mol_type = other DNA
actcagcgggacctgaat

SEQ ID NO: 151 - DNA, 18 bp, synthetic construct
  source 1..18: mol_type = other DNA
cgttgtaggccacaccta

SEQ ID NO: 152 - DNA, 41 bp, synthetic construct
  source 1..41: mol_type = other DNA
atcataattgcctgccaagtccacggcattgtggtctccat

SEQ ID NO: 153 - DNA, 41 bp, synthetic construct
  source 1..41: mol_type = other DNA
ccagttttgatgtcaatgaccagccgtgcctgttgtcattc

SEQ ID NO: 154 - DNA, 18 bp, synthetic construct
  source 1..18: mol_type = other DNA
cttctcgatgccatcgtc

SEQ ID NO: 155 - DNA, 19 bp, synthetic construct
  source 1..19: mol_type = other DNA
agcctcggtacacacagat

SEQ ID NO: 156 - DNA, 19 bp, synthetic construct
  source 1..19: mol_type = other DNA
cgacctccgcaaccttgag

SEQ ID NO: 157 - DNA, 23 bp, synthetic construct
  source 1..23: mol_type = other DNA
cttggtccactgtgcctctaaac

SEQ ID NO: 158 - DNA, 43 bp, synthetic construct; identical to SEQ ID NO: 86
  source 1..43: mol_type = other DNA

SEQ ID NO: 159 - DNA, 59 bp, synthetic construct; identical to SEQ ID NO: 87
  source 1..59: mol_type = other DNA

SEQ ID NO: 160 - DNA, 20 bp, synthetic construct
  source 1..20: mol_type = other DNA
ctcttccagtccctgtccgg

SEQ ID NO: 161 - DNA, 19 bp, synthetic construct
  source 1..19: mol_type = other DNA
gccttttgcctgggaattg

SEQ ID NO: 162 - DNA, 20 bp, synthetic construct
  source 1..20: mol_type = other DNA
cctgtgagtgagtgcagaaa

SEQ ID NO: 163 - DNA, 18 bp, synthetic construct
  source 1..18: mol_type = other DNA
accccgacagtgatcgtg

SEQ ID NO: 164 - DNA, 44 bp, synthetic construct
  source 1..44: mol_type = other DNA
ttcggtcttctgctctccatttttgcgacctgcagtttgcaata

SEQ ID NO: 165 - DNA, 42 bp, synthetic construct
  source 1..42: mol_type = other DNA
ggatggataaggtgggggatttttgctgactcagggctttgc

SEQ ID NO: 166 - DNA, 20 bp, synthetic construct
  source 1..20: mol_type = other DNA
gccgagaattcctccaaagt

SEQ ID NO: 167 - DNA, 18 bp, synthetic construct
  source 1..18: mol_type = other DNA
ccctggaggaagtgctca

SEQ ID NO: 168 - DNA, 20 bp, synthetic construct
  source 1..20: mol_type = other DNA
cctgtgagtgagtgcagaaa

SEQ ID NO: 169 - DNA, 18 bp, synthetic construct
  source 1..18: mol_type = other DNA
accccgacagtgatcgtg

SEQ ID NO: 170 - DNA, 39 bp, synthetic construct
  source 1..39: mol_type = other DNA
ttcggtcttctgctctccagcgacctgcagtttgcaata

SEQ ID NO: 171 - DNA, 37 bp, synthetic construct
  source 1..37: mol_type = other DNA
ggatggataaggtgggggagctgactcagggctttgc

SEQ ID NO: 172 - DNA, 20 bp, synthetic construct
  source 1..20: mol_type = other DNA
gccgagaattcctccaaagt

SEQ ID NO: 173 - DNA, 18 bp, synthetic construct
  source 1..18: mol_type = other DNA
ccctggaggaagtgctca

SEQ ID NO: 174 - DNA, 19 bp, synthetic construct
  source 1..19: mol_type = other DNA
cacttcaccctgatccagg

SEQ ID NO: 175 - DNA, 21 bp, synthetic construct
  source 1..21: mol_type = other DNA
tgatccatgtagcgactttcc

SEQ ID NO: 176 - DNA, 37 bp, synthetic construct
  source 1..37: mol_type = other DNA
tctccaagagcaggagctcgttttgctgcgagaacga

SEQ ID NO: 177 - DNA, 46 bp, synthetic construct
  source 1..46: mol_type = other DNA
gctggtgacgaatccacatcggcaaaaacaaataagttgacttaag

SEQ ID NO: 178 - DNA, 18 bp, synthetic construct
  source 1..18: mol_type = other DNA
gacgcgcaggatgttgat

SEQ ID NO: 179 - DNA, 22 bp, synthetic construct
  source 1..22: mol_type = other DNA
catctcaatggaaggatcctgc

SEQ ID NO: 180 - DNA, 20 bp, synthetic construct
  source 1..20: mol_type = other DNA
tgaactcccggcatctttac

SEQ ID NO: 181 - DNA, 18 bp, synthetic construct
  source 1..18: mol_type = other DNA
cgccaagctattcagcac

SEQ ID NO: 182 - DNA, 42 bp, synthetic construct
  source 1..42: mol_type = other DNA
aatcctttggagtcccagctttttatcacattcctgccgctg

SEQ ID NO: 183 - DNA, 45 bp, synthetic construct
  source 1..45: mol_type = other DNA
tactgcatctccttcaacaaggtttttaattcgcaaggggccatc

SEQ ID NO: 184 - DNA, 19 bp, synthetic construct
  source 1..19: mol_type = other DNA
gacaggtgctttccacatg

SEQ ID NO: 185 - DNA, 21 bp, synthetic construct
  source 1..21: mol_type = other DNA
gctgggatccagaggagaata

SEQ ID NO: 186 - DNA, 20 bp, synthetic construct
  source 1..20: mol_type = other DNA
tgaactcccggcatctttac

SEQ ID NO: 187 - DNA, 18 bp, synthetic construct
  source 1..18: mol_type = other DNA
cgccaagctattcagcac

SEQ ID NO: 188 - DNA, 37 bp, synthetic construct
  source 1..37: mol_type = other DNA
aatcctttggagtcccagcatcacattcctgccgctg

SEQ ID NO: 189 - DNA, 40 bp, synthetic construct
  source 1..40: mol_type = other DNA
tactgcatctccttcaacaaggaattcgcaaggggccatc

SEQ ID NO: 190 - DNA, 19 bp, synthetic construct
  source 1..19: mol_type = other DNA
gacaggtgctttccacatg

SEQ ID NO: 191 - DNA, 21 bp, synthetic construct
  source 1..21: mol_type = other DNA
gctgggatccagaggagaata

SEQ ID NO: 192 - DNA, 18 bp, synthetic construct
  source 1..18: mol_type = other DNA
tggcgaatgtcctctcac

SEQ ID NO: 193 - DNA, 24 bp, synthetic construct
  source 1..24: mol_type = other DNA
gatagtcacttctgctggatagaa

SEQ ID NO: 194 - DNA, 37 bp, synthetic construct
  source 1..37: mol_type = other DNA
cctgttggtcagtgatcccaagacaccctgatgcagc

SEQ ID NO: 195 - DNA, 37 bp, synthetic construct
  source 1..37: mol_type = other DNA
caccatctgtgcaagtagcatagcaggccagcatcac

SEQ ID NO: 196 - DNA, 20 bp, synthetic construct
  source 1..20: mol_type = other DNA
acaattctgaagcccattgc

SEQ ID NO: 197 - DNA, 21 bp, synthetic construct
  source 1..21: mol_type = other DNA
accactccttttaacacgagg

SEQ ID NO: 198 - DNA, 18 bp, synthetic construct
  source 1..18: mol_type = other DNA
tggcgaatgtcctctcac

SEQ ID NO: 199 - DNA, 24 bp, synthetic construct
  source 1..24: mol_type = other DNA
gatagtcacttctgctggatagaa

SEQ ID NO: 200 - DNA, 42 bp, synthetic construct
  source 1..42: mol_type = other DNA
cctgttggtcagtgatccctttttaagacaccctgatgcagc

SEQ ID NO: 201 - DNA, 42 bp, synthetic construct
  source 1..42: mol_type = other DNA
caccatctgtgcaagtagctttttatagcaggccagcatcac

SEQ ID NO: 202 - DNA, 20 bp, synthetic construct
  source 1..20: mol_type = other DNA
acaattctgaagcccattgc

SEQ ID NO: 203 - DNA, 21 bp, synthetic construct
  source 1..21: mol_type = other DNA
accactccttttaacacgagg

SEQ ID NO: 204 - DNA, 18 bp, synthetic construct
  source 1..18: mol_type = other DNA
aagacaccctgatgcagc

SEQ ID NO: 205 - DNA, 19 bp, synthetic construct
  source 1..19: mol_type = other DNA
catgacaagcttcccgttc

SEQ ID NO: 206 - DNA, 40 bp, synthetic construct
  source 1..40: mol_type = other DNA
cctcgtgttaaaaggagtggtgggatcactgaccaacagg

SEQ ID NO: 207 - DNA, 39 bp, synthetic construct
  source 1..39: mol_type = other DNA
gtgatgctggcctgctatcctccacgtgatagtcacttc

SEQ ID NO: 208 - DNA, 19 bp, synthetic construct
  source 1..19: mol_type = other DNA
gctacttgcacagatggtg

SEQ ID NO: 209 - DNA, 19 bp, synthetic construct
  source 1..19: mol_type = other DNA
gtgtggggcttctatccag

SEQ ID NO: 210 - DNA, 20 bp, synthetic construct
  source 1..20: mol_type = other DNA
gtgactatcacgtggaggaa

SEQ ID NO: 211 - DNA, 20 bp, synthetic construct
  source 1..20: mol_type = other DNA
gaaaccttcagggtctgcat

SEQ ID NO: 212 - DNA, 39 bp, synthetic construct
  source 1..39: mol_type = other DNA
ggtctggtatgtccagtctcgaacgggaagcttgtcatg

SEQ ID NO: 213 - DNA, 39 bp, synthetic construct
  source 1..39: mol_type = other DNA
ctcccatttagccttaacccctgtccagtcccgaaggat

SEQ ID NO: 214 - DNA, 18 bp, synthetic construct
  source 1..18: mol_type = other DNA
attgggctgggcagtctt

SEQ ID NO: 215 - DNA, 19 bp, synthetic construct
  source 1..19: mol_type = other DNA
ggggacacttacacctgtg

SEQ ID NO: 216 - DNA, 18 bp, synthetic construct
  source 1..18: mol_type = other DNA
aagacaccctgatgcagc

SEQ ID NO: 217 - DNA, 19 bp, synthetic construct
  source 1..19: mol_type = other DNA
catgacaagcttcccgttc

SEQ ID NO: 218 - DNA, 45 bp, synthetic construct
  source 1..45: mol_type = other DNA
cctcgtgttaaaaggagtggttttttgggatcactgaccaacagg

SEQ ID NO: 219 - DNA, 44 bp, synthetic construct
  source 1..44: mol_type = other DNA
gtgatgctggcctgctattttttcctccacgtgatagtcacttc

SEQ ID NO: 220 - DNA, 19 bp, synthetic construct
  source 1..19: mol_type = other DNA
gctacttgcacagatggtg

SEQ ID NO: 221 - DNA, 19 bp, synthetic construct
  source 1..19: mol_type = other DNA
gtgtggggcttctatccag

SEQ ID NO: 222 - DNA, 20 bp, synthetic construct
  source 1..20: mol_type = other DNA
gtgactatcacgtggaggaa

SEQ ID NO: 223 - DNA, 20 bp, synthetic construct
  source 1..20: mol_type = other DNA
gaaaccttcagggtctgcat

SEQ ID NO: 224 - DNA, 44 bp, synthetic construct
  source 1..44: mol_type = other DNA
ggtctggtatgtccagtctctttttgaacgggaagcttgtcatg

SEQ ID NO: 225 - DNA, 44 bp, synthetic construct
  source 1..44: mol_type = other DNA
ctcccatttagccttaaccccttttttgtccagtcccgaaggat

SEQ ID NO: 226 - DNA, 18 bp, synthetic construct
  source 1..18: mol_type = other DNA
attgggctgggcagtctt

SEQ ID NO: 227 - DNA, 19 bp, synthetic construct
  source 1..19: mol_type = other DNA
ggggacacttacacctgtg

SEQ ID NO: 228 - DNA, 20 bp, synthetic construct
  source 1..20: mol_type = other DNA
gagcatgatcacattcctgc

SEQ ID NO: 229 - DNA, 21 bp, synthetic construct
  source 1..21: mol_type = other DNA
gacattcgccaagctattcag

SEQ ID NO: 230 - DNA, 41 bp, synthetic construct
  source 1..41: mol_type = other DNA
gttgaaggagatgcagtatgtgcatgtggaaagcacctgtc

SEQ ID NO: 231 - DNA, 36 bp, synthetic construct
  source 1..36: mol_type = other DNA
aaggatctgctgacctgccaccccaaattcgcaagg

SEQ ID NO: 232 - DNA, 19 bp, synthetic construct
  source 1..19: mol_type = other DNA
aatcctttggagtcccagc

SEQ ID NO: 233 - DNA, 23 bp, synthetic construct
  source 1..23: mol_type = other DNA
ggatccagaggagaataagatgg

SEQ ID NO: 234 - DNA, 18 bp, synthetic construct
  source 1..18: mol_type = other DNA
atcacattcctgccgctg

SEQ ID NO: 235 - DNA, 18 bp, synthetic construct
  source 1..18: mol_type = other DNA
cgccaagctattcagcac

SEQ ID NO: 236 - DNA, 42 bp, synthetic construct
  source 1..42: mol_type = other DNA
ggagatgcagtatgtgaaatccgtggaaagcacctgtctgtt

SEQ ID NO: 237 - DNA, 39 bp, synthetic construct
  source 1..39: mol_type = other DNA
ttcaacaaggatctgctgaccaattcgcaaggggccatc

SEQ ID NO: 238 - DNA, 19 bp, synthetic construct
  source 1..19: mol_type = other DNA
ttggagtcccagcatcatc

SEQ ID NO: 239 - DNA, 21 bp, synthetic construct
  source 1..21: mol_type = other DNA
gctgggatccagaggagaata

SEQ ID NO: 240 - DNA, 20 bp, synthetic construct
  source 1..20: mol_type = other DNA
tttggggtgctgaatagctt

SEQ ID NO: 241 - DNA, 18 bp, synthetic construct
  source 1..18: mol_type = other DNA
atagcaggccagcatcac

SEQ ID NO: 242 - DNA, 38 bp, synthetic construct
  source 1..38: mol_type = other DNA
gcacaattctgaagcccattcgaatgtcctctcacagc

SEQ ID NO: 243 - DNA, 41 bp, synthetic construct
  source 1..41: mol_type = other DNA
ccttctggggatcactgacctcgtgttaaaaggagtggttt

SEQ ID NO: 244 - DNA, 19 bp, synthetic construct
  source 1..19: mol_type = other DNA
gctgcatcagggtgtcttt

SEQ ID NO: 245 - DNA, 19 bp, synthetic construct
  source 1..19: mol_type = other DNA
gccaccatctgtgcaagta

SEQ ID NO: 246 - DNA, 18 bp, synthetic construct
  source 1..18: mol_type = other DNA
cttgtcatgcctcacagc

SEQ ID NO: 247 - DNA, 18 bp, synthetic construct
  source 1..18: mol_type = other DNA
ccagctgatcacaccaag

SEQ ID NO: 248 - DNA, 41 bp, synthetic construct
  source 1..41: mol_type = other DNA
cacaggtgtaagtgtcccccaatggagactggacataccag

SEQ ID NO: 249 - DNA, 37 bp, synthetic construct
  source 1..37: mol_type = other DNA
atccttcgggactggacaagaagatgatgaggcccag

SEQ ID NO: 250 - DNA, 21 bp, synthetic construct
  source 1..21: mol_type = other DNA
ggggttaaggctaaatgggag

SEQ ID NO: 251 - DNA, 19 bp, synthetic construct
  source 1..19: mol_type = other DNA
tgcagaccctgaaggtttc

SEQ ID NO: 252 - DNA, 22 bp, synthetic construct
  source 1..22: mol_type = other DNA
caatggagactggacataccag

SEQ ID NO: 253 - DNA, 18 bp, synthetic construct
  source 1..18: mol_type = other DNA
ccagctgatcacaccaag

SEQ ID NO: 254 - DNA, 40 bp, synthetic construct
  source 1..40: mol_type = other DNA
ccagtgtgctctaccacacctctcccatttagccttaacc

SEQ ID NO: 255 - DNA, 37 bp, synthetic construct; identical to SEQ ID NO: 249
  source 1..37: mol_type = other DNA

SEQ ID NO: 256 - DNA, 20 bp, synthetic construct
  source 1..20: mol_type = other DNA
gtaagtgtccccgtaagagg

SEQ ID NO: 257 - DNA, 19 bp, synthetic construct
  source 1..19: mol_type = other DNA
tgcagaccctgaaggtttc

SEQ ID NO: 258 - DNA, 22 bp, synthetic construct
  source 1..22: mol_type = other DNA
gcgaactcatctttgccagtac

SEQ ID NO: 259 - DNA, 19 bp, synthetic construct
  source 1..19: mol_type = other DNA
cgccgatcttctgggtgat

SEQ ID NO: 260 - DNA, 39 bp, synthetic construct
  source 1..39: mol_type = other DNA
cgccagcatcttcaccgtttttaggagcttgtgccgtgg

SEQ ID NO: 261 - DNA, 42 bp, synthetic construct
  source 1..42: mol_type = other DNA
ggcaacgaattccaggtgtctttttgcgccttcagctctgac

SEQ ID NO: 262 - DNA, 17 bp, synthetic construct
  source 1..17: mol_type = other DNA
tcaggtcccagcccatg

SEQ ID NO: 263 - DNA, 18 bp, synthetic construct
  source 1..18: mol_type = other DNA
gagcagctccatgtcggt

SEQ ID NO: 264 - DNA, 22 bp, synthetic construct
  source 1..22: mol_type = other DNA
gcgaactcatctttgccagtac

SEQ ID NO: 265 - DNA, 19 bp, synthetic construct
  source 1..19: mol_type = other DNA
cgccgatcttctgggtgat

SEQ ID NO: 266 - DNA, 34 bp, synthetic construct
  source 1..34: mol_type = other DNA
cgccagcatcttcaccgaggagcttgtgccgtgg

SEQ ID NO: 267 - DNA, 37 bp, synthetic construct
  source 1..37: mol_type = other DNA
ggcaacgaattccaggtgtcgcgccttcagctctgac

SEQ ID NO: 268 - DNA, 17 bp, synthetic construct
  source 1..17: mol_type = other DNA
tcaggtcccagcccatg

SEQ ID NO: 269 - DNA, 18 bp, synthetic construct
  source 1..18: mol_type = other DNA
gagcagctccatgtcggt

SEQ ID NO: 270 - DNA, 21 bp, synthetic construct
  source 1..21: mol_type = other DNA
tctttgccagtacaggagctt

SEQ ID NO: 271 - DNA, 17 bp, synthetic construct
  source 1..17: mol_type = other DNA
aagggggaccctgtcct

SEQ ID NO: 272 - DNA, 35 bp, synthetic construct
  source 1..35: mol_type = other DNA
accgacatggagctgctccatgggctgggacctga

SEQ ID NO: 273 - DNA, 34 bp, synthetic construct
  source 1..34: mol_type = other DNA
gtcagagctgaaggcgcagacgctgctggaaggc

SEQ ID NO: 274 - DNA, 20 bp, synthetic construct
  source 1..20: mol_type = other DNA
gacacctggaattcgttgcc

SEQ ID NO: 275 - DNA, 18 bp, synthetic construct
  source 1..18: mol_type = other DNA
acccagaagatcggcgtg

SEQ ID NO: 276 - DNA, 21 bp, synthetic construct
  source 1..21: mol_type = other DNA
tctttgccagtacaggagctt

SEQ ID NO: 277 - DNA, 17 bp, synthetic construct
  source 1..17: mol_type = other DNA
aagggggaccctgtcct

SEQ ID NO: 278 - DNA, 40 bp, synthetic construct
  source 1..40: mol_type = other DNA
accgacatggagctgctctttttcatgggctgggacctga

SEQ ID NO: 279 - DNA, 39 bp, synthetic construct
  source 1..39: mol_type = other DNA
gtcagagctgaaggcgctttttagacgctgctggaaggc

SEQ ID NO: 280 - DNA, 20 bp, synthetic construct
  source 1..20: mol_type = other DNA
gacacctggaattcgttgcc

SEQ ID NO: 281 - DNA, 18 bp, synthetic construct
  source 1..18: mol_type = other DNA
acccagaagatcggcgtg

SEQ ID NO: 282 - DNA, 18 bp, synthetic construct
  source 1..18: mol_type = other DNA
accacgccgagctcagtt

SEQ ID NO: 283 - DNA, 22 bp, synthetic construct
  source 1..22: mol_type = other DNA
ggttttcttgagcgtgtactgg

SEQ ID NO: 284 - DNA, 46 bp, synthetic construct
  source 1..46: mol_type = other DNA
ttgataggctgctccatcaggttttttcggtcaggccccatactca

SEQ ID NO: 285 - DNA, 43 bp, synthetic construct
  source 1..43: mol_type = other DNA
gtgactgagtggcagcagatttttcctccatgatgcccttgct

SEQ ID NO: 286 - DNA, 21 bp, synthetic construct
  source 1..21: mol_type = other DNA
catcacctccatcgtggctac

SEQ ID NO: 287 - DNA, 23 bp, synthetic construct
  source 1..23: mol_type = other DNA
catacacctacgactcgggtatc

SEQ ID NO: 288 - DNA, 18 bp, synthetic construct
  source 1..18: mol_type = other DNA
accacgccgagctcagtt

SEQ ID NO: 289 - DNA, 22 bp, synthetic construct
  source 1..22: mol_type = other DNA
ggttttcttgagcgtgtactgg

SEQ ID NO: 290 - DNA, 41 bp, synthetic construct
  source 1..41: mol_type = other DNA
ttgataggctgctccatcaggtcggtcaggccccatactca

SEQ ID NO: 291 - DNA, 38 bp, synthetic construct
  source 1..38: mol_type = other DNA
gtgactgagtggcagcagacctccatgatgcccttgct

SEQ ID NO: 292 - DNA, 21 bp, synthetic construct
  source 1..21: mol_type = other DNA
catcacctccatcgtggctac

SEQ ID NO: 293 - DNA, 23 bp, synthetic construct
  source 1..23: mol_type = other DNA
catacacctacgactcgggtatc

SEQ ID NO: 294 - DNA, 18 bp, synthetic construct
  source 1..18: mol_type = other DNA
cgagctcagttcgctgtc

SEQ ID NO: 295 - DNA, 21 bp, synthetic construct
  source 1..21: mol_type = other DNA
gtggttttcttgagcgtgtac

SEQ ID NO: 296 - DNA, 39 bp, synthetic construct
  source 1..39: mol_type = other DNA
gtcaccttgataggctgctcaggccccatactcagtagc

SEQ ID NO: 297 - DNA, 38 bp, synthetic construct
  source 1..38: mol_type = other DNA
gagtggcagcagacatacaccctccatgatgcccttgc

SEQ ID NO: 298 - DNA, 22 bp, synthetic construct
  source 1..22: mol_type = other DNA
catcaggttcatcacctccatc

SEQ ID NO: 299 - DNA, 19 bp, synthetic construct
  source 1..19: mol_type = other DNA
acgactcgggtatccactc

SEQ ID NO: 300 - DNA, 18 bp, synthetic construct
  source 1..18: mol_type = other DNA
cgagctcagttcgctgtc

SEQ ID NO: 301 - DNA, 21 bp, synthetic construct
  source 1..21: mol_type = other DNA
gtggttttcttgagcgtgtac

SEQ ID NO: 302 - DNA, 44 bp, synthetic construct
  source 1..44: mol_type = other DNA
gtcaccttgataggctgctctttttaggccccatactcagtagc

SEQ ID NO: 303 - DNA, 43 bp, synthetic construct
  source 1..43: mol_type = other DNA
gagtggcagcagacatacactttttcctccatgatgcccttgc

SEQ ID NO: 304 - DNA, 22 bp, synthetic construct
  source 1..22: mol_type = other DNA
catcaggttcatcacctccatc

SEQ ID NO: 305 - DNA, 19 bp, synthetic construct
  source 1..19: mol_type = other DNA
acgactcgggtatccactc

SEQ ID NO: 306 - DNA, 21 bp, synthetic construct
  source 1..21: mol_type = other DNA
ccatactcagtagccacgatg

SEQ ID NO: 307 - DNA, 19 bp, synthetic construct
  source 1..19: mol_type = other DNA
ggctgttgtggacatctgg

SEQ ID NO: 308 - DNA, 40 bp, synthetic construct
  source 1..40: mol_type = other DNA
agtggatacccgagtcgtagagcagcctatcaaggtgact

SEQ ID NO: 309 - DNA, 39 bp, synthetic construct
  source 1..39: mol_type = other DNA
gcatcatggaggaggatgagctccagatcaccttggctg

SEQ ID NO: 310 - DNA, 20 bp, synthetic construct
  source 1..20: mol_type = other DNA
gtgtatgtctgctgccactc

SEQ ID NO: 311 - DNA, 20 bp, synthetic construct
  source 1..20: mol_type = other DNA
gccagtacacgctcaagaaa

SEQ ID NO: 312 - DNA, 21 bp, synthetic construct
  source 1..21: mol_type = other DNA
ccatactcagtagccacgatg

SEQ ID NO: 313 - DNA, 19 bp, synthetic construct
  source 1..19: mol_type = other DNA
ggctgttgtggacatctgg

SEQ ID NO: 314 - DNA, 45 bp, synthetic construct
  source 1..45: mol_type = other DNA
agtggatacccgagtcgtagtttttagcagcctatcaaggtgact

SEQ ID NO: 315 - DNA, 44 bp, synthetic construct
  source 1..44: mol_type = other DNA
gcatcatggaggaggatgagtttttctccagatcaccttggctg

SEQ ID NO: 316 - DNA, 20 bp, synthetic construct
  source 1..20: mol_type = other DNA
gtgtatgtctgctgccactc

SEQ ID NO: 317 - DNA, 20 bp, synthetic construct
  source 1..20: mol_type = other DNA
gccagtacacgctcaagaaa

SEQ ID NO: 318 - DNA, 18 bp, synthetic construct
  source 1..18: mol_type = other DNA
cgagctcagttcgctgtc

SEQ ID NO: 319 - DNA, 21 bp, synthetic construct
  source 1..21: mol_type = other DNA
gtggttttcttgagcgtgtac

SEQ ID NO: 320 - DNA, 44 bp, synthetic construct; identical to SEQ ID NO: 302
  source 1..44: mol_type = other DNA

SEQ ID NO: 321 - DNA, 43 bp, synthetic construct; identical to SEQ ID NO: 303
  source 1..43: mol_type = other DNA

SEQ ID NO: 322 - DNA, 22 bp, synthetic construct
  source 1..22: mol_type = other DNA
catcaggttcatcacctccatc

SEQ ID NO: 323 - DNA, 19 bp, synthetic construct
  source 1..19: mol_type = other DNA
acgactcgggtatccactc

SEQ ID NO: 324 - DNA, 22 bp, synthetic construct
  source 1..22: mol_type = other DNA
gcactgatgcaggaactgtata

SEQ ID NO: 325 - DNA, 18 bp, synthetic construct
  source 1..18: mol_type = other DNA
agctcagaaacgccacca

SEQ ID NO: 326 - DNA, 43 bp, synthetic construct
  source 1..43: mol_type = other DNA
ctgcctcagaaactcctccatttttactccttcgtggctcagt

SEQ ID NO: 327 - DNA, 43 bp, synthetic construct
  source 1..43: mol_type = other DNA
gagcatttccaggggaagctttttctgagaaccgtgccattcc

SEQ ID NO: 328 - DNA, 19 bp, synthetic construct
  source 1..19: mol_type = other DNA
cagcgtctagcacctcttc

SEQ ID NO: 329 - DNA, 19 bp, synthetic construct
  source 1..19: mol_type = other DNA
cgggtgctgaaggtagtca

SEQ ID NO: 330 - DNA, 22 bp, synthetic construct
  source 1..22: mol_type = other DNA
gcactgatgcaggaactgtata

SEQ ID NO: 331 - DNA, 18 bp, synthetic construct
  source 1..18: mol_type = other DNA
agctcagaaacgccacca

SEQ ID NO: 332 - DNA, 38 bp, synthetic construct
  source 1..38: mol_type = other DNA
ctgcctcagaaactcctccaactccttcgtggctcagt

SEQ ID NO: 333 - DNA, 38 bp, synthetic construct
  source 1..38: mol_type = other DNA
gagcatttccaggggaagcctgagaaccgtgccattcc

SEQ ID NO: 334 - DNA, 19 bp, synthetic construct
  source 1..19: mol_type = other DNA
cagcgtctagcacctcttc

SEQ ID NO: 335 - DNA, 19 bp, synthetic construct
  source 1..19: mol_type = other DNA
cgggtgctgaaggtagtca

SEQ ID NO: 336 - DNA, 18 bp, synthetic construct
  source 1..18: mol_type = other DNA
tctgtttccctgccagac

SEQ ID NO: 337 - DNA, 23 bp, synthetic construct
  source 1..23: mol_type = other DNA
tccttcacttctaccttgatcag

SEQ ID NO: 338 - DNA, 42 bp, synthetic construct
  source 1..42: mol_type = other DNA
ctcactttggaaagttctttctcaggacagagtggaacgctt

SEQ ID NO: 339 - DNA, 46 bp, synthetic construct
  source 1..46: mol_type = other DNA
gttaaacctaactgtccgaattgactcgaagtccagttcagtgtaa

SEQ ID NO: 340 - DNA, 22 bp, synthetic construct
  source 1..22: mol_type = other DNA
aagaacatgagctgtgaattcc

SEQ ID NO: 341 - DNA, 24 bp, synthetic construct
  source 1..24: mol_type = other DNA
catcatggagaaggataccatttc

SEQ ID NO: 342 - DNA, 24 bp, synthetic construct
  source 1..24: mol_type = other DNA
agtgagatggcttctaacaagttc

SEQ ID NO: 343 - DNA, 21 bp, synthetic construct
  source 1..21: mol_type = other DNA
gttgactccaaagcgtgagat

SEQ ID NO: 344 - DNA, 42 bp, synthetic construct
  source 1..42: mol_type = other DNA
ggatctccacatcattctgcttagctgacacacctctcagag

SEQ ID NO: 345 - DNA, 45 bp, synthetic construct
  source 1..45: mol_type = other DNA
ctacccagaaagacagggagtgtattgtttaggcttgaactatgc

SEQ ID NO: 346 - DNA, 19 bp, synthetic construct
  source 1..19: mol_type = other DNA
ggttccctgatcggctcat

SEQ ID NO: 347 - DNA, 18 bp, synthetic construct
  source 1..18: mol_type = other DNA
aagcagcagctcatgacc

SEQ ID NO: 348 - DNA, 24 bp, synthetic construct
  source 1..24: mol_type = other DNA
tgtttagaccagctagagaccata

SEQ ID NO: 349 - DNA, 21 bp, synthetic construct
  source 1..21: mol_type = other DNA
cttatctgggtcatgagctgc

SEQ ID NO: 350 - DNA, 39 bp, synthetic construct
  source 1..39: mol_type = other DNA
ctctgagaggtgtgtcagctcggtctgtcagtgagatgg

SEQ ID NO: 351 - DNA, 40 bp, synthetic construct
  source 1..40: mol_type = other DNA
gatcagggaaccaggtgtctctccctgtctttctgggtag

SEQ ID NO: 352 - DNA, 20 bp, synthetic construct
  source 1..20: mol_type = other DNA
cccggttcagcattcttttg

SEQ ID NO: 353 - DNA, 22 bp, synthetic construct
  source 1..22: mol_type = other DNA
cagaatgatgtggagatcccat

SEQ ID NO: 354 - DNA, 25 bp, synthetic construct
  source 1..25: mol_type = other DNA
gaggaattagactggtgtttagacc

SEQ ID NO: 355 - DNA, 20 bp, synthetic construct
  source 1..20: mol_type = other DNA
ctccctgtctttctgggtag

SEQ ID NO: 356 - DNA, 40 bp, synthetic construct
  source 1..40: mol_type = other DNA
ctcccggttcagcattctttaccatacagacctaccggtc

SEQ ID NO: 357 - DNA, 46 bp, synthetic construct
  source 1..46: mol_type = other DNA
ctctcagagatgagccgatcaacatcattctgcttgtctaagaaag

SEQ ID NO: 358 - DNA, 24 bp, synthetic construct
  source 1..24: mol_type = other DNA
tgaacttgttagaagccatctcac

SEQ ID NO: 359 - DNA, 23 bp, synthetic construct
  source 1..23: mol_type = other DNA
ggaaccaggtgtctgaatacatt

SEQ ID NO: 360 - DNA, 20 bp, synthetic construct
  source 1..20: mol_type = other DNA
ggtagcggtgactctgctat

SEQ ID NO: 361 - DNA, 23 bp, synthetic construct
  source 1..23: mol_type = other DNA
aatgtattcagacacctggttcc

SEQ ID NO: 362 - DNA, 44 bp, synthetic construct
  source 1..44: mol_type = other DNA
gctggtctaaacaccagtctaatcagcctaactacatgcctgtg

SEQ ID NO: 363 - DNA, 41 bp, synthetic construct
  source 1..41: mol_type = other DNA
cctaccggtctgtcagtgagtgatcggctcatctctgagag

SEQ ID NO: 364 - DNA, 21 bp, synthetic construct
  source 1..21: mol_type = other DNA
tccagcgtttccattgctaat

SEQ ID NO: 365 - DNA, 24 bp, synthetic construct
  source 1..24: mol_type = other DNA
acaagttcaaaagaatgctgaacc

SEQ ID NO: 366 - DNA, 21 bp, synthetic construct
  source 1..21: mol_type = other DNA
cagcctaactacatgcctgtg

SEQ ID NO: 367 - DNA, 25 bp, synthetic construct
  source 1..25: mol_type = other DNA
atttgaaatgtattcagacacctgg

SEQ ID NO: 368 - DNA, 42 bp, synthetic construct
  source 1..42: mol_type = other DNA
gaccggtaggtctgtatggtaaattagcaatggaaacgctgg

SEQ ID NO: 369 - DNA, 43 bp, synthetic construct
  source 1..43: mol_type = other DNA
cagtgagatggcttctaacaagtttccctgatcggctcatctc

SEQ ID NO: 370 - DNA, 24 bp, synthetic construct
  source 1..24: mol_type = other DNA
tggtctaaacaccagtctaattcc

SEQ ID NO: 371 - DNA, 20 bp, synthetic construct
  source 1..20: mol_type = other DNA
aaagaatgctgaaccgggag

SEQ ID NO: 372 - DNA, 23 bp, synthetic construct
  source 1..23: mol_type = other DNA
caatggaaacgctggaggaatta

SEQ ID NO: 373 - DNA, 20 bp, synthetic construct
  source 1..20: mol_type = other DNA
ctccctgtctttctgggtag

SEQ ID NO: 374 - DNA, 46 bp, synthetic construct
  source 1..46: mol_type = other DNA
ggttcagcattcttttgaacttgtggtgtttagaccagctagagac

SEQ ID NO: 375 - DNA, 46 bp, synthetic construct; identical to SEQ ID NO: 357
  source 1..46: mol_type = other DNA

SEQ ID NO: 376 - DNA, 20 bp, synthetic construct
  source 1..20: mol_type = other DNA
tctcactgacagaccggtag

SEQ ID NO: 377 - DNA, 23 bp, synthetic construct
  source 1..23: mol_type = other DNA
ggaaccaggtgtctgaatacatt

SEQ ID NO: 378 - DNA, 20 bp, synthetic construct
  source 1..20: mol_type = other DNA
gagtgtttgacaagctgtcc

SEQ ID NO: 379 - DNA, 19 bp, synthetic construct
  source 1..19: mol_type = other DNA
ctgacctccttcacgttgg

SEQ ID NO: 380 - DNA, 40 bp, synthetic construct
  source 1..40: mol_type = other DNA
ccaaaacaagtggaggttcctcacgagcgcatctactgtg

SEQ ID NO: 381 - DNA, 45 bp, synthetic construct
  source 1..45: mol_type = other DNA
ctgcaaatgtggtgagaaatatcagcagccagctaccatgagatg

SEQ ID NO: 382 - DNA, 21 bp, synthetic construct
  source 1..21: mol_type = other DNA
cagttctatcccatggagctc

SEQ ID NO: 383 - DNA, 23 bp, synthetic construct
  source 1..23: mol_type = other DNA
aaatatcgagaggacttgtctgc

SEQ ID NO: 384 - DNA, 20 bp, synthetic construct
  source 1..20: mol_type = other DNA
gaaccgagtgtttgacaagc

SEQ ID NO: 385 - DNA, 19 bp, synthetic construct
  source 1..19: mol_type = other DNA
gacctccttcacgttggtc

SEQ ID NO: 386 - DNA, 40 bp, synthetic construct
  source 1..40: mol_type = other DNA
caaaacaagtggaggttcctccacgagcgcatctactgtg

SEQ ID NO: 387 - DNA, 44 bp, synthetic construct
  source 1..44: mol_type = other DNA
gctgcaaatgtggtgagaaatatccagccagctaccatgagatg

SEQ ID NO: 388 - DNA, 21 bp, synthetic construct
  source 1..21: mol_type = other DNA
cagttctatcccatggagctc

SEQ ID NO: 389 - DNA, 25 bp, synthetic construct
  source 1..25: mol_type = other DNA
gctataaatatcgagaggacttgtc

SEQ ID NO: 390 - DNA, 20 bp, synthetic construct
  source 1..20: mol_type = other DNA
gatgggttctgattcccgag

SEQ ID NO: 391 - DNA, 20 bp, synthetic construct
  source 1..20: mol_type = other DNA
cagccagctaccatgagatg

SEQ ID NO: 392 - DNA, 41 bp, synthetic construct
  source 1..41: mol_type = other DNA
ccagttctatcccatggagctgagtgtttgacaagctgtcc

SEQ ID NO: 393 - DNA, 42 bp, synthetic construct
  source 1..42: mol_type = other DNA
ctccacttgttttggctgctgcagacaagtcctctcgatatt

SEQ ID NO: 394 - DNA, 18 bp, synthetic construct
  source 1..18: mol_type = other DNA
tgcacagtagatgcgctc

SEQ ID NO: 395 - DNA, 25 bp, synthetic construct
  source 1..25: mol_type = other DNA
caaatgtggtgagaaatatcagcta

SEQ ID NO: 396 - DNA, 18 bp, synthetic construct
  source 1..18: mol_type = other DNA
agcgcatctactgtgcac

SEQ ID NO: 397 - DNA, 19 bp, synthetic construct
  source 1..19: mol_type = other DNA
ccaatggcaaaaggctgtc

SEQ ID NO: 398 - DNA, 46 bp, synthetic construct
  source 1..46: mol_type = other DNA
tagctgatatttctcaccacatttgagctccatgggatagaactgg

SEQ ID NO: 399 - DNA, 42 bp, synthetic construct
  source 1..42: mol_type = other DNA
aatatcgagaggacttgtctgcagagtcagcattcctcccag

SEQ ID NO: 400 - DNA, 19 bp, synthetic construct
  source 1..19: mol_type = other DNA
gcagccaaaacaagtggag

SEQ ID NO: 401 - DNA, 21 bp, synthetic construct
  source 1..21: mol_type = other DNA
aacgtgaaggaggtcaggtat

SEQ ID NO: 402 - DNA, 18 bp, synthetic construct
  source 1..18: mol_type = other DNA
tgcactctctggttcagc

SEQ ID NO: 403 - DNA, 19 bp, synthetic construct
  source 1..19: mol_type = other DNA
ccaatggcaaaaggctgtc

SEQ ID NO: 404 - DNA, 44 bp, synthetic construct
  source 1..44: mol_type = other DNA
gcagacaagtcctctcgatatttagctccatgggatagaactgg

SEQ ID NO: 405 - DNA, 39 bp, synthetic construct
  source 1..39: mol_type = other DNA
catctcatggtagctggctggagtcagcattcctcccag

SEQ ID NO: 406 - DNA, 25 bp, synthetic construct
  source 1..25: mol_type = other DNA
ctgatatttctcaccacatttgcag

SEQ ID NO: 407 - DNA, 21 bp, synthetic construct
  source 1..21: mol_type = other DNA
aacgtgaaggaggtcaggtat

SEQ ID NO: 408 - DNA, 24 bp, synthetic construct
  source 1..24: mol_type = other DNA
gacttgtctgcacatctcatggta

SEQ ID NO: 409 - DNA, 25 bp, synthetic construct
  source 1..25: mol_type = other DNA
taatagtgaccaggtagatgacacc

SEQ ID NO: 410 - DNA, 43 bp, synthetic construct
  source 1..43: mol_type = other DNA
ggagccaccaatggcaaaagccaacgtgaaggaggtcaggtat

SEQ ID NO: 411 - DNA, 46 bp, synthetic construct
  source 1..46: mol_type = other DNA
gcagcacctttatctatggttatgtcaatagcgtctgtggtgaagc

SEQ ID NO: 412 - DNA, 20 bp, synthetic construct
  source 1..20: mol_type = other DNA
cagcattcctcccagggttc

SEQ ID NO: 413 - DNA, 23 bp, synthetic construct
  source 1..23: mol_type = other DNA
agcatataagccaggcatgtctc

SEQ ID NO: 414 - DNA, 25 bp, synthetic construct
  source 1..25: mol_type = other DNA
caaatgtggtgagaaatatcagcta

SEQ ID NO: 415 - DNA, 20 bp, synthetic construct
  source 1..20: mol_type = other DNA
tgcactcctcgggagacatg

SEQ ID NO: 416 - DNA, 43 bp, synthetic construct
  source 1..43: mol_type = other DNA
gtcagcattcctcccagggtggacttgtctgcacatctcatgg

SEQ ID NO: 417 - DNA, 43 bp, synthetic construct
  source 1..43: mol_type = other DNA
cgacagccttttgccattggtggcttatatgctgcatccacat

SEQ ID NO: 418 - DNA, 25 bp, synthetic construct
  source 1..25: mol_type = other DNA
catatacctgacctccttcacgttg

SEQ ID NO: 419 - DNA, 19 bp, synthetic construct
  source 1..19: mol_type = other DNA
gctccggcagcacctttat

SEQ ID NO: 420 - DNA, 25 bp, synthetic construct
  source 1..25: mol_type = other DNA
tgcaaatgtggtgagaaatatcagc

SEQ ID NO: 421 - DNA, 23 bp, synthetic construct
  source 1..23: mol_type = other DNA
ggagacatgcctggcttatatgc

SEQ ID NO: 422 - DNA, 48 bp, synthetic construct
  source 1..48: mol_type = other DNA
ccagggttccatatacctgacctataaatatcgagaggacttgtctgc

SEQ ID NO: 423 - DNA, 46 bp, synthetic construct
  source 1..46: mol_type = other DNA
gaggaatgctgactcgacagctccacataaccatagataaaggtgc

SEQ ID NO: 424 - DNA, 21 bp, synthetic construct
  source 1..21: mol_type = other DNA
cccagccagctaccatgagat

SEQ ID NO: 425 - DNA, 20 bp, synthetic construct
  source 1..20: mol_type = other DNA
cttttgccattggtggctcc

SEQ ID NO: 426 - DNA, 25 bp, synthetic construct
  source 1..25: mol_type = other DNA
gctactttaagaccattgtggacaa

SEQ ID NO: 427 - DNA, 21 bp, synthetic construct
  source 1..21: mol_type = other DNA
tgtcttctgagttcacgcctt

SEQ ID NO: 428 - DNA, 44 bp, synthetic construct
  source 1..44: mol_type = other DNA
gtgctgaattcgaggatcgttcagaaggtactggagatgcttcc

SEQ ID NO: 429 - DNA, 42 bp, synthetic construct
  source 1..42: mol_type = other DNA
ttcctgctatagccgagtgtaccagcatcacttggtcagacc

SEQ ID NO: 430 - DNA, 20 bp, synthetic construct
  source 1..20: mol_type = other DNA
aggggtaagatggcctccag

SEQ ID NO: 431 - DNA, 18 bp, synthetic construct
  source 1..18: mol_type = other DNA
agcctcgccaacctcatt

SEQ ID NO: 432 - DNA, 22 bp, synthetic construct
  source 1..22: mol_type = other DNA
agaaggtactggagatgcttcc

SEQ ID NO: 433 - DNA, 19 bp, synthetic construct
  source 1..19: mol_type = other DNA
cagcacagccttgatgacc

SEQ ID NO: 434 - DNA, 41 bp, synthetic construct
  source 1..41: mol_type = other DNA
gtacactcggctatagcaggaagtgctggaggccatcttac

SEQ ID NO: 435 - DNA, 41 bp, synthetic construct
  source 1..41: mol_type = other DNA
aacctcattcgctggtctgaagtcgtcaccatctccttgtc

SEQ ID NO: 436 - DNA, 19 bp, synthetic construct
  source 1..19: mol_type = other DNA
gaggatcgttctgcaccag

SEQ ID NO: 437 - DNA, 20 bp, synthetic construct
  source 1..20: mol_type = other DNA
tggaaggcgtgaactcagaa

SEQ ID NO: 438 - DNA, 19 bp, synthetic construct
  source 1..19: mol_type = other DNA
gtgctggaggccatcttac

SEQ ID NO: 439 - DNA, 20 bp, synthetic construct
  source 1..20: mol_type = other DNA
cctgaccagctccttcactc

SEQ ID NO: 440 - DNA, 40 bp, synthetic construct
  source 1..40: mol_type = other DNA
cactcggctatagcaggaagactggtgcagaacgatcctc

SEQ ID NO: 441 - DNA, 41 bp, synthetic construct; identical to SEQ ID NO: 435
  source 1..41: mol_type = other DNA

SEQ ID NO: 442 - DNA, 20 bp, synthetic construct
  source 1..20: mol_type = other DNA
ggctgagctgtgctgaattc

SEQ ID NO: 443 - DNA, 19 bp, synthetic construct
  source 1..19: mol_type = other DNA
gatgctggaaggcgtgaac

SEQ ID NO: 444 - DNA, 21 bp, synthetic construct
  source 1..21: mol_type = other DNA
gctgctattctgccctttcag

SEQ ID NO: 445 - DNA, 22 bp, synthetic construct
  source 1..22: mol_type = other DNA
ctccatgaggagcttgttcttc

SEQ ID NO: 446 - DNA, 39 bp, synthetic construct
  source 1..39: mol_type = other DNA
ctgcatgtaggccagcatgttcctcagcccctgtcgaat

SEQ ID NO: 447 - DNA, 40 bp, synthetic construct
  source 1..40: mol_type = other DNA
ttgctggaggactactcggatgctggtagatgtgctcgtt

SEQ ID NO: 448 - DNA, 23 bp, synthetic construct
  source 1..23: mol_type = other DNA
gactcaggagcagtaaaatcacc

SEQ ID NO: 449 - DNA, 21 bp, synthetic construct
  source 1..21: mol_type = other DNA
ccgcagccctctatgttctac

SEQ ID NO: 450 - DNA, 20 bp, synthetic construct
  source 1..20: mol_type = other DNA
gccctttcagcatggaggtt

SEQ ID NO: 451 - DNA, 20 bp, synthetic construct
  source 1..20: mol_type = other DNA
ggagtcgctgaagccgtata

SEQ ID NO: 452 - DNA, 44 bp, synthetic construct
  source 1..44: mol_type = other DNA
ccagcaactgcatgtaggcgtcgaatttgtgggtgattttactg

SEQ ID NO: 453 - DNA, 43 bp, synthetic construct
  source 1..43: mol_type = other DNA
gcagccctctatgttctaccactccatgaggagcttgttcttc

SEQ ID NO: 454 - DNA, 20 bp, synthetic construct
  source 1..20: mol_type = other DNA
gtcaccggttgactcaggag

SEQ ID NO: 455 - DNA, 21 bp, synthetic construct
  source 1..21: mol_type = other DNA
gccacagaacgagcacatcta

SEQ ID NO: 456 - DNA, 20 bp, synthetic construct
  source 1..20: mol_type = other DNA
ccctttcagcatggaggttc

SEQ ID NO: 457 - DNA, 22 bp, synthetic construct
  source 1..22: mol_type = other DNA
ctccatgaggagcttgttcttc

SEQ ID NO: 458 - DNA, 39 bp, synthetic construct
  source 1..39: mol_type = other DNA
ctgcatgtaggccagcatgctcagcccctgtcgaatttg

SEQ ID NO: 459 - DNA, 40 bp, synthetic construct
  source 1..40: mol_type = other DNA
ttgctggaggactactcggagctggtagatgtgctcgttc

SEQ ID NO: 460 - DNA, 23 bp, synthetic construct
  source 1..23: mol_type = other DNA
gactcaggagcagtaaaatcacc

SEQ ID NO: 461 - DNA, 21 bp, synthetic construct
  source 1..21: mol_type = other DNA
ccgcagccctctatgttctac

SEQ ID NO: 462 - DNA, 25 bp, synthetic construct
  source 1..25: mol_type = other DNA
gtcgaatttgtgggtgattttactg

SEQ ID NO: 463 - DNA, 20 bp, synthetic construct
  source 1..20: mol_type = other DNA
ggagtcgctgaagccgtata

SEQ ID NO: 464 - DNA, 40 bp, synthetic construct
  source 1..40: mol_type = other DNA
cgagtagtcctccagcaactctcctgagtcaaccggtgac

SEQ ID NO: 465 - DNA, 43 bp, synthetic construct; identical to SEQ ID NO: 453
  source 1..43: mol_type = other DNA

SEQ ID NO: 466 - DNA, 21 bp, synthetic construct
  source 1..21: mol_type = other DNA
catgtaggccagcatgtgttt

SEQ ID NO: 467 - DNA, 21 bp, synthetic construct
  source 1..21: mol_type = other DNA
gccacagaacgagcacatcta

SEQ ID NO: 468 - DNA, 20 bp, synthetic construct
  source 1..20: mol_type = other DNA
cccaaagtcaccagatgctc

SEQ ID NO: 469 - DNA, 22 bp, synthetic construct
  source 1..22: mol_type = other DNA
gtactgcagcttcttgatgagc

SEQ ID NO: 470 - DNA, 42 bp, synthetic construct
  source 1..42: mol_type = other DNA
cagtaagatgtccccagatcctcggacgagctgtccctcatt

SEQ ID NO: 471 - DNA, 43 bp, synthetic construct
  source 1..43: mol_type = other DNA
gtccatgctactgagactgacacctgtaggtggtcaggaatgc

SEQ ID NO: 472 - DNA, 22 bp, synthetic construct
  source 1..22: mol_type = other DNA
gcgtcagcctggacataatttc

SEQ ID NO: 473 - DNA, 22 bp, synthetic construct
  source 1..22: mol_type = other DNA
agatttggtgttgtactgcgag

SEQ ID NO: 474 - DNA, 21 bp, synthetic construct
  source 1..21: mol_type = other DNA
ccaaagtcaccagatgctctg

SEQ ID NO: 475 - DNA, 22 bp, synthetic construct
  source 1..22: mol_type = other DNA
gtactgcagcttcttgatgagc

SEQ ID NO: 476 - DNA, 44 bp, synthetic construct
  source 1..44: mol_type = other DNA
cagtagcatggaccagtaagatgtgaggaagtggacgagctgtc

SEQ ID NO: 477 - DNA, 45 bp, synthetic construct
  source 1..45: mol_type = other DNA
gactgacaggaaagatttggtgttggggagatgaaggtcctgtag

SEQ ID NO: 478 - DNA, 20 bp, synthetic construct
  source 1..20: mol_type = other DNA
gagcgtcagcctggacataa

SEQ ID NO: 479 - DNA, 19 bp, synthetic construct
  source 1..19: mol_type = other DNA
gtactgcgaggcattcctg

SEQ ID NO: 480 - DNA, 20 bp, synthetic construct
  source 1..20: mol_type = other DNA
cttggctcagtgcccttcac

SEQ ID NO: 481 - DNA, 23 bp, synthetic construct
  source 1..23: mol_type = other DNA
tgtttgcaagctcctttgtaagc

SEQ ID NO: 482 - DNA, 43 bp, synthetic construct
  source 1..43: mol_type = other DNA
cctccagatgctcttcaagttttggcgctgtagctccacattc

SEQ ID NO: 483 - DNA, 43 bp, synthetic construct
  source 1..43: mol_type = other DNA
cttccaccaatactcagttcggagctgcttcagctcaccctta

SEQ ID NO: 484 - DNA, 22 bp, synthetic construct
  source 1..22: mol_type = other DNA
cccagcctaatgttaacccctc

SEQ ID NO: 485 - DNA, 20 bp, synthetic construct
  source 1..20: mol_type = other DNA
aggggcattttgacaccctc

SEQ ID NO: 486 - DNA, 23 bp, synthetic construct
  source 1..23: mol_type = other DNA
ggctttttgctgtagctccacat

SEQ ID NO: 487 - DNA, 23 bp, synthetic construct
  source 1..23: mol_type = other DNA
tgtttgcaagctcctttgtaagc

SEQ ID NO: 488 - DNA, 45 bp, synthetic construct
  source 1..45: mol_type = other DNA
gacaattccctccagatgctctttcctgtgcattgaggggttaac

SEQ ID NO: 489 - DNA, 43 bp, synthetic construct; identical to SEQ ID NO: 483
  source 1..43: mol_type = other DNA

SEQ ID NO: 490 - DNA, 25 bp, synthetic construct
  source 1..25: mol_type = other DNA
agttttgtcatcttcccagcctaat

SEQ ID NO: 491 - DNA, 20 bp, synthetic construct
  source 1..20: mol_type = other DNA
aggggcattttgacaccctc

SEQ ID NO: 492 - DNA, 20 bp, synthetic construct
  source 1..20: mol_type = other DNA
gctgtagctccacattcctg

SEQ ID NO: 493 - DNA, 21 bp, synthetic construct
  source 1..21: mol_type = other DNA
tgcaagctcctttgtaagcag

SEQ ID NO: 494 - DNA, 45 bp, synthetic construct
  source 1..45: mol_type = other DNA
attgacaattccctccagatgctgcattgaggggttaacattagg

SEQ ID NO: 495 - DNA, 41 bp, synthetic construct
  source 1..41: mol_type = other DNA
cttccaccaatactcagttcggctgcttcagctcaccctta

SEQ ID NO: 496 - DNA, 24 bp, synthetic construct
  source 1..24: mol_type = other DNA
cttcaagttttgtcatcttcccag

SEQ ID NO: 497 - DNA, 19 bp, synthetic construct
  source 1..19: mol_type = other DNA
ggggcattttgacaccctc

SEQ ID NO: 498 - DNA, 20 bp, synthetic construct
  source 1..20: mol_type = other DNA
cttggctcagtgcccttcac

SEQ ID NO: 499 - DNA, 23 bp, synthetic construct
  source 1..23: mol_type = other DNA
tgtttgcaagctcctttgtaagc

SEQ ID NO: 500 - DNA, 43 bp, synthetic construct; identical to SEQ ID NO: 116
  source 1..43: mol_type = other DNA

SEQ ID NO: 501 - DNA, 46 bp, synthetic construct; identical to SEQ ID NO: 117
  source 1..46: mol_type = other DNA

SEQ ID NO: 502 - DNA, 22 bp, synthetic construct
  source 1..22: mol_type = other DNA
cccagcctaatgttaacccctc

SEQ ID NO: 503 - DNA, 20 bp, synthetic construct
  source 1..20: mol_type = other DNA
aggggcattttgacaccctc

SEQ ID NO: 504 - DNA, 23 bp, synthetic construct
  source 1..23: mol_type = other DNA
ggctttttgctgtagctccacat

SEQ ID NO: 505 - DNA, 23 bp, synthetic construct
  source 1..23: mol_type = other DNA
tgtttgcaagctcctttgtaagc

SEQ ID NO: 506 - DNA, 50 bp, synthetic construct
  source 1..50: mol_type = other DNA
gacaattccctccagatgctcttttttttcctgtgcattgaggggttaac

SEQ ID NO: 507 - DNA, 48 bp, synthetic construct
  source 1..48: mol_type = other DNA
cttccaccaatactcagttcggatttttgctgcttcagctcaccctta

SEQ ID NO: 508 - DNA, 25 bp, synthetic construct
  source 1..25: mol_type = other DNA
agttttgtcatcttcccagcctaat

SEQ ID NO: 509 - DNA, 20 bp, synthetic construct
  source 1..20: mol_type = other DNA
aggggcattttgacaccctc

SEQ ID NO: 510 - DNA, 20 bp, synthetic construct
  source 1..20: mol_type = other DNA
gctgtagctccacattcctg

SEQ ID NO: 511 - DNA, 21 bp, synthetic construct
  source 1..21: mol_type = other DNA
tgcaagctcctttgtaagcag

SEQ ID NO: 512 - DNA, 50 bp, synthetic construct
  source 1..50: mol_type = other DNA
attgacaattccctccagatgcttttttgcattgaggggttaacattagg

SEQ ID NO: 513 - DNA, 46 bp, synthetic construct
  source 1..46: mol_type = other DNA
cttccaccaatactcagttcggtttttctgcttcagctcaccctta

SEQ ID NO: 514 - DNA, 24 bp, synthetic construct
  source 1..24: mol_type = other DNA
cttcaagttttgtcatcttcccag

SEQ ID NO: 515 - DNA, 19 bp, synthetic construct
  source 1..19: mol_type = other DNA
ggggcattttgacaccctc

SEQ ID NO: 516 - DNA, 20 bp, synthetic construct
  source 1..20: mol_type = other DNA
ttccttggctcagtgccctt

SEQ ID NO: 517 - DNA, 22 bp, synthetic construct
  source 1..22: mol_type = other DNA
ctgcttcagctcacccttagag

SEQ ID NO: 518 - DNA, 46 bp, synthetic construct
  source 1..46: mol_type = other DNA
aagttttgtcatcttcccagcctggctttttgctgtagctccacat

SEQ ID NO: 519 - DNA, 44 bp, synthetic construct
  source 1..44: mol_type = other DNA
gaagagcatctggagggaattgtagggtgtcaaaatgccccttc

SEQ ID NO: 520 - DNA, 22 bp, synthetic construct
  source 1..22: mol_type = other DNA
tgttaacccctcaatgcacagg

SEQ ID NO: 521 - DNA, 25 bp, synthetic construct
  source 1..25: mol_type = other DNA
atatcttccaccaatactcagttcg

SEQ ID NO: 522 - DNA, 23 bp, synthetic construct
  source 1..23: mol_type = other DNA
aggctgggaagatgacaaaactt

SEQ ID NO: 523 - DNA, 23 bp, synthetic construct
  source 1..23: mol_type = other DNA
gcaatggctaccagggatatgaa

SEQ ID NO: 524 - DNA, 43 bp, synthetic construct
  source 1..43: mol_type = other DNA
gagggtgtcaaaatgcccctaagagcatctggagggaattgtc

SEQ ID NO: 525 - DNA, 48 bp, synthetic construct
  source 1..48: mol_type = other DNA
gcttacaaaggagcttgcaaacaagtcgacctgttcatcttgattagc

SEQ ID NO: 526 - DNA, 23 bp, synthetic construct
  source 1..23: mol_type = other DNA
tccgaactgagtattggtggaag

SEQ ID NO: 527 - DNA, 25 bp, synthetic construct
  source 1..25: mol_type = other DNA
cattgatgaaatattccaaggcctg

SEQ ID NO: 528 - DNA, 25 bp, synthetic construct
  source 1..25: mol_type = other DNA
gagggaattgtcaatatcttccacc

SEQ ID NO: 529 - DNA, 24 bp, synthetic construct
  source 1..24: mol_type = other DNA
cagagagctacctactctttgtgg

SEQ ID NO: 530 - DNA, 42 bp, synthetic construct
  source 1..42: mol_type = other DNA
caagctcctttgtaagcagctctcagttcggaaggggcattt

SEQ ID NO: 531 - DNA, 45 bp, synthetic construct
  source 1..45: mol_type = other DNA
aatattccaaggcctggatgctaatttcagcgcaatggctaccag

SEQ ID NO: 532 - DNA, 22 bp, synthetic construct
  source 1..22: mol_type = other DNA
gcttcagctcacccttagagag

SEQ ID NO: 533 - DNA, 25 bp, synthetic construct
  source 1..25: mol_type = other DNA
caagatgaacaggtcgactttcaag

SEQ ID NO: 534 - DNA, 20 bp, synthetic construct
  source 1..20: mol_type = other DNA
cggaaggggcattttgacac

SEQ ID NO: 535 - DNA, 24 bp, synthetic construct
  source 1..24: mol_type = other DNA
ggacattgctgggtaaaaagcctt

SEQ ID NO: 536 - DNA, 46 bp, synthetic construct
  source 1..46: mol_type = other DNA
attcttgaaagtcgacctgttcatcacaaaggagcttgcaaacacc

SEQ ID NO: 537 - DNA, 44 bp, synthetic construct
  source 1..44: mol_type = other DNA
tatccctggtagccattgcgcagagagctacctactctttgtgg

SEQ ID NO: 538 - DNA, 25 bp, synthetic construct
  source 1..25: mol_type = other DNA
attagcatccaggccttggaatatt

SEQ ID NO: 539 - DNA, 19 bp, synthetic construct
  source 1..19: mol_type = other DNA
ctgaaggctgcccattacc

SEQ ID NO: 540 - DNA, 20 bp, synthetic construct
  source 1..20: mol_type = other DNA
cttggctcagtgcccttcac

SEQ ID NO: 541 - DNA, 23 bp, synthetic construct
  source 1..23: mol_type = other DNA
tgtttgcaagctcctttgtaagc

SEQ ID NO: 542 - DNA, 43 bp, synthetic construct; identical to SEQ ID NO: 482
  source 1..43: mol_type = other DNA

SEQ ID NO: 543 - DNA, 43 bp, synthetic construct; identical to SEQ ID NO: 483
  source 1..43: mol_type = other DNA

SEQ ID NO: 544 - DNA, 22 bp, synthetic construct
  source 1..22: mol_type = other DNA
cccagcctaatgttaacccctc

SEQ ID NO: 545 - DNA, 20 bp, synthetic construct
  source 1..20: mol_type = other DNA
aggggcattttgacaccctc

SEQ ID NO: 546 - DNA, 27 bp, synthetic construct
  source 1..27: mol_type = other DNA
gtacctggtttgcatagatgattggca

SEQ ID NO: 547 - DNA, 20 bp, synthetic construct
  source 1..20: mol_type = other DNA
cacccaggaaggtaccacat

SEQ ID NO: 548 - DNA, 19 bp, synthetic construct
  source 1..19: mol_type = other DNA
ctccgggcattggccaaca

SEQ ID NO: 549 - DNA, 20 bp, synthetic construct
  source 1..20: mol_type = other DNA
caggccgggcacaacttcta

SEQ ID NO: 550 - DNA, 46 bp, synthetic construct
  source 1..46: mol_type = other DNA
acaggggatggaaagagggccatctaggatccggcttccaacatgt

SEQ ID NO: 551 - DNA, 57 bp, synthetic construct
  source 1..57: mol_type = other DNA
tggtcaactatgtcaacaaacggaataccacctcttcaagtagctcatgtccacgtt

SEQ ID NO: 552 - DNA, 26 bp, synthetic construct
  source 1..26: mol_type = other DNA
gcctgcagtacctggtttgcatagat

SEQ ID NO: 553 - DNA, 20 bp, synthetic construct
  source 1..20: mol_type = other DNA
cacccaggaaggtaccacat

SEQ ID NO: 554 - DNA, 19 bp, synthetic construct
  source 1..19: mol_type = other DNA
ctccgggcattggccaaca

SEQ ID NO: 555 - DNA, 20 bp, synthetic construct
  source 1..20: mol_type = other DNA
caggccgggcacaacttcta

SEQ ID NO: 556 - DNA, 46 bp, synthetic construct; identical to SEQ ID NO: 550
  source 1..46: mol_type = other DNA

SEQ ID NO: 557 - DNA, 52 bp, synthetic construct
  source 1..52: mol_type = other DNA
tgagctggtcaactatgtcaacaaacggcctcttcaagtagctcatgtccac

SEQ ID NO: 558 - DNA, 17 bp, synthetic construct
  source 1..17: mol_type = other DNA
ccctccgtcagcagcaa

SEQ ID NO: 559 - DNA, 17 bp, synthetic construct
  source 1..17: mol_type = other DNA
cacaccagggcacatgg

SEQ ID NO: 560 - DNA, 40 bp, synthetic construct
  source 1..40: mol_type = other DNA
accccctgggtgtaagtggtggggcatcatggaggaggat

SEQ ID NO: 561 - DNA, 35 bp, synthetic construct
  source 1..35: mol_type = other DNA
gccccccagccaaggtgatctcccgcacccgtttg

SEQ ID NO: 562 - DNA, 19 bp, synthetic construct
  source 1..19: mol_type = other DNA
atgaacctgatggagcagc

SEQ ID NO: 563 - DNA, 16 bp, synthetic construct
  source 1..16: mol_type = other DNA
gcaccccctgggtgta

SEQ ID NO: 564 - DNA, 40 bp, synthetic construct
  source 1..40: mol_type = other DNA
ttggcgcccgagtggatacccaaggtgactgagtggcagc

SEQ ID NO: 565 - DNA, 38 bp, synthetic construct
  source 1..38: mol_type = other DNA
gccctccgtcagcagcaaggtggttttcttgagcgtgt

SEQ ID NO: 566 - DNA, 20 bp, synthetic construct
  source 1..20: mol_type = other DNA
ccgagctcagttcgctgtcc

SEQ ID NO: 567 - DNA, 20 bp, synthetic construct
  source 1..20: mol_type = other DNA
tcctccatgatgcccttgct

SEQ ID NO: 568 - DNA, 45 bp, synthetic construct
  source 1..45: mol_type = other DNA
aggttcatcacctccatcgtggccggtcaggccccatactcagta

SEQ ID NO: 569 - DNA, 48 bp, synthetic construct
  source 1..48: mol_type = other DNA
tggagcagcctatcaaggtgactgtacccgagtcgtaggtgtatgtct

SEQ ID NO: 570 - DNA, 23 bp, synthetic construct
  source 1..23: mol_type = other DNA
gacctggggagacatgagtcgag

SEQ ID NO: 571 - DNA, 25 bp, synthetic construct
  source 1..25: mol_type = other DNA
ggggacattgacataatcatgcgaa

SEQ ID NO: 572 - DNA, 24 bp, synthetic construct
  source 1..24: mol_type = other DNA
gctgtatgctcctggaaggcattg

SEQ ID NO: 573 - DNA, 24 bp, synthetic construct
  source 1..24: mol_type = other DNA
cggcacactgcatcaatgtcagag

SEQ ID NO: 574 - DNA, 45 bp, synthetic construct
  source 1..45: mol_type = other DNA
gtcatagatacccaccgcgtactctcatgtgccctcccaagcagg

SEQ ID NO: 575 - DNA, 48 bp, synthetic construct
  source 1..48: mol_type = other DNA
ccagatgtgtgggaacctcactccgaaatgcttgcgcagtctctggaa

SEQ ID NO: 576 - DNA, 25 bp, synthetic construct
  source 1..25: mol_type = other DNA
cacactgcatcaatgtcagagcttc

SEQ ID NO: 577 - DNA, 22 bp, synthetic construct
  source 1..22: mol_type = other DNA
gtgcagtcaccagcatctccac

SEQ ID NO: 578 - DNA, 25 bp, synthetic construct
  source 1..25: mol_type = other DNA
tgtggggacattgacataatcatgc

SEQ ID NO: 579 - DNA, 25 bp, synthetic construct
  source 1..25: mol_type = other DNA
tctttgttcttcccagtacccagaa

SEQ ID NO: 580 - DNA, 48 bp, synthetic construct
  source 1..48: mol_type = other DNA
gctagagtctcagcaatctcttctggcgcaagcatttcttcagaggat

SEQ ID NO: 581 - DNA, 50 bp, synthetic construct
  source 1..50: mol_type = other DNA
ttctaccaaaagcccttccagaaatatctctttcctcagtaaactccagc

SEQ ID NO: 582 - DNA, 22 bp, synthetic construct
  source 1..22: mol_type = other DNA
tgaacggacgatgcggtcagta

SEQ ID NO: 583 - DNA, 20 bp, synthetic construct
  source 1..20: mol_type = other DNA
gggaagggcagtttccggga

SEQ ID NO: 584 - DNA, 20 bp, synthetic construct
  source 1..20: mol_type = other DNA
ccgagtcgcttgtgctcacc

SEQ ID NO: 585 - DNA, 29 bp, synthetic construct
  source 1..29: mol_type = other DNA
agacgatatctttggcccaataaatctgc

SEQ ID NO: 586 - DNA, 42 bp, synthetic construct
  source 1..42: mol_type = other DNA
gacaccatatagcgctcgtggggctggaggtctacacgcaga

SEQ ID NO: 587 - DNA, 43 bp, synthetic construct
  source 1..43: mol_type = other DNA
accaacgtgtacggcatcctgagcctggctgttggtagagtca

SEQ ID NO: 588 - DNA, 20 bp, synthetic construct
  source 1..20: mol_type = other DNA
cacggctcccatggcctctt

SEQ ID NO: 589 - DNA, 19 bp, synthetic construct
  source 1..19: mol_type = other DNA
ccgacagcgggcatgtaga

SEQ ID NO: 590 - DNA, 25 bp, synthetic construct
  source 1..25: mol_type = other DNA
ccaggtcatacttgtactggcggaa

SEQ ID NO: 591 - DNA, 17 bp, synthetic construct
  source 1..17: mol_type = other DNA
cgcctgcaccagtcctt

SEQ ID NO: 592 - DNA, 40 bp, synthetic construct
  source 1..40: mol_type = other DNA
tccaaagccttgcccactgcaccgtgtggaggccctaacc

SEQ ID NO: 593 - DNA, 39 bp, synthetic construct
  source 1..39: mol_type = other DNA
atgttccgcaagctcaaccaccgccggggagcaagtaga

SEQ ID NO: 594 - DNA, 19 bp, synthetic construct
  source 1..19: mol_type = other DNA
cagctgcccaaccttgacc

SEQ ID NO: 595 - DNA, 22 bp, synthetic construct
  source 1..22: mol_type = other DNA
tggcggaagctattgatgccac

SEQ ID NO: 596 - DNA, 26 bp, synthetic construct
  source 1..26: mol_type = other DNA
ggtccatccaatgatgtccagtcctc

SEQ ID NO: 597 - DNA, 20 bp, synthetic construct
  source 1..20: mol_type = other DNA
cacggctcccatggcctctt

SEQ ID NO: 598 - DNA, 43 bp, synthetic construct
  source 1..43: mol_type = other DNA
agtgtctgcaggccctgcaatctcttccagaccttctgccaga

SEQ ID NO: 599 - DNA, 41 bp, synthetic construct
  source 1..41: mol_type = other DNA
gctcatggttctgcggcaggggttagggcctccacacggta

SEQ ID NO: 600 - DNA, 22 bp, synthetic construct
  source 1..22: mol_type = other DNA
ctctccagggacagtggcccta

SEQ ID NO: 601 - DNA, 23 bp, synthetic construct
  source 1..23: mol_type = other DNA
gcaggccatccctacattgcgat

SEQ ID NO: 602 - DNA, 52 bp, synthetic construct
  source 1..52: mol_type = other DNA
actggcaaacatgttgaggacaatgatggcggcaggttcctatgcaagctca

SEQ ID NO: 603 - DNA, 56 bp, synthetic construct
  source 1..56: mol_type = other DNA
ttcctgcttactgccattagcctggagacaccagattggcttgaataccacaagac

SEQ ID NO: 604 - DNA, 24 bp, synthetic construct
  source 1..24: mol_type = other DNA
ctcacttggctctccagggacagt

SEQ ID NO: 605 - DNA, 23 bp, synthetic construct
  source 1..23: mol_type = other DNA
gcaggccatccctacattgcgat

SEQ ID NO: 606 - DNA, 53 bp, synthetic construct
  source 1..53: mol_type = other DNA
actggcaaacatgttgaggacaatgatggcctacggcaggttcctatgcaagc

SEQ ID NO: 607 - DNA, 56 bp, synthetic construct; identical to SEQ ID NO: 603
  source 1..56: mol_type = other DNA

SEQ ID NO: 608 - DNA, 22 bp, synthetic construct
  source 1..22: mol_type = other DNA
gaagcctggtggctctgacctc

SEQ ID NO: 609 - DNA, 30 bp, synthetic construct
  source 1..30: mol_type = other DNA
gggtctgagaagttgagtgaaacaagactg

SEQ ID NO: 610 - DNA, 20 bp, synthetic construct
  source 1..20: mol_type = other DNA
gggatggggctccttcacca

SEQ ID NO: 611 - DNA, 22 bp, synthetic construct
  source 1..22: mol_type = other DNA
cttgcaggtggctggctcagaa

SEQ ID NO: 612 - DNA, 51 bp, synthetic construct
  source 1..51: mol_type = other DNA
agcaaagaggctagggaggaaccagcagccatgacactcttaacggatgcc

SEQ ID NO: 613 - DNA, 49 bp, synthetic construct
  source 1..49: mol_type = other DNA
ccttcaatgtggtgctgctggtctctgtgataacaggaaaggcccactc

SEQ ID NO: 614 - DNA, 19 bp, synthetic construct
  source 1..19: mol_type = other DNA
gctggtgaaggagccccat

SEQ ID NO: 615 - DNA, 17 bp, synthetic construct
  source 1..17: mol_type = other DNA
cccgtggttcacccaca

SEQ ID NO: 616 - DNA, 22 bp, synthetic construct
  source 1..22: mol_type = other DNA
gcagcaccacattgaaggcagc

SEQ ID NO: 617 - DNA, 32 bp, synthetic construct
  source 1..32: mol_type = other DNA
tccttaccttcttcagtcttgtttcactcaac

SEQ ID NO: 618 - DNA, 40 bp, synthetic construct
  source 1..40: mol_type = other DNA
cgttctgagccagccacctggtccctggttcctccctagc

SEQ ID NO: 619 - DNA, 54 bp, synthetic construct
  source 1..54: mol_type = other DNA
gagtgggcctttcctgttatcacagccttgatgtaagatgccagggtctgagaa

SEQ ID NO: 620 - DNA, 20 bp, synthetic construct
  source 1..20: mol_type = other DNA
gtccctggttcctccctagc

SEQ ID NO: 621 - DNA, 17 bp, synthetic construct
  source 1..17: mol_type = other DNA
cccgtggttcacccaca

SEQ ID NO: 622 - DNA, 27 bp, synthetic construct
  source 1..27: mol_type = other DNA
gcgaagaagaggccactgaaaaagacc

SEQ ID NO: 623 - DNA, 32 bp, synthetic construct; identical to SEQ ID NO: 617
  source 1..32: mol_type = other DNA

SEQ ID NO: 624 - DNA, 47 bp, synthetic construct
  source 1..47: mol_type = other DNA
ttctgagccagccacctgcaagctctttgctgccttcaatgtggtgc

SEQ ID NO: 625 - DNA, 54 bp, synthetic construct; identical to SEQ ID NO: 619
  source 1..54: mol_type = other DNA

SEQ ID NO: 626 - DNA, 25 bp, synthetic construct
  source 1..25: mol_type = other DNA
ctgctggtctttttcagtggcctct

SEQ ID NO: 627 - DNA, 20 bp, synthetic construct
  source 1..20: mol_type = other DNA
aggggcagtggtaagtccgg

SEQ ID NO: 628 - DNA, 28 bp, synthetic construct
  source 1..28: mol_type = other DNA
aggacaaagagggagcctgtgataacag

SEQ ID NO: 629 - DNA, 20 bp, synthetic construct
  source 1..20: mol_type = other DNA
gccttccgcctgcaatggtg

SEQ ID NO: 630 - DNA, 52 bp, synthetic construct
  source 1..52: mol_type = other DNA
gatgtaagatgccagggtctgagaagttgatcagaacggggagtgggccttt

SEQ ID NO: 631 - DNA, 39 bp, synthetic construct
  source 1..39: mol_type = other DNA
tgtgggtgaaccacggggcggtggaagcagcactttgga

SEQ ID NO: 632 - DNA, 24 bp, synthetic construct
  source 1..24: mol_type = other DNA
acacatccaagcttaagacggtga

SEQ ID NO: 633 - DNA, 21 bp, synthetic construct
  source 1..21: mol_type = other DNA
caactccacccccattggcaa

SEQ ID NO: 634 - DNA, 48 bp, synthetic construct
  source 1..48: mol_type = other DNA
aactgtagcaatcctggccaaagagcttcacattctcaggaactctcc

SEQ ID NO: 635 - DNA, 44 bp, synthetic construct
  source 1..44: mol_type = other DNA
tcagtgccatgggcttcactatcatcttggctgctatggaggac

SEQ ID NO: 636 - DNA, 18 bp, synthetic construct
  source 1..18: mol_type = other DNA
tcgcctcgtcctccatag

SEQ ID NO: 637 - DNA, 20 bp, synthetic construct
  source 1..20: mol_type = other DNA
gcatggttctcttctctgca

SEQ ID NO: 638 - DNA, 39 bp, synthetic construct
  source 1..39: mol_type = other DNA
gagtagccacaaggctgccccagccaagatgatgtccgc

SEQ ID NO: 639 - DNA, 42 bp, synthetic construct
  source 1..42: mol_type = other DNA
tgaccaagttcatcctgggctcagggagctagtagaacctcg

SEQ ID NO: 640 - DNA, 18 bp, synthetic construct
  source 1..18: mol_type = other DNA
gtgccatgggcttcactg

SEQ ID NO: 641 - DNA, 18 bp, synthetic construct
  source 1..18: mol_type = other DNA
cgcaatgacagccgcaat

SEQ ID NO: 642 - DNA, 40 bp, synthetic construct
  source 1..40: mol_type = other DNA
tccacccccattggcaatggaatcgcctcgtcctccatag

SEQ ID NO: 643 - DNA, 40 bp, synthetic construct
  source 1..40: mol_type = other DNA
ggcagccttgtggctactctcccaggatgaacttggtcaa

SEQ ID NO: 644 - DNA, 24 bp, synthetic construct
  source 1..24: mol_type = other DNA
ggtgatgaaatcctggttagcgga

SEQ ID NO: 645 - DNA, 24 bp, synthetic construct
  source 1..24: mol_type = other DNA
cgctgatgcttgtttgaagatctc

SEQ ID NO: 646 - DNA, 22 bp, synthetic construct
  source 1..22: mol_type = other DNA
gctgacttccagcttgtcacct

SEQ ID NO: 647 - DNA, 23 bp, synthetic construct
  source 1..23: mol_type = other DNA
ctccagccaacagacctcaggaa

SEQ ID NO: 648 - DNA, 45 bp, synthetic construct
  source 1..45: mol_type = other DNA
aggctccttgttgacactcaccacgccctggtgcggctaaagtct

SEQ ID NO: 649 - DNA, 47 bp, synthetic construct
  source 1..47: mol_type = other DNA
tgacatcatggccacaaatggcgtcagagtctgcaagttcatcccct

SEQ ID NO: 650 - DNA, 24 bp, synthetic construct
  source 1..24: mol_type = other DNA
tgacatcatggccacaaatggcgt

SEQ ID NO: 651 - DNA, 23 bp, synthetic construct
  source 1..23: mol_type = other DNA
tgccgtggtgcattcctcctgta

SEQ ID NO: 652 - DNA, 24 bp, synthetic construct
  source 1..24: mol_type = other DNA
gttcatcccctctttcctgaggtc

SEQ ID NO: 653 - DNA, 24 bp, synthetic construct
  source 1..24: mol_type = other DNA
gtgcgactagcccctgtctatcaa

SEQ ID NO: 654 - DNA, 45 bp, synthetic construct
  source 1..45: mol_type = other DNA
agatctcaagcgcagagtctggtcatcaccaatgttctgcagcct

SEQ ID NO: 655 - DNA, 47 bp, synthetic construct
  source 1..47: mol_type = other DNA
agcgttttccagggcttcccagagcttcaagctaatgcttcatcctc

SEQ ID NO: 656 - DNA, 29 bp, synthetic construct
  source 1..29: mol_type = other DNA
ctacataagacctggaacacaggactgct

SEQ ID NO: 657 - DNA, 24 bp, synthetic construct
  source 1..24: mol_type = other DNA
atgccccttgttgagcctgaaacc

SEQ ID NO: 658 - DNA, 24 bp, synthetic construct
  source 1..24: mol_type = other DNA
aggagggcgataatcctcatggct

SEQ ID NO: 659 - DNA, 28 bp, synthetic construct
  source 1..28: mol_type = other DNA
cgtgggccagaagaccaggacatatcta

SEQ ID NO: 660 - DNA, 51 bp, synthetic construct
  source 1..51: mol_type = other DNA
gagggctaccaagagaatagcagcgccctgcctagcttgaggatctgtcac

SEQ ID NO: 661 - DNA, 53 bp, synthetic construct
  source 1..53: mol_type = other DNA
actccaggcaagaggtgatgaggcgagcagagcttttatcccatgcaaaggaa

SEQ ID NO: 662 - DNA, 24 bp, synthetic construct
  source 1..24: mol_type = other DNA
gggcactgttggcaagagtagcat

SEQ ID NO: 663 - DNA, 25 bp, synthetic construct
  source 1..25: mol_type = other DNA
caggaacctccatgccagatctcca

SEQ ID NO: 664 - DNA, 27 bp, synthetic construct
  source 1..27: mol_type = other DNA
actgaacattgtccacccacatgggaa

SEQ ID NO: 665 - DNA, 22 bp, synthetic construct
  source 1..22: mol_type = other DNA
ccctcggaggagacctggatca

SEQ ID NO: 666 - DNA, 49 bp, synthetic construct
  source 1..49: mol_type = other DNA
atgatgggcactgccacagcgtcaaccctgcatctttagacaaggccat

SEQ ID NO: 667 - DNA, 47 bp, synthetic construct
  source 1..47: mol_type = other DNA
tccatgggagaagagactggcccgtccagaacaggaagtgggtcctt

SEQ ID NO: 668 - DNA, 22 bp, synthetic construct
  source 1..22: mol_type = other DNA
gcccaagctctcctccacaggt

SEQ ID NO: 669 - DNA, 27 bp, synthetic construct
  source 1..27: mol_type = other DNA
cctacaatttgacggttgccatccact

SEQ ID NO: 670 - DNA, 21 bp, synthetic construct
  source 1..21: mol_type = other DNA
gtgggcgggttccagaaggtt

SEQ ID NO: 671 - DNA, 28 bp, synthetic construct
  source 1..28: mol_type = other DNA
gaggttcttctccttgtccacaatctgc

SEQ ID NO: 672 - DNA, 47 bp, synthetic construct
  source 1..47: mol_type = other DNA
ggattcagtagtgagctgggcagtgtctcagccccacttcacagagt

SEQ ID NO: 673 - DNA, 51 bp, synthetic construct
  source 1..51: mol_type = other DNA
atgccattcaatgttgcagaggggaagtaccagctgtagccaaaaagttgc

SEQ ID NO: 674 - DNA, 29 bp, synthetic construct
  source 1..29: mol_type = other DNA
ctcacagcctcacttctaaccttctggaa

SEQ ID NO: 675 - DNA, 30 bp, synthetic construct
  source 1..30: mol_type = other DNA
ggatgcattggggtatattgtctctcgacc

SEQ ID NO: 676 - DNA, 27 bp, synthetic construct
  source 1..27: mol_type = other DNA
cttcccctctgcaacattgaatggcat

SEQ ID NO: 677 - DNA, 30 bp, synthetic construct
  source 1..30: mol_type = other DNA
gaaagagtggatggcaaccgtcaaattgta

SEQ ID NO: 678 - DNA, 53 bp, synthetic construct
  source 1..53: mol_type = other DNA
gcagattgtggacaaggagaagaacctccactgcccagctcactactgaatcc

SEQ ID NO: 679 - DNA, 56 bp, synthetic construct
  source 1..56: mol_type = other DNA
gcaactttttggctacagctggtaggggtagcttgttgagttcctattgcatatcc

SEQ ID NO: 680 - DNA, 24 bp, synthetic construct
  source 1..24: mol_type = other DNA
tctcctgctatgcagcctctaacc

SEQ ID NO: 681 - DNA, 30 bp, synthetic construct
  source 1..30: mol_type = other DNA
ggtcaggttcacagagtccttatctcctgt

SEQ ID NO: 682 - DNA, 23 bp, synthetic construct
  source 1..23: mol_type = other DNA
tgtgtgctttgctggaatgttcc

SEQ ID NO: 683 - DNA, 22 bp, synthetic construct
  source 1..22: mol_type = other DNA
agtcactggctgcaacaggacc

SEQ ID NO: 684 - DNA, 56 bp, synthetic construct
  source 1..56: mol_type = other DNA
tcacagtgatgttagggataaagagctccacctgcacagtactcctggcttatcaa

SEQ ID NO: 685 - DNA, 57 bp, synthetic construct
  source 1..57: mol_type = other DNA
tagtggatcctatacctgccacgcccttagctcagtgactatgatcgtcttgactgt

SEQ ID NO: 686 - DNA, 32 bp, synthetic construct
  source 1..32: mol_type = other DNA
ctcattccagattccaaatgtcattctgtagc

SEQ ID NO: 687 - DNA, 20 bp, synthetic construct
  source 1..20: mol_type = other DNA
gtgtgggccaggctttccca

SEQ ID NO: 688 - DNA, 25 bp, synthetic construct
  source 1..25: mol_type = other DNA
caccatctgtttcccatctccagcc

SEQ ID NO: 689 - DNA, 22 bp, synthetic construct
  source 1..22: mol_type = other DNA
cccaagaggcccccaccatgaa

SEQ ID NO: 690 - DNA, 52 bp, synthetic construct
  source 1..52: mol_type = other DNA
ggtgaggctgtaaacttgggaaagacaaagggtccctgggtccatacacagc

SEQ ID NO: 691 - DNA, 46 bp, synthetic construct
  source 1..46: mol_type = other DNA
gtggaggctgttctgaggggccagaggaagagagtggctgtgaaac

SEQ ID NO: 692 - DNA, 22 bp, synthetic construct
  source 1..22: mol_type = other DNA
ggtccctgggtccatacacagc

SEQ ID NO: 693 - DNA, 22 bp, synthetic construct
  source 1..22: mol_type = other DNA
gctgtcgctacagaccacgtgt

SEQ ID NO: 694 - DNA, 25 bp, synthetic construct
  source 1..25: mol_type = other DNA
caccatctgtttcccatctccagcc

SEQ ID NO: 695 - DNA, 22 bp, synthetic construct
  source 1..22: mol_type = other DNA
ccaagaggcccccaccatgaag

SEQ ID NO: 696 - DNA, 51 bp, synthetic construct
  source 1..51: mol_type = other DNA
gctgtaaacttgggaaagacaaagcgctgcatcaagagcctgcagagactc

SEQ ID NO: 697 - DNA, 46 bp, synthetic construct; identical to SEQ ID NO: 691
  source 1..46: mol_type = other DNA

SEQ ID NO: 698 - DNA, 31 bp, synthetic construct
  source 1..31: mol_type = other DNA
ccgaacattcaaaactgtttctccaaagcgt

SEQ ID NO: 699 - DNA, 27 bp, synthetic construct
  source 1..27: mol_type = other DNA
tgatgaactgaacattacagtggccat

SEQ ID NO: 700 - DNA, 21 bp, synthetic construct
  source 1..21: mol_type = other DNA
cgcacactgcccatcgcttct

SEQ ID NO: 701 - DNA, 28 bp, synthetic construct
  source 1..28: mol_type = other DNA
ctttgggaacgggaagagtaaagtccac

SEQ ID NO: 702 - DNA, 52 bp, synthetic construct
  source 1..52: mol_type = other DNA
gagacgatgctgtagcggttggttgactgttttccaaagcagaagcactgga

SEQ ID NO: 703 - DNA, 41 bp, synthetic construct
  source 1..41: mol_type = other DNA
agacggtatgaagttggccacgcggctcctgcactgttgtc

SEQ ID NO: 704 - DNA, 22 bp, synthetic construct
  source 1..22: mol_type = other DNA
gcagtgtgcgaaccaaccgcta

SEQ ID NO: 705 - DNA, 25 bp, synthetic construct
  source 1..25: mol_type = other DNA
gccaggcagaagataaccagcatcc

SEQ ID NO: 706 - DNA, 23 bp, synthetic construct
  source 1..23: mol_type = other DNA
tcccgttcccaaagccatttgca

SEQ ID NO: 707 - DNA, 23 bp, synthetic construct
  source 1..23: mol_type = other DNA
ggtgagaaggggcaacggtacct

SEQ ID NO: 708 - DNA, 47 bp, synthetic construct
  source 1..47: mol_type = other DNA
gcactgttgtcgggtgtggactgtatgaagttggccaccatggcagt

SEQ ID NO: 709 - DNA, 50 bp, synthetic construct
  source 1..50: mol_type = other DNA
agaaagatggccactgtaatgttcagttcaccgccagcgaatgtccacac

SEQ ID NO: 710 - DNA, 20 bp, synthetic construct
  source 1..20: mol_type = other DNA
cggtccccaacgcatcatgc

SEQ ID NO: 711 - DNA, 30 bp, synthetic construct
  source 1..30: mol_type = other DNA
gacccactgatagactatgaaactttcccc

SEQ ID NO: 712 - DNA, 23 bp, synthetic construct
  source 1..23: mol_type = other DNA
ctcagagccaccactcctggaac

SEQ ID NO: 713 - DNA, 29 bp, synthetic construct
  source 1..29: mol_type = other DNA
agaagacaagaggtcaacacagactcatt

SEQ ID NO: 714 - DNA, 41 bp, synthetic construct
  source 1..41: mol_type = other DNA
tattggggggctgaacctggcggccgttgctccgactgtgt

SEQ ID NO: 715 - DNA, 59 bp, synthetic construct
  source 1..59: mol_type = other DNA
gggcagcttgataacacaaagaaaacagttgctgtagctatcattcacacggagtagaa

SEQ ID NO: 716 - DNA, 29 bp, synthetic construct
  source 1..29: mol_type = other DNA
gccattttgattccttttccggaacaagt

SEQ ID NO: 717 - DNA, 32 bp, synthetic construct; identical to SEQ ID NO: 43
  source 1..32: mol_type = other DNA

SEQ ID NO: 718 - DNA, 21 bp, synthetic construct
  source 1..21: mol_type = other DNA
ctccctggcatgatgcgttgg

SEQ ID NO: 719 - DNA, 21 bp, synthetic construct
  source 1..21: mol_type = other DNA
gccaggttcagccccccaata

SEQ ID NO: 720 - DNA, 42 bp, synthetic construct; identical to SEQ ID NO: 46
  source 1..42: mol_type = other DNA

SEQ ID NO: 721 - DNA, 52 bp, synthetic construct; identical to SEQ ID NO: 47
  source 1..52: mol_type = other DNA

SEQ ID NO: 722 - DNA, 21 bp, synthetic construct
  source 1..21: mol_type = other DNA
gccaggttcagccccccaata

SEQ ID NO: 723 - DNA, 29 bp, synthetic construct
  source 1..29: mol_type = other DNA
gatggaagataggtctgaaccttccaagc

SEQ ID NO: 724 - DNA, 32 bp, synthetic construct; identical to SEQ ID NO: 43
  source 1..32: mol_type = other DNA

SEQ ID NO: 725 - DNA, 30 bp, synthetic construct
  source 1..30: mol_type = other DNA
cttcttagaagcttaaacccctgtcccaat

SEQ ID NO: 726 - DNA, 61 bp, synthetic construct
  source 1..61: mol_type = other DNA
atgaaactttcccctgctgtagctatcattccaaagaaaacagtgtcaacgagtactacca

SEQ ID NO: 727 - DNA, 51 bp, synthetic construct
  source 1..51: mol_type = other DNA
cagtgggtcagaaaatggagttttatagcagacagcgggcgaacttgacgt

SEQ ID NO: 728 - DNA, 24 bp, synthetic construct
  source 1..24: mol_type = other DNA
atccaggcagtcatagactccgga

SEQ ID NO: 729 - DNA, 24 bp, synthetic construct
  source 1..24: mol_type = other DNA
atttgagccctgttgccagcagta

SEQ ID NO: 730 - DNA, 24 bp, synthetic construct
  source 1..24: mol_type = other DNA
cagggcaggagaagccactttgta

SEQ ID NO: 731 - DNA, 24 bp, synthetic construct
  source 1..24: mol_type = other DNA
ccacttgttgagcagtcccaagga

SEQ ID NO: 732 - DNA, 48 bp, synthetic construct; identical to SEQ ID NO: 34
  source 1..48: mol_type = other DNA

SEQ ID NO: 733 - DNA, 48 bp, synthetic construct; identical to SEQ ID NO: 35
  source 1..48: mol_type = other DNA

SEQ ID NO: 734 - DNA, 29 bp, synthetic construct
  source 1..29: mol_type = other DNA
tgtggagctgattaatgaagaagctgcca

SEQ ID NO: 735 - DNA, 25 bp, synthetic construct
  source 1..25: mol_type = other DNA
cgaaccgatccaccactcttggagt

SEQ ID NO: 736 - DNA, 25 bp, synthetic construct
  source 1..25: mol_type = other DNA
acactctccccagtggtagagctca

SEQ ID NO: 737 - DNA, 28 bp, synthetic construct
  source 1..28: mol_type = other DNA
acacagaaattccggaaactgctctcca

SEQ ID NO: 738 - DNA, 60 bp, synthetic construct
  source 1..60: mol_type = other DNA
aaagagcatctccaccatctctcttgtgattgttcgatagtcttctcatggactcttatg

SEQ ID NO: 739 - DNA, 56 bp, synthetic construct
  source 1..56: mol_type = other DNA
atgattctgacctgcagttagcaacctaacttcatctattggaggactaggctctt

SEQ ID NO: 740 - DNA, 28 bp, synthetic construct
  source 1..28: mol_type = other DNA
ccagccaatcatcagacattcctacgac

SEQ ID NO: 741 - DNA, 32 bp, synthetic construct
  source 1..32: mol_type = other DNA
aactgagggtttgttggtgattcagtgtaaga

SEQ ID NO: 742 - DNA, 57 bp, synthetic construct
  source 1..57: mol_type = other DNA
tttagaactctggaacctcagatgtatgaggaacatccgacacacagaagaacggtt

SEQ ID NO: 743 - DNA, 60 bp, synthetic construct
  source 1..60: mol_type = other DNA
tctggaaagatctacctccatagagacgtacggcaccgctatcaacctccattgactttc

SEQ ID NO: 744 - DNA, 18 bp, synthetic construct
  source 1..18: mol_type = other DNA
ctgtgcccatggtgctca

SEQ ID NO: 745 - DNA, 18 bp, synthetic construct
  source 1..18: mol_type = other DNA
ctccacccccattggcaa

SEQ ID NO: 746 - DNA, 22 bp, synthetic construct
  source 1..22: mol_type = other DNA
ctctccggattgaccaagttca

SEQ ID NO: 747 - DNA, 19 bp, synthetic construct
  source 1..19: mol_type = other DNA
cccctggcatggttctctt

SEQ ID NO: 748 - DNA, 40 bp, synthetic construct
  source 1..40: mol_type = other DNA
agtagccacaaggctgcccctccatagcagccaagatgat

SEQ ID NO: 749 - DNA, 41 bp, synthetic construct
  source 1..41: mol_type = other DNA
agtcactgggagcaactggagctagtagaacctcgcaatga

SEQ ID NO: 750 - DNA, 22 bp, synthetic construct
  source 1..22: mol_type = other DNA
tttggccaggattgctacagtt

SEQ ID NO: 751 - DNA, 19 bp, synthetic construct
  source 1..19: mol_type = other DNA
ccatggcactgagcaccat

SEQ ID NO: 752 - DNA, 19 bp, synthetic construct
  source 1..19: mol_type = other DNA
gcagccttgtggctactct

SEQ ID NO: 753 - DNA, 21 bp, synthetic construct
  source 1..21: mol_type = other DNA
gcccaggatgaacttggtcaa

SEQ ID NO: 754 - DNA, 42 bp, synthetic construct
  source 1..42: mol_type = other DNA
ttggctgctatggaggacgatgattggaggagttgtggccat

SEQ ID NO: 755 - DNA, 38 bp, synthetic construct
  source 1..38: mol_type = other DNA
ttgccaatgggggtggagtccagttgctcccagtgact

SEQ ID NO: 756 - DNA, 24 bp, synthetic construct
  source 1..24: mol_type = other DNA
acctgaggagagtgactagcttct

SEQ ID NO: 757 - DNA, 19 bp, synthetic construct
  source 1..19: mol_type = other DNA
ccgcaaccctgaagaccca

SEQ ID NO: 758 - DNA, 23 bp, synthetic construct
  source 1..23: mol_type = other DNA
gcagttcaaggtgacaagggcac

SEQ ID NO: 759 - DNA, 22 bp, synthetic construct
  source 1..22: mol_type = other DNA
gcctgctccatggaacccaaga

SEQ ID NO: 760 - DNA, 45 bp, synthetic construct; identical to SEQ ID NO: 17
  source 1..45: mol_type = other DNA

SEQ ID NO: 761 - DNA, 43 bp, synthetic construct; identical to SEQ ID NO: 18
  source 1..43: mol_type = other DNA

SEQ ID NO: 762 - DNA, 24 bp, synthetic construct
  source 1..24: mol_type = other DNA
acctgaggagagtgactagcttct

SEQ ID NO: 763 - DNA, 19 bp, synthetic construct
  source 1..19: mol_type = other DNA
ccgcaaccctgaagaccca

SEQ ID NO: 764 - DNA, 23 bp, synthetic construct
  source 1..23: mol_type = other DNA
gcagttcaaggtgacaagggcac

SEQ ID NO: 765 - DNA, 22 bp, synthetic construct
  source 1..22: mol_type = other DNA
gcctgctccatggaacccaaga

SEQ ID NO: 766 - DNA, 44 bp, synthetic construct
  source 1..44: mol_type = other DNA
gagcaactcagggtttcttccccccactgtggaagctcatggac

SEQ ID NO: 767 - DNA, 43 bp, synthetic construct; identical to SEQ ID NO: 18
  source 1..43: mol_type = other DNA

SEQ ID NO: 768 - DNA, 22 bp, synthetic construct
  source 1..22: mol_type = other DNA
cagttcaaggtgacaagggcac

SEQ ID NO: 769 - DNA, 21 bp, synthetic construct
  source 1..21: mol_type = other DNA
cttctgggctccaccctatgc

SEQ ID NO: 770 - DNA, 22 bp, synthetic construct
  source 1..22: mol_type = other DNA
cccacgtatgtaggcagcatcc

SEQ ID NO: 771 - DNA, 17 bp, synthetic construct
  source 1..17: mol_type = other DNA
gggcctcactgcgtgtt

SEQ ID NO: 772 - DNA, 42 bp, synthetic construct
  source 1..42: mol_type = other DNA
tcagtgccatgtggggtggacagccagcctcttgggttccat

SEQ ID NO: 773 - DNA, 45 bp, synthetic construct
  source 1..45: mol_type = other DNA
gagacttcgtggtgctggagcccctcaatgccagttagagtcacc

SEQ ID NO: 774 - DNA, 20 bp, synthetic construct
  source 1..20: mol_type = other DNA
ccgctgtcgtgcgtaccatg

SEQ ID NO: 775 - DNA, 21 bp, synthetic construct
  source 1..21: mol_type = other DNA
gccacccgacttgaagatggc

SEQ ID NO: 776 - DNA, 20 bp, synthetic construct
  source 1..20: mol_type = other DNA
tcaccacgctgcacaacctg

SEQ ID NO: 777 - DNA, 20 bp, synthetic construct
  source 1..20: mol_type = other DNA
gggcaccatcttttgcagcc

SEQ ID NO: 778 - DNA, 42 bp, synthetic construct
  source 1..42: mol_type = other DNA
gagctgagcatgcggaccagaccagcatcctgcacaacctct

SEQ ID NO: 779 - DNA, 41 bp, synthetic construct
  source 1..41: mol_type = other DNA
ccctgtggagtcggtcctgttcttggcgccctcctggtaca

SEQ ID NO: 780 - DNA, 27 bp, synthetic construct
  source 1..27: mol_type = other DNA
gccattgtgcatctcatcaactaccag

SEQ ID NO: 781 - DNA, 18 bp, synthetic construct
  source 1..18: mol_type = other DNA
gttgtgcaggatgctggt

SEQ ID NO: 782 - DNA, 21 bp, synthetic construct
  source 1..21: mol_type = other DNA
gggtcctcgtcgttgagcagt

SEQ ID NO: 783 - DNA, 20 bp, synthetic construct
  source 1..20: mol_type = other DNA
ccgctgtcgtgcgtaccatg

SEQ ID NO: 784 - DNA, 38 bp, synthetic construct
  source 1..38: mol_type = other DNA
caatcatggccgccttggtcactgcccgagctcaccaa

SEQ ID NO: 785 - DNA, 47 bp, synthetic construct
  source 1..47: mol_type = other DNA
tgaaccagctgtcgaagaaggagggtgtccaggtcgctggtattctg

SEQ ID NO: 786 - DNA, 20 bp, synthetic construct
  source 1..20: mol_type = other DNA
gaccacgccgagctcagttc

SEQ ID NO: 787 - DNA, 25 bp, synthetic construct
  source 1..25: mol_type = other DNA
ggtggttttcttgagcgtgtactgg

SEQ ID NO: 788 - DNA, 25 bp, synthetic construct
  source 1..25: mol_type = other DNA
gttcatcacctccatcgtggctact

SEQ ID NO: 789 - DNA, 23 bp, synthetic construct
  source 1..23: mol_type = other DNA
tacgactcgggtatccactcggg

SEQ ID NO: 790 - DNA, 41 bp, synthetic construct
  source 1..41: mol_type = other DNA
caccttgataggctgctccatcagcggtcaggccccatact

SEQ ID NO: 791 - DNA, 43 bp, synthetic construct
  source 1..43: mol_type = other DNA
actgagtggcagcagacatacaccctcctccatgatgcccttg

SEQ ID NO: 792 - DNA, 26 bp, synthetic construct
  source 1..26: mol_type = other DNA
agatgattggcaggtggatctaggat

SEQ ID NO: 793 - DNA, 20 bp, synthetic construct
  source 1..20: mol_type = other DNA
cacccaggaaggtaccacat

SEQ ID NO: 794 - DNA, 19 bp, synthetic construct
  source 1..19: mol_type = other DNA
ctccgggcattggccaaca

SEQ ID NO: 795 - DNA, 20 bp, synthetic construct
  source 1..20: mol_type = other DNA
caggccgggcacaacttcta

SEQ ID NO: 796 - DNA, 39 bp, synthetic construct
  source 1..39: mol_type = other DNA
agctcatccgacaggggatccggcttccaacatgtggca

SEQ ID NO: 797 - DNA, 55 bp, synthetic construct
  source 1..55: mol_type = other DNA
actatgtcaacaaacggaataccacgtgcctcttcaagtagctcatgtccacgtt

SEQ ID NO: 798 - DNA, 27 bp, synthetic construct
  source 1..27: mol_type = other DNA
atgacagcacttcgagagctcaagctt

SEQ ID NO: 799 - DNA, 26 bp, synthetic construct
  source 1..26: mol_type = other DNA
gctgcctgctccgaaatgtagacgat

SEQ ID NO: 800 - DNA, 27 bp, synthetic construct
  source 1..27: mol_type = other DNA
gctccactgctggtagtattcctggtt

SEQ ID NO: 801 - DNA, 28 bp, synthetic construct
  source 1..28: mol_type = other DNA
ggagcacatcacgtctgagtacacactt

SEQ ID NO: 802 - DNA, 40 bp, synthetic construct
  source 1..40: mol_type = other DNA
atggagcaaggctcgccctccgcactggcctgtgtcaagc

SEQ ID NO: 803 - DNA, 53 bp, synthetic construct
  source 1..53: mol_type = other DNA
atgtccacctataccctggaggagctcagccactgagaaggtatcctggttct

SEQ ID NO: 804 - DNA, 26 bp, synthetic construct
  source 1..26: mol_type = other DNA
gtgcagacaccttcagggatacaacc

SEQ ID NO: 805 - DNA, 23 bp, synthetic construct
  source 1..23: mol_type = other DNA
ccgtgtggaactcctggagagct

SEQ ID NO: 806 - DNA, 25 bp, synthetic construct
  source 1..25: mol_type = other DNA
gggtcccagtggtgcacaaattgtt

SEQ ID NO: 807 - DNA, 17 bp, synthetic construct
  source 1..17: mol_type = other DNA
agccccaacctctgggt

SEQ ID NO: 808 - DNA, 45 bp, synthetic construct
  source 1..45: mol_type = other DNA
atgtccagtcaggccccaccagggctgtgccagcaactggtattt

SEQ ID NO: 809 - DNA, 47 bp, synthetic construct
  source 1..47: mol_type = other DNA
atgccgaatctgcaccctccaatggggtcccttccctgctttgtagg

SEQ ID NO: 810 - DNA, 27 bp, synthetic construct
  source 1..27: mol_type = other DNA
acaccttcagggatacaaccccttcga

SEQ ID NO: 811 - DNA, 17 bp, synthetic construct
  source 1..17: mol_type = other DNA
gcccgtgtggaactcct

SEQ ID NO: 812 - DNA, 26 bp, synthetic construct
  source 1..26: mol_type = other DNA
tgggtcccagtggtgcacaaattgtt

SEQ ID NO: 813 - DNA, 17 bp, synthetic construct
  source 1..17: mol_type = other DNA
agccccaacctctgggt

SEQ ID NO: 814 - DNA, 46 bp, synthetic construct
  source 1..46: mol_type = other DNA
atgtccagtcaggccccaccaccagggctgtgccagcaactggtat

SEQ ID NO: 815 - DNA, 48 bp, synthetic construct
  source 1..48: mol_type = other DNA
atgccgaatctgcaccctccaatgtgggtcccttccctgctttgtagg

SEQ ID NO: 816 - DNA, 21 bp, synthetic construct
  source 1..21: mol_type = other DNA
gcccttctcgctggctcactt

SEQ ID NO: 817 - DNA, 27 bp, synthetic construct
  source 1..27: mol_type = other DNA
ggtacacgaacacaggaatgcacatca

SEQ ID NO: 818 - DNA, 25 bp, synthetic construct
  source 1..25: mol_type = other DNA
gggatgagcttgcataggaacctgc

SEQ ID NO: 819 - DNA, 28 bp, synthetic construct
  source 1..28: mol_type = other DNA
cgctgtcttgtggtattcaagccaatct

SEQ ID NO: 820 - DNA, 47 bp, synthetic construct
  source 1..47: mol_type = other DNA
tggcaaacatgttgaggacaatgatctctccagggacagtggcccta

SEQ ID NO: 821 - DNA, 55 bp, synthetic construct
  source 1..55: mol_type = other DNA
ttcctgcttactgccattagcctggacatccctacattgcgatgattctgacacc

SEQ ID NO: 822 - DNA, 24 bp, synthetic construct
  source 1..24: mol_type = other DNA
gcaggttcctatgcaagctcatcc

SEQ ID NO: 823 - DNA, 28 bp, synthetic construct
  source 1..28: mol_type = other DNA
tgagctggagagaccaaatttgtagcca

SEQ ID NO: 824 - DNA, 26 bp, synthetic construct
  source 1..26: mol_type = other DNA
tccaggctaatggcagtaagcaggaa

SEQ ID NO: 825 - DNA, 26 bp, synthetic construct
  source 1..26: mol_type = other DNA
tggtggcttttgtgatgtgcattcct

SEQ ID NO: 826 - DNA, 57 bp, synthetic construct
  source 1..57: mol_type = other DNA
agattggcttgaataccacaagacagcgccatcattgtcctcaacatgtttgccagt

SEQ ID NO: 827 - DNA, 59 bp, synthetic construct
  source 1..59: mol_type = other DNA
gtgtcagaatcatcgcaatgtagggatggctgtagtgaagatttcccggtacacgaaca

SEQ ID NO: 828 - DNA, 30 bp, synthetic construct
  source 1..30: mol_type = other DNA
ggcaaaacaacactggctaagaatttgcag

SEQ ID NO: 829 - DNA, 32 bp, synthetic construct
  source 1..32: mol_type = other DNA
gcttctattccatatagtgtcaaggggcttat

SEQ ID NO: 830 - DNA, 32 bp, synthetic construct
  source 1..32: mol_type = other DNA
ctatctcagactctggcttgaagaaatcatcc

SEQ ID NO: 831 - DNA, 27 bp, synthetic construct
  source 1..27: mol_type = other DNA
aagcgcaagacactctgtggtatcaac

SEQ ID NO: 832 - DNA, 59 bp, synthetic construct
  source 1..59: mol_type = other DNA
gcttcaagcacatcgtactgcaaaaatccacacctcccaaattgcagtgtcatatctca

SEQ ID NO: 833 - DNA, 51 bp, synthetic construct
  source 1..51: mol_type = other DNA
tgtcagccatttcctgctggatgggggaatttcctcagcactttcctggtc

SEQ ID NO: 834 - DNA, 21 bp, synthetic construct
  source 1..21: mol_type = other DNA
gtccatgcgggccatgatcga

SEQ ID NO: 835 - DNA, 22 bp, synthetic construct
  source 1..22: mol_type = other DNA
ggacgtagccctcctcggtgat

SEQ ID NO: 836 - DNA, 22 bp, synthetic construct
  source 1..22: mol_type = other DNA
cttgctctcgggcctgctgaat

SEQ ID NO: 837 - DNA, 20 bp, synthetic construct
  source 1..20: mol_type = other DNA
ggcatccgggcctgctatga

SEQ ID NO: 838 - DNA, 44 bp, synthetic construct
  source 1..44: mol_type = other DNA
ggctcatgaccaggctagcgtgccatggagcggctgcagattcc

SEQ ID NO: 839 - DNA, 50 bp, synthetic construct
  source 1..50: mol_type = other DNA
gtgaccacgtttgagaagcgctacgctgaatcgagcaggtggaattcccg

SEQ ID NO: 840 - DNA, 20 bp, synthetic construct
  source 1..20: mol_type = other DNA
ggcatccgggcctgctatga

SEQ ID NO: 841 - DNA, 27 bp, synthetic construct
  source 1..27: mol_type = other DNA
ccatttcttacgctctgacttctggcc

SEQ ID NO: 842 - DNA, 27 bp, synthetic construct
  source 1..27: mol_type = other DNA
ggacaggtagtacacggctgaatcgag

SEQ ID NO: 843 - DNA, 20 bp, synthetic construct
  source 1..20: mol_type = other DNA
tgcccaccactggcatcaac

SEQ ID NO: 844 - DNA, 40 bp, synthetic construct
  source 1..40: mol_type = other DNA
tcctcggtgatgcgctccagcgtcggcgggaattccacct

SEQ ID NO: 845 - DNA, 44 bp, synthetic construct
  source 1..44: mol_type = other DNA
cacagctcaggacgtgctccgatccgcaggttggttttctgcac

SEQ ID NO: 846 - DNA, 25 bp, synthetic construct
  source 1..25: mol_type = other DNA
ctgagtgtgagagcccggaagattt

SEQ ID NO: 847 - DNA, 25 bp, synthetic construct
  source 1..25: mol_type = other DNA
tgttcagcaccgacgtgaagtactt

SEQ ID NO: 848 - DNA, 25 bp, synthetic construct
  source 1..25: mol_type = other DNA
catcagatgagtcctgtttgccagg

SEQ ID NO: 849 - DNA, 21 bp, synthetic construct
  source 1..21: mol_type = other DNA
gcacctggagagcgaagacct

SEQ ID NO: 850 - DNA, 51 bp, synthetic construct
  source 1..51: mol_type = other DNA
tacgatttttctccctcctctgaactcttcagcagtgactccagcttcagc

SEQ ID NO: 851 - DNA, 43 bp, synthetic construct
  source 1..43: mol_type = other DNA
gaagagccgacagaggcagtgcttgatctccttgcgtagagcc

SEQ ID NO: 852 - DNA, 19 bp, synthetic construct
  source 1..19: mol_type = other DNA
cttccagccctgagcttcc

SEQ ID NO: 853 - DNA, 21 bp, synthetic construct
  source 1..21: mol_type = other DNA
tgccatgggacttgagcatct

SEQ ID NO: 854 - DNA, 20 bp, synthetic construct
  source 1..20: mol_type = other DNA
tccgggtctccaggtggaca

SEQ ID NO: 855 - DNA, 20 bp, synthetic construct
  source 1..20: mol_type = other DNA
cagtgccgcagcgtttcgag

SEQ ID NO: 856 - DNA, 44 bp, synthetic construct
  source 1..44: mol_type = other DNA
ttcactccttgtccaggcctctgtgggaggggcacacagactgt

SEQ ID NO: 857 - DNA, 43 bp, synthetic construct
  source 1..43: mol_type = other DNA
gaactgtcacgactggaagggcgtgggcctgcaacccacagtg

SEQ ID NO: 858 - DNA, 22 bp, synthetic construct
  source 1..22: mol_type = other DNA
gccaggattgctacagttgtga

SEQ ID NO: 859 - DNA, 23 bp, synthetic construct
  source 1..23: mol_type = other DNA
gctagtagaacctcgcaatgaca

SEQ ID NO: 860 - DNA, 19 bp, synthetic construct
  source 1..19: mol_type = other DNA
cggacatcatcttggctgc

SEQ ID NO: 861 - DNA, 22 bp, synthetic construct
  source 1..22: mol_type = other DNA
tctccggattgaccaagttcat

SEQ ID NO: 862 - DNA, 37 bp, synthetic construct
  source 1..37: mol_type = other DNA
agagtagccacaaggctgcagtgccatgggcttcact

SEQ ID NO: 863 - DNA, 37 bp, synthetic construct
  source 1..37: mol_type = other DNA
agtcactgggagcaactggcgcaatggcagacccaat

SEQ ID NO: 864 - DNA, 22 bp, synthetic construct
  source 1..22: mol_type = other DNA
ggaatcgcctcgtcctccatag

SEQ ID NO: 865 - DNA, 19 bp, synthetic construct
  source 1..19: mol_type = other DNA
ctcctcgctgggtcaggat

SEQ ID NO: 866 - DNA, 22 bp, synthetic construct
  source 1..22: mol_type = other DNA
gtcaatccggagagtccagttg

SEQ ID NO: 867 - DNA, 17 bp, synthetic construct
  source 1..17: mol_type = other DNA
cctcgccctgcagagaa

SEQ ID NO: 868 - DNA, 40 bp, synthetic construct
  source 1..40: mol_type = other DNA
caatggagcccaggatgaacttgggcagccttgtggctac

SEQ ID NO: 869 - DNA, 44 bp, synthetic construct
  source 1..44: mol_type = other DNA
attgcgaggttctactagctccctcttctcccctggcatggttc

SEQ ID NO: 870 - DNA, 20 bp, synthetic construct
  source 1..20: mol_type = other DNA
accccaagttcctggccatc

SEQ ID NO: 871 - DNA, 20 bp, synthetic construct
  source 1..20: mol_type = other DNA
tcccaccagcctccacaatg

SEQ ID NO: 872 - DNA, 21 bp, synthetic construct
  source 1..21: mol_type = other DNA
gatcagcttgctctcctggtt

SEQ ID NO: 873 - DNA, 21 bp, synthetic construct
  source 1..21: mol_type = other DNA
accaccagtcgtgtgctcaag

SEQ ID NO: 874 - DNA, 34 bp, synthetic construct; identical to SEQ ID NO: 29
  source 1..34: mol_type = other DNA

SEQ ID NO: 875 - DNA, 53 bp, synthetic construct; identical to SEQ ID NO: 30
  source 1..53: mol_type = other DNA

SEQ ID NO: 876 - DNA, 18 bp, synthetic construct
  source 1..18: mol_type = other DNA
cgacgggctgcaaaagat

SEQ ID NO: 877 - DNA, 19 bp, synthetic construct
  source 1..19: mol_type = other DNA
ctgggacacacggatagca

SEQ ID NO: 878 - DNA, 20 bp, synthetic construct
  source 1..20: mol_type = other DNA
gtggtgatggccaggaactt

SEQ ID NO: 879 - DNA, 23 bp, synthetic construct
  source 1..23: mol_type = other DNA
gttatgaaaagctgctctggacc

SEQ ID NO: 880 - DNA, 40 bp, synthetic construct
  source 1..40: mol_type = other DNA
atgatcagcttgctctcctgggtgcccctgctcaacaaga

SEQ ID NO: 881 - DNA, 39 bp, synthetic construct
  source 1..39: mol_type = other DNA
tcgtgcagatcatgcgtaacccttgagcacacgactggt

SEQ ID NO: 882 - DNA, 21 bp, synthetic construct
  source 1..21: mol_type = other DNA
caaccaggagagcaagctgat

SEQ ID NO: 883 - DNA, 19 bp, synthetic construct
  source 1..19: mol_type = other DNA
tggccacatctgagaggtt

SEQ ID NO: 884 - DNA, 25 bp, synthetic construct
  source 1..25: mol_type = other DNA
cagagcagcttttcataactgtagt

SEQ ID NO: 885 - DNA, 18 bp, synthetic construct
  source 1..18: mol_type = other DNA
agcacctgaccagcaaca

SEQ ID NO: 886 - DNA, 39 bp, synthetic construct
  source 1..39: mol_type = other DNA
ttgagcacacgactggtggcctcgtgcagatcatgcgta

SEQ ID NO: 887 - DNA, 38 bp, synthetic construct
  source 1..38: mol_type = other DNA
gcaataagcctgccattgtgacaggcagttctgcacca

SEQ ID NO: 888 - DNA, 20 bp, synthetic construct
  source 1..20: mol_type = other DNA
gctcctcttccggtttccat

SEQ ID NO: 889 - DNA, 21 bp, synthetic construct
  source 1..21: mol_type = other DNA
gacattggagggtgcagattc

SEQ ID NO: 890 - DNA, 20 bp, synthetic construct
  source 1..20: mol_type = other DNA
tcgaaggggttgtatccctg

SEQ ID NO: 891 - DNA, 20 bp, synthetic construct
  source 1..20: mol_type = other DNA
cccaagtacatggctgaagc

SEQ ID NO: 892 - DNA, 40 bp, synthetic construct
  source 1..40: mol_type = other DNA
aaataccagttgctggcacagcgaccagtgcagacacctt

SEQ ID NO: 893 - DNA, 39 bp, synthetic construct
  source 1..39: mol_type = other DNA
caatttgtgcaccactgggaggcatggatgtccagtcag

SEQ ID NO: 894 - DNA, 20 bp, synthetic construct
  source 1..20: mol_type = other DNA
cagggatacaaccccttcga

SEQ ID NO: 895 - DNA, 19 bp, synthetic construct
  source 1..19: mol_type = other DNA
cgtgtggaactcctggaga

SEQ ID NO: 896 - DNA, 20 bp, synthetic construct
  source 1..20: mol_type = other DNA
gtcccagtggtgcacaaatt

SEQ ID NO: 897 - DNA, 20 bp, synthetic construct
  source 1..20: mol_type = other DNA
atctgcaccctccaatgtcc

SEQ ID NO: 898 - DNA, 40 bp, synthetic construct
  source 1..40: mol_type = other DNA
tggacagcttcagccatgtctgtgccagcaactggtattt

SEQ ID NO: 899 - DNA, 40 bp, synthetic construct
  source 1..40: mol_type = other DNA
tgactggacatccatgccgctttgtagggacccagaggtt

SEQ ID NO: 900 - DNA, 19 bp, synthetic construct
  source 1..19: mol_type = other DNA
tacaaccccttcgaccagg

SEQ ID NO: 901 - DNA, 20 bp, synthetic construct
  source 1..20: mol_type = other DNA
tgtggaactcctggagagct

SEQ ID NO: 902 - DNA, 20 bp, synthetic construct
  source 1..20: mol_type = other DNA
tcccagtggtgcacaaattg

SEQ ID NO: 903 - DNA, 20 bp, synthetic construct
  source 1..20: mol_type = other DNA
atctgcaccctccaatgtcc

SEQ ID NO: 904 - DNA, 41 bp, synthetic construct
  source 1..41: mol_type = other DNA
gcttcagccatgtacttgggctgtgccagcaactggtattt

SEQ ID NO: 905 - DNA, 40 bp, synthetic construct
  source 1..40: mol_type = other DNA
ctgactggacatccatgcctttgtagggacccagaggttg

SEQ ID NO: 906 - DNA, 27 bp, synthetic construct
  source 1..27: mol_type = other DNA
ccaggaaatattaggaagctgtgattt

SEQ ID NO: 907 - DNA, 20 bp, synthetic construct
  source 1..20: mol_type = other DNA
ccatccagcaggaaatggct

SEQ ID NO: 908 - DNA, 25 bp, synthetic construct
  source 1..25: mol_type = other DNA
gtttctgcaaattcttagccagtgt

SEQ ID NO: 909 - DNA, 24 bp, synthetic construct
  source 1..24: mol_type = other DNA
ggatttttgcagtacgatgtgctt

SEQ ID NO: 910 - DNA, 52 bp, synthetic construct
  source 1..52: mol_type = other DNA
catcctgagatatgacactgcaattaacatttatcattggaatcagtggtgt

SEQ ID NO: 911 - DNA, 52 bp, synthetic construct
  source 1..52: mol_type = other DNA
cttcaagccagagtctgagatagaacatcattttttccatgttaagtgcttc

SEQ ID NO: 912 - DNA, 27 bp, synthetic construct
  source 1..27: mol_type = other DNA
aacatttatcattggaatcagtggtgt

SEQ ID NO: 913 - DNA, 25 bp, synthetic construct
  source 1..25: mol_type = other DNA
gtctgttgataccacagagtgtctt

SEQ ID NO: 914 - DNA, 25 bp, synthetic construct
  source 1..25: mol_type = other DNA
gagatatgacactgcaatttgggag

SEQ ID NO: 915 - DNA, 27 bp, synthetic construct
  source 1..27: mol_type = other DNA
cttaacatggaaaaaatgatgtcagcc

SEQ ID NO: 916 - DNA, 48 bp, synthetic construct
  source 1..48: mol_type = other DNA
agactctggcttgaagaaatcatccacaacactggctaagaatttgca

SEQ ID NO: 917 - DNA, 42 bp, synthetic construct
  source 1..42: mol_type = other DNA
ggatttttgcagtacgatgtgccgctttccatccagcaggaa

SEQ ID NO: 918 - DNA, 23 bp, synthetic construct
  source 1..23: mol_type = other DNA
acaacactggctaagaatttgca

SEQ ID NO: 919 - DNA, 28 bp, synthetic construct
  source 1..28: mol_type = other DNA
tcgatgattaaaatgggaatttcctcag

SEQ ID NO: 920 - DNA, 26 bp, synthetic construct
  source 1..26: mol_type = other DNA
tggcttgaagaaatcatcctgagata

SEQ ID NO: 921 - DNA, 20 bp, synthetic construct
  source 1..20: mol_type = other DNA
atggaaagcgcaagacactc

SEQ ID NO: 922 - DNA, 45 bp, synthetic construct
  source 1..45: mol_type = other DNA
tttccatgttaagtgcttcaagcgaaacacctcccaaattgcagt

SEQ ID NO: 923 - DNA, 46 bp, synthetic construct
  source 1..46: mol_type = other DNA
aatgatgtcagccatttcctgccactttcctggtctgttgatacca

SEQ ID NO: 924 - DNA, 24 bp, synthetic construct
  source 1..24: mol_type = other DNA
tgatttacgagatatgccagccaa

SEQ ID NO: 925 - DNA, 28 bp, synthetic construct
  source 1..28: mol_type = other DNA
tcagtgtaagatttgagttcatatgcag

SEQ ID NO: 926 - DNA, 25 bp, synthetic construct
  source 1..25: mol_type = other DNA
tctgtgtgtcggatgttctctttat

SEQ ID NO: 927 - DNA, 22 bp, synthetic construct
  source 1..22: mol_type = other DNA
gtacggctcctgttctctagaa

SEQ ID NO: 928 - DNA, 51 bp, synthetic construct
  source 1..51: mol_type = other DNA
tatgaggtagcgtaataaccgttctacgacaaagaactttttctgtacctg

SEQ ID NO: 929 - DNA, 49 bp, synthetic construct
  source 1..49: mol_type = other DNA
cagagttctaaatctggaaagatctaccaccgctatcaacctccattga

SEQ ID NO: 930 - DNA, 28 bp, synthetic construct
  source 1..28: mol_type = other DNA
gaactttttctgtacctgttaaacaaga

SEQ ID NO: 931 - DNA, 25 bp, synthetic construct
  source 1..25: mol_type = other DNA
ggtttgttggtgattcagtgtaaga

SEQ ID NO: 932 - DNA, 24 bp, synthetic construct
  source 1..24: mol_type = other DNA
ggtagcgtaataaccgttcttctg

SEQ ID NO: 933 - DNA, 26 bp, synthetic construct
  source 1..26: mol_type = other DNA
ctcctgttctctagaaagtcaatgga

SEQ ID NO: 934 - DNA, 54 bp, synthetic construct
  source 1..54: mol_type = other DNA
atttagaactctggaacctcagatgtagaagtgttaataaagagaacatccgac

SEQ ID NO: 935 - DNA, 45 bp, synthetic construct
  source 1..45: mol_type = other DNA
tggaaagatctacctccatagagacgatgcagcaccgctatcaac

SEQ ID NO: 936 - DNA, 23 bp, synthetic construct
  source 1..23: mol_type = other DNA
cacagaagaacggttattacgct

SEQ ID NO: 937 - DNA, 26 bp, synthetic construct
  source 1..26: mol_type = other DNA
tgcaaacttgggtactgagtaaatac

SEQ ID NO: 938 - DNA, 27 bp, synthetic construct
  source 1..27: mol_type = other DNA
ctctatggaggtagatctttccagatt

SEQ ID NO: 939 - DNA, 28 bp, synthetic construct
  source 1..28: mol_type = other DNA
gctgcatatgaactcaaatcttacactg

SEQ ID NO: 940 - DNA, 45 bp, synthetic construct
  source 1..45: mol_type = other DNA
ttctagagaacaggagccgtaccctcatacatctgaggttccaga

SEQ ID NO: 941 - DNA, 45 bp, synthetic construct
  source 1..45: mol_type = other DNA
tcaatggaggttgatagcggtcatcttggtgaaaactgagggttt

SEQ ID NO: 942 - DNA, 22 bp, synthetic construct
  source 1..22: mol_type = other DNA
cctagcttgaggatctgtcacc

SEQ ID NO: 943 - DNA, 24 bp, synthetic construct
  source 1..24: mol_type = other DNA
ccggcagaatactaatctgcaaga

SEQ ID NO: 944 - DNA, 22 bp, synthetic construct
  source 1..22: mol_type = other DNA
gctaccaagagaatagcagcga

SEQ ID NO: 945 - DNA, 24 bp, synthetic construct
  source 1..24: mol_type = other DNA
ggataaaagctctgctcttcaggt

SEQ ID NO: 946 - DNA, 40 bp, synthetic construct
  source 1..40: mol_type = other DNA
atcacctcttgcctggagtggccatgaggattatcgccct

SEQ ID NO: 947 - DNA, 45 bp, synthetic construct
  source 1..45: mol_type = other DNA
gaccaggacatatctatttcctttgcgaccatgccccttgttgag

SEQ ID NO: 948 - DNA, 21 bp, synthetic construct
  source 1..21: mol_type = other DNA
ttctcttggtagccctccagg

SEQ ID NO: 949 - DNA, 20 bp, synthetic construct
  source 1..20: mol_type = other DNA
cagaacgttaatcgacacgc

SEQ ID NO: 950 - DNA, 26 bp, synthetic construct
  source 1..26: mol_type = other DNA
gcaaaggaaatagatatgtcctggtc

SEQ ID NO: 951 - DNA, 20 bp, synthetic construct
  source 1..20: mol_type = other DNA
ggcgaacagaacttcgtgtt

SEQ ID NO: 952 - DNA, 44 bp, synthetic construct
  source 1..44: mol_type = other DNA
acctgaagagcagagcttttatcccactccaggcaagaggtgat

SEQ ID NO: 953 - DNA, 38 bp, synthetic construct
  source 1..38: mol_type = other DNA
ctcaacaaggggcatggtcccaccaatgaggcagttcc

SEQ ID NO: 954 - DNA, 20 bp, synthetic construct
  source 1..20: mol_type = other DNA
aagaggtgatgaggctccag

SEQ ID NO: 955 - DNA, 23 bp, synthetic construct
  source 1..23: mol_type = other DNA
ccagcatgacattctcttggaca

SEQ ID NO: 956 - DNA, 23 bp, synthetic construct
  source 1..23: mol_type = other DNA
gagcttttatcccatgcaaagga

SEQ ID NO: 957 - DNA, 19 bp, synthetic construct
  source 1..19: mol_type = other DNA
cttcgtgttgggaactgcc

SEQ ID NO: 958 - DNA, 47 bp, synthetic construct
  source 1..47: mol_type = other DNA
tgttgagcctgaaacctgaagagccagaagaccaggacatatctatt

SEQ ID NO: 959 - DNA, 39 bp, synthetic construct
  source 1..39: mol_type = other DNA
gtattctgccggcgaacagcagaacgttaatcgacacgc

SEQ ID NO: 960 - DNA, 23 bp, synthetic construct
  source 1..23: mol_type = other DNA
tgcagacaaagggaaaatcaacc

SEQ ID NO: 961 - DNA, 21 bp, synthetic construct
  source 1..21: mol_type = other DNA
agatctccacacgtccagaac

SEQ ID NO: 962 - DNA, 20 bp, synthetic construct
  source 1..20: mol_type = other DNA
ttgtccacccacatgggaat

SEQ ID NO: 963 - DNA, 20 bp, synthetic construct
  source 1..20: mol_type = other DNA
cggaggagacctggatcaca

SEQ ID NO: 964 - DNA, 45 bp, synthetic construct
  source 1..45: mol_type = other DNA
caggtccttttggacactgaacgcatctttagacaaggccatgtc

SEQ ID NO: 965 - DNA, 41 bp, synthetic construct
  source 1..41: mol_type = other DNA
ctccatgggagaagagactgggaagtgggtccttcctgaag

SEQ ID NO: 966 - DNA, 23 bp, synthetic construct
  source 1..23: mol_type = other DNA
gcatctttagacaaggccatgtc

SEQ ID NO: 967 - DNA, 19 bp, synthetic construct
  source 1..19: mol_type = other DNA
ccacctgagcatcgtccaa

SEQ ID NO: 968 - DNA, 21 bp, synthetic construct
  source 1..21: mol_type = other DNA
cgtgtcaggtccttttggaca

SEQ ID NO: 969 - DNA, 21 bp, synthetic construct
  source 1..21: mol_type = other DNA
cacttcctgttctggacgtgt

SEQ ID NO: 970 - DNA, 41 bp, synthetic construct
  source 1..41: mol_type = other DNA
tgtgatccaggtctcctccgccatgtgggtggacaatgttc

SEQ ID NO: 971 - DNA, 46 bp, synthetic construct
  source 1..46: mol_type = other DNA
aacaagataagacttcaggaaggaccggaacctccatgccagatct

SEQ ID NO: 972 - DNA, 20 bp, synthetic construct
  source 1..20: mol_type = other DNA
gacgtgtggagatctggcat

SEQ ID NO: 973 - DNA, 20 bp, synthetic construct
  source 1..20: mol_type = other DNA
tgtgcccacactcactatgg

SEQ ID NO: 974 - DNA, 19 bp, synthetic construct
  source 1..19: mol_type = other DNA
cacctgagcatcgtccaag

SEQ ID NO: 975 - DNA, 22 bp, synthetic construct
  source 1..22: mol_type = other DNA
gtgcaaagggaatgagtcttcc

SEQ ID NO: 976 - DNA, 44 bp, synthetic construct
  source 1..44: mol_type = other DNA
caaactctgcttctttgaatgctcagtgtgtgatgactcttggg

SEQ ID NO: 977 - DNA, 39 bp, synthetic construct
  source 1..39: mol_type = other DNA
gtcaggggactggaccgatatagcgtctggcaggacaat

SEQ ID NO: 978 - DNA, 17 bp, synthetic construct
  source 1..17: mol_type = other DNA
tgctcgaagcgttcctg

SEQ ID NO: 979 - DNA, 19 bp, synthetic construct
  source 1..19: mol_type = other DNA
ggggcagattgtggacaag

SEQ ID NO: 980 - DNA, 19 bp, synthetic construct
  source 1..19: mol_type = other DNA
cacgcactctgtgaagtgg

SEQ ID NO: 981 - DNA, 21 bp, synthetic construct
  source 1..21: mol_type = other DNA
gcccagctcactactgaatcc

SEQ ID NO: 982 - DNA, 37 bp, synthetic construct
  source 1..37: mol_type = other DNA
tgtgagcagaagcccctaagctctcctccacaggtga

SEQ ID NO: 983 - DNA, 40 bp, synthetic construct
  source 1..40: mol_type = other DNA
taaccttctggaacccgccccttcccctctgcaacattga

SEQ ID NO: 984 - DNA, 23 bp, synthetic construct
  source 1..23: mol_type = other DNA
gcctcacttctaaccttctggaa

SEQ ID NO: 985 - DNA, 19 bp, synthetic construct
  source 1..19: mol_type = other DNA
gtctctcgaccgctgtttg

SEQ ID NO: 986 - DNA, 21 bp, synthetic construct
  source 1..21: mol_type = other DNA
ccttcccctctgcaacattga

SEQ ID NO: 987 - DNA, 20 bp, synthetic construct
  source 1..20: mol_type = other DNA
ggaaagagtggatggcaacc

SEQ ID NO: 988 - DNA, 44 bp, synthetic construct
  source 1..44: mol_type = other DNA
attgtggacaaggagaagaacctgcccagctcactactgaatcc

SEQ ID NO: 989 - DNA, 49 bp, synthetic construct
  source 1..49: mol_type = other DNA
ccagcaactttttggctacagagttcctattgcatatcctacaatttga

SEQ ID NO: 990 - DNA, 21 bp, synthetic construct
  source 1..21: mol_type = other DNA
gcccagctcactactgaatcc

SEQ ID NO: 991 - DNA, 28 bp, synthetic construct
  source 1..28: mol_type = other DNA
gcttcttcattcacaagatctgacttta

SEQ ID NO: 992 - DNA, 21 bp, synthetic construct
  source 1..21: mol_type = other DNA
ctgtagccaaaaagttgctgg

SEQ ID NO: 993 - DNA, 21 bp, synthetic construct
  source 1..21: mol_type = other DNA
aacagcggtcgagagacaata

SEQ ID NO: 994 - DNA, 43 bp, synthetic construct
  source 1..43: mol_type = other DNA
ggttgccatccactctttccaggttcttctccttgtccacaat

SEQ ID NO: 995 - DNA, 48 bp, synthetic construct
  source 1..48: mol_type = other DNA
tcaaattgtaggatatgcaataggaacttgacgttctggatcagcagg

SEQ ID NO: 996 - DNA, 21 bp, synthetic construct
  source 1..21: mol_type = other DNA
tctgcagctggacaattcact

SEQ ID NO: 997 - DNA, 23 bp, synthetic construct
  source 1..23: mol_type = other DNA
aggctgtaaacttgggaaagaca

SEQ ID NO: 998 - DNA, 25 bp, synthetic construct
  source 1..25: mol_type = other DNA
catttggaatctggaatgaggcttt

SEQ ID NO: 999 - DNA, 20 bp, synthetic construct
  source 1..20: mol_type = other DNA
tcaagagcctgcagagactc

SEQ ID NO: 1000 - DNA, 45 bp, synthetic construct
  source 1..45: mol_type = other DNA
agttgatagcacgagggctacccagaggagaggaaagtgacaata

SEQ ID NO: 1001 - DNA, 39 bp, synthetic construct
  source 1..39: mol_type = other DNA
tgggtccatacacagcctgtctgtttcccatctccagcc

SEQ ID NO: 1002 - DNA, 24 bp, synthetic construct
  source 1..24: mol_type = other DNA
ccagaggagaggaaagtgacaata

SEQ ID NO: 1003 - DNA, 21 bp, synthetic construct
  source 1..21: mol_type = other DNA
ggctgtgaaacccttcatggt

SEQ ID NO: 1004 - DNA, 23 bp, synthetic construct
  source 1..23: mol_type = other DNA
acgagggctacagaatgacattt

SEQ ID NO: 1005 - DNA, 19 bp, synthetic construct
  source 1..19: mol_type = other DNA
gggctggagatgggaaaca

SEQ ID NO: 1006 - DNA, 42 bp, synthetic construct
  source 1..42: mol_type = other DNA
gtgtatggacccagggacctccaaagcctcattccagattcc

SEQ ID NO: 1007 - DNA, 42 bp, synthetic construct
  source 1..42: mol_type = other DNA
tgcatcaagagcctgcagaaggctgtaaacttgggaaagaca

SEQ ID NO: 1008 - DNA, 24 bp, synthetic construct
  source 1..24: mol_type = other DNA
cttgacctagctctcatgtctcaa

SEQ ID NO: 1009 - DNA, 23 bp, synthetic construct
  source 1..23: mol_type = other DNA
cacatgatagtagcattggcaca

SEQ ID NO: 1010 - DNA, 22 bp, synthetic construct
  source 1..22: mol_type = other DNA
ccacagaaagaaaacttgggca

SEQ ID NO: 1011 - DNA, 22 bp, synthetic construct
  source 1..22: mol_type = other DNA
cctcagggagaataccaggttt

SEQ ID NO: 1012 - DNA, 51 bp, synthetic construct
  source 1..51: mol_type = other DNA
gcatagtaaatctgctctcctttccggctcatctgttttgaatttctccta

SEQ ID NO: 1013 - DNA, 48 bp, synthetic construct
  source 1..48: mol_type = other DNA
ggcctgtcaataatcctgaatttactggtggaccgtttttcagtgtac

SEQ ID NO: 1014 - DNA, 22 bp, synthetic construct
  source 1..22: mol_type = other DNA
aaccactggatcttacatgcct

SEQ ID NO: 1015 - DNA, 24 bp, synthetic construct
  source 1..24: mol_type = other DNA
ctgctttggaaaacagtctgagtt

SEQ ID NO: 1016 - DNA, 27 bp, synthetic construct
  source 1..27: mol_type = other DNA
gttactttaatgactcagctgacatcc

SEQ ID NO: 1017 - DNA, 24 bp, synthetic construct
  source 1..24: mol_type = other DNA
gtgtgtcttcaccgaacattcaaa

SEQ ID NO: 1018 - DNA, 44 bp, synthetic construct
  source 1..44: mol_type = other DNA
accaaggtctgtctactgacatgccagcaacaggacatgttctc

SEQ ID NO: 1019 - DNA, 41 bp, synthetic construct
  source 1..41: mol_type = other DNA
aaccacaaggctcccagagtgcaaaacgctttggagaaaca

SEQ ID NO: 1020 - DNA, 23 bp, synthetic construct
  source 1..23: mol_type = other DNA
ctgcatgtcagtagacagacctt

SEQ ID NO: 1021 - DNA, 20 bp, synthetic construct
  source 1..20: mol_type = other DNA
ccatttgcaactgccatggt

SEQ ID NO: 1022 - DNA, 22 bp, synthetic construct
  source 1..22: mol_type = other DNA
tgcaaaacgctttggagaaaca

SEQ ID NO: 1023 - DNA, 18 bp, synthetic construct
  source 1..18: mol_type = other DNA
tgtgcgaaccaaccgcta

SEQ ID NO: 1024 - DNA, 47 bp, synthetic construct
  source 1..47: mol_type = other DNA
tctgctttggaaaacagtctgaggtgtgtcttcaccgaacattcaaa

SEQ ID NO: 1025 - DNA, 38 bp, synthetic construct
  source 1..38: mol_type = other DNA
agcagaagcgatgggcaggccaacttcataccgtcttc

SEQ ID NO: 1026 - DNA, 24 bp, synthetic construct
  source 1..24: mol_type = other DNA
gtgtgtcttcaccgaacattcaaa

SEQ ID NO: 1027 - DNA, 22 bp, synthetic construct
  source 1..22: mol_type = other DNA
gtgtggactttactcttcccgt

SEQ ID NO: 1028 - DNA, 19 bp, synthetic construct
  source 1..19: mol_type = other DNA
ggactccagtgcttctgct

SEQ ID NO: 1029 - DNA, 19 bp, synthetic construct
  source 1..19: mol_type = other DNA
cggtatgaagttggccacc

SEQ ID NO: 1030 - DNA, 39 bp, synthetic construct
  source 1..39: mol_type = other DNA
gttcgcacactgcccattgtttctccaaagcgttttgca

SEQ ID NO: 1031 - DNA, 41 bp, synthetic construct
  source 1..41: mol_type = other DNA
gctacagcatcgtctcttcagcaaagccatttgcaactgcc

SEQ ID NO: 1032 - DNA, 19 bp, synthetic construct
  source 1..19: mol_type = other DNA
atccaggcactgtccgtga

SEQ ID NO: 1033 - DNA, 21 bp, synthetic construct
  source 1..21: mol_type = other DNA
gacattggagggtgcagattc

SEQ ID NO: 1034 - DNA, 20 bp, synthetic construct
  source 1..20: mol_type = other DNA
tggtcgaaggggttgtatcc

SEQ ID NO: 1035 - DNA, 20 bp, synthetic construct
  source 1..20: mol_type = other DNA
cccaagtacatggctgaagc

SEQ ID NO: 1036 - DNA, 41 bp, synthetic construct
  source 1..41: mol_type = other DNA
aaataccagttgctggcacaggcaagtgcagacaccttcag

SEQ ID NO: 1037 - DNA, 39 bp, synthetic construct; identical to SEQ ID NO: 893
  source 1..39: mol_type = other DNA

SEQ ID NO: 1038 - DNA, 19 bp, synthetic construct
  source 1..19: mol_type = other DNA
tacaaccccttcgaccagg

SEQ ID NO: 1039 - DNA, 20 bp, synthetic construct
  source 1..20: mol_type = other DNA
tgtggaactcctggagagct

SEQ ID NO: 1040 - DNA, 20 bp, synthetic construct
  source 1..20: mol_type = other DNA
tcccagtggtgcacaaattg

SEQ ID NO: 1041 - DNA, 20 bp, synthetic construct
  source 1..20: mol_type = other DNA
atctgcaccctccaatgtcc

SEQ ID NO: 1042 - DNA, 41 bp, synthetic construct; identical to SEQ ID NO: 904
  source 1..41: mol_type = other DNA

SEQ ID NO: 1043 - DNA, 40 bp, synthetic construct; identical to SEQ ID NO: 905
  source 1..40: mol_type = other DNA

SEQ ID NO: 1044 - DNA, 25 bp, synthetic construct
  source 1..25: mol_type = other DNA
ggatgatttcttcaagccagagtct

SEQ ID NO: 1045 - DNA, 28 bp, synthetic construct
  source 1..28: mol_type = other DNA
catatggaatagtcaggaaatagcttct

SEQ ID NO: 1046 - DNA, 28 bp, synthetic construct
  source 1..28: mol_type = other DNA
acatcattttttccatgttaagtgcttc

SEQ ID NO: 1047 - DNA, 28 bp, synthetic construct
  source 1..28: mol_type = other DNA
ctgaggaaattcccattttaatcatcga

SEQ ID NO: 1048 - DNA, 42 bp, synthetic construct
  source 1..42: mol_type = other DNA
tgcgctttccatccagcaggatttttgcagtacgatgtgctt

SEQ ID NO: 1049 - DNA, 51 bp, synthetic construct
  source 1..51: mol_type = other DNA
cactctgtggtatcaacagaccaattccatatagtgtcaaggggcttataa

SEQ ID NO: 1050 - DNA, 24 bp, synthetic construct
  source 1..24: mol_type = other DNA
ggatttttgcagtacgatgtgctt

SEQ ID NO: 1051 - DNA, 22 bp, synthetic construct
  source 1..22: mol_type = other DNA
ccatcaaagtatcccggagagt

SEQ ID NO: 1052 - DNA, 23 bp, synthetic construct
  source 1..23: mol_type = other DNA
tggtctgttgataccacagagtg

SEQ ID NO: 1053 - DNA, 28 bp, synthetic construct
  source 1..28: mol_type = other DNA
agaagctatttcctgactattccatatg

SEQ ID NO: 1054 - DNA, 46 bp, synthetic construct
  source 1..46: mol_type = other DNA
aaaccttcgatgattaaaatgggaattttgctggatggaaagcgca

SEQ ID NO: 1055 - DNA, 51 bp, synthetic construct
  source 1..51: mol_type = other DNA
ttataagccccttgacactatatggaatctgatagacccttgtactcctcc

SEQ ID NO: 1056 - DNA, 23 bp, synthetic construct
  source 1..23: mol_type = other DNA
gctcttgctacataagacctgga

SEQ ID NO: 1057 - DNA, 19 bp, synthetic construct
  source 1..19: mol_type = other DNA
gcctgaaacctgaagagca

SEQ ID NO: 1058 - DNA, 19 bp, synthetic construct
  source 1..19: mol_type = other DNA
cgaggagggcgataatcct

SEQ ID NO: 1059 - DNA, 25 bp, synthetic construct
  source 1..25: mol_type = other DNA
gccagaagaccaggacatatctatt

SEQ ID NO: 1060 - DNA, 44 bp, synthetic construct
  source 1..44: mol_type = other DNA
agggctaccaagagaatagcagcctagcttgaggatctgtcacc

SEQ ID NO: 1061 - DNA, 43 bp, synthetic construct
  source 1..43: mol_type = other DNA
caagaggtgatgaggctccagagcttttatcccatgcaaagga

SEQ ID NO: 1062 - DNA, 20 bp, synthetic construct
  source 1..20: mol_type = other DNA
gccatgaggattatcgccct

SEQ ID NO: 1063 - DNA, 20 bp, synthetic construct
  source 1..20: mol_type = other DNA
gcagttcccaacacgaagtt

SEQ ID NO: 1064 - DNA, 20 bp, synthetic construct
  source 1..20: mol_type = other DNA
catcacctcttgcctggagt

SEQ ID NO: 1065 - DNA, 19 bp, synthetic construct
  source 1..19: mol_type = other DNA
aaggggcatggtctgctct

SEQ ID NO: 1066 - DNA, 48 bp, synthetic construct
  source 1..48: mol_type = other DNA
ccatgcaaaggaaatagatatgtcctgttctcttggtagccctccagg

SEQ ID NO: 1067 - DNA, 40 bp, synthetic construct
  source 1..40: mol_type = other DNA
tgctcttcaggtttcaggctgttcgccggcagaatactaa

SEQ ID NO: 1068 - DNA, 27 bp, synthetic construct
  source 1..27: mol_type = other DNA
caggacatatctatttcctttgcatgg

SEQ ID NO: 1069 - DNA, 26 bp, synthetic construct
  source 1..26: mol_type = other DNA
catttttctctattctgcaagctcag

SEQ ID NO: 1070 - DNA, 19 bp, synthetic construct
  source 1..19: mol_type = other DNA
gaccatgccccttgttgag

SEQ ID NO: 1071 - DNA, 21 bp, synthetic construct
  source 1..21: mol_type = other DNA
cgtgtcgattaacgttctgct

SEQ ID NO: 1072 - DNA, 43 bp, synthetic construct
  source 1..43: mol_type = other DNA
aacacgaagttctgttcgccaaaagctctgctcttcaggtttc

SEQ ID NO: 1073 - DNA, 39 bp, synthetic construct
  source 1..39: mol_type = other DNA
ggaactgcctcattggtggcgttcccagcatgacattct

SEQ ID NO: 1074 - DNA, 20 bp, synthetic construct
  source 1..20: mol_type = other DNA
gagactgaccagtgaagcca

SEQ ID NO: 1075 - DNA, 20 bp, synthetic construct
  source 1..20: mol_type = other DNA
cagcgtgtcaggtccttttg

SEQ ID NO: 1076 - DNA, 22 bp, synthetic construct
  source 1..22: mol_type = other DNA
acatgctactcttgccaacagt

SEQ ID NO: 1077 - DNA, 19 bp, synthetic construct
  source 1..19: mol_type = other DNA
caaggccatgtccattccc

SEQ ID NO: 1078 - DNA, 37 bp, synthetic construct
  source 1..37: mol_type = other DNA
tttgtctgcacagcccagcagggcgtctggaagtttt

SEQ ID NO: 1079 - DNA, 46 bp, synthetic construct
  source 1..46: mol_type = other DNA
ggaaaatcaaccctgcatctttagagacactgaacattgtccaccc

SEQ ID NO: 1080 - DNA, 17 bp, synthetic construct
  source 1..17: mol_type = other DNA
tgtggcagtgcccatca

SEQ ID NO: 1081 - DNA, 25 bp, synthetic construct
  source 1..25: mol_type = other DNA
ctgaccaaactctgcttctttgaat

SEQ ID NO: 1082 - DNA, 26 bp, synthetic construct
  source 1..26: mol_type = other DNA
ggtccttcctgaagtcttatcttgtt

SEQ ID NO: 1083 - DNA, 19 bp, synthetic construct
  source 1..19: mol_type = other DNA
gggacttggacgatgctca

SEQ ID NO: 1084 - DNA, 41 bp, synthetic construct
  source 1..41: mol_type = other DNA
acacgtccagaacaggaagtgcggaggagacctggatcaca

SEQ ID NO: 1085 - DNA, 40 bp, synthetic construct
  source 1..40: mol_type = other DNA
agatctggcatggaggttccaccacagccaagttgttgac

SEQ ID NO: 1086 - DNA, 20 bp, synthetic construct
  source 1..20: mol_type = other DNA
acccacttcctgttctggac

SEQ ID NO: 1087 - DNA, 18 bp, synthetic construct
  source 1..18: mol_type = other DNA
agcgtctggcaggacaat

SEQ ID NO: 1088 - DNA, 19 bp, synthetic construct
  source 1..19: mol_type = other DNA
ctgagcatcgtccaagtcc

SEQ ID NO: 1089 - DNA, 20 bp, synthetic construct
  source 1..20: mol_type = other DNA
gtcaggggactggaccgata

SEQ ID NO: 1090 - DNA, 41 bp, synthetic construct
  source 1..41: mol_type = other DNA
cagccaagttgttgacacaccgatctggcatggaggttcct

SEQ ID NO: 1091 - DNA, 48 bp, synthetic construct
  source 1..48: mol_type = other DNA
tgaaagcattcaaagaagcagagtttccacaaggaagactcattccct

SEQ ID NO: 1092 - DNA, 20 bp, synthetic construct
  source 1..20: mol_type = other DNA
caatctgccccagcaacttt

SEQ ID NO: 1093 - DNA, 22 bp, synthetic construct
  source 1..22: mol_type = other DNA
cgggtatacatggaactgtcca

SEQ ID NO: 1094 - DNA, 28 bp, synthetic construct
  source 1..28: mol_type = other DNA
agttcctattgcatatcctacaatttga

SEQ ID NO: 1095 - DNA, 26 bp, synthetic construct
  source 1..26: mol_type = other DNA
caggattctacaccctacaagtcata

SEQ ID NO: 1096 - DNA, 41 bp, synthetic construct
  source 1..41: mol_type = other DNA
tattgtctctcgaccgctgttggaaagagtggatggcaacc

SEQ ID NO: 1097 - DNA, 46 bp, synthetic construct
  source 1..46: mol_type = other DNA
cctgctgatccagaacgtcagttgcttcttcattcacaagatctga

SEQ ID NO: 1098 - DNA, 21 bp, synthetic construct
  source 1..21: mol_type = other DNA
aacagcggtcgagagacaata

SEQ ID NO: 1099 - DNA, 23 bp, synthetic construct
  source 1..23: mol_type = other DNA
gaggctctgattgtttatccacc

SEQ ID NO: 1100 - DNA, 26 bp, synthetic construct
  source 1..26: mol_type = other DNA
ccagttgcttcttcattcacaagatc

SEQ ID NO: 1101 - DNA, 19 bp, synthetic construct
  source 1..19: mol_type = other DNA
gtggccttcacctgtgaac

SEQ ID NO: 1102 - DNA, 47 bp, synthetic construct
  source 1..47: mol_type = other DNA
tgttgctggagatggaggggattctacaccctacaagtcataaagtc

SEQ ID NO: 1103 - DNA, 42 bp, synthetic construct
  source 1..42: mol_type = other DNA
tccaaccctgtggaggacaagtaggttgtgtcctgagtctca

SEQ ID NO: 1104 - DNA, 28 bp, synthetic construct
  source 1..28: mol_type = other DNA
ccctacaagtcataaagtcagatcttgt

SEQ ID NO: 1105 - DNA, 19 bp, synthetic construct
  source 1..19: mol_type = other DNA
ctgttgccattggacagct

SEQ ID NO: 1106 - DNA, 19 bp, synthetic construct
  source 1..19: mol_type = other DNA
ggcagctccgggtatacat

SEQ ID NO: 1107 - DNA, 21 bp, synthetic construct
  source 1..21: mol_type = other DNA
cacctgtgaacctgagactca

SEQ ID NO: 1108 - DNA, 44 bp, synthetic construct
  source 1..44: mol_type = other DNA
tccacagggttggagttgttggaatgaagaagcaactggacagt

SEQ ID NO: 1109 - DNA, 43 bp, synthetic construct
  source 1..43: mol_type = other DNA
gacaaggatgctgtggccttgaggctctgattgtttatccacc

SEQ ID NO: 1110 - DNA, 19 bp, synthetic construct
  source 1..19: mol_type = other DNA
ccaaccgctacagcatcgt

SEQ ID NO: 1111 - DNA, 21 bp, synthetic construct
  source 1..21: mol_type = other DNA
ggcagaagataaccagcatcc

SEQ ID NO: 1112 - DNA, 20 bp, synthetic construct
  source 1..20: mol_type = other DNA
caaagccatttgcaactgcc

SEQ ID NO: 1113 - DNA, 20 bp, synthetic construct
  source 1..20: mol_type = other DNA
caacggtacctcgcagacat

SEQ ID NO: 1114 - DNA, 41 bp, synthetic construct
  source 1..41: mol_type = other DNA
gtgtggactttactcttcccgtcggtatgaagttggccacc

SEQ ID NO: 1115 - DNA, 43 bp, synthetic construct
  source 1..43: mol_type = other DNA
aaagatggccactgtaatgttcagccagcgaatgtccacacac

SEQ ID NO: 1116 - DNA, 18 bp, synthetic construct
  source 1..18: mol_type = other DNA
caggagccgctttgtgaa

SEQ ID NO: 1117 - DNA, 20 bp, synthetic construct
  source 1..20: mol_type = other DNA
ctttgccctctttggatgca

SEQ ID NO: 1118 - DNA, 20 bp, synthetic construct
  source 1..20: mol_type = other DNA
gatgtctgcgaggtaccgtt

SEQ ID NO: 1119 - DNA, 19 bp, synthetic construct
  source 1..19: mol_type = other DNA
gctttcgtcctgtcatggc

SEQ ID NO: 1120 - DNA, 42 bp, synthetic construct
  source 1..42: mol_type = other DNA
agcgaatgtccacacacgtgatggccactgtaatgttcagtt

SEQ ID NO: 1121 - DNA, 43 bp, synthetic construct
  source 1..43: mol_type = other DNA
ggatgctggttatcttctgcccatggagcagagctatcaacca

SEQ ID NO: 1122 - DNA, 20 bp, synthetic construct
  source 1..20: mol_type = other DNA
cctggctttcgtcctgtcat

SEQ ID NO: 1123 - DNA, 21 bp, synthetic construct
  source 1..21: mol_type = other DNA
cacgattgactggaacaccac

SEQ ID NO: 1124 - DNA, 20 bp, synthetic construct
  source 1..20: mol_type = other DNA
ctttgccctctttggatgca

SEQ ID NO: 1125 - DNA, 23 bp, synthetic construct
  source 1..23: mol_type = other DNA
ggctatggtttcagatgtgtcac

SEQ ID NO: 1126 - DNA, 43 bp, synthetic construct
  source 1..43: mol_type = other DNA
gttgacctcggacacacaaggtgttttggttgatagctctgct

SEQ ID NO: 1127 - DNA, 47 bp, synthetic construct
  source 1..47: mol_type = other DNA
ttctccattgagacccagacaaccatgaaaacagcaattgggcattc

SEQ ID NO: 1128 - DNA, 25 bp, synthetic construct
  source 1..25: mol_type = other DNA
agatagtggtctagagcataagact

SEQ ID NO: 1129 - DNA, 23 bp, synthetic construct
  source 1..23: mol_type = other DNA
agagtaggtcagttgaattggtc

SEQ ID NO: 1130 - DNA, 47 bp, synthetic construct
  source 1..47: mol_type = other DNA
acacttagccacagtgaagtcatgacggaaacagagagagaacagaa

SEQ ID NO: 1131 - DNA, 40 bp, synthetic construct
  source 1..40: mol_type = other DNA
cgtggagacatccaggtgctgcagcagagaaagacgccat

SEQ ID NO: 1132 - DNA, 24 bp, synthetic construct
  source 1..24: mol_type = other DNA
ggcaagaaaatttgctgagctttc

SEQ ID NO: 1133 - DNA, 21 bp, synthetic construct
  source 1..21: mol_type = other DNA
aagccttcagctactgtctca

SEQ ID NO: 1134 - DNA, 24 bp, synthetic construct
  source 1..24: mol_type = other DNA
agagagaacagaagaagagaaagc

SEQ ID NO: 1135 - DNA, 18 bp, synthetic construct
  source 1..18: mol_type = other DNA
ctgggggctcattccatg

SEQ ID NO: 1136 - DNA, 46 bp, synthetic construct
  source 1..46: mol_type = other DNA
tccacgagtcctgtctggtccgcaaattttcttgccatacttcatg

SEQ ID NO: 1137 - DNA, 48 bp, synthetic construct
  source 1..48: mol_type = other DNA
gaagccttcagctactgtctcagttgagagtaggtcagttgaattggt

SEQ ID NO: 1138 - DNA, 22 bp, synthetic construct
  source 1..22: mol_type = other DNA
ccacacttagccacagtgaagt

SEQ ID NO: 1139 - DNA, 22 bp, synthetic construct
  source 1..22: mol_type = other DNA
ttgaagtttagcaatggcgtct

SEQ ID NO: 1140 - DNA, 25 bp, synthetic construct
  source 1..25: mol_type = other DNA
agagaaagctcagcaaattttcttg

SEQ ID NO: 1141 - DNA, 20 bp, synthetic construct
  source 1..20: mol_type = other DNA
aattactgggggctcattcc

SEQ ID NO: 1142 - DNA, 44 bp, synthetic construct
  source 1..44: mol_type = other DNA
gcacctggatgtctccacgagttcatgacttcactgtggctaag

SEQ ID NO: 1143 - DNA, 48 bp, synthetic construct; identical to SEQ ID NO: 1137
  source 1..48: mol_type = other DNA

SEQ ID NO: 1144 - DNA, 17 bp, synthetic construct
  source 1..17: mol_type = other DNA
cctgtctggtccccaca

SEQ ID NO: 1145 - DNA, 22 bp, synthetic construct
  source 1..22: mol_type = other DNA
ttttgaagtttagcaatggcgt

SEQ ID NO: 1146 - DNA, 19 bp, synthetic construct
  source 1..19: mol_type = other DNA
ggaccagacaggactcgtg

SEQ ID NO: 1147 - DNA, 15 bp, synthetic construct
  source 1..15: mol_type = other DNA
ggccagccacccaca

SEQ ID NO: 1148 - DNA, 41 bp, synthetic construct
  source 1..41: mol_type = other DNA
attggtctcagcagagaaagacgctccaggtgctgaagcct

SEQ ID NO: 1149 - DNA, 49 bp, synthetic construct
  source 1..49: mol_type = other DNA
gacctactctcacagccatggaatggcaatcccagtaaaaaagtaaggc

SEQ ID NO: 1150 - DNA, 25 bp, synthetic construct
  source 1..25: mol_type = other DNA
cttcaaaaaactgagacagtagctg

SEQ ID NO: 1151 - DNA, 20 bp, synthetic construct
  source 1..20: mol_type = other DNA
agcccccagtaattctctcc

SEQ ID NO: 1152 - DNA, 19 bp, synthetic construct
  source 1..19: mol_type = other DNA
gacatccaggtgctgaagc

SEQ ID NO: 1153 - DNA, 20 bp, synthetic construct
  source 1..20: mol_type = other DNA
aattgtgttcactgtccgct

SEQ ID NO: 1154 - DNA, 50 bp, synthetic construct
  source 1..50: mol_type = other DNA
atggctgtgagagtaggtcagttgagctactgtctcagttttttgaagtt

SEQ ID NO: 1155 - DNA, 40 bp, synthetic construct
  source 1..40: mol_type = other DNA
gcccccagtaattctctccatggcacagcaccagcccatt

SEQ ID NO: 1156 - DNA, 22 bp, synthetic construct
  source 1..22: mol_type = other DNA
gcagagaaagacgccattgcta

SEQ ID NO: 1157 - DNA, 25 bp, synthetic construct
  source 1..25: mol_type = other DNA
ttctcagccttacttttttactggg

SEQ ID NO: 1158 - DNA, 19 bp, synthetic construct
  source 1..19: mol_type = other DNA
catggctgctcttgctaca

SEQ ID NO: 1159 - DNA, 18 bp, synthetic construct
  source 1..18: mol_type = other DNA
tgtcctggtcttctggcc

SEQ ID NO: 1160 - DNA, 42 bp, synthetic construct
  source 1..42: mol_type = other DNA
tggggtgacagatcctcaagctagacctggaacacaggactg

SEQ ID NO: 1161 - DNA, 38 bp, synthetic construct
  source 1..38: mol_type = other DNA
ttatcgccctcctcgctgctgcctcatcacctcttgcc

SEQ ID NO: 1162 - DNA, 16 bp, synthetic construct
  source 1..16: mol_type = other DNA
gcgagcagagagggca

SEQ ID NO: 1163 - DNA, 16 bp, synthetic construct
  source 1..16: mol_type = other DNA
cctccaggtccgggca

SEQ ID NO: 1164 - DNA, 19 bp, synthetic construct
  source 1..19: mol_type = other DNA
catggctgctcttgctaca

SEQ ID NO: 1165 - DNA, 18 bp, synthetic construct
  source 1..18: mol_type = other DNA
tgtcctggtcttctggcc

SEQ ID NO: 1166 - DNA, 40 bp, synthetic construct
  source 1..40: mol_type = other DNA
ctcatggctggggtgacagatcgactgctgtctgccctct

SEQ ID NO: 1167 - DNA, 38 bp, synthetic construct; identical to SEQ ID NO: 1161
  source 1..38: mol_type = other DNA

SEQ ID NO: 1168 - DNA, 16 bp, synthetic construct
  source 1..16: mol_type = other DNA
ctaggcagggcgagca

SEQ ID NO: 1169 - DNA, 20 bp, synthetic construct
  source 1..20: mol_type = other DNA
attctcttggtagccctcca

SEQ ID NO: 1170 - DNA, 20 bp, synthetic construct
  source 1..20: mol_type = other DNA
gcctagcttgaggatctgtc

SEQ ID NO: 1171 - DNA, 18 bp, synthetic construct
  source 1..18: mol_type = other DNA
catgccccttgttgagcc

SEQ ID NO: 1172 - DNA, 39 bp, synthetic construct
  source 1..39: mol_type = other DNA
cacctcttgcctggagtgggtgaggattatcgccctcct

SEQ ID NO: 1173 - DNA, 40 bp, synthetic construct
  source 1..40: mol_type = other DNA
cgtgggccagaagaccaggatgaaacctgaagagcagagc

SEQ ID NO: 1174 - DNA, 21 bp, synthetic construct
  source 1..21: mol_type = other DNA
gctaccaagagaatagcagcg

SEQ ID NO: 1175 - DNA, 25 bp, synthetic construct
  source 1..25: mol_type = other DNA
catatctatttcctttgcatgggat

SEQ ID NO: 1176 - DNA, 15 bp, synthetic construct
  source 1..15: mol_type = other DNA
cagcgcgcagccttc

SEQ ID NO: 1177 - DNA, 25 bp, synthetic construct
  source 1..25: mol_type = other DNA
tgacatgcagagttactttaatgac

SEQ ID NO: 1178 - DNA, 43 bp, synthetic construct
  source 1..43: mol_type = other DNA
tgtaagatccagtggtttgtaaaaagcgggcgggctgggtctt

SEQ ID NO: 1179 - DNA, 42 bp, synthetic construct
  source 1..42: mol_type = other DNA
tgcctctgtaccccccacttctcagctgacatccagagaaca

SEQ ID NO: 1180 - DNA, 22 bp, synthetic construct
  source 1..22: mol_type = other DNA
gccaaagcaaaccagaattccc

SEQ ID NO: 1181 - DNA, 21 bp, synthetic construct
  source 1..21: mol_type = other DNA
actccatgtccccatgctcct

SEQ ID NO: 1182 - DNA, 15 bp, synthetic construct
  source 1..15: mol_type = other DNA
ggcgggctgggtctt

SEQ ID NO: 1183 - DNA, 23 bp, synthetic construct
  source 1..23: mol_type = other DNA
gttctaccaaggtctgtctactg

SEQ ID NO: 1184 - DNA, 43 bp, synthetic construct
  source 1..43: mol_type = other DNA
tggggggtacagaggcatgtaagtctggtttgctttggctcac

SEQ ID NO: 1185 - DNA, 45 bp, synthetic construct
  source 1..45: mol_type = other DNA
ccatgtccccatgctcctgccatgcagagttactttaatgactca

SEQ ID NO: 1186 - DNA, 22 bp, synthetic construct
  source 1..22: mol_type = other DNA
tccagtggtttgtaaaaagcga

SEQ ID NO: 1187 - DNA, 20 bp, synthetic construct
  source 1..20: mol_type = other DNA
gccagcaacaggacatgttc

SEQ ID NO: 1188 - DNA, 18 bp, synthetic construct
  source 1..18: mol_type = other DNA
gggctgggtcttgggaat

SEQ ID NO: 1189 - DNA, 19 bp, synthetic construct
  source 1..19: mol_type = other DNA
ggagccttgtggttctacc

SEQ ID NO: 1190 - DNA, 44 bp, synthetic construct
  source 1..44: mol_type = other DNA
agcatggggacatggagtggaaggctcactcgctttttacaaac

SEQ ID NO: 1191 - DNA, 40 bp, synthetic construct
  source 1..40: mol_type = other DNA
cctgcgccagcaacaggactgtctactgacatgcagagtt

SEQ ID NO: 1192 - DNA, 18 bp, synthetic construct
  source 1..18: mol_type = other DNA
tggggggtacagaggcat

SEQ ID NO: 1193 - DNA, 25 bp, synthetic construct
  source 1..25: mol_type = other DNA
catgttctctggatgtcagctgagt

SEQ ID NO: 1194 - DNA, 23 bp, synthetic construct
  source 1..23: mol_type = other DNA
gctttttacaaaccactggatct

SEQ ID NO: 1195 - DNA, 22 bp, synthetic construct
  source 1..22: mol_type = other DNA
tgaatgttcggtgaagacacac

SEQ ID NO: 1196 - DNA, 41 bp, synthetic construct
  source 1..41: mol_type = other DNA
agaacatgtcctgttgctggcgcctctgtaccccccacttc

SEQ ID NO: 1197 - DNA, 52 bp, synthetic construct
  source 1..52: mol_type = other DNA
agtaactctgcatgtcagtagacagaccacaccaaaaaaatgaggagagatg

SEQ ID NO: 1198 - DNA, 19 bp, synthetic construct
  source 1..19: mol_type = other DNA
agcatggggacatggagtg

SEQ ID NO: 1199 - DNA, 21 bp, synthetic construct
  source 1..21: mol_type = other DNA
ttggtagaaccacaaggctcc

SEQ ID NO: 1200 - DNA, 19 bp, synthetic construct
  source 1..19: mol_type = other DNA
tccactccatgtccccatg

SEQ ID NO: 1201 - DNA, 23 bp, synthetic construct
  source 1..23: mol_type = other DNA
tggaaaacagtctgagtttttgc

SEQ ID NO: 1202 - DNA, 44 bp, synthetic construct
  source 1..44: mol_type = other DNA
gtctgtctactgacatgcagagttacttctcctgcgccagcaac

SEQ ID NO: 1203 - DNA, 46 bp, synthetic construct
  source 1..46: mol_type = other DNA
gtagaaccacaaggctcccagagaaacagttttgaatgttcggtga

SEQ ID NO: 1204 - DNA, 22 bp, synthetic construct
  source 1..22: mol_type = other DNA
tgacatccagagaacatgtcct

SEQ ID NO: 1205 - DNA, 25 bp, synthetic construct
  source 1..25: mol_type = other DNA
cacccatctctcctcatttttttgg

SEQ ID NO: 1206 - DNA, 19 bp, synthetic construct
  source 1..19: mol_type = other DNA
ccactgggacccaagtaca

SEQ ID NO: 1207 - DNA, 17 bp, synthetic construct
  source 1..17: mol_type = other DNA
gcccgtgtggaactcct

SEQ ID NO: 1208 - DNA, 40 bp, synthetic construct
  source 1..40: mol_type = other DNA
agggtgcagattcggcatggaagctgtccagctgcagaga

SEQ ID NO: 1209 - DNA, 38 bp, synthetic construct
  source 1..38: mol_type = other DNA
ctcaaccccccagccccaacggagagctgcagcctcac

SEQ ID NO: 1210 - DNA, 17 bp, synthetic construct
  source 1..17: mol_type = other DNA
tgtccagtcaggcccca

SEQ ID NO: 1211 - DNA, 22 bp, synthetic construct
  source 1..22: mol_type = other DNA
gggtccctacaaagcagggaag

SEQ ID NO: 1212 - DNA, 19 bp, synthetic construct
  source 1..19: mol_type = other DNA
gggacccaagtacatggct

SEQ ID NO: 1213 - DNA, 16 bp, synthetic construct
  source 1..16: mol_type = other DNA
gctcctgcagcagcag

SEQ ID NO: 1214 - DNA, 39 bp, synthetic construct
  source 1..39: mol_type = other DNA
agagggggacattggagggtggagagtggtggggcctga

SEQ ID NO: 1215 - DNA, 41 bp, synthetic construct
  source 1..41: mol_type = other DNA
acctctgggtccctacaaagcagtggaactcctggagagct

SEQ ID NO: 1216 - DNA, 19 bp, synthetic construct
  source 1..19: mol_type = other DNA
attcggcatggatgtccag

SEQ ID NO: 1217 - DNA, 16 bp, synthetic construct
  source 1..16: mol_type = other DNA
ggggcgtggtgaggct

SEQ ID NO: 1218 - DNA, 18 bp, synthetic construct
  source 1..18: mol_type = other DNA
cagctgcagagagtggtg

SEQ ID NO: 1219 - DNA, 17 bp, synthetic construct
  source 1..17: mol_type = other DNA
gcccgtgtggaactcct

SEQ ID NO: 1220 - DNA, 39 bp, synthetic construct
  source 1..39: mol_type = other DNA
ggttgagagcagagggggacagggcctgactggacatcc

SEQ ID NO: 1221 - DNA, 37 bp, synthetic construct
  source 1..37: mol_type = other DNA
ccagccccaacctctgggtggagagctgcagcctcac

SEQ ID NO: 1222 - DNA, 18 bp, synthetic construct
  source 1..18: mol_type = other DNA
agggtgcagattcggcat

SEQ ID NO: 1223 - DNA, 20 bp, synthetic construct
  source 1..20: mol_type = other DNA
ccctacaaagcagggaaggg

SEQ ID NO: 1224 - DNA, 19 bp, synthetic construct
  source 1..19: mol_type = other DNA
acatccatgccgaatctgc

SEQ ID NO: 1225 - DNA, 16 bp, synthetic construct
  source 1..16: mol_type = other DNA
ggcatctccgggtgca

SEQ ID NO: 1226 - DNA, 41 bp, synthetic construct
  source 1..41: mol_type = other DNA
ttccctgctttgtagggacccatccaatgtccccctctgct

SEQ ID NO: 1227 - DNA, 39 bp, synthetic construct
  source 1..39: mol_type = other DNA
cagctctccaggagttccacacaggaaggccgaacctcg

SEQ ID NO: 1228 - DNA, 16 bp, synthetic construct
  source 1..16: mol_type = other DNA
gggctggggggttgag

SEQ ID NO: 1229 - DNA, 18 bp, synthetic construct
  source 1..18: mol_type = other DNA
cccctgctgctgcaggag

SEQ ID NO: 1230 - DNA, 20 bp, synthetic construct
  source 1..20: mol_type = other DNA
aaacagcagaggtgacagag

SEQ ID NO: 1231 - DNA, 19 bp, synthetic construct
  source 1..19: mol_type = other DNA
gcgggttccagaaggttag

SEQ ID NO: 1232 - DNA, 38 bp, synthetic construct
  source 1..38: mol_type = other DNA
tcctgctggccctgtcttcagtgctcgaagcgttcctg

SEQ ID NO: 1233 - DNA, 39 bp, synthetic construct
  source 1..39: mol_type = other DNA
gacaccatggggcacctctcagtgaggctgtgagcagaa

SEQ ID NO: 1234 - DNA, 18 bp, synthetic construct
  source 1..18: mol_type = other DNA
tgtggaggagagcttggg

SEQ ID NO: 1235 - DNA, 19 bp, synthetic construct
  source 1..19: mol_type = other DNA
agccccacttcacagagtg

SEQ ID NO: 1236 - DNA, 20 bp, synthetic construct
  source 1..20: mol_type = other DNA
cttcacagagtgcgtgtacc

SEQ ID NO: 1237 - DNA, 20 bp, synthetic construct
  source 1..20: mol_type = other DNA
ggttgccatccactctttcc

SEQ ID NO: 1238 - DNA, 41 bp, synthetic construct
  source 1..41: mol_type = other DNA
ttcagtagtgagctgggcagtgtgctcacagcctcacttct

SEQ ID NO: 1239 - DNA, 42 bp, synthetic construct
  source 1..42: mol_type = other DNA
atgttgcagaggggaaggaggtctttgtaccagctgtagcca

SEQ ID NO: 1240 - DNA, 18 bp, synthetic construct
  source 1..18: mol_type = other DNA
ggcgggttccagaaggtt

SEQ ID NO: 1241 - DNA, 23 bp, synthetic construct
  source 1..23: mol_type = other DNA
tcttctccttgtccacaatctgc

SEQ ID NO: 1242 - DNA, 20 bp, synthetic construct
  source 1..20: mol_type = other DNA
cccagctcactactgaatcc

SEQ ID NO: 1243 - DNA, 19 bp, synthetic construct
  source 1..19: mol_type = other DNA
gtctctcgaccgctgtttg

SEQ ID NO: 1244 - DNA, 41 bp, synthetic construct
  source 1..41: mol_type = other DNA
gccaaaaagttgctggggcagtcaatgttgcagaggggaag

SEQ ID NO: 1245 - DNA, 42 bp, synthetic construct
  source 1..42: mol_type = other DNA
aaggggaaagagtggatggcaatggggtagcttgttgagttc

SEQ ID NO: 1246 - DNA, 22 bp, synthetic construct
  source 1..22: mol_type = other DNA
tgtggacaaggagaagaacctc

SEQ ID NO: 1247 - DNA, 26 bp, synthetic construct
  source 1..26: mol_type = other DNA
ccgtcaaattgtaggatatgcaatag

SEQ ID NO: 1248 - DNA, 20 bp, synthetic construct
  source 1..20: mol_type = other DNA
ggggaaggaggttcttctcc

SEQ ID NO: 1249 - DNA, 20 bp, synthetic construct
  source 1..20: mol_type = other DNA
ctgtgtcattctgggtgacg

SEQ ID NO: 1250 - DNA, 41 bp, synthetic construct
  source 1..41: mol_type = other DNA
ggttgccatccactctttcccccaatctgccccagcaactt

SEQ ID NO: 1251 - DNA, 40 bp, synthetic construct
  source 1..40: mol_type = other DNA
aactcaacaagctaccccagggtggatcagcagggatgca

SEQ ID NO: 1252 - DNA, 21 bp, synthetic construct
  source 1..21: mol_type = other DNA
tttgtaccagctgtagccaaa

SEQ ID NO: 1253 - DNA, 16 bp, synthetic construct
  source 1..16: mol_type = other DNA
cccgcaaacagcggtc

SEQ ID NO: 1254 - DNA, 18 bp, synthetic construct
  source 1..18: mol_type = other DNA
tcctccctccagtgagtg

SEQ ID NO: 1255 - DNA, 20 bp, synthetic construct
  source 1..20: mol_type = other DNA
gggttgtatccctgaaggtg

SEQ ID NO: 1256 - DNA, 39 bp, synthetic construct
  source 1..39: mol_type = other DNA
gtgtcccatgtccccgctctggagtcctggttcctttgg

SEQ ID NO: 1257 - DNA, 40 bp, synthetic construct
  source 1..40: mol_type = other DNA
ggagttggtggtgggcagagacttcaagagcctttgctgt

SEQ ID NO: 1258 - DNA, 25 bp, synthetic construct
  source 1..25: mol_type = other DNA
ctgagattttcaacaccattctcca

SEQ ID NO: 1259 - DNA, 16 bp, synthetic construct
  source 1..16: mol_type = other DNA
atgctgctgggccact

SEQ ID NO: 1260 - DNA, 18 bp, synthetic construct
  source 1..18: mol_type = other DNA
tggtggtgggcagagatg

SEQ ID NO: 1261 - DNA, 18 bp, synthetic construct
  source 1..18: mol_type = other DNA
actctctgcagctggaca

SEQ ID NO: 1262 - DNA, 41 bp, synthetic construct
  source 1..41: mol_type = other DNA
tccctgaaggtgtctgcactgactgggccactttcagtgac

SEQ ID NO: 1263 - DNA, 39 bp, synthetic construct
  source 1..39: mol_type = other DNA
cagggctgtgccagcaactgcagccatgtacttgggtcc

SEQ ID NO: 1264 - DNA, 22 bp, synthetic construct
  source 1..22: mol_type = other DNA
agagcctttgctgtcatgtttt

SEQ ID NO: 1265 - DNA, 24 bp, synthetic construct
  source 1..24: mol_type = other DNA
gtatttaacaatttgtgcaccact

SEQ ID NO: 1266 - DNA, 18 bp, synthetic construct
  source 1..18: mol_type = other DNA
ttggtagccctccaggtc

SEQ ID NO: 1267 - DNA, 20 bp, synthetic construct
  source 1..20: mol_type = other DNA
caatgaggcagttcccaaca

SEQ ID NO: 1268 - DNA, 39 bp, synthetic construct
  source 1..39: mol_type = other DNA
atgtcctggtcttctggcccaccactccaggcaagaggt

SEQ ID NO: 1269 - DNA, 40 bp, synthetic construct
  source 1..40: mol_type = other DNA
ggtttcaggctcaacaaggggccgaagttctgttcgccgg

SEQ ID NO: 1270 - DNA, 17 bp, synthetic construct
  source 1..17: mol_type = other DNA
tggcctggagcctcatc

SEQ ID NO: 1271 - DNA, 23 bp, synthetic construct
  source 1..23: mol_type = other DNA
atggtctgctcttgcagattagt

SEQ ID NO: 1272 - DNA, 20 bp, synthetic construct
  source 1..20: mol_type = other DNA
agctctgctcttcaggtttc

SEQ ID NO: 1273 - DNA, 20 bp, synthetic construct
  source 1..20: mol_type = other DNA
gaagctaacaccaccgatga

SEQ ID NO: 1274 - DNA, 38 bp, synthetic construct
  source 1..38: mol_type = other DNA
agttctgttcgccggcagaaaggctcaacaaggggcat

SEQ ID NO: 1275 - DNA, 40 bp, synthetic construct
  source 1..40: mol_type = other DNA
cacatactgctgcacgcgtgtgcgttcccagcatgacatt

SEQ ID NO: 1276 - DNA, 21 bp, synthetic construct
  source 1..21: mol_type = other DNA
cgattaacgttctgctgtcca

SEQ ID NO: 1277 - DNA, 19 bp, synthetic construct
  source 1..19: mol_type = other DNA
aatctgcaagagcagacca

SEQ ID NO: 1278 - DNA, 20 bp, synthetic construct
  source 1..20: mol_type = other DNA
ggggaaggaggttcttctcc

SEQ ID NO: 1279 - DNA, 20 bp, synthetic construct
  source 1..20: mol_type = other DNA
ctgtgtcattctgggtgacg

SEQ ID NO: 1280 - DNA, 41 bp, synthetic construct
  source 1..41: mol_type = other DNA
ttgacggttgccatccactctttgccccagcaactttttgg

SEQ ID NO: 1281 - DNA, 39 bp, synthetic construct
  source 1..39: mol_type = other DNA
ctcaacaagctaccccagggctggatcagcagggatgca

SEQ ID NO: 1282 - DNA, 21 bp, synthetic construct
  source 1..21: mol_type = other DNA
tcccctttgtaccagctgtag

SEQ ID NO: 1283 - DNA, 17 bp, synthetic construct
  source 1..17: mol_type = other DNA
ccgcaaacagcggtcga

SEQ ID NO: 1284 - DNA, 20 bp, synthetic construct
  source 1..20: mol_type = other DNA
ccatctccagcaacaactcc

SEQ ID NO: 1285 - DNA, 20 bp, synthetic construct
  source 1..20: mol_type = other DNA
gtgctttgctggaatgttcc

SEQ ID NO: 1286 - DNA, 39 bp, synthetic construct
  source 1..39: mol_type = other DNA
ggaaatggtgggggtgtccgccctgtggaggacaaggat

SEQ ID NO: 1287 - DNA, 42 bp, synthetic construct
  source 1..42: mol_type = other DNA
acacctattaccgtccaggggcagtactgtgcaggtgggtta

SEQ ID NO: 1288 - DNA, 18 bp, synthetic construct
  source 1..18: mol_type = other DNA
tcacaggtgaaggccaca

SEQ ID NO: 1289 - DNA, 21 bp, synthetic construct
  source 1..21: mol_type = other DNA
aaacctcagcctctcctgcta

SEQ ID NO: 1290 - DNA, 18 bp, synthetic construct
  source 1..18: mol_type = other DNA
acctgacctgctccacaa

SEQ ID NO: 1291 - DNA, 18 bp, synthetic construct
  source 1..18: mol_type = other DNA
atgatggggtcgctttgg

SEQ ID NO: 1292 - DNA, 40 bp, synthetic construct
  source 1..40: mol_type = other DNA
tcctctccgaggacgggagagacactggaatctccatccg

SEQ ID NO: 1293 - DNA, 42 bp, synthetic construct
  source 1..42: mol_type = other DNA
accaccctcagcataaaccctgggttgaagacctcacaccaa

SEQ ID NO: 1294 - DNA, 22 bp, synthetic construct
  source 1..22: mol_type = other DNA
ctctggtttttgaagaaccaac

SEQ ID NO: 1295 - DNA, 19 bp, synthetic construct
  source 1..19: mol_type = other DNA
tcaagagggaggatgctgg

SEQ ID NO: 1296 - DNA, 20 bp, synthetic construct
  source 1..20: mol_type = other DNA
ttggtgtgaggtcttcaacc

SEQ ID NO: 1297 - DNA, 19 bp, synthetic construct
  source 1..19: mol_type = other DNA
gtgctctgtgagatcacgc

SEQ ID NO: 1298 - DNA, 38 bp, synthetic construct
  source 1..38: mol_type = other DNA
gcaatggccccaggtgagagaaagcgaccccatcatgc

SEQ ID NO: 1299 - DNA, 42 bp, synthetic construct
  source 1..42: mol_type = other DNA
gtgattggagtagtggccctggggtcttcccgaaatgcagaa

SEQ ID NO: 1300 - DNA, 27 bp, synthetic construct
  source 1..27: mol_type = other DNA
gtggtagagcattatagtttacgttca

SEQ ID NO: 1301 - DNA, 21 bp, synthetic construct
  source 1..21: mol_type = other DNA
ttgctctgatagcagtagccc

SEQ ID NO: 1302 - DNA, 20 bp, synthetic construct
  source 1..20: mol_type = other DNA
agagcacaaaccctcagtct

SEQ ID NO: 1303 - DNA, 20 bp, synthetic construct
  source 1..20: mol_type = other DNA
agtgatgagggtgagagact

SEQ ID NO: 1304 - DNA, 42 bp, synthetic construct
  source 1..42: mol_type = other DNA
tgtgggttgctgggcttcaaagtcaggaccactccaatgacc

SEQ ID NO: 1305 - DNA, 40 bp, synthetic construct
  source 1..40: mol_type = other DNA
gcctccccatccctaacagcagtgagcaggacaggtttca

SEQ ID NO: 1306 - DNA, 30 bp, synthetic construct
  source 1..30: mol_type = other DNA
aataagtaacttcattcatcttgttaggtg

SEQ ID NO: 1307 - DNA, 30 bp, synthetic construct
  source 1..30: mol_type = other DNA
cagaaataatttattcagaagtaaaaaagc

SEQ ID NO: 1308 - DNA, 20 bp, synthetic construct
  source 1..20: mol_type = other DNA
ctcctcttccggtttccatc

SEQ ID NO: 1309 - DNA, 18 bp, synthetic construct
  source 1..18: mol_type = other DNA
agggtgcagattcggcat

SEQ ID NO: 1310 - DNA, 36 bp, synthetic construct
  source 1..36: mol_type = other DNA
ttgctggcacagccctggtcccgaccagtgcagaca

SEQ ID NO: 1311 - DNA, 40 bp, synthetic construct
  source 1..40: mol_type = other DNA
caatttgtgcaccactgggaccaggccccaccactctctg

SEQ ID NO: 1312 - DNA, 20 bp, synthetic construct
  source 1..20: mol_type = other DNA
aggggttgtatccctgaagg

SEQ ID NO: 1313 - DNA, 23 bp, synthetic construct
  source 1..23: mol_type = other DNA
caagtacatggctgaagctgtcc

SEQ ID NO: 1314 - DNA, 20 bp, synthetic construct
  source 1..20: mol_type = other DNA
ggatgcgtattgccctatgg

SEQ ID NO: 1315 - DNA, 18 bp, synthetic construct
  source 1..18: mol_type = other DNA
ctgaaagtggcccagcag

SEQ ID NO: 1316 - DNA, 40 bp, synthetic construct
  source 1..40: mol_type = other DNA
gcactcactggagggaggaaaacaacagtgagtggcgaca

SEQ ID NO: 1317 - DNA, 40 bp, synthetic construct
  source 1..40: mol_type = other DNA
ggagtcctggttcctttggtggtcccctgtgtcccatgtc

SEQ ID NO: 1318 - DNA, 21 bp, synthetic construct
  source 1..21: mol_type = other DNA
ggacagctccataacagctca

SEQ ID NO: 1319 - DNA, 24 bp, synthetic construct
  source 1..24: mol_type = other DNA
agaatggtgttgaaaatctcagcc

SEQ ID NO: 1320 - DNA, 20 bp, synthetic construct
  source 1..20: mol_type = other DNA
ggagctgtccagacttttcc

SEQ ID NO: 1321 - DNA, 20 bp, synthetic construct
  source 1..20: mol_type = other DNA
aaggtgtctgcactgacttc

SEQ ID NO: 1322 - DNA, 40 bp, synthetic construct
  source 1..40: mol_type = other DNA
cgccgcaaaggctgagattttcgtgagtgccagctgtcct

SEQ ID NO: 1323 - DNA, 40 bp, synthetic construct
  source 1..40: mol_type = other DNA
ggacacaggggagttggtggaagagcctttgctgtcatgt

SEQ ID NO: 1324 - DNA, 19 bp, synthetic construct
  source 1..19: mol_type = other DNA
ggaaccaggactccatgca

SEQ ID NO: 1325 - DNA, 17 bp, synthetic construct
  source 1..17: mol_type = other DNA
tgggcagagatgctgct

SEQ ID NO: 1326 - DNA, 18 bp, synthetic construct
  source 1..18: mol_type = other DNA
tcctccctccagtgagtg

SEQ ID NO: 1327 - DNA, 20 bp, synthetic construct
  source 1..20: mol_type = other DNA
aggtgtctgcactgacttca

SEQ ID NO: 1328 - DNA, 41 bp, synthetic construct
  source 1..41: mol_type = other DNA
cgccgcaaaggctgagattttcgctgtccttgcatggagtc

SEQ ID NO: 1329 - DNA, 41 bp, synthetic construct
  source 1..41: mol_type = other DNA
catgggacacaggggagttggagagcctttgctgtcatgtt

SEQ ID NO: 1330 - DNA, 22 bp, synthetic construct
  source 1..22: mol_type = other DNA
cattctccaccaaaggaaccag

SEQ ID NO: 1331 - DNA, 18 bp, synthetic construct
  source 1..18: mol_type = other DNA
tggtgggcagagatgctg

SEQ ID NO: 1332 - DNA, 41 bp, synthetic construct
  source 1..41: mol_type = other DNA
gtgtctgtgttgtgctggcgaataactgccccctgtgtgag

SEQ ID NO: 1333 - DNA, 20 bp, synthetic construct
  source 1..20: mol_type = other DNA
cagaaggaaccaggaaacga

SEQ ID NO: 1334 - DNA, 38 bp, synthetic construct
  source 1..38: mol_type = other DNA
gagccgaccactcatgcagccaccagcatgtggcgatc

SEQ ID NO: 1335 - DNA, 19 bp, synthetic construct
  source 1..19: mol_type = other DNA
gcacttgtaaggcctctcg

SEQ ID NO: 1336 - DNA, 21 bp, synthetic construct
  source 1..21: mol_type = other DNA
gtgctgggtagtgcaaatctt

SEQ ID NO: 1337 - DNA, 21 bp, synthetic construct
  source 1..21: mol_type = other DNA
atctgcggaaagtcactgagc

SEQ ID NO: 1338 - DNA, 42 bp, synthetic construct
  source 1..42: mol_type = other DNA
tcgtggaagccagcaggaattgaacttcccagggatgcaatg

SEQ ID NO: 1339 - DNA, 18 bp, synthetic construct
  source 1..18: mol_type = other DNA
acgcgcttgtccacaaac

SEQ ID NO: 1340 - DNA, 40 bp, synthetic construct
  source 1..40: mol_type = other DNA
ctcgcatttctcaggcctggtgtcgcagacaaaacggtgt

SEQ ID NO: 1341 - DNA, 18 bp, synthetic construct
  source 1..18: mol_type = other DNA
gggaacgccttgtcacag

SEQ ID NO: 1342 - DNA, 23 bp, synthetic construct
  source 1..23: mol_type = other DNA
gggttgttctgtatgaaaggtct

SEQ ID NO: 1343 - DNA, 19 bp, synthetic construct
  source 1..19: mol_type = other DNA
gcgaaacaaacctgcggag

SEQ ID NO: 1344 - DNA, 40 bp, synthetic construct
  source 1..40: mol_type = other DNA
ctgtgcatgttcccattggtggacatgctggtgcactctg

SEQ ID NO: 1345 - DNA, 18 bp, synthetic construct
  source 1..18: mol_type = other DNA
aagtcactgagctcggcc

SEQ ID NO: 1346 - DNA, 41 bp, synthetic construct
  source 1..41: mol_type = other DNA
aaggaccctaacagtgccacagtcttggaggacaatcgcct

SEQ ID NO: 1347 - DNA, 20 bp, synthetic construct
  source 1..20: mol_type = other DNA
ggcatcgtgactcagtttcc

SEQ ID NO: 1348 - DNA, 20 bp, synthetic construct
  source 1..20: mol_type = other DNA
cacagtgcacttgtaaggcc

SEQ ID NO: 1349 - DNA, 20 bp, synthetic construct
  source 1..20: mol_type = other DNA
ccacagcccctccatctcct

SEQ ID NO: 1350 - DNA, 41 bp, synthetic construct
  source 1..41: mol_type = other DNA
aatcttctcacacagggggcagggattggcagaaggaacca

SEQ ID NO: 1351 - DNA, 18 bp, synthetic construct
  source 1..18: mol_type = other DNA
cgaagcctccaggaccaa

SEQ ID NO: 1352 - DNA, 41 bp, synthetic construct
  source 1..41: mol_type = other DNA
cacattcgccagcacaacacaggagctcagtgactttccgc

SEQ ID NO: 1353 - DNA, 18 bp, synthetic construct
  source 1..18: mol_type = other DNA
atgtggcgatcgagggag

SEQ ID NO: 1354 - DNA, 23 bp, synthetic construct
  source 1..23: mol_type = other DNA
aagacttctcctctttcgtttcc

SEQ ID NO: 1355 - DNA, 19 bp, synthetic construct
  source 1..19: mol_type = other DNA
acactggaggagccgacca

SEQ ID NO: 1356 - DNA, 42 bp, synthetic construct
  source 1..42: mol_type = other DNA
ttcagggagagaccgaggagagctttgcacatccagaaacgc

SEQ ID NO: 1357 - DNA, 18 bp, synthetic construct
  source 1..18: mol_type = other DNA
ggaggaggggaagtgact

SEQ ID NO: 1358 - DNA, 39 bp, synthetic construct
  source 1..39: mol_type = other DNA
ccggtccccaacgcatcatgctcctggaacacacagtcg

SEQ ID NO: 1359 - DNA, 18 bp, synthetic construct
  source 1..18: mol_type = other DNA
cacacctcagagccacca

SEQ ID NO: 1360 - DNA, 22 bp, synthetic construct
  source 1..22: mol_type = other DNA
tgcaaacttgttccggaaaagg

SEQ ID NO: 1361 - DNA, 17 bp, synthetic construct
  source 1..17: mol_type = other DNA
agggagccaagcgtcct

SEQ ID NO: 1362 - DNA, 42 bp, synthetic construct
  source 1..42: mol_type = other DNA
aatcttctcacacagggggcagggattggcagaaggaaccag

SEQ ID NO: 1363 - DNA, 18 bp, synthetic construct
  source 1..18: mol_type = other DNA
cgaagcctccaggaccaa

SEQ ID NO: 1364 - DNA, 41 bp, synthetic construct; identical to SEQ ID NO: 1352
  source 1..41: mol_type = other DNA

SEQ ID NO: 1365 - DNA, 18 bp, synthetic construct
  source 1..18: mol_type = other DNA
gagtgcaccagcatgtgg

SEQ ID NO: 1366 - DNA, 24 bp, synthetic construct
  source 1..24: mol_type = other DNA
ggaagacttctcctctttcgtttc

SEQ ID NO: 1367 - DNA, 19 bp, synthetic construct
  source 1..19: mol_type = other DNA
acactggaggagccgacca

SEQ ID NO: 1368 - DNA, 45 bp, synthetic construct; identical to SEQ ID NO: 54
  source 1..45: mol_type = other DNA

SEQ ID NO: 1369 - DNA, 40 bp, synthetic construct; identical to SEQ ID NO: 56
  source 1..40: mol_type = other DNA

SEQ ID NO: 1370 - DNA, 18 bp, synthetic construct
  source 1..18: mol_type = other DNA
ctgaaaaggcctgtagcc

SEQ ID NO: 1371 - DNA, 19 bp, synthetic construct
  source 1..19: mol_type = other DNA
cagagtgacactgaacaga

SEQ ID NO: 1372 - DNA, 22 bp, synthetic construct
  source 1..22: mol_type = other DNA
tcagcgtatccaattcagcaat

SEQ ID NO: 1373 - DNA, 20 bp, synthetic construct
  source 1..20: mol_type = other DNA
gagacgaaggagacgctggg

SEQ ID NO: 1374 - DNA, 18 bp, synthetic construct
  source 1..18: mol_type = other DNA
gcaggccctctttccatc

SEQ ID NO: 1375 - DNA, 18 bp, synthetic construct
  source 1..18: mol_type = other DNA
tgttcccgtgcatcgaag

SEQ ID NO: 1376 - DNA, 39 bp, synthetic construct
  source 1..39: mol_type = other DNA
tagaagttgtgcccggcctgctgtcggatgagctggtca

SEQ ID NO: 1377 - DNA, 40 bp, synthetic construct
  source 1..40: mol_type = other DNA
tgtggtaccttcctgggtgggcagcttcaggtcctcggta

SEQ ID NO: 1378 - DNA, 19 bp, synthetic construct
  source 1..19: mol_type = other DNA
tctgtgagcctggctacag

SEQ ID NO: 1379 - DNA, 19 bp, synthetic construct
  source 1..19: mol_type = other DNA
tggccacccatcatctctc

SEQ ID NO: 1380 - DNA, 41 bp, synthetic construct; identical to SEQ ID NO: 1
  source 1..41: mol_type = other DNA

SEQ ID NO: 1381 - DNA, 41 bp, synthetic construct
  source 1..41: mol_type = other DNA
ccgtggagggagctttctctgggtgacgtgttggtacactc

SEQ ID NO: 1382 - DNA, 18 bp, synthetic construct
  source 1..18: mol_type = other DNA
ggctgtggaagccatctc

SEQ ID NO: 1383 - DNA, 20 bp, synthetic construct
  source 1..20: mol_type = other DNA
ggattcatagaggccaccag

SEQ ID NO: 1384 - DNA, 39 bp, synthetic construct
  source 1..39: mol_type = other DNA
acagcatgtgagcaggtcctctgaccggatctgcatcca

SEQ ID NO: 1385 - DNA, 41 bp, synthetic construct
  source 1..41: mol_type = other DNA
tgtgtggggacggctgtaatgcaggccttttcttgtccaga

SEQ ID NO: 1386 - DNA, 19 bp, synthetic construct
  source 1..19: mol_type = other DNA
gcaagatctgtgagcctgg

SEQ ID NO: 1387 - DNA, 19 bp, synthetic construct
  source 1..19: mol_type = other DNA
catggccacccatcatctc

SEQ ID NO: 1388 - DNA, 39 bp, synthetic construct
  source 1..39: mol_type = other DNA
cggccatgatgtccttctcgcgcccgacctacaaacagg

SEQ ID NO: 1389 - DNA, 40 bp, synthetic construct; identical to SEQ ID NO: 3
  source 1..40: mol_type = other DNA

SEQ ID NO: 1390 - DNA, 19 bp, synthetic construct
  source 1..19: mol_type = other DNA
acccccaagtgtagcaaga

SEQ ID NO: 1391 - DNA, 18 bp, synthetic construct
  source 1..18: mol_type = other DNA
tccggtgacgtgttggta

SEQ ID NO: 1392 - DNA, 39 bp, synthetic construct; identical to SEQ ID NO: 1388
  source 1..39: mol_type = other DNA

SEQ ID NO: 1393 - DNA, 42 bp, synthetic construct
  source 1..42: mol_type = other DNA
atctacaaaaacggccccgtggttgtagagcaggaagtccga

SEQ ID NO: 1394 - DNA, 19 bp, synthetic construct
  source 1..19: mol_type = other DNA
acccccaagtgtagcaaga

SEQ ID NO: 1395 - DNA, 18 bp, synthetic construct
  source 1..18: mol_type = other DNA
tccggtgacgtgttggta

SEQ ID NO: 1396 - DNA, 39 bp, synthetic construct; identical to SEQ ID NO: 1388
  source 1..39: mol_type = other DNA

SEQ ID NO: 1397 - DNA, 42 bp, synthetic construct
  source 1..42: mol_type = other DNA
atctacaaaaacggccccgtggcttgtagagcaggaagtccg

SEQ ID NO: 1398 - DNA, 19 bp, synthetic construct
  source 1..19: mol_type = other DNA
gcaagatctgtgagcctgg

SEQ ID NO: 1399 - DNA, 19 bp, synthetic construct
  source 1..19: mol_type = other DNA
catggccacccatcatctc

SEQ ID NO: 1400 - DNA, 39 bp, synthetic construct; identical to SEQ ID NO: 1388
  source 1..39: mol_type = other DNA

SEQ ID NO: 1401 - DNA, 40 bp, synthetic construct
  source 1..40: mol_type = other DNA
caaaaacggccccgtggaggacgtgttggtacactcctga

SEQ ID NO: 1402 - DNA, 19 bp, synthetic construct
  source 1..19: mol_type = other DNA
gcaagatctgtgagcctgg

SEQ ID NO: 1403 - DNA, 19 bp, synthetic construct
  source 1..19: mol_type = other DNA
catggccacccatcatctc

SEQ ID NO: 1404 - DNA, 39 bp, synthetic construct; identical to SEQ ID NO: 1388
  source 1..39: mol_type = other DNA

SEQ ID NO: 1405 - DNA, 41 bp, synthetic construct
  source 1..41: mol_type = other DNA
ccgtggagggagctttctctgacgtgttggtacactcctga

SEQ ID NO: 1406 - DNA, 20 bp, synthetic construct
  source 1..20: mol_type = other DNA
tacccccaagtgtagcaaga

SEQ ID NO: 1407 - DNA, 19 bp, synthetic construct
  source 1..19: mol_type = other DNA
catggccacccatcatctc

SEQ ID NO: 1408 - DNA, 39 bp, synthetic construct; identical to SEQ ID NO: 1388
  source 1..39: mol_type = other DNA

SEQ ID NO: 1409 - DNA, 39 bp, synthetic construct
  source 1..39: mol_type = other DNA
ccgtggagggagctttctctgtccggtgacgtgttggta

SEQ ID NO: 1410 - DNA, 38 bp, synthetic construct
  source 1..38: mol_type = other DNA
ctgtgcccatggtgctcagttggaggacgaggcgattc

SEQ ID NO: 1411 - DNA, 18 bp, synthetic construct
  source 1..18: mol_type = other DNA
gccaaagtggtcagggtg

SEQ ID NO: 1412 - DNA, 20 bp, synthetic construct
  source 1..20: mol_type = other DNA
ggacatcatcttggctgcta

SEQ ID NO: 1413 - DNA, 39 bp, synthetic construct
  source 1..39: mol_type = other DNA
cgccatggccacaactcctcggctctgccgtagttttgc

SEQ ID NO: 1414 - DNA, 38 bp, synthetic construct; identical to SEQ ID NO: 1410
  source 1..38: mol_type = other DNA

SEQ ID NO: 1415 - DNA, 18 bp, synthetic construct
  source 1..18: mol_type = other DNA
gccaaagtggtcagggtg

SEQ ID NO: 1416 - DNA, 20 bp, synthetic construct
  source 1..20: mol_type = other DNA
ggacatcatcttggctgcta

SEQ ID NO: 1417 - DNA, 39 bp, synthetic construct
  source 1..39: mol_type = other DNA
cgccatggccacaactcctcgctctgccgtagttttgcc

SEQ ID NO: 1418 - DNA, 38 bp, synthetic construct; identical to SEQ ID NO: 1410
  source 1..38: mol_type = other DNA

SEQ ID NO: 1419 - DNA, 18 bp, synthetic construct
  source 1..18: mol_type = other DNA
caatgggggtggagttgc

SEQ ID NO: 1420 - DNA, 19 bp, synthetic construct
  source 1..19: mol_type = other DNA
gatagttggctcctcgctg

SEQ ID NO: 1421 - DNA, 42 bp, synthetic construct
  source 1..42: mol_type = other DNA
tggagcccaggatgaacttggtttgtggctactctgcagtca

SEQ ID NO: 1422 - DNA, 40 bp, synthetic construct; identical to SEQ ID NO: 20
  source 1..40: mol_type = other DNA

SEQ ID NO: 1423 - DNA, 19 bp, synthetic construct
  source 1..19: mol_type = other DNA
cagatgggaagggactcga

SEQ ID NO: 1424 - DNA, 18 bp, synthetic construct
  source 1..18: mol_type = other DNA
atggcactgagcaccatg

SEQ ID NO: 1425 - DNA, 41 bp, synthetic construct
  source 1..41: mol_type = other DNA
gctgatgaggtgagagcagaggctctggaatgccacggaat

SEQ ID NO: 1426 - DNA, 42 bp, synthetic construct
  source 1..42: mol_type = other DNA
ctggctctgccgtagttttgccccacaactcctccaatcaca

SEQ ID NO: 1427 - DNA, 18 bp, synthetic construct
  source 1..18: mol_type = other DNA
gccaagatgatgtccgcg

SEQ ID NO: 1428 - DNA, 18 bp, synthetic construct
  source 1..18: mol_type = other DNA
tcttctctgcagggcgag

SEQ ID NO: 1429 - DNA, 40 bp, synthetic construct; identical to SEQ ID NO: 19
  source 1..40: mol_type = other DNA

SEQ ID NO: 1430 - DNA, 42 bp, synthetic construct
  source 1..42: mol_type = other DNA
gaccaagttcatcctgggctccgcagggagctagtagaacct

SEQ ID NO: 1431 - DNA, 19 bp, synthetic construct
  source 1..19: mol_type = other DNA
cagatgggaagggactcga

SEQ ID NO: 1432 - DNA, 18 bp, synthetic construct
  source 1..18: mol_type = other DNA
atggcactgagcaccatg

SEQ ID NO: 1433 - DNA, 41 bp, synthetic construct; identical to SEQ ID NO: 1425
  source 1..41: mol_type = other DNA

SEQ ID NO: 1434 - DNA, 41 bp, synthetic construct
  source 1..41: mol_type = other DNA
ggctctgccgtagttttgcccgccacaactcctccaatcac

SEQ ID NO: 1435 - DNA, 19 bp, synthetic construct
  source 1..19: mol_type = other DNA
gcagccaagatgatgtccg

SEQ ID NO: 1436 - DNA, 18 bp, synthetic construct
  source 1..18: mol_type = other DNA
tctcccctggcatggttc

SEQ ID NO: 1437 - DNA, 40 bp, synthetic construct; identical to SEQ ID NO: 19
  source 1..40: mol_type = other DNA

SEQ ID NO: 1438 - DNA, 38 bp, synthetic construct
  source 1..38: mol_type = other DNA
attgggtctgccattgcggctcttctctgcagggcgag

SEQ ID NO: 1439 - DNA, 40 bp, synthetic construct; identical to SEQ ID NO: 19
  source 1..40: mol_type = other DNA

SEQ ID NO: 1440 - DNA, 19 bp, synthetic construct
  source 1..19: mol_type = other DNA
agcagccaagatgatgtcc

SEQ ID NO: 1441 - DNA, 42 bp, synthetic construct; identical to SEQ ID NO: 1421
  source 1..42: mol_type = other DNA

SEQ ID NO: 1442 - DNA, 18 bp, synthetic construct
  source 1..18: mol_type = other DNA
attgccaatgggggtgga

SEQ ID NO: 1443 - DNA, 40 bp, synthetic construct
  source 1..40: mol_type = other DNA
actgcagagtagccacaaggctgccaagatgatgtccgcg

SEQ ID NO: 1444 - DNA, 18 bp, synthetic construct
  source 1..18: mol_type = other DNA
gcctcgtcctccatagca

SEQ ID NO: 1445 - DNA, 20 bp, synthetic construct
  source 1..20: mol_type = other DNA
actgagggttgactgactgg

SEQ ID NO: 1446 - DNA, 31 bp, synthetic construct; identical to SEQ ID NO: 61
  source 1..31: mol_type = other DNA

SEQ ID NO: 1447 - DNA, 54 bp, synthetic construct; identical to SEQ ID NO: 62
  source 1..54: mol_type = other DNA

SEQ ID NO: 1448 - DNA, 51 bp, synthetic construct; identical to SEQ ID NO: 63
  source 1..51: mol_type = other DNA

SEQ ID NO: 1449 - DNA, 20 bp, synthetic construct
  source 1..20: mol_type = other DNA
gcgctcatgctctgacactt

SEQ ID NO: 1450 - DNA, 20 bp, synthetic construct
  source 1..20: mol_type = other DNA
aggggtggaagaaggcccta

SEQ ID NO: 1451 - DNA, 25 bp, synthetic construct
  source 1..25: mol_type = other DNA
ctgagtgtgagagcccggaagattt

SEQ ID NO: 1452 - DNA, 25 bp, synthetic construct
  source 1..25: mol_type = other DNA
tgttcagcaccgacgtgaagtactt

SEQ ID NO: 1453 - DNA, 51 bp, synthetic construct; identical to SEQ ID NO: 850
  source 1..51: mol_type = other DNA

SEQ ID NO: 1454 - DNA, 43 bp, synthetic construct; identical to SEQ ID NO: 851
  source 1..43: mol_type = other DNA

SEQ ID NO: 1455 - DNA, 25 bp, synthetic construct
  source 1..25: mol_type = other DNA
catcagatgagtcctgtttgccagg

SEQ ID NO: 1456 - DNA, 21 bp, synthetic construct
  source 1..21: mol_type = other DNA
gcacctggagagcgaagacct

SEQ ID NO: 1457 - DNA, 19 bp, synthetic construct
  source 1..19: mol_type = other DNA
ggaccagacaggactcgtg

SEQ ID NO: 1458 - DNA, 18 bp, synthetic construct
  source 1..18: mol_type = other DNA
cgctgcatcttcaggcca

SEQ ID NO: 1459 - DNA, 44 bp, synthetic construct; identical to SEQ ID NO: 80
  source 1..44: mol_type = other DNA

SEQ ID NO: 1460 - DNA, 41 bp, synthetic construct; identical to SEQ ID NO: 81
  source 1..41: mol_type = other DNA

SEQ ID NO: 1461 - DNA, 24 bp, synthetic construct
  source 1..24: mol_type = other DNA
gaaagacgccattgctaaacttca

SEQ ID NO: 1462 - DNA, 23 bp, synthetic construct
  source 1..23: mol_type = other DNA
ccatggtcattctcagccttact

SEQ ID NO: 1463 - DNA, 25 bp, synthetic construct
  source 1..25: mol_type = other DNA
ggatgatttcttcaagccagagtct

SEQ ID NO: 1464 - DNA, 28 bp, synthetic construct
  source 1..28: mol_type = other DNA
catatggaatagtcaggaaatagcttct

SEQ ID NO: 1465 - DNA, 42 bp, synthetic construct; identical to SEQ ID NO: 1048
  source 1..42: mol_type = other DNA

SEQ ID NO: 1466 - DNA, 51 bp, synthetic construct; identical to SEQ ID NO: 1049
  source 1..51: mol_type = other DNA

SEQ ID NO: 1467 - DNA, 28 bp, synthetic construct
  source 1..28: mol_type = other DNA
acatcattttttccatgttaagtgcttc

SEQ ID NO: 1468 - DNA, 28 bp, synthetic construct
  source 1..28: mol_type = other DNA
ctgaggaaattcccattttaatcatcga

SEQ ID NO: 1469 - DNA, 17 bp, synthetic construct
  source 1..17: mol_type = other DNA
tgtggcagtgcccatca

SEQ ID NO: 1470 - DNA, 25 bp, synthetic construct
  source 1..25: mol_type = other DNA
ctgaccaaactctgcttctttgaat

SEQ ID NO: 1471 - DNA, 41 bp, synthetic construct; identical to SEQ ID NO: 1084
  source 1..41: mol_type = other DNA

SEQ ID NO: 1472 - DNA, 40 bp, synthetic construct; identical to SEQ ID NO: 1085
  source 1..40: mol_type = other DNA

SEQ ID NO: 1473 - DNA, 26 bp, synthetic construct
  source 1..26: mol_type = other DNA
ggtccttcctgaagtcttatcttgtt

SEQ ID NO: 1474 - DNA, 19 bp, synthetic construct
  source 1..19: mol_type = other DNA
gggacttggacgatgctca

SEQ ID NO: 1475 - DNA, 28 bp, synthetic construct
  source 1..28: mol_type = other DNA
ccctacaagtcataaagtcagatcttgt

SEQ ID NO: 1476 - DNA, 19 bp, synthetic construct
  source 1..19: mol_type = other DNA
ctgttgccattggacagct

SEQ ID NO: 1477 - DNA, 44 bp, synthetic construct; identical to SEQ ID NO: 1108
  source 1..44: mol_type = other DNA

SEQ ID NO: 1478 - DNA, 43 bp, synthetic construct; identical to SEQ ID NO: 1109
  source 1..43: mol_type = other DNA

SEQ ID NO: 1479 - DNA, 19 bp, synthetic construct
  source 1..19: mol_type = other DNA
ggcagctccgggtatacat

SEQ ID NO: 1480 - DNA, 21 bp, synthetic construct
  source 1..21: mol_type = other DNA
cacctgtgaacctgagactca

SEQ ID NO: 1481 - DNA, 19 bp, synthetic construct
  source 1..19: mol_type = other DNA
tctgtgagcctggctacag

SEQ ID NO: 1482 - DNA, 19 bp, synthetic construct
  source 1..19: mol_type = other DNA
catggccacccatcatctc

SEQ ID NO: 1483 - DNA, 41 bp, synthetic construct; identical to SEQ ID NO: 1
  source 1..41: mol_type = other DNA

SEQ ID NO: 1484 - DNA, 40 bp, synthetic construct; identical to SEQ ID NO: 3
  source 1..40: mol_type = other DNA

SEQ ID NO: 1485 - DNA, 22 bp, synthetic construct
  source 1..22: mol_type = other DNA
atgttaaggatgtcgcagaggt

SEQ ID NO: 1486 - DNA, 22 bp, synthetic construct
  source 1..22: mol_type = other DNA
ggagctttctctgtgtattcgg

SEQ ID NO: 1487 - DNA, 19 bp, synthetic construct
  source 1..19: mol_type = other DNA
cgacctccgcaaccttgag

SEQ ID NO: 1488 - DNA, 23 bp, synthetic construct
  source 1..23: mol_type = other DNA
cttggtccactgtgcctctaaac

SEQ ID NO: 1489 - DNA, 43 bp, synthetic construct; identical to SEQ ID NO: 86
  source 1..43: mol_type = other DNA

SEQ ID NO: 1490 - DNA, 59 bp, synthetic construct; identical to SEQ ID NO: 87
  source 1..59: mol_type = other DNA

SEQ ID NO: 1491 - DNA, 20 bp, synthetic construct
  source 1..20: mol_type = other DNA
ctcttccagtccctgtccgg

SEQ ID NO: 1492 - DNA, 19 bp, synthetic construct
  source 1..19: mol_type = other DNA
gccttttgcctgggaattg

SEQ ID NO: 1493 - DNA, 18 bp, synthetic construct
  source 1..18: mol_type = other DNA
aggtgatgaggctccagg

SEQ ID NO: 1494 - DNA, 21 bp, synthetic construct
  source 1..21: mol_type = other DNA
tgaaactcacaccaccaatga

SEQ ID NO: 1495 - DNA, 42 bp, synthetic construct
  source 1..42: mol_type = other DNA
acctgaagagcagagcttttatcccagcgtgggccagaagac

SEQ ID NO: 1496 - DNA, 41 bp, synthetic construct
  source 1..41: mol_type = other DNA
tcaggctcaacaaggggcatggcagttcccaacacgaagtt

SEQ ID NO: 1497 - DNA, 26 bp, synthetic construct
  source 1..26: mol_type = other DNA
gctcttgcagattagtattctgccgg

SEQ ID NO: 1498 - DNA, 25 bp, synthetic construct
  source 1..25: mol_type = other DNA
gtcctgtatagataaaggaaacgta

SEQ ID NO: 1499 - DNA, 18 bp, synthetic construct
  source 1..18: mol_type = other DNA
tgaggccctggttgctat

SEQ ID NO: 1500 - DNA, 19 bp, synthetic construct
  source 1..19: mol_type = other DNA
tcactcctcgatgccagaa

SEQ ID NO: 1501 - DNA, 38 bp, synthetic construct; identical to SEQ ID NO: 98
  source 1..38: mol_type = other DNA

SEQ ID NO: 1502 - DNA, 41 bp, synthetic construct; identical to SEQ ID NO: 99
  source 1..41: mol_type = other DNA

SEQ ID NO: 1503 - DNA, 19 bp, synthetic construct
  source 1..19: mol_type = other DNA
cctgagcatcagctgctag

SEQ ID NO: 1504 - DNA, 19 bp, synthetic construct
  source 1..19: mol_type = other DNA
gcatgggttcctcaacctg

SEQ ID NO: 1505 - DNA, 20 bp, synthetic construct
  source 1..20: mol_type = other DNA
cctgtgagtgagtgcagaaa

SEQ ID NO: 1506 - DNA, 18 bp, synthetic construct
  source 1..18: mol_type = other DNA
accccgacagtgatcgtg

SEQ ID NO: 1507 - DNA, 39 bp, synthetic construct; identical to SEQ ID NO: 170
  source 1..39: mol_type = other DNA

SEQ ID NO: 1508 - DNA, 37 bp, synthetic construct; identical to SEQ ID NO: 171
  source 1..37: mol_type = other DNA

SEQ ID NO: 1509 - DNA, 20 bp, synthetic construct
  source 1..20: mol_type = other DNA
gccgagaattcctccaaagt

SEQ ID NO: 1510 - DNA, 18 bp, synthetic construct
  source 1..18: mol_type = other DNA
ccctggaggaagtgctca

SEQ ID NO: 1511 - DNA, 20 bp, synthetic construct
  source 1..20: mol_type = other DNA
ggcatccgggcctgctatga

SEQ ID NO: 1512 - DNA, 20 bp, synthetic construct
  source 1..20: mol_type = other DNA
tgcccaccactggcatcaac

SEQ ID NO: 1513 - DNA, 40 bp, synthetic construct; identical to SEQ ID NO: 844
  source 1..40: mol_type = other DNA

SEQ ID NO: 1514 - DNA, 44 bp, synthetic construct; identical to SEQ ID NO: 845
  source 1..44: mol_type = other DNA

SEQ ID NO: 1515 - DNA, 27 bp, synthetic construct
  source 1..27: mol_type = other DNA
ccatttcttacgctctgacttctggcc

SEQ ID NO: 1516 - DNA, 27 bp, synthetic construct
  source 1..27: mol_type = other DNA
ggacaggtagtacacggctgaatcgag

SEQ ID NO: 1517 - DNA, 24 bp, synthetic construct
  source 1..24: mol_type = other DNA
acctgaggagagtgactagcttct

SEQ ID NO: 1518 - DNA, 22 bp, synthetic construct
  source 1..22: mol_type = other DNA
gcctgctccatggaacccaaga

SEQ ID NO: 1519 - DNA, 45 bp, synthetic construct; identical to SEQ ID NO: 17
  source 1..45: mol_type = other DNA

SEQ ID NO: 1520 - DNA, 43 bp, synthetic construct; identical to SEQ ID NO: 18
  source 1..43: mol_type = other DNA

SEQ ID NO: 1521 - DNA, 19 bp, synthetic construct
  source 1..19: mol_type = other DNA
ccgcaaccctgaagaccca

SEQ ID NO: 1522 - DNA, 23 bp, synthetic construct
  source 1..23: mol_type = other DNA
gcagttcaaggtgacaagggcac

SEQ ID NO: 1523 - DNA, 18 bp, synthetic construct
  source 1..18: mol_type = other DNA
atcacattcctgccgctg

SEQ ID NO: 1524 - DNA, 18 bp, synthetic construct
  source 1..18: mol_type = other DNA
cgccaagctattcagcac

SEQ ID NO: 1525 - DNA, 42 bp, synthetic construct; identical to SEQ ID NO: 236
  source 1..42: mol_type = other DNA

SEQ ID NO: 1526 - DNA, 39 bp, synthetic construct; identical to SEQ ID NO: 237
  source 1..39: mol_type = other DNA

SEQ ID NO: 1527 - DNA, 19 bp, synthetic construct
  source 1..19: mol_type = other DNA
ttggagtcccagcatcatc

SEQ ID NO: 1528 - DNA, 21 bp, synthetic construct
  source 1..21: mol_type = other DNA
gctgggatccagaggagaata

SEQ ID NO: 1529 - DNA, 19 bp, synthetic construct
  source 1..19: mol_type = other DNA
agcagccaagatgatgtcc

SEQ ID NO: 1530 - DNA, 19 bp, synthetic construct
  source 1..19: mol_type = other DNA
gatagttggctcctcgctg

SEQ ID NO: 1531 - DNA, 40 bp, synthetic construct; identical to SEQ ID NO: 19
  source 1..40: mol_type = other DNA

SEQ ID NO: 1532 - DNA, 40 bp, synthetic construct; identical to SEQ ID NO: 20
  source 1..40: mol_type = other DNA

SEQ ID NO: 1533 - DNA, 17 bp, synthetic construct
  source 1..17: mol_type = other DNA
tgggtctgccattgcgg

SEQ ID NO: 1534 - DNA, 20 bp, synthetic construct
  source 1..20: mol_type = other DNA
ccctcgccctgcagagaaga

SEQ ID NO: 1535 - DNA, 39 bp, synthetic construct; identical to SEQ ID NO: 260
  source 1..39: mol_type = other DNA

SEQ ID NO: 1536 - DNA, 42 bp, synthetic construct; identical to SEQ ID NO: 261
  source 1..42: mol_type = other DNA

SEQ ID NO: 1537 - DNA, 22 bp, synthetic construct
  source 1..22: mol_type = other DNA
gcgaactcatctttgccagtac

SEQ ID NO: 1538 - DNA, 19 bp, synthetic construct
  source 1..19: mol_type = other DNA
cgccgatcttctgggtgat

SEQ ID NO: 1539 - DNA, 17 bp, synthetic construct
  source 1..17: mol_type = other DNA
tcaggtcccagcccatg

SEQ ID NO: 1540 - DNA, 18 bp, synthetic construct
  source 1..18: mol_type = other DNA
gagcagctccatgtcggt

SEQ ID NO: 1541 - DNA, 44 bp, synthetic construct; identical to SEQ ID NO: 302
  source 1..44: mol_type = other DNA

SEQ ID NO: 1542 - DNA, 43 bp, synthetic construct; identical to SEQ ID NO: 303
  source 1..43: mol_type = other DNA

SEQ ID NO: 1543 - DNA, 18 bp, synthetic construct
  source 1..18: mol_type = other DNA
cgagctcagttcgctgtc

SEQ ID NO: 1544 - DNA, 21 bp, synthetic construct
  source 1..21: mol_type = other DNA
gtggttttcttgagcgtgtac

SEQ ID NO: 1545 - DNA, 22 bp, synthetic construct
  source 1..22: mol_type = other DNA
catcaggttcatcacctccatc

SEQ ID NO: 1546 - DNA, 19 bp, synthetic construct
  source 1..19: mol_type = other DNA
acgactcgggtatccactc

SEQ ID NO: 1547 - DNA, 18 bp, synthetic construct
  source 1..18: mol_type = other DNA
gggctgggtcttgggaat

SEQ ID NO: 1548 - DNA, 19 bp, synthetic construct
  source 1..19: mol_type = other DNA
ggagccttgtggttctacc

SEQ ID NO: 1549 - DNA, 44 bp, synthetic construct; identical to SEQ ID NO: 1190
  source 1..44: mol_type = other DNA

SEQ ID NO: 1550 - DNA, 40 bp, synthetic construct; identical to SEQ ID NO: 1191
  source 1..40: mol_type = other DNA

SEQ ID NO: 1551 - DNA, 18 bp, synthetic construct
  source 1..18: mol_type = other DNA
tggggggtacagaggcat

SEQ ID NO: 1552 - DNA, 25 bp, synthetic construct
  source 1..25: mol_type = other DNA
catgttctctggatgtcagctgagt

SEQ ID NO: 1553 - DNA, 28 bp, synthetic construct
  source 1..28: mol_type = other DNA
gaactttttctgtacctgttaaacaaga

SEQ ID NO: 1554 - DNA, 25 bp, synthetic construct
  source 1..25: mol_type = other DNA
ggtttgttggtgattcagtgtaaga

SEQ ID NO: 1555 - DNA, 54 bp, synthetic construct; identical to SEQ ID NO: 934
  source 1..54: mol_type = other DNA

SEQ ID NO: 1556 - DNA, 45 bp, synthetic construct; identical to SEQ ID NO: 935
  source 1..45: mol_type = other DNA

SEQ ID NO: 1557 - DNA, 24 bp, synthetic construct
  source 1..24: mol_type = other DNA
ggtagcgtaataaccgttcttctg

SEQ ID NO: 1558 - DNA, 26 bp, synthetic construct
  source 1..26: mol_type = other DNA
ctcctgttctctagaaagtcaatgga

SEQ ID NO: 1559 - DNA, 24 bp, synthetic construct
  source 1..24: mol_type = other DNA
atccaggcagtcatagactccgga

SEQ ID NO: 1560 - DNA, 24 bp, synthetic construct
  source 1..24: mol_type = other DNA
ccacttgttgagcagtcccaagga

SEQ ID NO: 1561 - DNA, 48 bp, synthetic construct; identical to SEQ ID NO: 34
  source 1..48: mol_type = other DNA

SEQ ID NO: 1562 - DNA, 48 bp, synthetic construct; identical to SEQ ID NO: 35
  source 1..48: mol_type = other DNA

SEQ ID NO: 1563 - DNA, 24 bp, synthetic construct
  source 1..24: mol_type = other DNA
atttgagccctgttgccagcagta

SEQ ID NO: 1564 - DNA, 24 bp, synthetic construct
  source 1..24: mol_type = other DNA
cagggcaggagaagccactttgta

SEQ ID NO: 1565 - DNA, 24 bp, synthetic construct
  source 1..24: mol_type = other DNA
cttgacctagctctcatgtctcaa

SEQ ID NO: 1566 - DNA, 23 bp, synthetic construct
  source 1..23: mol_type = other DNA
cacatgatagtagcattggcaca

SEQ ID NO: 1567 - DNA, 51 bp, synthetic construct; identical to SEQ ID NO: 1012
  source 1..51: mol_type = other DNA

SEQ ID NO: 1568 - DNA, 48 bp, synthetic construct; identical to SEQ ID NO: 1013
  source 1..48: mol_type = other DNA

SEQ ID NO: 1569 - DNA, 22 bp, synthetic construct
  source 1..22: mol_type = other DNA
ccacagaaagaaaacttgggca

SEQ ID NO: 1570 - DNA, 22 bp, synthetic construct
  source 1..22: mol_type = other DNA
cctcagggagaataccaggttt

SEQ ID NO: 1571 - DNA, 22 bp, synthetic construct
  source 1..22: mol_type = other DNA
gcactgatgcaggaactgtata

SEQ ID NO: 1572 - DNA, 18 bp, synthetic construct
  source 1..18: mol_type = other DNA
agctcagaaacgccacca

SEQ ID NO: 1573 - DNA, 38 bp, synthetic construct; identical to SEQ ID NO: 332
  source 1..38: mol_type = other DNA

SEQ ID NO: 1574 - DNA, 38 bp, synthetic construct; identical to SEQ ID NO: 333
  source 1..38: mol_type = other DNA

SEQ ID NO: 1575 - DNA, 19 bp, synthetic construct
  source 1..19: mol_type = other DNA
cagcgtctagcacctcttc

SEQ ID NO: 1576 - DNA, 19 bp, synthetic construct
  source 1..19: mol_type = other DNA
cgggtgctgaaggtagtca

SEQ ID NO: 1577 - DNA, 18 bp, synthetic construct
  source 1..18: mol_type = other DNA
tctgtttccctgccagac

SEQ ID NO: 1578 - DNA, 23 bp, synthetic construct
  source 1..23: mol_type = other DNA
tccttcacttctaccttgatcag

SEQ ID NO: 1579 - DNA, 42 bp, synthetic construct; identical to SEQ ID NO: 338
  source 1..42: mol_type = other DNA

SEQ ID NO: 1580 - DNA, 46 bp, synthetic construct; identical to SEQ ID NO: 339
  source 1..46: mol_type = other DNA

SEQ ID NO: 1581 - DNA, 22 bp, synthetic construct
  source 1..22: mol_type = other DNA
aagaacatgagctgtgaattcc

SEQ ID NO: 1582 - DNA, 24 bp, synthetic construct
  source 1..24: mol_type = other DNA
catcatggagaaggataccatttc

SEQ ID NO: 1583 - DNA, 21 bp, synthetic construct
  source 1..21: mol_type = other DNA
cagcctaactacatgcctgtg

SEQ ID NO: 1584 - DNA, 25 bp, synthetic construct
  source 1..25: mol_type = other DNA
atttgaaatgtattcagacacctgg

SEQ ID NO: 1585 - DNA, 42 bp, synthetic construct; identical to SEQ ID NO: 368
  source 1..42: mol_type = other DNA

SEQ ID NO: 1586 - DNA, 43 bp, synthetic construct; identical to SEQ ID NO: 369
  source 1..43: mol_type = other DNA

SEQ ID NO: 1587 - DNA, 24 bp, synthetic construct
  source 1..24: mol_type = other DNA
tggtctaaacaccagtctaattcc

SEQ ID NO: 1588 - DNA, 20 bp, synthetic construct
  source 1..20: mol_type = other DNA
aaagaatgctgaaccgggag

SEQ ID NO: 1589 - DNA, 14 bp, synthetic construct
  source 1..14: mol_type = other DNA
ggcaggcgtgcgga

SEQ ID NO: 1590 - DNA, 17 bp, synthetic construct
  source 1..17: mol_type = other DNA
agggggccactcatgtc

SEQ ID NO: 1591 - DNA, 43 bp, synthetic construct
  source 1..43: mol_type = other DNA
agggagtgaggtggaagatctaagtggacactcctgcgaccag

SEQ ID NO: 1592 - DNA, 35 bp, synthetic construct
  source 1..35: mol_type = other DNA
agccaagcctccaggccctgggccatggggagaac

SEQ ID NO: 1593 - DNA, 21 bp, synthetic construct
  source 1..21: mol_type = other DNA
agttcgatcacagccagtacc

SEQ ID NO: 1594 - DNA, 19 bp, synthetic construct
  source 1..19: mol_type = other DNA
catccgtggtggcctctct

SEQ ID NO: 1595 - DNA, 25 bp, synthetic construct
  source 1..25: mol_type = other DNA
caaatgtggtgagaaatatcagcta

SEQ ID NO: 1596 - DNA, 20 bp, synthetic construct
  source 1..20: mol_type = other DNA
tgcactcctcgggagacatg

SEQ ID NO: 1597 - DNA, 43 bp, synthetic construct; identical to SEQ ID NO: 416
  source 1..43: mol_type = other DNA

SEQ ID NO: 1598 - DNA, 43 bp, synthetic construct; identical to SEQ ID NO: 417
  source 1..43: mol_type = other DNA

SEQ ID NO: 1599 - DNA, 25 bp, synthetic construct
  source 1..25: mol_type = other DNA
catatacctgacctccttcacgttg

SEQ ID NO: 1600 - DNA, 19 bp, synthetic construct
  source 1..19: mol_type = other DNA
gctccggcagcacctttat

SEQ ID NO: 1601 - DNA, 25 bp, synthetic construct
  source 1..25: mol_type = other DNA
gtcgaatttgtgggtgattttactg

SEQ ID NO: 1602 - DNA, 20 bp, synthetic construct
  source 1..20: mol_type = other DNA
ggagtcgctgaagccgtata

SEQ ID NO: 1603 - DNA, 40 bp, synthetic construct; identical to SEQ ID NO: 464
  source 1..40: mol_type = other DNA

SEQ ID NO: 1604 - DNA, 43 bp, synthetic construct; identical to SEQ ID NO: 453
  source 1..43: mol_type = other DNA

SEQ ID NO: 1605 - DNA, 21 bp, synthetic construct
  source 1..21: mol_type = other DNA
catgtaggccagcatgtgttt

SEQ ID NO: 1606 - DNA, 21 bp, synthetic construct
  source 1..21: mol_type = other DNA
gccacagaacgagcacatcta

SEQ ID NO: 1607 - DNA, 27 bp, synthetic construct
  source 1..27: mol_type = other DNA
attccttttccggaacaagtttgcatc

SEQ ID NO: 1608 - DNA, 32 bp, synthetic construct; identical to SEQ ID NO: 43
  source 1..32: mol_type = other DNA

SEQ ID NO: 1609 - DNA, 42 bp, synthetic construct; identical to SEQ ID NO: 46
  source 1..42: mol_type = other DNA

SEQ ID NO: 1610 - DNA, 52 bp, synthetic construct; identical to SEQ ID NO: 47
  source 1..52: mol_type = other DNA

SEQ ID NO: 1611 - DNA, 21 bp, synthetic construct
  source 1..21: mol_type = other DNA
ctccctggcatgatgcgttgg

SEQ ID NO: 1612 - DNA, 21 bp, synthetic construct
  source 1..21: mol_type = other DNA
gccaggttcagccccccaata

SEQ ID NO: 1613 - DNA, 20 bp, synthetic construct
  source 1..20: mol_type = other DNA
cttggctcagtgcccttcac

SEQ ID NO: 1614 - DNA, 23 bp, synthetic construct
  source 1..23: mol_type = other DNA
tgtttgcaagctcctttgtaagc

SEQ ID NO: 1615 - DNA, 43 bp, synthetic construct; identical to SEQ ID NO: 482
  source 1..43: mol_type = other DNA

SEQ ID NO: 1616 - DNA, 43 bp, synthetic construct; identical to SEQ ID NO: 483
  source 1..43: mol_type = other DNA

SEQ ID NO: 1617 - DNA, 22 bp, synthetic construct
  source 1..22: mol_type = other DNA
cccagcctaatgttaacccctc

SEQ ID NO: 1618 - DNA, 20 bp, synthetic construct
  source 1..20: mol_type = other DNA
aggggcattttgacaccctc

SEQ ID NO: 1619 - DNA, 24 bp, synthetic construct
  source 1..24: mol_type = other DNA
ggtgatgaaatcctggttagcgga

SEQ ID NO: 1620 - DNA, 24 bp, synthetic construct
  source 1..24: mol_type = other DNA
cgctgatgcttgtttgaagatctc

SEQ ID NO: 1621 - DNA, 45 bp, synthetic construct; identical to SEQ ID NO: 648
  source 1..45: mol_type = other DNA

SEQ ID NO: 1622 - DNA, 47 bp, synthetic construct; identical to SEQ ID NO: 649
  source 1..47: mol_type = other DNA

SEQ ID NO: 1623 - DNA, 22 bp, synthetic construct
  source 1..22: mol_type = other DNA
gctgacttccagcttgtcacct

SEQ ID NO: 1624 - DNA, 23 bp, synthetic construct
  source 1..23: mol_type = other DNA
ctccagccaacagacctcaggaa

SEQ ID NO: 1625 - DNA, 18 bp, synthetic construct
  source 1..18: mol_type = other DNA
ctgaaaaggcctgtagcc

SEQ ID NO: 1626 - DNA, 19 bp, synthetic construct
  source 1..19: mol_type = other DNA
cagagtgacactgaacaga

SEQ ID NO: 1627 - DNA, 45 bp, synthetic construct; identical to SEQ ID NO: 54
  source 1..45: mol_type = other DNA

SEQ ID NO: 1628 - DNA, 40 bp, synthetic construct; identical to SEQ ID NO: 56
  source 1..40: mol_type = other DNA

SEQ ID NO: 1629 - DNA, 22 bp, synthetic construct
  source 1..22: mol_type = other DNA
tcagcgtatccaattcagcaat

SEQ ID NO: 1630 - DNA, 20 bp, synthetic construct
  source 1..20: mol_type = other DNA
gagacgaaggagacgctggg

SEQ ID NO: 1631 - DNA, 16 bp, synthetic construct
  source 1..16: mol_type = other DNA
tgtccgtgagctcggc

SEQ ID NO: 1632 - DNA, 17 bp, synthetic construct
  source 1..17: mol_type = other DNA
ggggggttgagagcaga

SEQ ID NO: 1633 - DNA, 42 bp, synthetic construct
  source 1..42: mol_type = other DNA
ttgggtcccagtggtgcacaaactacaagggcaagtgcagac

SEQ ID NO: 1634 - DNA, 42 bp, synthetic construct
  source 1..42: mol_type = other DNA
tacatggctgaagctgtccagccattggagggtgcagattcg

SEQ ID NO: 1635 - DNA, 20 bp, synthetic construct
  source 1..20: mol_type = other DNA
ggggttgtatccctgaaggt

SEQ ID NO: 1636 - DNA, 20 bp, synthetic construct
  source 1..20: mol_type = other DNA
cagagagtggtggggcctga